SEQUENCE LISTING (936 sequences)

SEQ ID NO: 1 - DNA, 9763 bp, synthetic construct
  source 1..9763: mol_type = other DNA
ccctgcgcgcgccccggccgcccgccgcctccgccccgcgcccctgagctgccgccgccgccgccgccgctgccgccgccgccgccgccgccgcgggtccgagggcgccgcgcccttgccctgggcccggccgtgcccgccgccgcccggccgcgtccccgctgccgcgccgcccggccgggtgcatgcattgtgggcctcccgacatggtctgcgagacgaagatcgtggccgccgaggaccatgaggcgctgccgggggccaagaaggacgcgctgctcgccgccgccggcgccatgtggcccccgctgcccgccgcgcccgggccggccgccgcgccccccgcgcccccacctgccccggtggctcagcctcacggcggggcggggggcgcggggccgccgggggggcgcggcgtgtgcatccgcgagttccgtgcggccgagcaggaggcggcgcgccgcatcttctacgacggcatcatggagcgcatccctaacacggccttccgcggcctgcggcagcacccgcgcgcgcagctgctctacgccctgctggcgggtcagtgcgccgggcccccggctgccgcagtccctcgggccggcgcggagctcccccgccccggcgtccacgcggaccccgcgcccggctcccggggaccagcctgggaagccccccgctttctgccgcgccgggccccgcgcagggctggctatgggcgtggggatgggcgcagggccccgagcgggccggagccggggagggtccggggtccgcacctgcgtccccgccgcgcagccccccacccccaccgcctcccctgtcccctcccgggcatcctgggtgggggcgggtgccagggcaggtagcgcccgccgcggctgggccccggcgagtgcctaaatataatctgcgggcgggggaggccgcctcccgctcggtgctaatttatgaggggaagacagccggcgccgcggggggtgcgcgcgcctggcacagccggggcggtcgcagagggcggccccttcagcgtctttgggtgcccttcccctcagagcttcctgccggggtgagggggttcgctcccattttacagatgaggcgcttgaggccgcgtagggaggacgcgactcccccaaggtcacggcagccgaaatgcggctggaagcggtgtggggcctccctgccctgtccctgccctcagagctcccggacgctggccaggaagggctcttgctggggccctgggaagagggggtcctggagcccgaggctggaacgtgggacaccccccttcctcctcccccctcccccctcccccgcgcggggcctggtctgcggggcagctccctgccaggatctgcccaccttggcttcagtaggccagcgtgggcagagtccttcccggcccggccatcccgagggtgtcctgccgtgtttggaagctcagagacccgccgaggctcattccaggcggtgggggtgggtggggtggctgtcttcaccgcaggaagcttggaaatagggagaagtagaaagcacccgagatgaccccggctcctccaccaggagcgcggccgggcgcggcgtccctagcgggcttcgccggggtggcgtctctggggcctggggacccccgccgcgccgctgacccgcgccctctgcccccagcgctgtgcttcgccgtgagccgctcgctgctgctgacgtgcctggtgccggccgcgctgctgggcctgcgctactactacagccgcaaggtgatccgcgcctacctggagtgcgcgctgcacacggacatggcggacatcgagcagtactacatgaagccgcccggtgagtcccgctcccgccgctccccgacctccgccccagacagccctcgggggcacgcactccagcgacctcagggcaggcctgggcgagggccggcgtgggtgggcctgagccggggcccctgtgacctgagccttctgggtgaccgaggcctccggtgtcagcggggctgggggaggcacggccggttcctcccgctgagtgacctgagctggccctgcgacgcagtggctccaccgttccctgctgtggatgcgtctccctggcgcctgctgcggtggggcaagggccgctgggctctgtgggtcccatctggtgtgccttgcggggatggggttgcctgcagtgccagggtcctggggggagagggcttgtgtggcaggggccggggaccctattggggcacagaggatggggtgggggcaggggacctgaccttgagccttggccttcacttctggcctgatcgctgggccgcttagctgaagggttttgtcccagcctctgtggccttctatagctggaggagctgggtctgggctctagggggggtgggggttgttctccaccctcgggctggcccccagccgctgagccacaccggttcccccatgccggatgccgcaggggtgaagactctgctctctggggtgggaggctctctgcgatcctggagcgtctgtaggccaacgagagggacagagtgtggcccaaagggttgggcagggtctgtcaggagcccggtcagtgtcaggggaggggcagtgtctggcaccgctggctgccatgctccacccccgcgccacggccttggtgcctgctgctctccctgaccctttcctaaacccaaacagagcatctgagcagcccctgagccctggccagcctctgcttgggaggggaaacgggccatgaggaagcacaggccacagcaacacagggggctccggtgggcacacggggcatggccctgctccctctgggccgtggcctccctggtgtgagggagtggaggggtagctggcagggctgcaggaacaccccacgagggcagaggctggatagctgctgatggcccgtggtggattctgggagctgcttgtgcacatggggaaaccgagtcttgggaggagtgaggtgtgcccagggccacatggtgctgggggttgctacttctgggatggccctgatctctcgtcttgcctgggtaggtagaggcagaacccctgggtggtgggcaggccagctggtgggagggatactggtccaggatcccctccttggaggtagagatgctgtgtgggtagggagatggggggttgggtgtgggagaccccaggggctccaggacagggggccgaggaggggcagccctcctgggaaccctggagctgcacaaagcttggagggcccagctgctcgtgtctgtggcgatggcagcggtgcccagtggctcccccccacgctgcttctccttgaagggccttcagtacccctttagctgcctgccaggcctcagtgtccagcagtgaaggagacagaggtccccttcatggagccagcaggctggtggctgtgcagaggggcaatggggagggatgggagggcttcccggggctcggggcaggtgcagagcctccttgagtaggaggctggatgggccacaggcaggctgtgtggccctccactggccaaacccttggtcttcagagaggtctctgggtgccacctctgcctttggctgctggaggtggctgggcttcccctgtggccagtgcctgcaggttccctcccacaaccgccccaacctgcccctgcacctctcctcctcccctgccggttcccctcccccacgaccgccacctcctggcttcgtgcctgactcgaggtgagaattgggccgtgctgtggatggggtgatggcctggccacttggcctactgccaggagaggccggggccagaggggccatcccctgtcccccacttggatttataggaaagctcctcctgggctgcagggagccttctggacgcactttgctgacccctgccctttgcggccctctgctgtgggtgctggagtttgccgtgatggcccgggtgaccctgggtggccagaagccacagcagtttggctgcaccatggcctgttcagggtggcccgggccatcaaggcatttggtctgggagtagctgggcagagggcagcggcctgactgggcaaagcctgccccagctcttggtctcagaggagggactcgggcctttgaccactactctgagcagcgcagcctccaggcttgagtgggtcctggctgttggcctgttgggtaacaggtgttgggctggctttctctgcttaaggagctctggaggggggctctacctgcttgtcctgaatagtggctgccccctcccttggctccagcagagctggtagctaggccccgggggctgccgggattgggtgtcccactgccctggcttggtgtccagctggagggggcagtgccaagggccctgtctcaccccagaggtggaggggtctccccagccttcctcaccgggtgtccctgaccgccctatcccctgcaggctcctgcttctgggtggccgtgctggatggcaacgtggtgggcattgtggctgcacgggcccacgaggaggacaacacggtggagctgctgcggatgtctgtggactcacgtttccgaggcaagggcatcgccaaggcgctgggccggaaggtgctggagttcgccgtggtgcacaactactccgcggtggtgctgggcacgacggccgtcaaggtggccgcccacaagctctacgagtcgctgggcttcagacacatgggcgccagtgaccactacgtgctgccgggcatgaccctctcgctggctgagcgcctcttcttccaggtccgctaccaccgctaccgcctgcagctgcgcgaggagtgaccgccgccgctcgcccgcccgcccccccggccgccctgtccgcctttgcccgcctgcccgccgcccggcgcggcctgctttcagacgctcaattggcgtttgtgttgggtttccccttttcaacatcctgcggttgtctggctggttccggggggtgcggggctgttgttcttcggcgacactttggtgggggtgggttgtttgtcgccatagcccctcgcccttccccacctgcctgggcggcttgccacctgaagagtggcatcttggaccaccgcggctgtccatgacgctgccctgcccgccgccactggggaaggcccagccttgctcaccaagcacagaacctctgcagcagatcccggggccaggctccggccccgcctgcggccccagcgccactgcctgtggaggccccagctggccacggcgctgctttgctccgcgcatgccgagggtgtggcccggctgagcatgccgcatgcacacagccccgccctgccgccctgcccagactggacccggagacccgggctggtgagcgcccctgtccccagcccccagctggctgtgggagggcctgcccctgcccccacctcctggagggcctggtctgccccgcgccgcccggctctgtccacacctgctttgctctgacgccctccatttctctggctccggcccctcccctgcctgggctgtgctgactggtgtcatcacccaggtgactcccatggcgtccgtggcacagccagggtgggggtccatgggacccctctccccagtgcccactggatcgtgctggcctctcccagatgtccccggggacctcctgcctctggctgacggtccaccctgtgaatcttatcagcccaggctgctgccaacagcgcccagcccacagcttctcccagcctgaaaccaacacattttctaataagttatttagacagaatagcactctgcatgactttaattcttgggacaaaacggtagtttgtaccctaagacacagtttctggcccagtgtgatgggggtgggtggccgggtgggtggagcgttttgctgttggaaacctggagggaaaccctgattggatgtcatttcctgccatggagcacgcctcccagccctggcctgcagtgtgggcagggtggggggcaagggagtccgcagcctccgggaggaggggcagggcgctgccttgggctgggtgggaagaggggtggccgcctcggcttccgctggccatgctcctggtctctccttcctgaggtcacaggcaggggctgccctggacggggggcggggggggtggcctggaaggggagacagaggtggagggtggcacaggctgcacattcagcttagaagtggacctggctttggtggcaggagaagaataaacacttgcccagacccctttgtgtgggggaattggggaggggtcgtggcaggcagggtgggccacggaactgggtcccaggcatcaaggccacgtgcagggccatggagggatgcttctcacgaggcgcttcagaagcgagcgaagggacagagaagccctgcgtccaagggccttttgtcctgttagcaattgaggtgtgcagagcactgtacagaccccactcccctgtacattcctccctggaggtgcccggtccccgcttggggatgggagttttgtagactgtacagaaatcggcaccctattttcttgcagctcagattttgttaatctggaatatacagacagacgtaaagtgttttagcaaaatggaaacaaacagttgtgcctttttcctcttttggtttggtttgggtttggcctggggctggtcccagttggtgcggggcatgctgggggcaggaggggcagggcgggccaggtggagtcaggtcttgggggtgtcatgtcggggtgctgccagcgtcccttggtcctgtgcctttggagcctcgggctcctggggtgcagggtgcttgggggtggctgttggagcccaccgacgccagggcagggcctggaggcccagggactgccagtgtctccttgatattgatcctagcagatccccctcctgggggttctgagagtcctgggcagtgtggccttctctcattctggtggcatctgcgcccgtgagtgacctcttccttggctgcactgccctgtgggtggtgagacgcttggccttttttgttgctgccaggacttggtagagatggcaggaagggtgtgcggggtggttgtgtgcagaaccctgccgcccctgaggtgagcagaggcactggtgtgcctgccaggctggggcggagctgcccggaacccttgccaggccaatgtgtagcttggtggcgcccttgaaggccactggggggtaggtgtgtcctcccccggggctggaggccgggtgcctggtgggtgggcctgacctggcccacctcatccctccagcctgggatctcactgctctgcacatgtgcactgacgtgaattttatacgtttgtagaaactctgatgtaacttcttctacctctgaagcgccctcctgggccctgctgtcacgtggctggtgcggcttccccgagtgctggccccctgcccgctccccatgggcacccaccactcaaactctgcgtggtgaggcccggggaaagccaggccgggccctagcactgctcaggggcctggggctcctgggatttctgtgtgtttggaagcctctgtttttggagtggggggcgtggaaggcgggaggggctgacagtgcctggggccagagctgggcgaaggaggacatcctcactggacgagcactgcaggcctgacgagaggctccgggcagctgagggctgaagcgcctcgggatgccccacactgcccagagccacgggtcttgctgtgctgggggtggggtggccccaggcagctgccactgcccaggtgtgagtcgctgcagacctggtccctgagggtgggaggcctgcttgtgctgaagctgccatgtggtctggcccccagaagggctcctgagcgtagggagttgagccccgcagccgtggcggcatctcctcctccttcctccgccccatgcccagccttgcttccggctggtggcatgccttggggccgggcagcaggtgagaagctgcccccggagggaggggccaagggctgggtcagtctggcctgaggggtggatggctggccctgggggaaggctgggcctgggaggccctgtggacagcaggcctgaggccacgaggtcctggggccacccctctcctgctctgggtgtccttctcagagaagatggcagaaggctgcaggccctagaggggacgacgggctggggacgccctgagcttcctggctgtgtggcaggtgctggggaagctctctgggccatagggacctggctcccctctgagaggcccgcctgccctggggcacccagggctgtgctggtggcctgaggctgggaacttgggttcctgggctctgctggcgcctgggctttgtgggggccgaaggtggttgccccgccgggcagggggctgtgtgggccctcgtggggccaccttcctgtcagtgcccgagggacggtgggacagttgcccctgcgccaagggagcccactgccatcccccgtgggctggcgctgcagtcgcgcaccctgtgttgcctgctgacttgcttatgtctgtttggaagctgcttctgtttggccacaggcctgtgttcctgggctgatggccacagagctgacacttggggtttgagttccagacttgcagaccctgggatccgcttactcctccgcgccacggctgctgtgcttccagaggtggcgtgatgcctcccggggccatggtggggtgaaggatggcgagccggacataccattgttggatgtattttttttaaaacagcaataattagccattttaaaggagggatgtacctgtgtgtgtgcatgtgcacgtgtttgagcgtgtgtgtgtgtgcaagtgggttcttggatatgtgtagtgtgagcatgtgtgcatgtgtgagcctgtgcacgtgcatgttgtaggtgcataagcatgtacacgcgtgagcatgcatgtgtgtgtacacgtgtataggtgtacatgtgcatgagttgtgtacatgcgtgagcacgtgtatagatgtacgtgtgtgtgcgcatgtgtgcgtgtacatgtgtttgtgtcttgggtatgcataagcatgcacgtgtgtatgaatgtgcgtgtgtatgcatgagcacgtatgcatgtgtgatgcgcatgttcttgtgtacttctctgagggggcacatatgtatgagtgtatgagtgcatacatatgcatatggtgtacgtgtgtgtactttgtgtgtgggcatgtatgcgtatgagtacttgtgtgtggctggtgtatgggtgagcatgcacatgtgtatggatgcatatgtgagctgtacatgtagagtgtacatgtgtgttgcatgagcatgcatgcgtgcgagtgcctgtgtgtgtacttgcatatatgtggctgtgcaggtacccggcggtggtgggctggcagtgcccggggaagcaggtcctgctcacggcttccctgctgggccccaggccccgggctgagcctccctggcccctcttgcccctcagtggagcagggcctgggtggagggcctgggcagctccagggcaggtccggggaggccaggcttggggagcacagggggtggggttcacctgctgctgtttgttcccgaaactgtactgtgagcccacggcgggggcagattgttctgaggccccagatgtgccaaaacagcccccagggtgggggcgagcgagttctgaggagaggggctgcgggggccgggggtgggtgtgtgcccctcccctgaatggcccccaatgctgctgtttttcaataaaaccagagttgaaggca

SEQ ID NO: 2 - DNA, 6056 bp, synthetic construct
  source 1..6056: mol_type = other DNA
ccctgcgcgcgccccggccgcccgccgcctccgccccgcgcccctgagctgccgccgccgccgccgccgctgccgccgccgccgccgccgccgcgggtccgagggcgccgcgcccttgccctgggcccggccgtgcccgccgccgcccggccgcgtccccgctgccgcgccgcccggccgggtgcatgcattgtgggcctcccgacatggtctgcgagacgaagatcgtggccgccgaggaccatgaggcgctgccgggggccaagaaggacgcgctgctcgccgccgccggcgccatgtggcccccgctgcccgccgcgcccgggccggccgccgcgccccccgcgcccccacctgccccggtggctcagcctcacggcggggcggggggcgcggggccgccgggggggcgcggcgtgtgcatccgcgagttccgtgcggccgagcaggaggcggcgcgccgcatcttctacgacggcatcatggagcgcatccctaacacggccttccgcggcctgcggcagcacccgcgcgcgcagctgctctacgccctgctggcggcgctgtgcttcgccgtgagccgctcgctgctgctgacgtgcctggtgccggccgcgctgctgggcctgcgctactactacagccgcaaggtgatccgcgcctacctggagtgcgcgctgcacacggacatggcggacatcgagcagtactacatgaagccgcccggctcctgcttctgggtggccgtgctggatggcaacgtggtgggcattgtggctgcacgggcccacgaggaggacaacacggtggagctgctgcggatgtctgtggactcacgtttccgaggcaagggcatcgccaaggcgctgggccggaaggtgctggagttcgccgtggtgcacaactactccgcggtggtgctgggcacgacggccgtcaaggtggccgcccacaagctctacgagtcgctgggcttcagacacatgggcgccagtgaccactacgtgctgccgggcatgaccctctcgctggctgagcgcctcttcttccaggtccgctaccaccgctaccgcctgcagctgcgcgaggagtgaccgccgccgctcgcccgcccgcccccccggccgccctgtccgcctttgcccgcctgcccgccgcccggcgcggcctgctttcagacgctcaattggcgtttgtgttgggtttccccttttcaacatcctgcggttgtctggctggttccggggggtgcggggctgttgttcttcggcgacactttggtgggggtgggttgtttgtcgccatagcccctcgcccttccccacctgcctgggcggcttgccacctgaagagtggcatcttggaccaccgcggctgtccatgacgctgccctgcccgccgccactggggaaggcccagccttgctcaccaagcacagaacctctgcagcagatcccggggccaggctccggccccgcctgcggccccagcgccactgcctgtggaggccccagctggccacggcgctgctttgctccgcgcatgccgagggtgtggcccggctgagcatgccgcatgcacacagccccgccctgccgccctgcccagactggacccggagacccgggctggtgagcgcccctgtccccagcccccagctggctgtgggagggcctgcccctgcccccacctcctggagggcctggtctgccccgcgccgcccggctctgtccacacctgctttgctctgacgccctccatttctctggctccggcccctcccctgcctgggctgtgctgactggtgtcatcacccaggtgactcccatggcgtccgtggcacagccagggtgggggtccatgggacccctctccccagtgcccactggatcgtgctggcctctcccagatgtccccggggacctcctgcctctggctgacggtccaccctgtgaatcttatcagcccaggctgctgccaacagcgcccagcccacagcttctcccagcctgaaaccaacacattttctaataagttatttagacagaatagcactctgcatgactttaattcttgggacaaaacggtagtttgtaccctaagacacagtttctggcccagtgtgatgggggtgggtggccgggtgggtggagcgttttgctgttggaaacctggagggaaaccctgattggatgtcatttcctgccatggagcacgcctcccagccctggcctgcagtgtgggcagggtggggggcaagggagtccgcagcctccgggaggaggggcagggcgctgccttgggctgggtgggaagaggggtggccgcctcggcttccgctggccatgctcctggtctctccttcctgaggtcacaggcaggggctgccctggacggggggcggggggggtggcctggaaggggagacagaggtggagggtggcacaggctgcacattcagcttagaagtggacctggctttggtggcaggagaagaataaacacttgcccagacccctttgtgtgggggaattggggaggggtcgtggcaggcagggtgggccacggaactgggtcccaggcatcaaggccacgtgcagggccatggagggatgcttctcacgaggcgcttcagaagcgagcgaagggacagagaagccctgcgtccaagggccttttgtcctgttagcaattgaggtgtgcagagcactgtacagaccccactcccctgtacattcctccctggaggtgcccggtccccgcttggggatgggagttttgtagactgtacagaaatcggcaccctattttcttgcagctcagattttgttaatctggaatatacagacagacgtaaagtgttttagcaaaatggaaacaaacagttgtgcctttttcctcttttggtttggtttgggtttggcctggggctggtcccagttggtgcggggcatgctgggggcaggaggggcagggcgggccaggtggagtcaggtcttgggggtgtcatgtcggggtgctgccagcgtcccttggtcctgtgcctttggagcctcgggctcctggggtgcagggtgcttgggggtggctgttggagcccaccgacgccagggcagggcctggaggcccagggactgccagtgtctccttgatattgatcctagcagatccccctcctgggggttctgagagtcctgggcagtgtggccttctctcattctggtggcatctgcgcccgtgagtgacctcttccttggctgcactgccctgtgggtggtgagacgcttggccttttttgttgctgccaggacttggtagagatggcaggaagggtgtgcggggtggttgtgtgcagaaccctgccgcccctgaggtgagcagaggcactggtgtgcctgccaggctggggcggagctgcccggaacccttgccaggccaatgtgtagcttggtggcgcccttgaaggccactggggggtaggtgtgtcctcccccggggctggaggccgggtgcctggtgggtgggcctgacctggcccacctcatccctccagcctgggatctcactgctctgcacatgtgcactgacgtgaattttatacgtttgtagaaactctgatgtaacttcttctacctctgaagcgccctcctgggccctgctgtcacgtggctggtgcggcttccccgagtgctggccccctgcccgctccccatgggcacccaccactcaaactctgcgtggtgaggcccggggaaagccaggccgggccctagcactgctcaggggcctggggctcctgggatttctgtgtgtttggaagcctctgtttttggagtggggggcgtggaaggcgggaggggctgacagtgcctggggccagagctgggcgaaggaggacatcctcactggacgagcactgcaggcctgacgagaggctccgggcagctgagggctgaagcgcctcgggatgccccacactgcccagagccacgggtcttgctgtgctgggggtggggtggccccaggcagctgccactgcccaggtgtgagtcgctgcagacctggtccctgagggtgggaggcctgcttgtgctgaagctgccatgtggtctggcccccagaagggctcctgagcgtagggagttgagccccgcagccgtggcggcatctcctcctccttcctccgccccatgcccagccttgcttccggctggtggcatgccttggggccgggcagcaggtgagaagctgcccccggagggaggggccaagggctgggtcagtctggcctgaggggtggatggctggccctgggggaaggctgggcctgggaggccctgtggacagcaggcctgaggccacgaggtcctggggccacccctctcctgctctgggtgtccttctcagagaagatggcagaaggctgcaggccctagaggggacgacgggctggggacgccctgagcttcctggctgtgtggcaggtgctggggaagctctctgggccatagggacctggctcccctctgagaggcccgcctgccctggggcacccagggctgtgctggtggcctgaggctgggaacttgggttcctgggctctgctggcgcctgggctttgtgggggccgaaggtggttgccccgccgggcagggggctgtgtgggccctcgtggggccaccttcctgtcagtgcccgagggacggtgggacagttgcccctgcgccaagggagcccactgccatcccccgtgggctggcgctgcagtcgcgcaccctgtgttgcctgctgacttgcttatgtctgtttggaagctgcttctgtttggccacaggcctgtgttcctgggctgatggccacagagctgacacttggggtttgagttccagacttgcagaccctgggatccgcttactcctccgcgccacggctgctgtgcttccagaggtggcgtgatgcctcccggggccatggtggggtgaaggatggcgagccggacataccattgttggatgtattttttttaaaacagcaataattagccattttaaaggagggatgtacctgtgtgtgtgcatgtgcacgtgtttgagcgtgtgtgtgtgtgcaagtgggttcttggatatgtgtagtgtgagcatgtgtgcatgtgtgagcctgtgcacgtgcatgttgtaggtgcataagcatgtacacgcgtgagcatgcatgtgtgtgtacacgtgtataggtgtacatgtgcatgagttgtgtacatgcgtgagcacgtgtatagatgtacgtgtgtgtgcgcatgtgtgcgtgtacatgtgtttgtgtcttgggtatgcataagcatgcacgtgtgtatgaatgtgcgtgtgtatgcatgagcacgtatgcatgtgtgatgcgcatgttcttgtgtacttctctgagggggcacatatgtatgagtgtatgagtgcatacatatgcatatggtgtacgtgtgtgtactttgtgtgtgggcatgtatgcgtatgagtacttgtgtgtggctggtgtatgggtgagcatgcacatgtgtatggatgcatatgtgagctgtacatgtagagtgtacatgtgtgttgcatgagcatgcatgcgtgcgagtgcctgtgtgtgtacttgcatatatgtggctgtgcaggtacccggcggtggtgggctggcagtgcccggggaagcaggtcctgctcacggcttccctgctgggccccaggccccgggctgagcctccctggcccctcttgcccctcagtggagcagggcctgggtggagggcctgggcagctccagggcaggtccggggaggccaggcttggggagcacagggggtggggttcacctgctgctgtttgttcccgaaactgtactgtgagcccacggcgggggcagattgttctgaggccccagatgtgccaaaacagcccccagggtgggggcgagcgagttctgaggagaggggctgcgggggccgggggtgggtgtgtgcccctcccctgaatggcccccaatgctgctgtttttcaataaaaccagagttgaaggca

SEQ ID NO: 3 - protein, 302 aa, synthetic construct
MHCGPPDMVCETKIVAAEDHEALPGAKKDALLAAAGAMWPPLPAAPGPAAAPPAPPPAPVAQPHGGAGGAGPPGGRGVCIREFRAAEQEAARRIFYDGIMERIPNTAFRGLRQHPRAQLLYALLAALCFAVSRSLLLTCLVPAALLGLRYYYSRKVIRAYLECALHTDMADIEQYYMKPPGSCFWVAVLDGNVVGIVAARAHEEDNTVELLRMSVDSRFRGKGIAKALGRKVLEFAVVHNYSAVVLGTTAVKVAAHKLYESLGFRHMGASDHYVLPGMTLSLAERLFFQVRYHRYRLQLREE

SEQ ID NO: 4 - DNA, 18 bp, synthetic construct
  source 1..18: mol_type = other DNA
agtgcctaaatataatct

SEQ ID NO: 5 - DNA, 18 bp, synthetic construct
  source 1..18: mol_type = other DNA
aattcttgggacaaaacg

SEQ ID NO: 6 - DNA, 36 bp, synthetic construct
  source 1..36: mol_type = other DNA
aagtgttttagcaaaatggaaacaaacagttgtgcc

SEQ ID NO: 7 - DNA, 17 bp, synthetic construct
  source 1..17: mol_type = other DNA
gatgtaacttcttctac

SEQ ID NO: 8 - DNA, 18 bp, synthetic construct
  source 1..18: mol_type = other DNA
ggacataccattgttgga

SEQ ID NO: 9 - DNA, 17 bp, synthetic construct
  source 1..17: mol_type = other DNA
aaacagcaataattagc

SEQ ID NO: 10 - DNA, 16 bp, synthetic construct
  source 1..16: mol_type = other DNA
agatgtgccaaaacag

SEQ ID NO: 11 - DNA, 20 bp, synthetic construct
  source 1..20: mol_type = other DNA
gagtgcctaaatataatctg

SEQ ID NO: 12 - DNA, 30 bp, synthetic construct
  source 1..30: mol_type = other DNA
gaaatagggagaagtagaaagcacccgaga

SEQ ID NO: 13 - DNA, 29 bp, synthetic construct
  source 1..29: mol_type = other DNA
atggcggacatcgagcagtactacatgaa

SEQ ID NO: 14 - DNA, 18 bp, synthetic construct
  source 1..18: mol_type = other DNA
ggatttataggaaagctc

SEQ ID NO: 15 - DNA, 16 bp, synthetic construct
  source 1..16: mol_type = other DNA
gtgctggagtttgccg

SEQ ID NO: 16 - DNA, 16 bp, synthetic construct
  source 1..16: mol_type = other DNA
cctgtgaatcttatca

SEQ ID NO: 17 - DNA, 31 bp, synthetic construct
  source 1..31: mol_type = other DNA
attttctaataagttatttagacagaatagc

SEQ ID NO: 18 - DNA, 29 bp, synthetic construct
  source 1..29: mol_type = other DNA
aattcttgggacaaaacggtagtttgtac

SEQ ID NO: 19 - DNA, 18 bp, synthetic construct
  source 1..18: mol_type = other DNA
gttagcaattgaggtgtg

SEQ ID NO: 20 - DNA, 20 bp, synthetic construct
  source 1..20: mol_type = other DNA
gatgggagttttgtagactg

SEQ ID NO: 21 - DNA, 18 bp, synthetic construct
  source 1..18: mol_type = other DNA
ctggaatatacagacaga

SEQ ID NO: 22 - DNA, 42 bp, synthetic construct
  source 1..42: mol_type = other DNA
aagtgttttagcaaaatggaaacaaacagttgtgcctttttc

SEQ ID NO: 23 - DNA, 19 bp, synthetic construct
  source 1..19: mol_type = other DNA
atgtgcactgacgtgaatt

SEQ ID NO: 24 - DNA, 34 bp, synthetic construct
  source 1..34: mol_type = other DNA
acgtttgtagaaactctgatgtaacttcttctac

SEQ ID NO: 25 - DNA, 21 bp, synthetic construct
  source 1..21: mol_type = other DNA
ggacataccattgttggatgt

SEQ ID NO: 26 - DNA, 18 bp, synthetic construct
  source 1..18: mol_type = other DNA
aaacagcaataattagcc

SEQ ID NO: 27 - DNA, 17 bp, synthetic construct
  source 1..17: mol_type = other DNA
gtacttgcatatatgtg

SEQ ID NO: 28 - DNA, 16 bp, synthetic construct
  source 1..16: mol_type = other DNA
agatgtgccaaaacag

SEQ ID NO: 29 - DNA, 37 bp, synthetic construct
  source 1..37: mol_type = other DNA
aatgctgctgtttttcaataaaaccagagttgaaggc

SEQ ID NO: 30 - DNA, 16 bp, synthetic construct
  source 1..16: mol_type = other DNA
cgggtgcatgcattgt

SEQ ID NO: 31 - DNA, 30 bp, synthetic construct
  source 1..30: mol_type = other DNA
cggcgagtgcctaaatataatctgcgggcg

SEQ ID NO: 32 - DNA, 24 bp, synthetic construct
  source 1..24: mol_type = other DNA
ctcggtgctaatttatgaggggaa

SEQ ID NO: 33 - DNA, 44 bp, synthetic construct
  source 1..44: mol_type = other DNA
caggaagcttggaaatagggagaagtagaaagcacccgagatga

SEQ ID NO: 34 - DNA, 16 bp, synthetic construct
  source 1..16: mol_type = other DNA
cgcaaggtgatccgcg

SEQ ID NO: 35 - DNA, 33 bp, synthetic construct
  source 1..33: mol_type = other DNA
atggcggacatcgagcagtactacatgaagccg

SEQ ID NO: 36 - DNA, 16 bp, synthetic construct
  source 1..16: mol_type = other DNA
ctgagtgacctgagct

SEQ ID NO: 37 - DNA, 17 bp, synthetic construct
  source 1..17: mol_type = other DNA
cttagctgaagggtttt

SEQ ID NO: 38 - DNA, 17 bp, synthetic construct
  source 1..17: mol_type = other DNA
gccttctatagctggag

SEQ ID NO: 39 - DNA, 15 bp, synthetic construct
  source 1..15: mol_type = other DNA
cgtcttgcctgggta

SEQ ID NO: 40 - DNA, 20 bp, synthetic construct
  source 1..20: mol_type = other DNA
ggatttataggaaagctcct

SEQ ID NO: 41 - DNA, 23 bp, synthetic construct
  source 1..23: mol_type = other DNA
gtgctggagtttgccgtgatggc

SEQ ID NO: 42 - DNA, 16 bp, synthetic construct
  source 1..16: mol_type = other DNA
atggcctgttcagggt

SEQ ID NO: 43 - DNA, 16 bp, synthetic construct
  source 1..16: mol_type = other DNA
gtgggtcctggctgtt

SEQ ID NO: 44 - DNA, 24 bp, synthetic construct
  source 1..24: mol_type = other DNA
gtaacaggtgttgggctggctttc

SEQ ID NO: 45 - DNA, 17 bp, synthetic construct
  source 1..17: mol_type = other DNA
gcttgtcctgaatagtg

SEQ ID NO: 46 - DNA, 15 bp, synthetic construct
  source 1..15: mol_type = other DNA
gaggacaacacggtg

SEQ ID NO: 47 - DNA, 33 bp, synthetic construct
  source 1..33: mol_type = other DNA
gaaggtgctggagttcgccgtggtgcacaacta

SEQ ID NO: 48 - DNA, 14 bp, synthetic construct
  source 1..14: mol_type = other DNA
cggccgtcaaggtg

SEQ ID NO: 49 - DNA, 14 bp, synthetic construct
  source 1..14: mol_type = other DNA
ctacgagtcgctgg

SEQ ID NO: 50 - DNA, 20 bp, synthetic construct
  source 1..20: mol_type = other DNA
ctcaattggcgtttgtgttg

SEQ ID NO: 51 - DNA, 16 bp, synthetic construct
  source 1..16: mol_type = other DNA
gttgtctggctggttc

SEQ ID NO: 52 - DNA, 18 bp, synthetic construct
  source 1..18: mol_type = other DNA
aagagtggcatcttggac

SEQ ID NO: 53 - DNA, 16 bp, synthetic construct
  source 1..16: mol_type = other DNA
cctgtgaatcttatca

SEQ ID NO: 54 - DNA, 76 bp, synthetic construct
  source 1..76: mol_type = other DNA
attttctaataagttatttagacagaatagcactctgcatgactttaattcttgggacaaaacggtagtttgtacc

SEQ ID NO: 55 - DNA, 23 bp, synthetic construct
  source 1..23: mol_type = other DNA
gttagcaattgaggtgtgcagag

SEQ ID NO: 56 - DNA, 24 bp, synthetic construct
  source 1..24: mol_type = other DNA
gggatgggagttttgtagactgta

SEQ ID NO: 57 - DNA, 18 bp, synthetic construct
  source 1..18: mol_type = other DNA
ctggaatatacagacaga

SEQ ID NO: 58 - DNA, 42 bp, synthetic construct; identical to SEQ ID NO: 22
  source 1..42: mol_type = other DNA

SEQ ID NO: 59 - DNA, 24 bp, synthetic construct
  source 1..24: mol_type = other DNA
cttttggtttggtttgggtttggc

SEQ ID NO: 60 - DNA, 16 bp, synthetic construct
  source 1..16: mol_type = other DNA
gtgtcatgtcggggtg

SEQ ID NO: 61 - DNA, 23 bp, synthetic construct
  source 1..23: mol_type = other DNA
ctccttgatattgatcctagcag

SEQ ID NO: 62 - DNA, 15 bp, synthetic construct
  source 1..15: mol_type = other DNA
gtgcggggtggttgt

SEQ ID NO: 63 - DNA, 57 bp, synthetic construct
  source 1..57: mol_type = other DNA
atgtgcactgacgtgaattttatacgtttgtagaaactctgatgtaacttcttctac

SEQ ID NO: 64 - DNA, 20 bp, synthetic construct
  source 1..20: mol_type = other DNA
gtgtgtttggaagcctctgt

SEQ ID NO: 65 - DNA, 17 bp, synthetic construct
  source 1..17: mol_type = other DNA
ctgacacttggggtttg

SEQ ID NO: 66 - DNA, 29 bp, synthetic construct
  source 1..29: mol_type = other DNA
ggacataccattgttggatgtattttttt

SEQ ID NO: 67 - DNA, 30 bp, synthetic construct
  source 1..30: mol_type = other DNA
aaacagcaataattagccattttaaaggag

SEQ ID NO: 68 - DNA, 61 bp, synthetic construct
  source 1..61: mol_type = other DNA
atgtgcacgtgtttgagcgtgtgtgtgtgtgcaagtgggttcttggatatgtgtagtgtga

SEQ ID NO: 69 - DNA, 40 bp, synthetic construct
  source 1..40: mol_type = other DNA
gcatgttgtaggtgcataagcatgtacacgcgtgagcatg

SEQ ID NO: 70 - DNA, 21 bp, synthetic construct
  source 1..21: mol_type = other DNA
aggtgtacatgtgcatgagtt

SEQ ID NO: 71 - DNA, 19 bp, synthetic construct
  source 1..19: mol_type = other DNA
acgtgtatagatgtacgtg

SEQ ID NO: 72 - DNA, 21 bp, synthetic construct
  source 1..21: mol_type = other DNA
gcgtgtacatgtgtttgtgtc

SEQ ID NO: 73 - DNA, 39 bp, synthetic construct
  source 1..39: mol_type = other DNA
cgtgtgtatgaatgtgcgtgtgtatgcatgagcacgtat

SEQ ID NO: 74 - DNA, 29 bp, synthetic construct
  source 1..29: mol_type = other DNA
cgcatgttcttgtgtacttctctgagggg

SEQ ID NO: 75 - DNA, 18 bp, synthetic construct
  source 1..18: mol_type = other DNA
catatgcatatggtgtac

SEQ ID NO: 76 - DNA, 24 bp, synthetic construct
  source 1..24: mol_type = other DNA
catgcacatgtgtatggatgcata

SEQ ID NO: 77 - DNA, 22 bp, synthetic construct
  source 1..22: mol_type = other DNA
atgtgtgttgcatgagcatgca

SEQ ID NO: 78 - DNA, 39 bp, synthetic construct
  source 1..39: mol_type = other DNA
agtgcctgtgtgtgtacttgcatatatgtggctgtgcag

SEQ ID NO: 79 - DNA, 17 bp, synthetic construct
  source 1..17: mol_type = other DNA
gggcagattgttctgag

SEQ ID NO: 80 - DNA, 17 bp, synthetic construct
  source 1..17: mol_type = other DNA
agatgtgccaaaacagc

SEQ ID NO: 81 - DNA, 38 bp, synthetic construct
  source 1..38: mol_type = other DNA
caatgctgctgtttttcaataaaaccagagttgaaggc

SEQ ID NO: 82 - DNA, 16 bp, synthetic construct
  source 1..16: mol_type = other DNA
cgggtgcatgcattgt

SEQ ID NO: 83 - DNA, 14 bp, synthetic construct
  source 1..14: mol_type = other DNA
cggcgagtgcctaa

SEQ ID NO: 84 - DNA, 14 bp, synthetic construct
  source 1..14: mol_type = other DNA
gcgagtgcctaaat

SEQ ID NO: 85 - DNA, 15 bp, synthetic construct
  source 1..15: mol_type = other DNA
cgagtgcctaaatat

SEQ ID NO: 86 - DNA, 16 bp, synthetic construct
  source 1..16: mol_type = other DNA
gagtgcctaaatataa

SEQ ID NO: 87 - DNA, 17 bp, synthetic construct
  source 1..17: mol_type = other DNA
gagtgcctaaatataat

SEQ ID NO: 88 - DNA, 18 bp, synthetic construct
  source 1..18: mol_type = other DNA
gagtgcctaaatataatc

SEQ ID NO: 89 - DNA, 19 bp, synthetic construct
  source 1..19: mol_type = other DNA
gagtgcctaaatataatct

SEQ ID NO: 90 - DNA, 20 bp, synthetic construct
  source 1..20: mol_type = other DNA
gagtgcctaaatataatctg

SEQ ID NO: 91 - DNA, 17 bp, synthetic construct
  source 1..17: mol_type = other DNA
agtgcctaaatataatc

SEQ ID NO: 92 - DNA, 18 bp, synthetic construct
  source 1..18: mol_type = other DNA
agtgcctaaatataatct

SEQ ID NO: 93 - DNA, 19 bp, synthetic construct
  source 1..19: mol_type = other DNA
agtgcctaaatataatctg

SEQ ID NO: 94 - DNA, 16 bp, synthetic construct
  source 1..16: mol_type = other DNA
gtgcctaaatataatc

SEQ ID NO: 95 - DNA, 17 bp, synthetic construct
  source 1..17: mol_type = other DNA
gtgcctaaatataatct

SEQ ID NO: 96 - DNA, 18 bp, synthetic construct
  source 1..18: mol_type = other DNA
gtgcctaaatataatctg

SEQ ID NO: 97 - DNA, 19 bp, synthetic construct
  source 1..19: mol_type = other DNA
gtgcctaaatataatctgc

SEQ ID NO: 98 - DNA, 16 bp, synthetic construct
  source 1..16: mol_type = other DNA
aaatataatctgcggg

SEQ ID NO: 99 - DNA, 17 bp, synthetic construct
  source 1..17: mol_type = other DNA
aaatataatctgcgggc

SEQ ID NO: 100 - DNA, 15 bp, synthetic construct
  source 1..15: mol_type = other DNA
aatataatctgcggg

SEQ ID NO: 101 - DNA, 16 bp, synthetic construct
  source 1..16: mol_type = other DNA
aatataatctgcgggc

SEQ ID NO: 102 - DNA, 17 bp, synthetic construct
  source 1..17: mol_type = other DNA
aatataatctgcgggcg

SEQ ID NO: 103 - DNA, 16 bp, synthetic construct
  source 1..16: mol_type = other DNA
ctcggtgctaatttat

SEQ ID NO: 104 - DNA, 16 bp, synthetic construct
  source 1..16: mol_type = other DNA
ggtgctaatttatgag

SEQ ID NO: 105 - DNA, 17 bp, synthetic construct
  source 1..17: mol_type = other DNA
ctaatttatgaggggaa

SEQ ID NO: 106 - DNA, 17 bp, synthetic construct
  source 1..17: mol_type = other DNA
caggaagcttggaaata

SEQ ID NO: 107 - DNA, 17 bp, synthetic construct
  source 1..17: mol_type = other DNA
aggaagcttggaaatag

SEQ ID NO: 108 - DNA, 17 bp, synthetic construct
  source 1..17: mol_type = other DNA
aagcttggaaataggga

SEQ ID NO: 109 - DNA, 18 bp, synthetic construct
  source 1..18: mol_type = other DNA
gaaatagggagaagtaga

SEQ ID NO: 110 - DNA, 19 bp, synthetic construct
  source 1..19: mol_type = other DNA
gaaatagggagaagtagaa

SEQ ID NO: 111 - DNA, 17 bp, synthetic construct
  source 1..17: mol_type = other DNA
aaatagggagaagtaga

SEQ ID NO: 112 - DNA, 18 bp, synthetic construct
  source 1..18: mol_type = other DNA
aaatagggagaagtagaa

SEQ ID NO: 113 - DNA, 19 bp, synthetic construct
  source 1..19: mol_type = other DNA
aaatagggagaagtagaaa

SEQ ID NO: 114 - DNA, 18 bp, synthetic construct
  source 1..18: mol_type = other DNA
aatagggagaagtagaaa

SEQ ID NO: 115 - DNA, 17 bp, synthetic construct
  source 1..17: mol_type = other DNA
gaagtagaaagcacccg

SEQ ID NO: 116 - DNA, 16 bp, synthetic construct
  source 1..16: mol_type = other DNA
aagtagaaagcacccg

SEQ ID NO: 117 - DNA, 17 bp, synthetic construct
  source 1..17: mol_type = other DNA
aagtagaaagcacccga

SEQ ID NO: 118 - DNA, 17 bp, synthetic construct
  source 1..17: mol_type = other DNA
gtagaaagcacccgaga

SEQ ID NO: 119 - DNA, 16 bp, synthetic construct
  source 1..16: mol_type = other DNA
agaaagcacccgagat

SEQ ID NO: 120 - DNA, 15 bp, synthetic construct
  source 1..15: mol_type = other DNA
gaaagcacccgagat

SEQ ID NO: 121 - DNA, 16 bp, synthetic construct
  source 1..16: mol_type = other DNA
gaaagcacccgagatg

SEQ ID NO: 122 - DNA, 16 bp, synthetic construct
  source 1..16: mol_type = other DNA
aaagcacccgagatga

SEQ ID NO: 123 - DNA, 14 bp, synthetic construct
  source 1..14: mol_type = other DNA
cgcaaggtgatccg

SEQ ID NO: 124 - DNA, 14 bp, synthetic construct
  source 1..14: mol_type = other DNA
caaggtgatccgcg

SEQ ID NO: 125 - DNA, 14 bp, synthetic construct
  source 1..14: mol_type = other DNA
atggcggacatcga

SEQ ID NO: 126 - DNA, 16 bp, synthetic construct
  source 1..16: mol_type = other DNA
cggacatcgagcagta

SEQ ID NO: 127 - DNA, 15 bp, synthetic construct
  source 1..15: mol_type = other DNA
ggacatcgagcagta

SEQ ID NO: 128 - DNA, 16 bp, synthetic construct
  source 1..16: mol_type = other DNA
gagcagtactacatga

SEQ ID NO: 129 - DNA, 17 bp, synthetic construct
  source 1..17: mol_type = other DNA
gagcagtactacatgaa

SEQ ID NO: 130 - DNA, 18 bp, synthetic construct
  source 1..18: mol_type = other DNA
gagcagtactacatgaag

SEQ ID NO: 131 - DNA, 16 bp, synthetic construct
  source 1..16: mol_type = other DNA
agtactacatgaagcc

SEQ ID NO: 132 - DNA, 16 bp, synthetic construct
  source 1..16: mol_type = other DNA
gtactacatgaagccg

SEQ ID NO: 133 - DNA, 16 bp, synthetic construct
  source 1..16: mol_type = other DNA
ctgagtgacctgagct

SEQ ID NO: 134 - DNA, 17 bp, synthetic construct
  source 1..17: mol_type = other DNA
cttagctgaagggtttt

SEQ ID NO: 135 - DNA, 17 bp, synthetic construct
  source 1..17: mol_type = other DNA
gccttctatagctggag

SEQ ID NO: 136 - DNA, 15 bp, synthetic construct
  source 1..15: mol_type = other DNA
cgtcttgcctgggta

SEQ ID NO: 137 - DNA, 18 bp, synthetic construct
  source 1..18: mol_type = other DNA
ggatttataggaaagctc

SEQ ID NO: 138 - DNA, 17 bp, synthetic construct
  source 1..17: mol_type = other DNA
gatttataggaaagctc

SEQ ID NO: 139 - DNA, 18 bp, synthetic construct
  source 1..18: mol_type = other DNA
gatttataggaaagctcc

SEQ ID NO: 140 - DNA, 19 bp, synthetic construct
  source 1..19: mol_type = other DNA
gatttataggaaagctcct

SEQ ID NO: 141 - DNA, 16 bp, synthetic construct
  source 1..16: mol_type = other DNA
atttataggaaagctc

SEQ ID NO: 142 - DNA, 17 bp, synthetic construct
  source 1..17: mol_type = other DNA
atttataggaaagctcc

SEQ ID NO: 143 - DNA, 18 bp, synthetic construct
  source 1..18: mol_type = other DNA
atttataggaaagctcct

SEQ ID NO: 144 - DNA, 16 bp, synthetic construct
  source 1..16: mol_type = other DNA
gtgctggagtttgccg

SEQ ID NO: 145 - DNA, 17 bp, synthetic construct
  source 1..17: mol_type = other DNA
gtgctggagtttgccgt

SEQ ID NO: 146 - DNA, 16 bp, synthetic construct
  source 1..16: mol_type = other DNA
ggagtttgccgtgatg

SEQ ID NO: 147 - DNA, 16 bp, synthetic construct
  source 1..16: mol_type = other DNA
gagtttgccgtgatgg

SEQ ID NO: 148 - DNA, 17 bp, synthetic construct
  source 1..17: mol_type = other DNA
gagtttgccgtgatggc

SEQ ID NO: 149 - DNA, 15 bp, synthetic construct
  source 1..15: mol_type = other DNA
agtttgccgtgatgg

SEQ ID NO: 150 - DNA, 16 bp, synthetic construct
  source 1..16: mol_type = other DNA
agtttgccgtgatggc

SEQ ID NO: 151 - DNA, 16 bp, synthetic construct
  source 1..16: mol_type = other DNA
atggcctgttcagggt

SEQ ID NO: 152 - DNA, 16 bp, synthetic construct
  source 1..16: mol_type = other DNA
gtgggtcctggctgtt

SEQ ID NO: 153 - DNA, 16 bp, synthetic construct
  source 1..16: mol_type = other DNA
gtaacaggtgttgggc

SEQ ID NO: 154 - DNA, 16 bp, synthetic construct
  source 1..16: mol_type = other DNA
gtgttgggctggcttt

SEQ ID NO: 155 - DNA, 17 bp, synthetic construct
  source 1..17: mol_type = other DNA
gtgttgggctggctttc

SEQ ID NO: 156 - DNA, 16 bp, synthetic construct
  source 1..16: mol_type = other DNA
gcttgtcctgaatagt

SEQ ID NO: 157 - DNA, 16 bp, synthetic construct
  source 1..16: mol_type = other DNA
cttgtcctgaatagtg

SEQ ID NO: 158 - DNA, 15 bp, synthetic construct
  source 1..15: mol_type = other DNA
gaggacaacacggtg

SEQ ID NO: 159 - DNA, 16 bp, synthetic construct
  source 1..16: mol_type = other DNA
gaaggtgctggagttc

SEQ ID NO: 160 - DNA, 16 bp, synthetic construct
  source 1..16: mol_type = other DNA
aaggtgctggagttcg

SEQ ID NO: 161 - DNA, 15 bp, synthetic construct
  source 1..15: mol_type = other DNA
ggagttcgccgtggt

SEQ ID NO: 162 - DNA, 14 bp, synthetic construct
  source 1..14: mol_type = other DNA
gagttcgccgtggt

SEQ ID NO: 163 - DNA, 15 bp, synthetic construct
  source 1..15: mol_type = other DNA
gagttcgccgtggtg

SEQ ID NO: 164 - DNA, 15 bp, synthetic construct
  source 1..15: mol_type = other DNA
cgtggtgcacaacta

SEQ ID NO: 165 - DNA, 14 bp, synthetic construct
  source 1..14: mol_type = other DNA
cggccgtcaaggtg

SEQ ID NO: 166 - DNA, 14 bp, synthetic construct
  source 1..14: mol_type = other DNA
ctacgagtcgctgg

SEQ ID NO: 167 - DNA, 20 bp, synthetic construct
  source 1..20: mol_type = other DNA
ctcaattggcgtttgtgttg

SEQ ID NO: 168 - DNA, 16 bp, synthetic construct
  source 1..16: mol_type = other DNA
gttgtctggctggttc

SEQ ID NO: 169 - DNA, 17 bp, synthetic construct
  source 1..17: mol_type = other DNA
aagagtggcatcttgga

SEQ ID NO: 170 - DNA, 16 bp, synthetic construct
  source 1..16: mol_type = other DNA
gagtggcatcttggac

SEQ ID NO: 171 - DNA, 16 bp, synthetic construct
  source 1..16: mol_type = other DNA
cctgtgaatcttatca

SEQ ID NO: 172 - DNA, 20 bp, synthetic construct
  source 1..20: mol_type = other DNA
attttctaataagttattta

SEQ ID NO: 173 - DNA, 17 bp, synthetic construct
  source 1..17: mol_type = other DNA
aataagttatttagaca

SEQ ID NO: 174 - DNA, 18 bp, synthetic construct
  source 1..18: mol_type = other DNA
aataagttatttagacag

SEQ ID NO: 175 - DNA, 17 bp, synthetic construct
  source 1..17: mol_type = other DNA
ataagttatttagacag

SEQ ID NO: 176 - DNA, 18 bp, synthetic construct
  source 1..18: mol_type = other DNA
ataagttatttagacaga

SEQ ID NO: 177 - DNA, 19 bp, synthetic construct
  source 1..19: mol_type = other DNA
ataagttatttagacagaa

SEQ ID NO: 178 - DNA, 20 bp, synthetic construct
  source 1..20: mol_type = other DNA
ataagttatttagacagaat

SEQ ID NO: 179 - DNA, 18 bp, synthetic construct
  source 1..18: mol_type = other DNA
aagttatttagacagaat

SEQ ID NO: 180 - DNA, 19 bp, synthetic construct
  source 1..19: mol_type = other DNA
aagttatttagacagaata

SEQ ID NO: 181 - DNA, 20 bp, synthetic construct
  source 1..20: mol_type = other DNA
aagttatttagacagaatag

SEQ ID NO: 182 - DNA, 18 bp, synthetic construct
  source 1..18: mol_type = other DNA
agttatttagacagaata

SEQ ID NO: 183 - DNA, 19 bp, synthetic construct
  source 1..19: mol_type = other DNA
agttatttagacagaatag

SEQ ID NO: 184 - DNA, 20 bp, synthetic construct
  source 1..20: mol_type = other DNA
agttatttagacagaatagc

SEQ ID NO: 185 - DNA, 17 bp, synthetic construct
  source 1..17: mol_type = other DNA
gttatttagacagaata

SEQ ID NO: 186 - DNA, 18 bp, synthetic construct
  source 1..18: mol_type = other DNA
gttatttagacagaatag

SEQ ID NO: 187 - DNA, 18 bp, synthetic construct
  source 1..18: mol_type = other DNA
atttagacagaatagcac

SEQ ID NO: 188 - DNA, 19 bp, synthetic construct
  source 1..19: mol_type = other DNA
atttagacagaatagcact

SEQ ID NO: 189 - DNA, 17 bp, synthetic construct
  source 1..17: mol_type = other DNA
agacagaatagcactct

SEQ ID NO: 190 - DNA, 17 bp, synthetic construct
  source 1..17: mol_type = other DNA
agaatagcactctgcat

SEQ ID NO: 191 - DNA, 17 bp, synthetic construct
  source 1..17: mol_type = other DNA
atgactttaattcttgg

SEQ ID NO: 192 - DNA, 18 bp, synthetic construct
  source 1..18: mol_type = other DNA
atgactttaattcttggg

SEQ ID NO: 193 - DNA, 19 bp, synthetic construct
  source 1..19: mol_type = other DNA
atgactttaattcttggga

SEQ ID NO: 194 - DNA, 20 bp, synthetic construct
  source 1..20: mol_type = other DNA
atgactttaattcttgggac

SEQ ID NO: 195 - DNA, 17 bp, synthetic construct
  source 1..17: mol_type = other DNA
aattcttgggacaaaac

SEQ ID NO: 196 - DNA, 18 bp, synthetic construct
  source 1..18: mol_type = other DNA
aattcttgggacaaaacg

SEQ ID NO: 197 - DNA, 15 bp, synthetic construct
  source 1..15: mol_type = other DNA
gggacaaaacggtag

SEQ ID NO: 198 - DNA, 17 bp, synthetic construct
  source 1..17: mol_type = other DNA
gggacaaaacggtagtt

SEQ ID NO: 199 - DNA, 18 bp, synthetic construct
  source 1..18: mol_type = other DNA
gggacaaaacggtagttt

SEQ ID NO: 200 - DNA, 15 bp, synthetic construct
  source 1..15: mol_type = other DNA
ggacaaaacggtagt

SEQ ID NO: 201 - DNA, 16 bp, synthetic construct
  source 1..16: mol_type = other DNA
ggacaaaacggtagtt

SEQ ID NO: 202 - DNA, 17 bp, synthetic construct
  source 1..17: mol_type = other DNA
ggacaaaacggtagttt

SEQ ID NO: 203 - DNA, 18 bp, synthetic construct
  source 1..18: mol_type = other DNA
ggacaaaacggtagtttg

SEQ ID NO: 204 - DNA, 17 bp, synthetic construct
  source 1..17: mol_type = other DNA
aaaacggtagtttgtac

SEQ ID NO: 205 - DNA, 18 bp, synthetic construct
  source 1..18: mol_type = other DNA
aaaacggtagtttgtacc

SEQ ID NO: 206 - DNA, 16 bp, synthetic construct
  source 1..16: mol_type = other DNA
aaacggtagtttgtac

SEQ ID NO: 207 - DNA, 17 bp, synthetic construct
  source 1..17: mol_type = other DNA
aaacggtagtttgtacc

SEQ ID NO: 208 - DNA, 16 bp, synthetic construct
  source 1..16: mol_type = other DNA
gttagcaattgaggtg

SEQ ID NO: 209 - DNA, 17 bp, synthetic construct
  source 1..17: mol_type = other DNA
gttagcaattgaggtgt

SEQ ID NO: 210 - DNA, 18 bp, synthetic construct
  source 1..18: mol_type = other DNA
gttagcaattgaggtgtg

SEQ ID NO: 211 - DNA, 19 bp, synthetic construct
  source 1..19: mol_type = other DNA
gttagcaattgaggtgtgc

SEQ ID NO: 212 - DNA, 17 bp, synthetic construct
  source 1..17: mol_type = other DNA
aattgaggtgtgcagag

SEQ ID NO: 213 - DNA, 16 bp, synthetic construct
  source 1..16: mol_type = other DNA
attgaggtgtgcagag

SEQ ID NO: 214 - DNA, 17 bp, synthetic construct
  source 1..17: mol_type = other DNA
gggatgggagttttgta

SEQ ID NO: 215 - DNA, 17 bp, synthetic construct
  source 1..17: mol_type = other DNA
ggatgggagttttgtag

SEQ ID NO: 216 - DNA, 18 bp, synthetic construct
  source 1..18: mol_type = other DNA
ggatgggagttttgtaga

SEQ ID NO: 217 - DNA, 16 bp, synthetic construct
  source 1..16: mol_type = other DNA
gatgggagttttgtag

SEQ ID NO: 218 - DNA, 17 bp, synthetic construct
  source 1..17: mol_type = other DNA
gatgggagttttgtaga

SEQ ID NO: 219 - DNA, 18 bp, synthetic construct
  source 1..18: mol_type = other DNA
gatgggagttttgtagac

SEQ ID NO: 220 - DNA, 19 bp, synthetic construct
  source 1..19: mol_type = other DNA
gatgggagttttgtagact

SEQ ID NO: 221 - DNA, 16 bp, synthetic construct
  source 1..16: mol_type = other DNA
atgggagttttgtaga

SEQ ID NO: 222 - DNA, 17 bp, synthetic construct
  source 1..17: mol_type = other DNA
atgggagttttgtagac

SEQ ID NO: 223 - DNA, 18 bp, synthetic construct
  source 1..18: mol_type = other DNA
atgggagttttgtagact

SEQ ID NO: 224 - DNA, 19 bp, synthetic construct
  source 1..19: mol_type = other DNA
atgggagttttgtagactg

SEQ ID NO: 225 - DNA, 20 bp, synthetic construct
  source 1..20: mol_type = other DNA
atgggagttttgtagactgt

SEQ ID NO: 226 - DNA, 16 bp, synthetic construct
  source 1..16: mol_type = other DNA
gggagttttgtagact

SEQ ID NO: 227 - DNA, 17 bp, synthetic construct
  source 1..17: mol_type = other DNA
gggagttttgtagactg

SEQ ID NO: 228 - DNA, 18 bp, synthetic construct
  source 1..18: mol_type = other DNA
gggagttttgtagactgt

SEQ ID NO: 229 - DNA, 19 bp, synthetic construct
  source 1..19: mol_type = other DNA
gggagttttgtagactgta

SEQ ID NO: 230 - DNA, 16 bp, synthetic construct
  source 1..16: mol_type = other DNA
ggagttttgtagactg

SEQ ID NO: 231 - DNA, 17 bp, synthetic construct
  source 1..17: mol_type = other DNA
ggagttttgtagactgt

SEQ ID NO: 232 - DNA, 18 bp, synthetic construct
  source 1..18: mol_type = other DNA
ggagttttgtagactgta

SEQ ID NO: 233 - DNA, 17 bp, synthetic construct
  source 1..17: mol_type = other DNA
gagttttgtagactgta

SEQ ID NO: 234 - DNA, 18 bp, synthetic construct
  source 1..18: mol_type = other DNA
ctggaatatacagacaga

SEQ ID NO: 235 - DNA, 18 bp, synthetic construct
  source 1..18: mol_type = other DNA
aagtgttttagcaaaatg

SEQ ID NO: 236 - DNA, 19 bp, synthetic construct
  source 1..19: mol_type = other DNA
aagtgttttagcaaaatgg

SEQ ID NO: 237 - DNA, 20 bp, synthetic construct
  source 1..20: mol_type = other DNA
aagtgttttagcaaaatgga

SEQ ID NO: 238 - DNA, 17 bp, synthetic construct
  source 1..17: mol_type = other DNA
agtgttttagcaaaatg

SEQ ID NO: 239 - DNA, 18 bp, synthetic construct
  source 1..18: mol_type = other DNA
agtgttttagcaaaatgg

SEQ ID NO: 240 - DNA, 19 bp, synthetic construct
  source 1..19: mol_type = other DNA
agtgttttagcaaaatgga

SEQ ID NO: 241 - DNA, 20 bp, synthetic construct
  source 1..20: mol_type = other DNA
agtgttttagcaaaatggaa

SEQ ID NO: 242 - DNA, 17 bp, synthetic construct
  source 1..17: mol_type = other DNA
gtgttttagcaaaatgg

SEQ ID NO: 243 - DNA, 18 bp, synthetic construct
  source 1..18: mol_type = other DNA
gtgttttagcaaaatgga

SEQ ID NO: 244 - DNA, 19 bp, synthetic construct
  source 1..19: mol_type = other DNA
gtgttttagcaaaatggaa

SEQ ID NO: 245 - DNA, 20 bp, synthetic construct
  source 1..20: mol_type = other DNA
gtgttttagcaaaatggaaa

SEQ ID NO: 246 - DNA, 18 bp, synthetic construct
  source 1..18: mol_type = other DNA
gttttagcaaaatggaaa

SEQ ID NO: 247 - DNA, 19 bp, synthetic construct
  source 1..19: mol_type = other DNA
gttttagcaaaatggaaac

SEQ ID NO: 248 - DNA, 20 bp, synthetic construct
  source 1..20: mol_type = other DNA
gttttagcaaaatggaaaca

SEQ ID NO: 249 - DNA, 18 bp, synthetic construct
  source 1..18: mol_type = other DNA
aaaatggaaacaaacagt

SEQ ID NO: 250 - DNA, 19 bp, synthetic construct
  source 1..19: mol_type = other DNA
aaaatggaaacaaacagtt

SEQ ID NO: 251 - DNA, 20 bp, synthetic construct
  source 1..20: mol_type = other DNA
aaaatggaaacaaacagttg

SEQ ID NO: 252 - DNA, 18 bp, synthetic construct
  source 1..18: mol_type = other DNA
aaatggaaacaaacagtt

SEQ ID NO: 253 - DNA, 19 bp, synthetic construct
  source 1..19: mol_type = other DNA
aaatggaaacaaacagttg

SEQ ID NO: 254 - DNA, 20 bp, synthetic construct
  source 1..20: mol_type = other DNA
aaatggaaacaaacagttgt

SEQ ID NO: 255 - DNA, 18 bp, synthetic construct
  source 1..18: mol_type = other DNA
aatggaaacaaacagttg

SEQ ID NO: 256 - DNA, 19 bp, synthetic construct
  source 1..19: mol_type = other DNA
aatggaaacaaacagttgt

SEQ ID NO: 257 - DNA, 20 bp, synthetic construct
  source 1..20: mol_type = other DNA
aatggaaacaaacagttgtg

SEQ ID NO: 258 - DNA, 17 bp, synthetic construct
  source 1..17: mol_type = other DNA
atggaaacaaacagttg

SEQ ID NO: 259 - DNA, 18 bp, synthetic construct
  source 1..18: mol_type = other DNA
atggaaacaaacagttgt

SEQ ID NO: 260 - DNA, 19 bp, synthetic construct
  source 1..19: mol_type = other DNA
atggaaacaaacagttgtg

SEQ ID NO: 261 - DNA, 17 bp, synthetic construct
  source 1..17: mol_type = other DNA
ggaaacaaacagttgtg

SEQ ID NO: 262 - DNA, 17 bp, synthetic construct
  source 1..17: mol_type = other DNA
gaaacaaacagttgtgc

SEQ ID NO: 263 - DNA, 17 bp, synthetic construct
  source 1..17: mol_type = other DNA
aaacaaacagttgtgcc

SEQ ID NO: 264 - DNA, 18 bp, synthetic construct
  source 1..18: mol_type = other DNA
aaacaaacagttgtgcct

SEQ ID NO: 265 - DNA, 19 bp, synthetic construct
  source 1..19: mol_type = other DNA
aaacaaacagttgtgcctt

SEQ ID NO: 266 - DNA, 20 bp, synthetic construct
  source 1..20: mol_type = other DNA
aaacaaacagttgtgccttt

SEQ ID NO: 267 - DNA, 17 bp, synthetic construct
  source 1..17: mol_type = other DNA
aaacagttgtgcctttt

SEQ ID NO: 268 - DNA, 18 bp, synthetic construct
  source 1..18: mol_type = other DNA
aaacagttgtgccttttt

SEQ ID NO: 269 - DNA, 19 bp, synthetic construct
  source 1..19: mol_type = other DNA
aaacagttgtgcctttttc

SEQ ID NO: 270 - DNA, 19 bp, synthetic construct
  source 1..19: mol_type = other DNA
cttttggtttggtttgggt

SEQ ID NO: 271 - DNA, 18 bp, synthetic construct
  source 1..18: mol_type = other DNA
gtttggtttgggtttggc

SEQ ID NO: 272 - DNA, 15 bp, synthetic construct
  source 1..15: mol_type = other DNA
gtgtcatgtcggggt

SEQ ID NO: 273 - DNA, 16 bp, synthetic construct
  source 1..16: mol_type = other DNA
gtgtcatgtcggggtg

SEQ ID NO: 274 - DNA, 16 bp, synthetic construct
  source 1..16: mol_type = other DNA
ctccttgatattgatc

SEQ ID NO: 275 - DNA, 20 bp, synthetic construct
  source 1..20: mol_type = other DNA
cttgatattgatcctagcag

SEQ ID NO: 276 - DNA, 15 bp, synthetic construct
  source 1..15: mol_type = other DNA
gtgcggggtggttgt

SEQ ID NO: 277 - DNA, 15 bp, synthetic construct
  source 1..15: mol_type = other DNA
atgtgcactgacgtg

SEQ ID NO: 278 - DNA, 16 bp, synthetic construct
  source 1..16: mol_type = other DNA
atgtgcactgacgtga

SEQ ID NO: 279 - DNA, 17 bp, synthetic construct
  source 1..17: mol_type = other DNA
atgtgcactgacgtgaa

SEQ ID NO: 280 - DNA, 18 bp, synthetic construct
  source 1..18: mol_type = other DNA
atgtgcactgacgtgaat

SEQ ID NO: 281 - DNA, 19 bp, synthetic construct
  source 1..19: mol_type = other DNA
atgtgcactgacgtgaatt

SEQ ID NO: 282 - DNA, 20 bp, synthetic construct
  source 1..20: mol_type = other DNA
atgtgcactgacgtgaattt

SEQ ID NO: 283 - DNA, 15 bp, synthetic construct
  source 1..15: mol_type = other DNA
gtgcactgacgtgaa

SEQ ID NO: 284 - DNA, 16 bp, synthetic construct
  source 1..16: mol_type = other DNA
gtgcactgacgtgaat

SEQ ID NO: 285 - DNA, 17 bp, synthetic construct
  source 1..17: mol_type = other DNA
gtgcactgacgtgaatt

SEQ ID NO: 286 - DNA, 18 bp, synthetic construct
  source 1..18: mol_type = other DNA
gtgcactgacgtgaattt

SEQ ID NO: 287 - DNA, 19 bp, synthetic construct
  source 1..19: mol_type = other DNA
gtgcactgacgtgaatttt

SEQ ID NO: 288 - DNA, 20 bp, synthetic construct
  source 1..20: mol_type = other DNA
gtgcactgacgtgaatttta

SEQ ID NO: 289 - DNA, 15 bp, synthetic construct
  source 1..15: mol_type = other DNA
cgtgaattttatacg

SEQ ID NO: 290 - DNA, 19 bp, synthetic construct
  source 1..19: mol_type = other DNA
acgtttgtagaaactctga

SEQ ID NO: 291 - DNA, 17 bp, synthetic construct
  source 1..17: mol_type = other DNA
cgtttgtagaaactctg

SEQ ID NO: 292 - DNA, 16 bp, synthetic construct
  source 1..16: mol_type = other DNA
gatgtaacttcttcta

SEQ ID NO: 293 - DNA, 17 bp, synthetic construct
  source 1..17: mol_type = other DNA
gatgtaacttcttctac

SEQ ID NO: 294 - DNA, 17 bp, synthetic construct
  source 1..17: mol_type = other DNA
gtgtgtttggaagcctc

SEQ ID NO: 295 - DNA, 18 bp, synthetic construct
  source 1..18: mol_type = other DNA
gtgtgtttggaagcctct

SEQ ID NO: 296 - DNA, 18 bp, synthetic construct
  source 1..18: mol_type = other DNA
gtgtttggaagcctctgt

SEQ ID NO: 297 - DNA, 17 bp, synthetic construct
  source 1..17: mol_type = other DNA
ctgacacttggggtttg

SEQ ID NO: 298 - DNA, 16 bp, synthetic construct
  source 1..16: mol_type = other DNA
ggacataccattgttg

SEQ ID NO: 299 - DNA, 17 bp, synthetic construct
  source 1..17: mol_type = other DNA
ggacataccattgttgg

SEQ ID NO: 300 - DNA, 18 bp, synthetic construct
  source 1..18: mol_type = other DNA
ggacataccattgttgga

SEQ ID NO: 301 - DNA, 20 bp, synthetic construct
  source 1..20: mol_type = other DNA
acataccattgttggatgta

SEQ ID NO: 302 - DNA, 17 bp, synthetic construct
  source 1..17: mol_type = other DNA
ataccattgttggatgt

SEQ ID NO: 303 - DNA, 18 bp, synthetic construct
  source 1..18: mol_type = other DNA
ataccattgttggatgta

SEQ ID NO: 304 - DNA, 19 bp, synthetic construct
  source 1..19: mol_type = other DNA
ataccattgttggatgtat

SEQ ID NO: 305 - DNA, 20 bp, synthetic construct
  source 1..20: mol_type = other DNA
ataccattgttggatgtatt

SEQ ID NO: 306 - DNA, 18 bp, synthetic construct
  source 1..18: mol_type = other DNA
attgttggatgtattttt

SEQ ID NO: 307 - DNA, 19 bp, synthetic construct
  source 1..19: mol_type = other DNA
attgttggatgtatttttt

SEQ ID NO: 308 - DNA, 20 bp, synthetic construct
  source 1..20: mol_type = other DNA
attgttggatgtattttttt

SEQ ID NO: 309 - DNA, 17 bp, synthetic construct
  source 1..17: mol_type = other DNA
aaacagcaataattagc

SEQ ID NO: 310 - DNA, 18 bp, synthetic construct
  source 1..18: mol_type = other DNA
aaacagcaataattagcc

SEQ ID NO: 311 - DNA, 19 bp, synthetic construct
  source 1..19: mol_type = other DNA
aaacagcaataattagcca

SEQ ID NO: 312 - DNA, 17 bp, synthetic construct
  source 1..17: mol_type = other DNA
aataattagccatttta

SEQ ID NO: 313 - DNA, 18 bp, synthetic construct
  source 1..18: mol_type = other DNA
aataattagccattttaa

SEQ ID NO: 314 - DNA, 19 bp, synthetic construct
  source 1..19: mol_type = other DNA
aataattagccattttaaa

SEQ ID NO: 315 - DNA, 20 bp, synthetic construct
  source 1..20: mol_type = other DNA
aataattagccattttaaag

SEQ ID NO: 316 - DNA, 18 bp, synthetic construct
  source 1..18: mol_type = other DNA
ataattagccattttaaa

SEQ ID NO: 317 - DNA, 19 bp, synthetic construct
  source 1..19: mol_type = other DNA
ataattagccattttaaag

SEQ ID NO: 318 - DNA, 20 bp, synthetic construct
  source 1..20: mol_type = other DNA
ataattagccattttaaagg

SEQ ID NO: 319 - DNA, 19 bp, synthetic construct
  source 1..19: mol_type = other DNA
aattagccattttaaagga

SEQ ID NO: 320 - DNA, 20 bp, synthetic construct
  source 1..20: mol_type = other DNA
aattagccattttaaaggag

SEQ ID NO: 321 - DNA, 18 bp, synthetic construct
  source 1..18: mol_type = other DNA
attagccattttaaagga

SEQ ID NO: 322 - DNA, 19 bp, synthetic construct
  source 1..19: mol_type = other DNA
attagccattttaaaggag

SEQ ID NO: 323 - DNA, 16 bp, synthetic construct
  source 1..16: mol_type = other DNA
atgtgcacgtgtttga

SEQ ID NO: 324 - DNA, 17 bp, synthetic construct
  source 1..17: mol_type = other DNA
atgtgcacgtgtttgag

SEQ ID NO: 325 - DNA, 18 bp, synthetic construct
  source 1..18: mol_type = other DNA
atgtgcacgtgtttgagc

SEQ ID NO: 326 - DNA, 16 bp, synthetic construct
  source 1..16: mol_type = other DNA
gtgcacgtgtttgagc

SEQ ID NO: 327 - DNA, 17 bp, synthetic construct
  source 1..17: mol_type = other DNA
cgtgtttgagcgtgtgt

SEQ ID NO: 328 - DNA, 17 bp, synthetic construct
  source 1..17: mol_type = other DNA
gtgtgtgcaagtgggtt

SEQ ID NO: 329 - DNA, 18 bp, synthetic construct
  source 1..18: mol_type = other DNA
gtgtgcaagtgggttctt

SEQ ID NO: 330 - DNA, 16 bp, synthetic construct
  source 1..16: mol_type = other DNA
gtgcaagtgggttctt

SEQ ID NO: 331 - DNA, 17 bp, synthetic construct
  source 1..17: mol_type = other DNA
gtgcaagtgggttcttg

SEQ ID NO: 332 - DNA, 16 bp, synthetic construct
  source 1..16: mol_type = other DNA
gcaagtgggttcttgg

SEQ ID NO: 333 - DNA, 18 bp, synthetic construct
  source 1..18: mol_type = other DNA
caagtgggttcttggata

SEQ ID NO: 334 - DNA, 18 bp, synthetic construct
  source 1..18: mol_type = other DNA
cttggatatgtgtagtgt

SEQ ID NO: 335 - DNA, 16 bp, synthetic construct
  source 1..16: mol_type = other DNA
gatatgtgtagtgtga

SEQ ID NO: 336 - DNA, 17 bp, synthetic construct
  source 1..17: mol_type = other DNA
gcatgttgtaggtgcat

SEQ ID NO: 337 - DNA, 19 bp, synthetic construct
  source 1..19: mol_type = other DNA
catgttgtaggtgcataag

SEQ ID NO: 338 - DNA, 16 bp, synthetic construct
  source 1..16: mol_type = other DNA
gtgcataagcatgtac

SEQ ID NO: 339 - DNA, 17 bp, synthetic construct
  source 1..17: mol_type = other DNA
gtgcataagcatgtaca

SEQ ID NO: 340 - DNA, 15 bp, synthetic construct
  source 1..15: mol_type = other DNA
ataagcatgtacacg

SEQ ID NO: 341 - DNA, 15 bp, synthetic construct
  source 1..15: mol_type = other DNA
acacgcgtgagcatg

SEQ ID NO: 342 - DNA, 18 bp, synthetic construct
  source 1..18: mol_type = other DNA
aggtgtacatgtgcatga

SEQ ID NO: 343 - DNA, 19 bp, synthetic construct
  source 1..19: mol_type = other DNA
gtgtacatgtgcatgagtt

SEQ ID NO: 344 - DNA, 17 bp, synthetic construct
  source 1..17: mol_type = other DNA
gtacatgtgcatgagtt

SEQ ID NO: 345 - DNA, 19 bp, synthetic construct
  source 1..19: mol_type = other DNA
acgtgtatagatgtacgtg

SEQ ID NO: 346 - DNA, 17 bp, synthetic construct
  source 1..17: mol_type = other DNA
gcgtgtacatgtgtttg

SEQ ID NO: 347 - DNA, 19 bp, synthetic construct
  source 1..19: mol_type = other DNA
gtgtacatgtgtttgtgtc

SEQ ID NO: 348 - DNA, 18 bp, synthetic construct
  source 1..18: mol_type = other DNA
cgtgtgtatgaatgtgcg

SEQ ID NO: 349 - DNA, 17 bp, synthetic construct
  source 1..17: mol_type = other DNA
gcgtgtgtatgcatgag

SEQ ID NO: 350 - DNA, 16 bp, synthetic construct
  source 1..16: mol_type = other DNA
gtatgcatgagcacgt

SEQ ID NO: 351 - DNA, 17 bp, synthetic construct
  source 1..17: mol_type = other DNA
gtatgcatgagcacgta

SEQ ID NO: 352 - DNA, 18 bp, synthetic construct
  source 1..18: mol_type = other DNA
gtatgcatgagcacgtat

SEQ ID NO: 353 - DNA, 16 bp, synthetic construct
  source 1..16: mol_type = other DNA
cgcatgttcttgtgta

SEQ ID NO: 354 - DNA, 18 bp, synthetic construct
  source 1..18: mol_type = other DNA
cttgtgtacttctctgag

SEQ ID NO: 355 - DNA, 16 bp, synthetic construct
  source 1..16: mol_type = other DNA
gtacttctctgagggg

SEQ ID NO: 356 - DNA, 18 bp, synthetic construct
  source 1..18: mol_type = other DNA
catatgcatatggtgtac

SEQ ID NO: 357 - DNA, 18 bp, synthetic construct
  source 1..18: mol_type = other DNA
catgcacatgtgtatgga

SEQ ID NO: 358 - DNA, 18 bp, synthetic construct
  source 1..18: mol_type = other DNA
catgtgtatggatgcata

SEQ ID NO: 359 - DNA, 17 bp, synthetic construct
  source 1..17: mol_type = other DNA
atgtgtgttgcatgagc

SEQ ID NO: 360 - DNA, 18 bp, synthetic construct
  source 1..18: mol_type = other DNA
atgtgtgttgcatgagca

SEQ ID NO: 361 - DNA, 16 bp, synthetic construct
  source 1..16: mol_type = other DNA
gttgcatgagcatgca

SEQ ID NO: 362 - DNA, 17 bp, synthetic construct
  source 1..17: mol_type = other DNA
agtgcctgtgtgtgtac

SEQ ID NO: 363 - DNA, 19 bp, synthetic construct
  source 1..19: mol_type = other DNA
agtgcctgtgtgtgtactt

SEQ ID NO: 364 - DNA, 19 bp, synthetic construct
  source 1..19: mol_type = other DNA
gtgcctgtgtgtgtacttg

SEQ ID NO: 365 - DNA, 18 bp, synthetic construct
  source 1..18: mol_type = other DNA
gtgtgtgtacttgcatat

SEQ ID NO: 366 - DNA, 19 bp, synthetic construct
  source 1..19: mol_type = other DNA
gtgtgtgtacttgcatata

SEQ ID NO: 367 - DNA, 20 bp, synthetic construct
  source 1..20: mol_type = other DNA
gtgtgtgtacttgcatatat

SEQ ID NO: 368 - DNA, 17 bp, synthetic construct
  source 1..17: mol_type = other DNA
gtgtgtacttgcatata

SEQ ID NO: 369 - DNA, 18 bp, synthetic construct
  source 1..18: mol_type = other DNA
gtgtgtacttgcatatat

SEQ ID NO: 370 - DNA, 19 bp, synthetic construct
  source 1..19: mol_type = other DNA
gtgtgtacttgcatatatg

SEQ ID NO: 371 - DNA, 20 bp, synthetic construct
  source 1..20: mol_type = other DNA
gtgtgtacttgcatatatgt

SEQ ID NO: 372 - DNA, 17 bp, synthetic construct
  source 1..17: mol_type = other DNA
gtgtacttgcatatatg

SEQ ID NO: 373 - DNA, 18 bp, synthetic construct
  source 1..18: mol_type = other DNA
gtgtacttgcatatatgt

SEQ ID NO: 374 - DNA, 19 bp, synthetic construct
  source 1..19: mol_type = other DNA
gtgtacttgcatatatgtg

SEQ ID NO: 375 - DNA, 20 bp, synthetic construct
  source 1..20: mol_type = other DNA
gtgtacttgcatatatgtgg

SEQ ID NO: 376 - DNA, 17 bp, synthetic construct
  source 1..17: mol_type = other DNA
gtacttgcatatatgtg

SEQ ID NO: 377 - DNA, 18 bp, synthetic construct
  source 1..18: mol_type = other DNA
gtacttgcatatatgtgg

SEQ ID NO: 378 - DNA, 19 bp, synthetic construct
  source 1..19: mol_type = other DNA
gtacttgcatatatgtggc

SEQ ID NO: 379 - DNA, 20 bp, synthetic construct
  source 1..20: mol_type = other DNA
gtacttgcatatatgtggct

SEQ ID NO: 380 - DNA, 17 bp, synthetic construct
  source 1..17: mol_type = other DNA
gcatatatgtggctgtg

SEQ ID NO: 381 - DNA, 17 bp, synthetic construct
  source 1..17: mol_type = other DNA
atatatgtggctgtgca

SEQ ID NO: 382 - DNA, 18 bp, synthetic construct
  source 1..18: mol_type = other DNA
atatatgtggctgtgcag

SEQ ID NO: 383 - DNA, 16 bp, synthetic construct
  source 1..16: mol_type = other DNA
atatgtggctgtgcag

SEQ ID NO: 384 - DNA, 16 bp, synthetic construct
  source 1..16: mol_type = other DNA
gggcagattgttctga

SEQ ID NO: 385 - DNA, 17 bp, synthetic construct
  source 1..17: mol_type = other DNA
gggcagattgttctgag

SEQ ID NO: 386 - DNA, 16 bp, synthetic construct
  source 1..16: mol_type = other DNA
agatgtgccaaaacag

SEQ ID NO: 387 - DNA, 16 bp, synthetic construct
  source 1..16: mol_type = other DNA
gatgtgccaaaacagc

SEQ ID NO: 388 - DNA, 18 bp, synthetic construct
  source 1..18: mol_type = other DNA
caatgctgctgtttttca

SEQ ID NO: 389 - DNA, 17 bp, synthetic construct
  source 1..17: mol_type = other DNA
aatgctgctgtttttca

SEQ ID NO: 390 - DNA, 18 bp, synthetic construct
  source 1..18: mol_type = other DNA
aatgctgctgtttttcaa

SEQ ID NO: 391 - DNA, 19 bp, synthetic construct
  source 1..19: mol_type = other DNA
aatgctgctgtttttcaat

SEQ ID NO: 392 - DNA, 20 bp, synthetic construct
  source 1..20: mol_type = other DNA
aatgctgctgtttttcaata

SEQ ID NO: 393 - DNA, 19 bp, synthetic construct
  source 1..19: mol_type = other DNA
atgctgctgtttttcaata

SEQ ID NO: 394 - DNA, 20 bp, synthetic construct
  source 1..20: mol_type = other DNA
atgctgctgtttttcaataa

SEQ ID NO: 395 - DNA, 17 bp, synthetic construct
  source 1..17: mol_type = other DNA
gtttttcaataaaacca

SEQ ID NO: 396 - DNA, 18 bp, synthetic construct
  source 1..18: mol_type = other DNA
gtttttcaataaaaccag

SEQ ID NO: 397 - DNA, 19 bp, synthetic construct
  source 1..19: mol_type = other DNA
gtttttcaataaaaccaga

SEQ ID NO: 398 - DNA, 20 bp, synthetic construct
  source 1..20: mol_type = other DNA
gtttttcaataaaaccagag

SEQ ID NO: 399 - DNA, 19 bp, synthetic construct
  source 1..19: mol_type = other DNA
aataaaaccagagttgaag

SEQ ID NO: 400 - DNA, 18 bp, synthetic construct
  source 1..18: mol_type = other DNA
ataaaaccagagttgaag

SEQ ID NO: 401 - DNA, 17 bp, synthetic construct
  source 1..17: mol_type = other DNA
aaaaccagagttgaagg

SEQ ID NO: 402 - DNA, 17 bp, synthetic construct
  source 1..17: mol_type = other DNA
aaaccagagttgaaggc

SEQ ID NO: 403 - DNA, 16 bp, synthetic construct
  source 1..16: mol_type = other DNA
acaatgcatgcacccg

SEQ ID NO: 404 - DNA, 14 bp, synthetic construct
  source 1..14: mol_type = other DNA
ttaggcactcgccg

SEQ ID NO: 405 - DNA, 14 bp, synthetic construct
  source 1..14: mol_type = other DNA
atttaggcactcgc

SEQ ID NO: 406 - DNA, 15 bp, synthetic construct
  source 1..15: mol_type = other DNA
atatttaggcactcg

SEQ ID NO: 407 - DNA, 16 bp, synthetic construct
  source 1..16: mol_type = other DNA
ttatatttaggcactc

SEQ ID NO: 408 - DNA, 17 bp, synthetic construct
  source 1..17: mol_type = other DNA
attatatttaggcactc

SEQ ID NO: 409 - DNA, 18 bp, synthetic construct
  source 1..18: mol_type = other DNA
gattatatttaggcactc

SEQ ID NO: 410 - DNA, 19 bp, synthetic construct
  source 1..19: mol_type = other DNA
agattatatttaggcactc

SEQ ID NO: 411 - DNA, 20 bp, synthetic construct
  source 1..20: mol_type = other DNA
cagattatatttaggcactc

SEQ ID NO: 412 - DNA, 17 bp, synthetic construct
  source 1..17: mol_type = other DNA
gattatatttaggcact

SEQ ID NO: 413 - DNA, 18 bp, synthetic construct
  source 1..18: mol_type = other DNA
agattatatttaggcact

SEQ ID NO: 414 - DNA, 19 bp, synthetic construct
  source 1..19: mol_type = other DNA
cagattatatttaggcact

SEQ ID NO: 415 - DNA, 16 bp, synthetic construct
  source 1..16: mol_type = other DNA
gattatatttaggcac

SEQ ID NO: 416 - DNA, 17 bp, synthetic construct
  source 1..17: mol_type = other DNA
agattatatttaggcac

SEQ ID NO: 417 - DNA, 18 bp, synthetic construct
  source 1..18: mol_type = other DNA
cagattatatttaggcac

SEQ ID NO: 418 - DNA, 19 bp, synthetic construct
  source 1..19: mol_type = other DNA
gcagattatatttaggcac

SEQ ID NO: 419 - DNA, 16 bp, synthetic construct
  source 1..16: mol_type = other DNA
cccgcagattatattt

SEQ ID NO: 420 - DNA, 17 bp, synthetic construct
  source 1..17: mol_type = other DNA
gcccgcagattatattt

SEQ ID NO: 421 - DNA, 15 bp, synthetic construct
  source 1..15: mol_type = other DNA
cccgcagattatatt

SEQ ID NO: 422 - DNA, 16 bp, synthetic construct
  source 1..16: mol_type = other DNA
gcccgcagattatatt

SEQ ID NO: 423 - DNA, 17 bp, synthetic construct
  source 1..17: mol_type = other DNA
cgcccgcagattatatt

SEQ ID NO: 424 - DNA, 16 bp, synthetic construct
  source 1..16: mol_type = other DNA
ataaattagcaccgag

SEQ ID NO: 425 - DNA, 16 bp, synthetic construct
  source 1..16: mol_type = other DNA
ctcataaattagcacc

SEQ ID NO: 426 - DNA, 17 bp, synthetic construct
  source 1..17: mol_type = other DNA
ttcccctcataaattag

SEQ ID NO: 427 - DNA, 17 bp, synthetic construct
  source 1..17: mol_type = other DNA
tatttccaagcttcctg

SEQ ID NO: 428 - DNA, 17 bp, synthetic construct
  source 1..17: mol_type = other DNA
ctatttccaagcttcct

SEQ ID NO: 429 - DNA, 17 bp, synthetic construct
  source 1..17: mol_type = other DNA
tccctatttccaagctt

SEQ ID NO: 430 - DNA, 18 bp, synthetic construct
  source 1..18: mol_type = other DNA
tctacttctccctatttc

SEQ ID NO: 431 - DNA, 19 bp, synthetic construct
  source 1..19: mol_type = other DNA
ttctacttctccctatttc

SEQ ID NO: 432 - DNA, 17 bp, synthetic construct
  source 1..17: mol_type = other DNA
tctacttctccctattt

SEQ ID NO: 433 - DNA, 18 bp, synthetic construct
  source 1..18: mol_type = other DNA
ttctacttctccctattt

SEQ ID NO: 434 - DNA, 19 bp, synthetic construct
  source 1..19: mol_type = other DNA
tttctacttctccctattt

SEQ ID NO: 435 - DNA, 18 bp, synthetic construct
  source 1..18: mol_type = other DNA
tttctacttctccctatt

SEQ ID NO: 436 - DNA, 17 bp, synthetic construct
  source 1..17: mol_type = other DNA
cgggtgctttctacttc

SEQ ID NO: 437 - DNA, 16 bp, synthetic construct
  source 1..16: mol_type = other DNA
cgggtgctttctactt

SEQ ID NO: 438 - DNA, 17 bp, synthetic construct
  source 1..17: mol_type = other DNA
tcgggtgctttctactt

SEQ ID NO: 439 - DNA, 17 bp, synthetic construct
  source 1..17: mol_type = other DNA
tctcgggtgctttctac

SEQ ID NO: 440 - DNA, 16 bp, synthetic construct
  source 1..16: mol_type = other DNA
atctcgggtgctttct

SEQ ID NO: 441 - DNA, 15 bp, synthetic construct
  source 1..15: mol_type = other DNA
atctcgggtgctttc

SEQ ID NO: 442 - DNA, 16 bp, synthetic construct
  source 1..16: mol_type = other DNA
catctcgggtgctttc

SEQ ID NO: 443 - DNA, 16 bp, synthetic construct
  source 1..16: mol_type = other DNA
tcatctcgggtgcttt

SEQ ID NO: 444 - DNA, 14 bp, synthetic construct
  source 1..14: mol_type = other DNA
cggatcaccttgcg

SEQ ID NO: 445 - DNA, 14 bp, synthetic construct
  source 1..14: mol_type = other DNA
cgcggatcaccttg

SEQ ID NO: 446 - DNA, 14 bp, synthetic construct
  source 1..14: mol_type = other DNA
tcgatgtccgccat

SEQ ID NO: 447 - DNA, 16 bp, synthetic construct
  source 1..16: mol_type = other DNA
tactgctcgatgtccg

SEQ ID NO: 448 - DNA, 15 bp, synthetic construct
  source 1..15: mol_type = other DNA
tactgctcgatgtcc

SEQ ID NO: 449 - DNA, 16 bp, synthetic construct
  source 1..16: mol_type = other DNA
tcatgtagtactgctc

SEQ ID NO: 450 - DNA, 17 bp, synthetic construct
  source 1..17: mol_type = other DNA
ttcatgtagtactgctc

SEQ ID NO: 451 - DNA, 18 bp, synthetic construct
  source 1..18: mol_type = other DNA
cttcatgtagtactgctc

SEQ ID NO: 452 - DNA, 16 bp, synthetic construct
  source 1..16: mol_type = other DNA
ggcttcatgtagtact

SEQ ID NO: 453 - DNA, 16 bp, synthetic construct
  source 1..16: mol_type = other DNA
cggcttcatgtagtac

SEQ ID NO: 454 - DNA, 16 bp, synthetic construct
  source 1..16: mol_type = other DNA
agctcaggtcactcag

SEQ ID NO: 455 - DNA, 17 bp, synthetic construct
  source 1..17: mol_type = other DNA
aaaacccttcagctaag

SEQ ID NO: 456 - DNA, 17 bp, synthetic construct
  source 1..17: mol_type = other DNA
ctccagctatagaaggc

SEQ ID NO: 457 - DNA, 15 bp, synthetic construct
  source 1..15: mol_type = other DNA
tacccaggcaagacg

SEQ ID NO: 458 - DNA, 18 bp, synthetic construct
  source 1..18: mol_type = other DNA
gagctttcctataaatcc

SEQ ID NO: 459 - DNA, 17 bp, synthetic construct
  source 1..17: mol_type = other DNA
gagctttcctataaatc

SEQ ID NO: 460 - DNA, 18 bp, synthetic construct
  source 1..18: mol_type = other DNA
ggagctttcctataaatc

SEQ ID NO: 461 - DNA, 19 bp, synthetic construct
  source 1..19: mol_type = other DNA
aggagctttcctataaatc

SEQ ID NO: 462 - DNA, 16 bp, synthetic construct
  source 1..16: mol_type = other DNA
gagctttcctataaat

SEQ ID NO: 463 - DNA, 17 bp, synthetic construct
  source 1..17: mol_type = other DNA
ggagctttcctataaat

SEQ ID NO: 464 - DNA, 18 bp, synthetic construct
  source 1..18: mol_type = other DNA
aggagctttcctataaat

SEQ ID NO: 465 - DNA, 16 bp, synthetic construct
  source 1..16: mol_type = other DNA
cggcaaactccagcac

SEQ ID NO: 466 - DNA, 17 bp, synthetic construct
  source 1..17: mol_type = other DNA
acggcaaactccagcac

SEQ ID NO: 467 - DNA, 16 bp, synthetic construct
  source 1..16: mol_type = other DNA
catcacggcaaactcc

SEQ ID NO: 468 - DNA, 16 bp, synthetic construct
  source 1..16: mol_type = other DNA
ccatcacggcaaactc

SEQ ID NO: 469 - DNA, 17 bp, synthetic construct
  source 1..17: mol_type = other DNA
gccatcacggcaaactc

SEQ ID NO: 470 - DNA, 15 bp, synthetic construct
  source 1..15: mol_type = other DNA
ccatcacggcaaact

SEQ ID NO: 471 - DNA, 16 bp, synthetic construct
  source 1..16: mol_type = other DNA
gccatcacggcaaact

SEQ ID NO: 472 - DNA, 16 bp, synthetic construct
  source 1..16: mol_type = other DNA
accctgaacaggccat

SEQ ID NO: 473 - DNA, 16 bp, synthetic construct
  source 1..16: mol_type = other DNA
aacagccaggacccac

SEQ ID NO: 474 - DNA, 16 bp, synthetic construct
  source 1..16: mol_type = other DNA
gcccaacacctgttac

SEQ ID NO: 475 - DNA, 16 bp, synthetic construct
  source 1..16: mol_type = other DNA
aaagccagcccaacac

SEQ ID NO: 476 - DNA, 17 bp, synthetic construct
  source 1..17: mol_type = other DNA
gaaagccagcccaacac

SEQ ID NO: 477 - DNA, 16 bp, synthetic construct
  source 1..16: mol_type = other DNA
actattcaggacaagc

SEQ ID NO: 478 - DNA, 16 bp, synthetic construct
  source 1..16: mol_type = other DNA
cactattcaggacaag

SEQ ID NO: 479 - DNA, 15 bp, synthetic construct
  source 1..15: mol_type = other DNA
caccgtgttgtcctc

SEQ ID NO: 480 - DNA, 16 bp, synthetic construct
  source 1..16: mol_type = other DNA
gaactccagcaccttc

SEQ ID NO: 481 - DNA, 16 bp, synthetic construct
  source 1..16: mol_type = other DNA
cgaactccagcacctt

SEQ ID NO: 482 - DNA, 15 bp, synthetic construct
  source 1..15: mol_type = other DNA
accacggcgaactcc

SEQ ID NO: 483 - DNA, 14 bp, synthetic construct
  source 1..14: mol_type = other DNA
accacggcgaactc

SEQ ID NO: 484 - DNA, 15 bp, synthetic construct
  source 1..15: mol_type = other DNA
caccacggcgaactc

SEQ ID NO: 485 - DNA, 15 bp, synthetic construct
  source 1..15: mol_type = other DNA
tagttgtgcaccacg

SEQ ID NO: 486 - DNA, 14 bp, synthetic construct
  source 1..14: mol_type = other DNA
caccttgacggccg

SEQ ID NO: 487 - DNA, 14 bp, synthetic construct
  source 1..14: mol_type = other DNA
ccagcgactcgtag

SEQ ID NO: 488 - DNA, 20 bp, synthetic construct
  source 1..20: mol_type = other DNA
caacacaaacgccaattgag

SEQ ID NO: 489 - DNA, 16 bp, synthetic construct
  source 1..16: mol_type = other DNA
gaaccagccagacaac

SEQ ID NO: 490 - DNA, 17 bp, synthetic construct
  source 1..17: mol_type = other DNA
tccaagatgccactctt

SEQ ID NO: 491 - DNA, 16 bp, synthetic construct
  source 1..16: mol_type = other DNA
gtccaagatgccactc

SEQ ID NO: 492 - DNA, 16 bp, synthetic construct
  source 1..16: mol_type = other DNA
tgataagattcacagg

SEQ ID NO: 493 - DNA, 20 bp, synthetic construct
  source 1..20: mol_type = other DNA
taaataacttattagaaaat

SEQ ID NO: 494 - DNA, 17 bp, synthetic construct
  source 1..17: mol_type = other DNA
tgtctaaataacttatt

SEQ ID NO: 495 - DNA, 18 bp, synthetic construct
  source 1..18: mol_type = other DNA
ctgtctaaataacttatt

SEQ ID NO: 496 - DNA, 17 bp, synthetic construct
  source 1..17: mol_type = other DNA
ctgtctaaataacttat

SEQ ID NO: 497 - DNA, 18 bp, synthetic construct
  source 1..18: mol_type = other DNA
tctgtctaaataacttat

SEQ ID NO: 498 - DNA, 19 bp, synthetic construct
  source 1..19: mol_type = other DNA
ttctgtctaaataacttat

SEQ ID NO: 499 - DNA, 20 bp, synthetic construct
  source 1..20: mol_type = other DNA
attctgtctaaataacttat

SEQ ID NO: 500 - DNA, 18 bp, synthetic construct
  source 1..18: mol_type = other DNA
attctgtctaaataactt

SEQ ID NO: 501 - DNA, 19 bp, synthetic construct
  source 1..19: mol_type = other DNA
tattctgtctaaataactt

SEQ ID NO: 502 - DNA, 20 bp, synthetic construct
  source 1..20: mol_type = other DNA
ctattctgtctaaataactt

SEQ ID NO: 503 - DNA, 18 bp, synthetic construct
  source 1..18: mol_type = other DNA
tattctgtctaaataact

SEQ ID NO: 504 - DNA, 19 bp, synthetic construct
  source 1..19: mol_type = other DNA
ctattctgtctaaataact

SEQ ID NO: 505 - DNA, 20 bp, synthetic construct
  source 1..20: mol_type = other DNA
gctattctgtctaaataact

SEQ ID NO: 506 - DNA, 17 bp, synthetic construct
  source 1..17: mol_type = other DNA
tattctgtctaaataac

SEQ ID NO: 507 - DNA, 18 bp, synthetic construct
  source 1..18: mol_type = other DNA
ctattctgtctaaataac

SEQ ID NO: 508 - DNA, 18 bp, synthetic construct
  source 1..18: mol_type = other DNA
gtgctattctgtctaaat

SEQ ID NO: 509 - DNA, 19 bp, synthetic construct
  source 1..19: mol_type = other DNA
agtgctattctgtctaaat

SEQ ID NO: 510 - DNA, 17 bp, synthetic construct
  source 1..17: mol_type = other DNA
agagtgctattctgtct

SEQ ID NO: 511 - DNA, 17 bp, synthetic construct
  source 1..17: mol_type = other DNA
atgcagagtgctattct

SEQ ID NO: 512 - DNA, 17 bp, synthetic construct
  source 1..17: mol_type = other DNA
ccaagaattaaagtcat

SEQ ID NO: 513 - DNA, 18 bp, synthetic construct
  source 1..18: mol_type = other DNA
cccaagaattaaagtcat

SEQ ID NO: 514 - DNA, 19 bp, synthetic construct
  source 1..19: mol_type = other DNA
tcccaagaattaaagtcat

SEQ ID NO: 515 - DNA, 20 bp, synthetic construct
  source 1..20: mol_type = other DNA
gtcccaagaattaaagtcat

SEQ ID NO: 516 - DNA, 17 bp, synthetic construct
  source 1..17: mol_type = other DNA
gttttgtcccaagaatt

SEQ ID NO: 517 - DNA, 18 bp, synthetic construct
  source 1..18: mol_type = other DNA
cgttttgtcccaagaatt

SEQ ID NO: 518 - DNA, 15 bp, synthetic construct
  source 1..15: mol_type = other DNA
ctaccgttttgtccc

SEQ ID NO: 519 - DNA, 17 bp, synthetic construct
  source 1..17: mol_type = other DNA
aactaccgttttgtccc

SEQ ID NO: 520 - DNA, 18 bp, synthetic construct
  source 1..18: mol_type = other DNA
aaactaccgttttgtccc

SEQ ID NO: 521 - DNA, 15 bp, synthetic construct
  source 1..15: mol_type = other DNA
actaccgttttgtcc

SEQ ID NO: 522 - DNA, 16 bp, synthetic construct
  source 1..16: mol_type = other DNA
aactaccgttttgtcc

SEQ ID NO: 523 - DNA, 17 bp, synthetic construct
  source 1..17: mol_type = other DNA
aaactaccgttttgtcc

SEQ ID NO: 524 - DNA, 18 bp, synthetic construct
  source 1..18: mol_type = other DNA
caaactaccgttttgtcc

SEQ ID NO: 525 - DNA, 17 bp, synthetic construct
  source 1..17: mol_type = other DNA
gtacaaactaccgtttt

SEQ ID NO: 526 - DNA, 18 bp, synthetic construct
  source 1..18: mol_type = other DNA
ggtacaaactaccgtttt

SEQ ID NO: 527 - DNA, 16 bp, synthetic construct
  source 1..16: mol_type = other DNA
gtacaaactaccgttt

SEQ ID NO: 528 - DNA, 17 bp, synthetic construct
  source 1..17: mol_type = other DNA
ggtacaaactaccgttt

SEQ ID NO: 529 - DNA, 16 bp, synthetic construct
  source 1..16: mol_type = other DNA
cacctcaattgctaac

SEQ ID NO: 530 - DNA, 17 bp, synthetic construct
  source 1..17: mol_type = other DNA
acacctcaattgctaac

SEQ ID NO: 531 - DNA, 18 bp, synthetic construct
  source 1..18: mol_type = other DNA
cacacctcaattgctaac

SEQ ID NO: 532 - DNA, 19 bp, synthetic construct
  source 1..19: mol_type = other DNA
gcacacctcaattgctaac

SEQ ID NO: 533 - DNA, 17 bp, synthetic construct
  source 1..17: mol_type = other DNA
ctctgcacacctcaatt

SEQ ID NO: 534 - DNA, 16 bp, synthetic construct
  source 1..16: mol_type = other DNA
ctctgcacacctcaat

SEQ ID NO: 535 - DNA, 17 bp, synthetic construct
  source 1..17: mol_type = other DNA
tacaaaactcccatccc

SEQ ID NO: 536 - DNA, 17 bp, synthetic construct
  source 1..17: mol_type = other DNA
ctacaaaactcccatcc

SEQ ID NO: 537 - DNA, 18 bp, synthetic construct
  source 1..18: mol_type = other DNA
tctacaaaactcccatcc

SEQ ID NO: 538 - DNA, 16 bp, synthetic construct
  source 1..16: mol_type = other DNA
ctacaaaactcccatc

SEQ ID NO: 539 - DNA, 17 bp, synthetic construct
  source 1..17: mol_type = other DNA
tctacaaaactcccatc

SEQ ID NO: 540 - DNA, 18 bp, synthetic construct
  source 1..18: mol_type = other DNA
gtctacaaaactcccatc

SEQ ID NO: 541 - DNA, 19 bp, synthetic construct
  source 1..19: mol_type = other DNA
agtctacaaaactcccatc

SEQ ID NO: 542 - DNA, 16 bp, synthetic construct
  source 1..16: mol_type = other DNA
tctacaaaactcccat

SEQ ID NO: 543 - DNA, 17 bp, synthetic construct
  source 1..17: mol_type = other DNA
gtctacaaaactcccat

SEQ ID NO: 544 - DNA, 18 bp, synthetic construct
  source 1..18: mol_type = other DNA
agtctacaaaactcccat

SEQ ID NO: 545 - DNA, 19 bp, synthetic construct
  source 1..19: mol_type = other DNA
cagtctacaaaactcccat

SEQ ID NO: 546 - DNA, 20 bp, synthetic construct
  source 1..20: mol_type = other DNA
acagtctacaaaactcccat

SEQ ID NO: 547 - DNA, 16 bp, synthetic construct
  source 1..16: mol_type = other DNA
agtctacaaaactccc

SEQ ID NO: 548 - DNA, 17 bp, synthetic construct
  source 1..17: mol_type = other DNA
cagtctacaaaactccc

SEQ ID NO: 549 - DNA, 18 bp, synthetic construct
  source 1..18: mol_type = other DNA
acagtctacaaaactccc

SEQ ID NO: 550 - DNA, 19 bp, synthetic construct
  source 1..19: mol_type = other DNA
tacagtctacaaaactccc

SEQ ID NO: 551 - DNA, 16 bp, synthetic construct
  source 1..16: mol_type = other DNA
cagtctacaaaactcc

SEQ ID NO: 552 - DNA, 17 bp, synthetic construct
  source 1..17: mol_type = other DNA
acagtctacaaaactcc

SEQ ID NO: 553 - DNA, 18 bp, synthetic construct
  source 1..18: mol_type = other DNA
tacagtctacaaaactcc

SEQ ID NO: 554 - DNA, 17 bp, synthetic construct
  source 1..17: mol_type = other DNA
tacagtctacaaaactc

SEQ ID NO: 555 - DNA, 18 bp, synthetic construct
  source 1..18: mol_type = other DNA
tctgtctgtatattccag

SEQ ID NO: 556 - DNA, 18 bp, synthetic construct
  source 1..18: mol_type = other DNA
cattttgctaaaacactt

SEQ ID NO: 557 - DNA, 19 bp, synthetic construct
  source 1..19: mol_type = other DNA
ccattttgctaaaacactt

SEQ ID NO: 558 - DNA, 20 bp, synthetic construct
  source 1..20: mol_type = other DNA
tccattttgctaaaacactt

SEQ ID NO: 559 - DNA, 17 bp, synthetic construct
  source 1..17: mol_type = other DNA
cattttgctaaaacact

SEQ ID NO: 560 - DNA, 18 bp, synthetic construct
  source 1..18: mol_type = other DNA
ccattttgctaaaacact

SEQ ID NO: 561 - DNA, 19 bp, synthetic construct
  source 1..19: mol_type = other DNA
tccattttgctaaaacact

SEQ ID NO: 562 - DNA, 20 bp, synthetic construct
  source 1..20: mol_type = other DNA
ttccattttgctaaaacact

SEQ ID NO: 563 - DNA, 17 bp, synthetic construct
  source 1..17: mol_type = other DNA
ccattttgctaaaacac

SEQ ID NO: 564 - DNA, 18 bp, synthetic construct
  source 1..18: mol_type = other DNA
tccattttgctaaaacac

SEQ ID NO: 565 - DNA, 19 bp, synthetic construct
  source 1..19: mol_type = other DNA
ttccattttgctaaaacac

SEQ ID NO: 566 - DNA, 20 bp, synthetic construct
  source 1..20: mol_type = other DNA
tttccattttgctaaaacac

SEQ ID NO: 567 - DNA, 18 bp, synthetic construct
  source 1..18: mol_type = other DNA
tttccattttgctaaaac

SEQ ID NO: 568 - DNA, 19 bp, synthetic construct
  source 1..19: mol_type = other DNA
gtttccattttgctaaaac

SEQ ID NO: 569 - DNA, 20 bp, synthetic construct
  source 1..20: mol_type = other DNA
tgtttccattttgctaaaac

SEQ ID NO: 570 - DNA, 18 bp, synthetic construct
  source 1..18: mol_type = other DNA
actgtttgtttccatttt

SEQ ID NO: 571 - DNA, 19 bp, synthetic construct
  source 1..19: mol_type = other DNA
aactgtttgtttccatttt

SEQ ID NO: 572 - DNA, 20 bp, synthetic construct
  source 1..20: mol_type = other DNA
caactgtttgtttccatttt

SEQ ID NO: 573 - DNA, 18 bp, synthetic construct
  source 1..18: mol_type = other DNA
aactgtttgtttccattt

SEQ ID NO: 574 - DNA, 19 bp, synthetic construct
  source 1..19: mol_type = other DNA
caactgtttgtttccattt

SEQ ID NO: 575 - DNA, 20 bp, synthetic construct
  source 1..20: mol_type = other DNA
acaactgtttgtttccattt

SEQ ID NO: 576 - DNA, 18 bp, synthetic construct
  source 1..18: mol_type = other DNA
caactgtttgtttccatt

SEQ ID NO: 577 - DNA, 19 bp, synthetic construct
  source 1..19: mol_type = other DNA
acaactgtttgtttccatt

SEQ ID NO: 578 - DNA, 20 bp, synthetic construct
  source 1..20: mol_type = other DNA
cacaactgtttgtttccatt

SEQ ID NO: 579 - DNA, 17 bp, synthetic construct
  source 1..17: mol_type = other DNA
caactgtttgtttccat

SEQ ID NO: 580 - DNA, 18 bp, synthetic construct
  source 1..18: mol_type = other DNA
acaactgtttgtttccat

SEQ ID NO: 581 - DNA, 19 bp, synthetic construct
  source 1..19: mol_type = other DNA
cacaactgtttgtttccat

SEQ ID NO: 582 - DNA, 17 bp, synthetic construct
  source 1..17: mol_type = other DNA
cacaactgtttgtttcc

SEQ ID NO: 583 - DNA, 17 bp, synthetic construct
  source 1..17: mol_type = other DNA
gcacaactgtttgtttc

SEQ ID NO: 584 - DNA, 17 bp, synthetic construct
  source 1..17: mol_type = other DNA
ggcacaactgtttgttt

SEQ ID NO: 585 - DNA, 18 bp, synthetic construct
  source 1..18: mol_type = other DNA
aggcacaactgtttgttt

SEQ ID NO: 586 - DNA, 19 bp, synthetic construct
  source 1..19: mol_type = other DNA
aaggcacaactgtttgttt

SEQ ID NO: 587 - DNA, 20 bp, synthetic construct
  source 1..20: mol_type = other DNA
aaaggcacaactgtttgttt

SEQ ID NO: 588 - DNA, 17 bp, synthetic construct
  source 1..17: mol_type = other DNA
aaaaggcacaactgttt

SEQ ID NO: 589 - DNA, 18 bp, synthetic construct
  source 1..18: mol_type = other DNA
aaaaaggcacaactgttt

SEQ ID NO: 590 - DNA, 19 bp, synthetic construct
  source 1..19: mol_type = other DNA
gaaaaaggcacaactgttt

SEQ ID NO: 591 - DNA, 19 bp, synthetic construct
  source 1..19: mol_type = other DNA
acccaaaccaaaccaaaag

SEQ ID NO: 592 - DNA, 18 bp, synthetic construct
  source 1..18: mol_type = other DNA
gccaaacccaaaccaaac

SEQ ID NO: 593 - DNA, 15 bp, synthetic construct
  source 1..15: mol_type = other DNA
accccgacatgacac

SEQ ID NO: 594 - DNA, 16 bp, synthetic construct
  source 1..16: mol_type = other DNA
caccccgacatgacac

SEQ ID NO: 595 - DNA, 16 bp, synthetic construct
  source 1..16: mol_type = other DNA
gatcaatatcaaggag

SEQ ID NO: 596 - DNA, 20 bp, synthetic construct
  source 1..20: mol_type = other DNA
ctgctaggatcaatatcaag

SEQ ID NO: 597 - DNA, 15 bp, synthetic construct
  source 1..15: mol_type = other DNA
acaaccaccccgcac

SEQ ID NO: 598 - DNA, 15 bp, synthetic construct
  source 1..15: mol_type = other DNA
cacgtcagtgcacat

SEQ ID NO: 599 - DNA, 16 bp, synthetic construct
  source 1..16: mol_type = other DNA
tcacgtcagtgcacat

SEQ ID NO: 600 - DNA, 17 bp, synthetic construct
  source 1..17: mol_type = other DNA
ttcacgtcagtgcacat

SEQ ID NO: 601 - DNA, 18 bp, synthetic construct
  source 1..18: mol_type = other DNA
attcacgtcagtgcacat

SEQ ID NO: 602 - DNA, 19 bp, synthetic construct
  source 1..19: mol_type = other DNA
aattcacgtcagtgcacat

SEQ ID NO: 603 - DNA, 20 bp, synthetic construct
  source 1..20: mol_type = other DNA
aaattcacgtcagtgcacat

SEQ ID NO: 604 - DNA, 15 bp, synthetic construct
  source 1..15: mol_type = other DNA
ttcacgtcagtgcac

SEQ ID NO: 605 - DNA, 16 bp, synthetic construct
  source 1..16: mol_type = other DNA
attcacgtcagtgcac

SEQ ID NO: 606 - DNA, 17 bp, synthetic construct
  source 1..17: mol_type = other DNA
aattcacgtcagtgcac

SEQ ID NO: 607 - DNA, 18 bp, synthetic construct
  source 1..18: mol_type = other DNA
aaattcacgtcagtgcac

SEQ ID NO: 608 - DNA, 19 bp, synthetic construct
  source 1..19: mol_type = other DNA
aaaattcacgtcagtgcac

SEQ ID NO: 609 - DNA, 20 bp, synthetic construct
  source 1..20: mol_type = other DNA
taaaattcacgtcagtgcac

SEQ ID NO: 610 - DNA, 15 bp, synthetic construct
  source 1..15: mol_type = other DNA
cgtataaaattcacg

SEQ ID NO: 611 - DNA, 19 bp, synthetic construct
  source 1..19: mol_type = other DNA
tcagagtttctacaaacgt

SEQ ID NO: 612 - DNA, 17 bp, synthetic construct
  source 1..17: mol_type = other DNA
cagagtttctacaaacg

SEQ ID NO: 613 - DNA, 16 bp, synthetic construct
  source 1..16: mol_type = other DNA
tagaagaagttacatc

SEQ ID NO: 614 - DNA, 17 bp, synthetic construct
  source 1..17: mol_type = other DNA
gtagaagaagttacatc

SEQ ID NO: 615 - DNA, 17 bp, synthetic construct
  source 1..17: mol_type = other DNA
gaggcttccaaacacac

SEQ ID NO: 616 - DNA, 18 bp, synthetic construct
  source 1..18: mol_type = other DNA
agaggcttccaaacacac

SEQ ID NO: 617 - DNA, 18 bp, synthetic construct
  source 1..18: mol_type = other DNA
acagaggcttccaaacac

SEQ ID NO: 618 - DNA, 17 bp, synthetic construct
  source 1..17: mol_type = other DNA
caaaccccaagtgtcag

SEQ ID NO: 619 - DNA, 16 bp, synthetic construct
  source 1..16: mol_type = other DNA
caacaatggtatgtcc

SEQ ID NO: 620 - DNA, 17 bp, synthetic construct
  source 1..17: mol_type = other DNA
ccaacaatggtatgtcc

SEQ ID NO: 621 - DNA, 18 bp, synthetic construct
  source 1..18: mol_type = other DNA
tccaacaatggtatgtcc

SEQ ID NO: 622 - DNA, 20 bp, synthetic construct
  source 1..20: mol_type = other DNA
tacatccaacaatggtatgt

SEQ ID NO: 623 - DNA, 17 bp, synthetic construct
  source 1..17: mol_type = other DNA
acatccaacaatggtat

SEQ ID NO: 624 - DNA, 18 bp, synthetic construct
  source 1..18: mol_type = other DNA
tacatccaacaatggtat

SEQ ID NO: 625 - DNA, 19 bp, synthetic construct
  source 1..19: mol_type = other DNA
atacatccaacaatggtat

SEQ ID NO: 626 - DNA, 20 bp, synthetic construct
  source 1..20: mol_type = other DNA
aatacatccaacaatggtat

SEQ ID NO: 627 - DNA, 18 bp, synthetic construct
  source 1..18: mol_type = other DNA
aaaaatacatccaacaat

SEQ ID NO: 628 - DNA, 19 bp, synthetic construct
  source 1..19: mol_type = other DNA
aaaaaatacatccaacaat

SEQ ID NO: 629 - DNA, 20 bp, synthetic construct
  source 1..20: mol_type = other DNA
aaaaaaatacatccaacaat

SEQ ID NO: 630 - DNA, 17 bp, synthetic construct
  source 1..17: mol_type = other DNA
gctaattattgctgttt

SEQ ID NO: 631 - DNA, 18 bp, synthetic construct
  source 1..18: mol_type = other DNA
ggctaattattgctgttt

SEQ ID NO: 632 - DNA, 19 bp, synthetic construct
  source 1..19: mol_type = other DNA
tggctaattattgctgttt

SEQ ID NO: 633 - DNA, 18 bp, synthetic construct
  source 1..18: mol_type = other DNA
ttaaaatggctaattatt

SEQ ID NO: 634 - DNA, 18 bp, synthetic construct
  source 1..18: mol_type = other DNA
ttaaaatggctaattatt

SEQ ID NO: 635 - DNA, 19 bp, synthetic construct
  source 1..19: mol_type = other DNA
tttaaaatggctaattatt

SEQ ID NO: 636 - DNA, 20 bp, synthetic construct
  source 1..20: mol_type = other DNA
ctttaaaatggctaattatt

SEQ ID NO: 637 - DNA, 18 bp, synthetic construct
  source 1..18: mol_type = other DNA
tttaaaatggctaattat

SEQ ID NO: 638 - DNA, 19 bp, synthetic construct
  source 1..19: mol_type = other DNA
ctttaaaatggctaattat

SEQ ID NO: 639 - DNA, 20 bp, synthetic construct
  source 1..20: mol_type = other DNA
cctttaaaatggctaattat

SEQ ID NO: 640 - DNA, 19 bp, synthetic construct
  source 1..19: mol_type = other DNA
tcctttaaaatggctaatt

SEQ ID NO: 641 - DNA, 20 bp, synthetic construct
  source 1..20: mol_type = other DNA
ctcctttaaaatggctaatt

SEQ ID NO: 642 - DNA, 18 bp, synthetic construct
  source 1..18: mol_type = other DNA
tcctttaaaatggctaat

SEQ ID NO: 643 - DNA, 19 bp, synthetic construct
  source 1..19: mol_type = other DNA
ctcctttaaaatggctaat

SEQ ID NO: 644 - DNA, 16 bp, synthetic construct
  source 1..16: mol_type = other DNA
tcaaacacgtgcacat

SEQ ID NO: 645 - DNA, 17 bp, synthetic construct
  source 1..17: mol_type = other DNA
ctcaaacacgtgcacat

SEQ ID NO: 646 - DNA, 18 bp, synthetic construct
  source 1..18: mol_type = other DNA
gctcaaacacgtgcacat

SEQ ID NO: 647 - DNA, 16 bp, synthetic construct
  source 1..16: mol_type = other DNA
gctcaaacacgtgcac

SEQ ID NO: 648 - DNA, 17 bp, synthetic construct
  source 1..17: mol_type = other DNA
acacacgctcaaacacg

SEQ ID NO: 649 - DNA, 17 bp, synthetic construct
  source 1..17: mol_type = other DNA
aacccacttgcacacac

SEQ ID NO: 650 - DNA, 18 bp, synthetic construct
  source 1..18: mol_type = other DNA
aagaacccacttgcacac

SEQ ID NO: 651 - DNA, 16 bp, synthetic construct
  source 1..16: mol_type = other DNA
aagaacccacttgcac

SEQ ID NO: 652 - DNA, 17 bp, synthetic construct
  source 1..17: mol_type = other DNA
caagaacccacttgcac

SEQ ID NO: 653 - DNA, 16 bp, synthetic construct
  source 1..16: mol_type = other DNA
ccaagaacccacttgc

SEQ ID NO: 654 - DNA, 18 bp, synthetic construct
  source 1..18: mol_type = other DNA
tatccaagaacccacttg

SEQ ID NO: 655 - DNA, 18 bp, synthetic construct
  source 1..18: mol_type = other DNA
acactacacatatccaag

SEQ ID NO: 656 - DNA, 16 bp, synthetic construct
  source 1..16: mol_type = other DNA
tcacactacacatatc

SEQ ID NO: 657 - DNA, 17 bp, synthetic construct
  source 1..17: mol_type = other DNA
atgcacctacaacatgc

SEQ ID NO: 658 - DNA, 19 bp, synthetic construct
  source 1..19: mol_type = other DNA
cttatgcacctacaacatg

SEQ ID NO: 659 - DNA, 16 bp, synthetic construct
  source 1..16: mol_type = other DNA
gtacatgcttatgcac

SEQ ID NO: 660 - DNA, 17 bp, synthetic construct
  source 1..17: mol_type = other DNA
tgtacatgcttatgcac

SEQ ID NO: 661 - DNA, 15 bp, synthetic construct
  source 1..15: mol_type = other DNA
cgtgtacatgcttat

SEQ ID NO: 662 - DNA, 15 bp, synthetic construct
  source 1..15: mol_type = other DNA
catgctcacgcgtgt

SEQ ID NO: 663 - DNA, 18 bp, synthetic construct
  source 1..18: mol_type = other DNA
tcatgcacatgtacacct

SEQ ID NO: 664 - DNA, 19 bp, synthetic construct
  source 1..19: mol_type = other DNA
aactcatgcacatgtacac

SEQ ID NO: 665 - DNA, 17 bp, synthetic construct
  source 1..17: mol_type = other DNA
aactcatgcacatgtac

SEQ ID NO: 666 - DNA, 19 bp, synthetic construct
  source 1..19: mol_type = other DNA
cacgtacatctatacacgt

SEQ ID NO: 667 - DNA, 17 bp, synthetic construct
  source 1..17: mol_type = other DNA
caaacacatgtacacgc

SEQ ID NO: 668 - DNA, 19 bp, synthetic construct
  source 1..19: mol_type = other DNA
gacacaaacacatgtacac

SEQ ID NO: 669 - DNA, 18 bp, synthetic construct
  source 1..18: mol_type = other DNA
cgcacattcatacacacg

SEQ ID NO: 670 - DNA, 17 bp, synthetic construct
  source 1..17: mol_type = other DNA
ctcatgcatacacacgc

SEQ ID NO: 671 - DNA, 16 bp, synthetic construct
  source 1..16: mol_type = other DNA
acgtgctcatgcatac

SEQ ID NO: 672 - DNA, 17 bp, synthetic construct
  source 1..17: mol_type = other DNA
tacgtgctcatgcatac

SEQ ID NO: 673 - DNA, 18 bp, synthetic construct
  source 1..18: mol_type = other DNA
atacgtgctcatgcatac

SEQ ID NO: 674 - DNA, 16 bp, synthetic construct
  source 1..16: mol_type = other DNA
tacacaagaacatgcg

SEQ ID NO: 675 - DNA, 18 bp, synthetic construct
  source 1..18: mol_type = other DNA
ctcagagaagtacacaag

SEQ ID NO: 676 - DNA, 16 bp, synthetic construct
  source 1..16: mol_type = other DNA
cccctcagagaagtac

SEQ ID NO: 677 - DNA, 18 bp, synthetic construct
  source 1..18: mol_type = other DNA
gtacaccatatgcatatg

SEQ ID NO: 678 - DNA, 18 bp, synthetic construct
  source 1..18: mol_type = other DNA
tccatacacatgtgcatg

SEQ ID NO: 679 - DNA, 18 bp, synthetic construct
  source 1..18: mol_type = other DNA
tatgcatccatacacatg

SEQ ID NO: 680 - DNA, 17 bp, synthetic construct
  source 1..17: mol_type = other DNA
gctcatgcaacacacat

SEQ ID NO: 681 - DNA, 18 bp, synthetic construct
  source 1..18: mol_type = other DNA
tgctcatgcaacacacat

SEQ ID NO: 682 - DNA, 16 bp, synthetic construct
  source 1..16: mol_type = other DNA
tgcatgctcatgcaac

SEQ ID NO: 683 - DNA, 17 bp, synthetic construct
  source 1..17: mol_type = other DNA
gtacacacacaggcact

SEQ ID NO: 684 - DNA, 19 bp, synthetic construct
  source 1..19: mol_type = other DNA
aagtacacacacaggcact

SEQ ID NO: 685 - DNA, 19 bp, synthetic construct
  source 1..19: mol_type = other DNA
caagtacacacacaggcac

SEQ ID NO: 686 - DNA, 18 bp, synthetic construct
  source 1..18: mol_type = other DNA
atatgcaagtacacacac

SEQ ID NO: 687 - DNA, 19 bp, synthetic construct
  source 1..19: mol_type = other DNA
tatatgcaagtacacacac

SEQ ID NO: 688 - DNA, 20 bp, synthetic construct
  source 1..20: mol_type = other DNA
atatatgcaagtacacacac

SEQ ID NO: 689 - DNA, 17 bp, synthetic construct
  source 1..17: mol_type = other DNA
tatatgcaagtacacac

SEQ ID NO: 690 - DNA, 18 bp, synthetic construct
  source 1..18: mol_type = other DNA
atatatgcaagtacacac

SEQ ID NO: 691 - DNA, 19 bp, synthetic construct
  source 1..19: mol_type = other DNA
catatatgcaagtacacac

SEQ ID NO: 692 - DNA, 20 bp, synthetic construct
  source 1..20: mol_type = other DNA
acatatatgcaagtacacac

SEQ ID NO: 693 - DNA, 17 bp, synthetic construct
  source 1..17: mol_type = other DNA
catatatgcaagtacac

SEQ ID NO: 694 - DNA, 18 bp, synthetic construct
  source 1..18: mol_type = other DNA
acatatatgcaagtacac

SEQ ID NO: 695 - DNA, 19 bp, synthetic construct
  source 1..19: mol_type = other DNA
cacatatatgcaagtacac

SEQ ID NO: 696 - DNA, 20 bp, synthetic construct
  source 1..20: mol_type = other DNA
ccacatatatgcaagtacac

SEQ ID NO: 697 - DNA, 17 bp, synthetic construct
  source 1..17: mol_type = other DNA
cacatatatgcaagtac

SEQ ID NO: 698 - DNA, 18 bp, synthetic construct
  source 1..18: mol_type = other DNA
ccacatatatgcaagtac

SEQ ID NO: 699 - DNA, 19 bp, synthetic construct
  source 1..19: mol_type = other DNA
gccacatatatgcaagtac

SEQ ID NO: 700 - DNA, 20 bp, synthetic construct
  source 1..20: mol_type = other DNA
agccacatatatgcaagtac

SEQ ID NO: 701 - DNA, 17 bp, synthetic construct
  source 1..17: mol_type = other DNA
cacagccacatatatgc

SEQ ID NO: 702 - DNA, 17 bp, synthetic construct
  source 1..17: mol_type = other DNA
tgcacagccacatatat

SEQ ID NO: 703 - DNA, 18 bp, synthetic construct
  source 1..18: mol_type = other DNA
ctgcacagccacatatat

SEQ ID NO: 704 - DNA, 16 bp, synthetic construct
  source 1..16: mol_type = other DNA
ctgcacagccacatat

SEQ ID NO: 705 - DNA, 16 bp, synthetic construct
  source 1..16: mol_type = other DNA
tcagaacaatctgccc

SEQ ID NO: 706 - DNA, 17 bp, synthetic construct
  source 1..17: mol_type = other DNA
ctcagaacaatctgccc

SEQ ID NO: 707 - DNA, 16 bp, synthetic construct
  source 1..16: mol_type = other DNA
ctgttttggcacatct

SEQ ID NO: 708 - DNA, 16 bp, synthetic construct
  source 1..16: mol_type = other DNA
gctgttttggcacatc

SEQ ID NO: 709 - DNA, 18 bp, synthetic construct
  source 1..18: mol_type = other DNA
tgaaaaacagcagcattg

SEQ ID NO: 710 - DNA, 17 bp, synthetic construct
  source 1..17: mol_type = other DNA
tgaaaaacagcagcatt

SEQ ID NO: 711 - DNA, 18 bp, synthetic construct
  source 1..18: mol_type = other DNA
ttgaaaaacagcagcatt

SEQ ID NO: 712 - DNA, 19 bp, synthetic construct
  source 1..19: mol_type = other DNA
attgaaaaacagcagcatt

SEQ ID NO: 713 - DNA, 20 bp, synthetic construct
  source 1..20: mol_type = other DNA
tattgaaaaacagcagcatt

SEQ ID NO: 714 - DNA, 19 bp, synthetic construct
  source 1..19: mol_type = other DNA
tattgaaaaacagcagcat

SEQ ID NO: 715 - DNA, 20 bp, synthetic construct
  source 1..20: mol_type = other DNA
ttattgaaaaacagcagcat

SEQ ID NO: 716 - DNA, 17 bp, synthetic construct
  source 1..17: mol_type = other DNA
tggttttattgaaaaac

SEQ ID NO: 717 - DNA, 18 bp, synthetic construct
  source 1..18: mol_type = other DNA
ctggttttattgaaaaac

SEQ ID NO: 718 - DNA, 19 bp, synthetic construct
  source 1..19: mol_type = other DNA
tctggttttattgaaaaac

SEQ ID NO: 719 - DNA, 20 bp, synthetic construct
  source 1..20: mol_type = other DNA
ctctggttttattgaaaaac

SEQ ID NO: 720 - DNA, 19 bp, synthetic construct
  source 1..19: mol_type = other DNA
cttcaactctggttttatt

SEQ ID NO: 721 - DNA, 18 bp, synthetic construct
  source 1..18: mol_type = other DNA
cttcaactctggttttat

SEQ ID NO: 722 - DNA, 17 bp, synthetic construct
  source 1..17: mol_type = other DNA
ccttcaactctggtttt

SEQ ID NO: 723 - DNA, 17 bp, synthetic construct
  source 1..17: mol_type = other DNA
gccttcaactctggttt

SEQ ID NO: 724 - DNA, 16 bp, synthetic construct
  source 1..16: mol_type = other DNA
ccacgatcttcgtctc

SEQ ID NO: 725 - DNA, 18 bp, synthetic construct
  source 1..18: mol_type = other DNA
agattatatttaggcact

SEQ ID NO: 726 - DNA, 16 bp, synthetic construct
  source 1..16: mol_type = other DNA
ccgcagattatattta

SEQ ID NO: 727 - DNA, 17 bp, synthetic construct
  source 1..17: mol_type = other DNA
cccgcagattatattta

SEQ ID NO: 728 - DNA, 17 bp, synthetic construct
  source 1..17: mol_type = other DNA
ccctcataaattagcac

SEQ ID NO: 729 - DNA, 16 bp, synthetic construct
  source 1..16: mol_type = other DNA
cctcataaattagcac

SEQ ID NO: 730 - DNA, 16 bp, synthetic construct
  source 1..16: mol_type = other DNA
cttccagccgcatttc

SEQ ID NO: 731 - DNA, 17 bp, synthetic construct
  source 1..17: mol_type = other DNA
gttctgcctctacctac

SEQ ID NO: 732 - DNA, 17 bp, synthetic construct
  source 1..17: mol_type = other DNA
acctctctgaagaccaa

SEQ ID NO: 733 - DNA, 17 bp, synthetic construct
  source 1..17: mol_type = other DNA
cacctccagcagccaaa

SEQ ID NO: 734 - DNA, 16 bp, synthetic construct
  source 1..16: mol_type = other DNA
ccactattcaggacaa

SEQ ID NO: 735 - DNA, 19 bp, synthetic construct
  source 1..19: mol_type = other DNA
ctgtctaaataacttatta

SEQ ID NO: 736 - DNA, 20 bp, synthetic construct
  source 1..20: mol_type = other DNA
tctgtctaaataacttatta

SEQ ID NO: 737 - DNA, 18 bp, synthetic construct
  source 1..18: mol_type = other DNA
tgtctaaataacttatta

SEQ ID NO: 738 - DNA, 20 bp, synthetic construct
  source 1..20: mol_type = other DNA
ttctgtctaaataacttatt

SEQ ID NO: 739 - DNA, 19 bp, synthetic construct
  source 1..19: mol_type = other DNA
tctgtctaaataacttatt

SEQ ID NO: 740 - DNA, 19 bp, synthetic construct
  source 1..19: mol_type = other DNA
attctgtctaaataactta

SEQ ID NO: 741 - DNA, 20 bp, synthetic construct
  source 1..20: mol_type = other DNA
tattctgtctaaataactta

SEQ ID NO: 742 - DNA, 18 bp, synthetic construct
  source 1..18: mol_type = other DNA
ttctgtctaaataactta

SEQ ID NO: 743 - DNA, 19 bp, synthetic construct
  source 1..19: mol_type = other DNA
gctattctgtctaaataac

SEQ ID NO: 744 - DNA, 20 bp, synthetic construct
  source 1..20: mol_type = other DNA
tgctattctgtctaaataac

SEQ ID NO: 745 - DNA, 18 bp, synthetic construct
  source 1..18: mol_type = other DNA
gctattctgtctaaataa

SEQ ID NO: 746 - DNA, 19 bp, synthetic construct
  source 1..19: mol_type = other DNA
tgctattctgtctaaataa

SEQ ID NO: 747 - DNA, 17 bp, synthetic construct
  source 1..17: mol_type = other DNA
ctattctgtctaaataa

SEQ ID NO: 748 - DNA, 18 bp, synthetic construct
  source 1..18: mol_type = other DNA
tgctattctgtctaaata

SEQ ID NO: 749 - DNA, 17 bp, synthetic construct
  source 1..17: mol_type = other DNA
tgctattctgtctaaat

SEQ ID NO: 750 - DNA, 18 bp, synthetic construct
  source 1..18: mol_type = other DNA
tcccaagaattaaagtca

SEQ ID NO: 751 - DNA, 17 bp, synthetic construct
  source 1..17: mol_type = other DNA
cccaagaattaaagtca

SEQ ID NO: 752 - DNA, 19 bp, synthetic construct
  source 1..19: mol_type = other DNA
ttttgtcccaagaattaaa

SEQ ID NO: 753 - DNA, 18 bp, synthetic construct
  source 1..18: mol_type = other DNA
ttttgtcccaagaattaa

SEQ ID NO: 754 - DNA, 17 bp, synthetic construct
  source 1..17: mol_type = other DNA
ttttgtcccaagaatta

SEQ ID NO: 755 - DNA, 18 bp, synthetic construct
  source 1..18: mol_type = other DNA
ttcttctcctgccaccaa

SEQ ID NO: 756 - DNA, 17 bp, synthetic construct
  source 1..17: mol_type = other DNA
tattcttctcctgccac

SEQ ID NO: 757 - DNA, 18 bp, synthetic construct
  source 1..18: mol_type = other DNA
ttattcttctcctgccac

SEQ ID NO: 758 - DNA, 17 bp, synthetic construct
  source 1..17: mol_type = other DNA
cacctcaattgctaaca

SEQ ID NO: 759 - DNA, 18 bp, synthetic construct
  source 1..18: mol_type = other DNA
acacctcaattgctaaca

SEQ ID NO: 760 - DNA, 18 bp, synthetic construct
  source 1..18: mol_type = other DNA
gcacacctcaattgctaa

SEQ ID NO: 761 - DNA, 17 bp, synthetic construct
  source 1..17: mol_type = other DNA
cacacctcaattgctaa

SEQ ID NO: 762 - DNA, 16 bp, synthetic construct
  source 1..16: mol_type = other DNA
acacctcaattgctaa

SEQ ID NO: 763 - DNA, 16 bp, synthetic construct
  source 1..16: mol_type = other DNA
gtctacaaaactccca

SEQ ID NO: 764 - DNA, 17 bp, synthetic construct
  source 1..17: mol_type = other DNA
agtctacaaaactccca

SEQ ID NO: 765 - DNA, 18 bp, synthetic construct
  source 1..18: mol_type = other DNA
gtacagtctacaaaactc

SEQ ID NO: 766 - DNA, 19 bp, synthetic construct
  source 1..19: mol_type = other DNA
tgtacagtctacaaaactc

SEQ ID NO: 767 - DNA, 20 bp, synthetic construct
  source 1..20: mol_type = other DNA
ctgtacagtctacaaaactc

SEQ ID NO: 768 - DNA, 20 bp, synthetic construct
  source 1..20: mol_type = other DNA
tctgtacagtctacaaaact

SEQ ID NO: 769 - DNA, 20 bp, synthetic construct
  source 1..20: mol_type = other DNA
ttctgtacagtctacaaaac

SEQ ID NO: 770 - DNA, 19 bp, synthetic construct
  source 1..19: mol_type = other DNA
tctgtacagtctacaaaac

SEQ ID NO: 771 - DNA, 19 bp, synthetic construct
  source 1..19: mol_type = other DNA
ttctgtacagtctacaaaa

SEQ ID NO: 772 - DNA, 20 bp, synthetic construct
  source 1..20: mol_type = other DNA
tttctgtacagtctacaaaa

SEQ ID NO: 773 - DNA, 18 bp, synthetic construct
  source 1..18: mol_type = other DNA
tctgtacagtctacaaaa

SEQ ID NO: 774 - DNA, 19 bp, synthetic construct
  source 1..19: mol_type = other DNA
tttctgtacagtctacaaa

SEQ ID NO: 775 - DNA, 20 bp, synthetic construct
  source 1..20: mol_type = other DNA
atttctgtacagtctacaaa

SEQ ID NO: 776 - DNA, 18 bp, synthetic construct
  source 1..18: mol_type = other DNA
tttctgtacagtctacaa

SEQ ID NO: 777 - DNA, 19 bp, synthetic construct
  source 1..19: mol_type = other DNA
atttctgtacagtctacaa

SEQ ID NO: 778 - DNA, 18 bp, synthetic construct
  source 1..18: mol_type = other DNA
atttctgtacagtctaca

SEQ ID NO: 779 - DNA, 17 bp, synthetic construct
  source 1..17: mol_type = other DNA
atttctgtacagtctac

SEQ ID NO: 780 - DNA, 20 bp, synthetic construct
  source 1..20: mol_type = other DNA
attccagattaacaaaatct

SEQ ID NO: 781 - DNA, 19 bp, synthetic construct
  source 1..19: mol_type = other DNA
attccagattaacaaaatc

SEQ ID NO: 782 - DNA, 20 bp, synthetic construct
  source 1..20: mol_type = other DNA
atattccagattaacaaaat

SEQ ID NO: 783 - DNA, 20 bp, synthetic construct
  source 1..20: mol_type = other DNA
tatattccagattaacaaaa

SEQ ID NO: 784 - DNA, 19 bp, synthetic construct
  source 1..19: mol_type = other DNA
tatattccagattaacaaa

SEQ ID NO: 785 - DNA, 20 bp, synthetic construct
  source 1..20: mol_type = other DNA
gtatattccagattaacaaa

SEQ ID NO: 786 - DNA, 18 bp, synthetic construct
  source 1..18: mol_type = other DNA
atattccagattaacaaa

SEQ ID NO: 787 - DNA, 20 bp, synthetic construct
  source 1..20: mol_type = other DNA
tgtatattccagattaacaa

SEQ ID NO: 788 - DNA, 18 bp, synthetic construct
  source 1..18: mol_type = other DNA
tatattccagattaacaa

SEQ ID NO: 789 - DNA, 19 bp, synthetic construct
  source 1..19: mol_type = other DNA
gtatattccagattaacaa

SEQ ID NO: 790 - DNA, 19 bp, synthetic construct
  source 1..19: mol_type = other DNA
tgtatattccagattaaca

SEQ ID NO: 791 - DNA, 18 bp, synthetic construct
  source 1..18: mol_type = other DNA
gtatattccagattaaca

SEQ ID NO: 792 - DNA, 20 bp, synthetic construct
  source 1..20: mol_type = other DNA
tctgtatattccagattaac

SEQ ID NO: 793 - DNA, 18 bp, synthetic construct
  source 1..18: mol_type = other DNA
tgtatattccagattaac

SEQ ID NO: 794 - DNA, 17 bp, synthetic construct
  source 1..17: mol_type = other DNA
gtatattccagattaac

SEQ ID NO: 795 - DNA, 19 bp, synthetic construct
  source 1..19: mol_type = other DNA
ctgtatattccagattaac

SEQ ID NO: 796 - DNA, 18 bp, synthetic construct
  source 1..18: mol_type = other DNA
ctgtatattccagattaa

SEQ ID NO: 797 - DNA, 19 bp, synthetic construct
  source 1..19: mol_type = other DNA
tctgtatattccagattaa

SEQ ID NO: 798 - DNA, 17 bp, synthetic construct
  source 1..17: mol_type = other DNA
tgtatattccagattaa

SEQ ID NO: 799 - DNA, 17 bp, synthetic construct
  source 1..17: mol_type = other DNA
ctgtatattccagatta

SEQ ID NO: 800 - DNA, 18 bp, synthetic construct
  source 1..18: mol_type = other DNA
tctgtatattccagatta

SEQ ID NO: 801 - DNA, 17 bp, synthetic construct
  source 1..17: mol_type = other DNA
tctgtctgtatattcca

SEQ ID NO: 802 - DNA, 18 bp, synthetic construct
  source 1..18: mol_type = other DNA
aacactttacgtctgtct

SEQ ID NO: 803 - DNA, 17 bp, synthetic construct
  source 1..17: mol_type = other DNA
acactttacgtctgtct

SEQ ID NO: 804 - DNA, 20 bp, synthetic construct
  source 1..20: mol_type = other DNA
aaaacactttacgtctgtct

SEQ ID NO: 805 - DNA, 18 bp, synthetic construct
  source 1..18: mol_type = other DNA
ctaaaacactttacgtct

SEQ ID NO: 806 - DNA, 17 bp, synthetic construct
  source 1..17: mol_type = other DNA
taaaacactttacgtct

SEQ ID NO: 807 - DNA, 18 bp, synthetic construct
  source 1..18: mol_type = other DNA
tttgctaaaacactttac

SEQ ID NO: 808 - DNA, 20 bp, synthetic construct
  source 1..20: mol_type = other DNA
attttgctaaaacactttac

SEQ ID NO: 809 - DNA, 19 bp, synthetic construct
  source 1..19: mol_type = other DNA
ttttgctaaaacactttac

SEQ ID NO: 810 - DNA, 19 bp, synthetic construct
  source 1..19: mol_type = other DNA
attttgctaaaacacttta

SEQ ID NO: 811 - DNA, 20 bp, synthetic construct
  source 1..20: mol_type = other DNA
cattttgctaaaacacttta

SEQ ID NO: 812 - DNA, 18 bp, synthetic construct
  source 1..18: mol_type = other DNA
ttttgctaaaacacttta

SEQ ID NO: 813 - DNA, 19 bp, synthetic construct
  source 1..19: mol_type = other DNA
cattttgctaaaacacttt

SEQ ID NO: 814 - DNA, 18 bp, synthetic construct
  source 1..18: mol_type = other DNA
attttgctaaaacacttt

SEQ ID NO: 815 - DNA, 20 bp, synthetic construct
  source 1..20: mol_type = other DNA
tccattttgctaaaacactt

SEQ ID NO: 816 - DNA, 19 bp, synthetic construct
  source 1..19: mol_type = other DNA
ccattttgctaaaacactt

SEQ ID NO: 817 - DNA, 19 bp, synthetic construct
  source 1..19: mol_type = other DNA
tccattttgctaaaacact

SEQ ID NO: 818 - DNA, 19 bp, synthetic construct
  source 1..19: mol_type = other DNA
tttccattttgctaaaaca

SEQ ID NO: 819 - DNA, 18 bp, synthetic construct
  source 1..18: mol_type = other DNA
gtttccattttgctaaaa

SEQ ID NO: 820 - DNA, 19 bp, synthetic construct
  source 1..19: mol_type = other DNA
tgtttccattttgctaaaa

SEQ ID NO: 821 - DNA, 20 bp, synthetic construct
  source 1..20: mol_type = other DNA
ttgtttccattttgctaaaa

SEQ ID NO: 822 - DNA, 19 bp, synthetic construct
  source 1..19: mol_type = other DNA
ttgtttccattttgctaaa

SEQ ID NO: 823 - DNA, 18 bp, synthetic construct
  source 1..18: mol_type = other DNA
tgtttccattttgctaaa

SEQ ID NO: 824 - DNA, 20 bp, synthetic construct
  source 1..20: mol_type = other DNA
tttgtttccattttgctaaa

SEQ ID NO: 825 - DNA, 18 bp, synthetic construct
  source 1..18: mol_type = other DNA
ttgtttccattttgctaa

SEQ ID NO: 826 - DNA, 19 bp, synthetic construct
  source 1..19: mol_type = other DNA
tttgtttccattttgctaa

SEQ ID NO: 827 - DNA, 18 bp, synthetic construct
  source 1..18: mol_type = other DNA
actgtttgtttccatttt

SEQ ID NO: 828 - DNA, 18 bp, synthetic construct
  source 1..18: mol_type = other DNA
aactgtttgtttccattt

SEQ ID NO: 829 - DNA, 18 bp, synthetic construct
  source 1..18: mol_type = other DNA
acaactgtttgtttccat

SEQ ID NO: 830 - DNA, 17 bp, synthetic construct
  source 1..17: mol_type = other DNA
acaactgtttgtttcca

SEQ ID NO: 831 - DNA, 18 bp, synthetic construct
  source 1..18: mol_type = other DNA
cacaactgtttgtttcca

SEQ ID NO: 832 - DNA, 17 bp, synthetic construct
  source 1..17: mol_type = other DNA
cacaactgtttgtttcc

SEQ ID NO: 833 - DNA, 17 bp, synthetic construct
  source 1..17: mol_type = other DNA
catctctaccaagtcct

SEQ ID NO: 834 - DNA, 18 bp, synthetic construct
  source 1..18: mol_type = other DNA
tcctgccatctctaccaa

SEQ ID NO: 835 - DNA, 17 bp, synthetic construct
  source 1..17: mol_type = other DNA
gtataaaattcacgtca

SEQ ID NO: 836 - DNA, 19 bp, synthetic construct
  source 1..19: mol_type = other DNA
acgtataaaattcacgtca

SEQ ID NO: 837 - DNA, 18 bp, synthetic construct
  source 1..18: mol_type = other DNA
cgtataaaattcacgtca

SEQ ID NO: 838 - DNA, 18 bp, synthetic construct
  source 1..18: mol_type = other DNA
tccaaacacacagaaatc

SEQ ID NO: 839 - DNA, 19 bp, synthetic construct
  source 1..19: mol_type = other DNA
ttccaaacacacagaaatc

SEQ ID NO: 840 - DNA, 19 bp, synthetic construct
  source 1..19: mol_type = other DNA
cttccaaacacacagaaat

SEQ ID NO: 841 - DNA, 20 bp, synthetic construct
  source 1..20: mol_type = other DNA
gcttccaaacacacagaaat

SEQ ID NO: 842 - DNA, 18 bp, synthetic construct
  source 1..18: mol_type = other DNA
cttccaaacacacagaaa

SEQ ID NO: 843 - DNA, 19 bp, synthetic construct
  source 1..19: mol_type = other DNA
gcttccaaacacacagaaa

SEQ ID NO: 844 - DNA, 19 bp, synthetic construct
  source 1..19: mol_type = other DNA
tgctgttttaaaaaaaata

SEQ ID NO: 845 - DNA, 19 bp, synthetic construct
  source 1..19: mol_type = other DNA
ttgctgttttaaaaaaaat

SEQ ID NO: 846 - DNA, 20 bp, synthetic construct
  source 1..20: mol_type = other DNA
attgctgttttaaaaaaaat

SEQ ID NO: 847 - DNA, 20 bp, synthetic construct
  source 1..20: mol_type = other DNA
aattattgctgttttaaaaa

SEQ ID NO: 848 - DNA, 20 bp, synthetic construct
  source 1..20: mol_type = other DNA
ctaattattgctgttttaaa

SEQ ID NO: 849 - DNA, 19 bp, synthetic construct
  source 1..19: mol_type = other DNA
ctaattattgctgttttaa

SEQ ID NO: 850 - DNA, 18 bp, synthetic construct
  source 1..18: mol_type = other DNA
ctaattattgctgtttta

SEQ ID NO: 851 - DNA, 19 bp, synthetic construct
  source 1..19: mol_type = other DNA
cctttaaaatggctaatta

SEQ ID NO: 852 - DNA, 20 bp, synthetic construct
  source 1..20: mol_type = other DNA
tcctttaaaatggctaatta

SEQ ID NO: 853 - DNA, 18 bp, synthetic construct
  source 1..18: mol_type = other DNA
ctcctttaaaatggctaa

SEQ ID NO: 854 - DNA, 17 bp, synthetic construct
  source 1..17: mol_type = other DNA
ctcctttaaaatggcta

SEQ ID NO: 855 - DNA, 17 bp, synthetic construct
  source 1..17: mol_type = other DNA
catccctcctttaaaat

SEQ ID NO: 856 - DNA, 17 bp, synthetic construct
  source 1..17: mol_type = other DNA
aagaacccacttgcaca

SEQ ID NO: 857 - DNA, 17 bp, synthetic construct
  source 1..17: mol_type = other DNA
tacatgcttatgcacct

SEQ ID NO: 858 - DNA, 16 bp, synthetic construct
  source 1..16: mol_type = other DNA
tacatgcttatgcacc

SEQ ID NO: 859 - DNA, 18 bp, synthetic construct
  source 1..18: mol_type = other DNA
aactcatgcacatgtaca

SEQ ID NO: 860 - DNA, 18 bp, synthetic construct
  source 1..18: mol_type = other DNA
caactcatgcacatgtac

SEQ ID NO: 861 - DNA, 19 bp, synthetic construct
  source 1..19: mol_type = other DNA
acaactcatgcacatgtac

SEQ ID NO: 862 - DNA, 16 bp, synthetic construct
  source 1..16: mol_type = other DNA
ctatacacgtgctcac

SEQ ID NO: 863 - DNA, 17 bp, synthetic construct
  source 1..17: mol_type = other DNA
tctatacacgtgctcac

SEQ ID NO: 864 - DNA, 18 bp, synthetic construct
  source 1..18: mol_type = other DNA
atctatacacgtgctcac

SEQ ID NO: 865 - DNA, 18 bp, synthetic construct
  source 1..18: mol_type = other DNA
caccatatgcatatgtat

SEQ ID NO: 866 - DNA, 19 bp, synthetic construct
  source 1..19: mol_type = other DNA
acaccatatgcatatgtat

SEQ ID NO: 867 - DNA, 18 bp, synthetic construct
  source 1..18: mol_type = other DNA
cgtacaccatatgcatat

SEQ ID NO: 868 - DNA, 17 bp, synthetic construct
  source 1..17: mol_type = other DNA
gtacaccatatgcatat

SEQ ID NO: 869 - DNA, 16 bp, synthetic construct
  source 1..16: mol_type = other DNA
gtacaccatatgcata

SEQ ID NO: 870 - DNA, 17 bp, synthetic construct
  source 1..17: mol_type = other DNA
cgtacaccatatgcata

SEQ ID NO: 871 - DNA, 18 bp, synthetic construct
  source 1..18: mol_type = other DNA
acgtacaccatatgcata

SEQ ID NO: 872 - DNA, 16 bp, synthetic construct
  source 1..16: mol_type = other DNA
cgtacaccatatgcat

SEQ ID NO: 873 - DNA, 17 bp, synthetic construct
  source 1..17: mol_type = other DNA
acgtacaccatatgcat

SEQ ID NO: 874 - DNA, 19 bp, synthetic construct
  source 1..19: mol_type = other DNA
atatatgcaagtacacaca

SEQ ID NO: 875 - DNA, 18 bp, synthetic construct
  source 1..18: mol_type = other DNA
tatatgcaagtacacaca

SEQ ID NO: 876 - DNA, 18 bp, synthetic construct
  source 1..18: mol_type = other DNA
catatatgcaagtacaca

SEQ ID NO: 877 - DNA, 19 bp, synthetic construct
  source 1..19: mol_type = other DNA
acatatatgcaagtacaca

SEQ ID NO: 878 - DNA, 18 bp, synthetic construct
  source 1..18: mol_type = other DNA
cacatatatgcaagtaca

SEQ ID NO: 879 - DNA, 18 bp, synthetic construct
  source 1..18: mol_type = other DNA
acagccacatatatgcaa

SEQ ID NO: 880 - DNA, 17 bp, synthetic construct
  source 1..17: mol_type = other DNA
cagccacatatatgcaa

SEQ ID NO: 881 - DNA, 17 bp, synthetic construct
  source 1..17: mol_type = other DNA
cttcaactctggtttta

SEQ ID NO: 882 - RNA, 16 nt, synthetic construct
  source 1..16: mol_type = other RNA
gagacgaagatcgtgg

SEQ ID NO: 883 - RNA, 23 nt, synthetic construct
  source 1..23: mol_type = other RNA
agtgcctaaatataatctgcggg

SEQ ID NO: 884 - RNA, 17 nt, synthetic construct
  source 1..17: mol_type = other RNA
gtgctaatttatgaggg

SEQ ID NO: 885 - RNA, 16 nt, synthetic construct
  source 1..16: mol_type = other RNA
gaaatgcggctggaag

SEQ ID NO: 886 - RNA, 17 nt, synthetic construct
  source 1..17: mol_type = other RNA
gtaggtagaggcagaac

SEQ ID NO: 887 - RNA, 17 nt, synthetic construct
  source 1..17: mol_type = other RNA
ttggtcttcagagaggt

SEQ ID NO: 888 - RNA, 17 nt, synthetic construct
  source 1..17: mol_type = other RNA
tttggctgctggaggtg

SEQ ID NO: 889 - RNA, 16 nt, synthetic construct
  source 1..16: mol_type = other RNA
ttgtcctgaatagtgg

SEQ ID NO: 890 - RNA, 26 nt, synthetic construct
  source 1..26: mol_type = other RNA
taataagttatttagacagaatagca

SEQ ID NO: 891 - RNA, 23 nt, synthetic construct
  source 1..23: mol_type = other RNA
tgactttaattcttgggacaaaa

SEQ ID NO: 892 - RNA, 21 nt, synthetic construct
  source 1..21: mol_type = other RNA
ttggtggcaggagaagaataa

SEQ ID NO: 893 - RNA, 20 nt, synthetic construct
  source 1..20: mol_type = other RNA
tgttagcaattgaggtgtgc

SEQ ID NO: 894 - RNA, 27 nt, synthetic construct
  source 1..27: mol_type = other RNA
tgggagttttgtagactgtacagaaat

SEQ ID NO: 895 - RNA, 69 nt, synthetic construct
  source 1..69: mol_type = other RNA
agattttgttaatctggaatatacagacagacgtaaagtgttttagcaaaatggaaacaaacagttgtg

SEQ ID NO: 896 - RNA, 23 nt, synthetic construct
  source 1..23: mol_type = other RNA
aggacttggtagagatggcagga

SEQ ID NO: 897 - RNA, 19 nt, synthetic construct
  source 1..19: mol_type = other RNA
tgacgtgaattttatacgt

SEQ ID NO: 898 - RNA, 21 nt, synthetic construct
  source 1..21: mol_type = other RNA
gatttctgtgtgtttggaagc

SEQ ID NO: 899 - RNA, 46 nt, synthetic construct
  source 1..46: mol_type = other RNA
tattttttttaaaacagcaataattagccattttaaaggagggatg

SEQ ID NO: 900 - RNA, 17 nt, synthetic construct
  source 1..17: mol_type = other RNA
tgtgcaagtgggttctt

SEQ ID NO: 901 - RNA, 17 nt, synthetic construct
  source 1..17: mol_type = other RNA
aggtgcataagcatgta

SEQ ID NO: 902 - RNA, 20 nt, synthetic construct
  source 1..20: mol_type = other RNA
tgtacatgtgcatgagttgt

SEQ ID NO: 903 - RNA, 18 nt, synthetic construct
  source 1..18: mol_type = other RNA
gtgagcacgtgtatagat

SEQ ID NO: 904 - RNA, 23 nt, synthetic construct
  source 1..23: mol_type = other RNA
atacatatgcatatggtgtacgt

SEQ ID NO: 905 - RNA, 27 nt, synthetic construct
  source 1..27: mol_type = other RNA
tgtgtgtacttgcatatatgtggctgt

SEQ ID NO: 906 - RNA, 17 nt, synthetic construct
  source 1..17: mol_type = other RNA
taaaaccagagttgaag

SEQ ID NO: 907 - RNA, 19 nt, synthetic construct
  source 1..19: mol_type = other RNA
gagtgcctaaatataatct

SEQ ID NO: 908 - RNA, 18 nt, synthetic construct
  source 1..18: mol_type = other RNA
aattcttgggacaaaacg

SEQ ID NO: 909 - RNA, 20 nt, synthetic construct
  source 1..20: mol_type = other RNA
aagtgttttagcaaaatgga

SEQ ID NO: 910 - RNA, 18 nt, synthetic construct
  source 1..18: mol_type = other RNA
atggaaacaaacagttgt

SEQ ID NO: 911 - RNA, 17 nt, synthetic construct
  source 1..17: mol_type = other RNA
ggaaacaaacagttgtg

SEQ ID NO: 912 - RNA, 17 nt, synthetic construct
  source 1..17: mol_type = other RNA
aaacaaacagttgtgcc

SEQ ID NO: 913 - RNA, 19 nt, synthetic construct
  source 1..19: mol_type = other RNA
agtgttttagcaaaatgga

SEQ ID NO: 914 - RNA, 19 nt, synthetic construct
  source 1..19: mol_type = other RNA
aagtgttttagcaaaatgg

SEQ ID NO: 915 - RNA, 18 nt, synthetic construct
  source 1..18: mol_type = other RNA
aaaatggaaacaaacagt

SEQ ID NO: 916 - RNA, 18 nt, synthetic construct
  source 1..18: mol_type = other RNA
aaatggaaacaaacagtt

SEQ ID NO: 917 - RNA, 16 nt, synthetic construct
  source 1..16: mol_type = other RNA
gatgtaacttcttcta

SEQ ID NO: 918 - RNA, 17 nt, synthetic construct
  source 1..17: mol_type = other RNA
gatgtaacttcttctac

SEQ ID NO: 919 - RNA, 18 nt, synthetic construct
  source 1..18: mol_type = other RNA
ggacataccattgttgga

SEQ ID NO: 920 - RNA, 17 nt, synthetic construct
  source 1..17: mol_type = other RNA
aaacagcaataattagc

SEQ ID NO: 921 - RNA, 16 nt, synthetic construct
  source 1..16: mol_type = other RNA
agatgtgccaaaacag

SEQ ID NO: 922 - RNA, 17 nt, synthetic construct
  source 1..17: mol_type = other RNA
taatctggaatatacag

SEQ ID NO: 923 - RNA, 17 nt, synthetic construct
  source 1..17: mol_type = other RNA
tggaatatacagacaga

SEQ ID NO: 924 - RNA, 18 nt, synthetic construct
  source 1..18: mol_type = other RNA
agacagacgtaaagtgtt

SEQ ID NO: 925 - RNA, 17 nt, synthetic construct
  source 1..17: mol_type = other RNA
agacagacgtaaagtgt

SEQ ID NO: 926 - RNA, 18 nt, synthetic construct
  source 1..18: mol_type = other RNA
agacgtaaagtgttttag

SEQ ID NO: 927 - RNA, 20 nt, synthetic construct
  source 1..20: mol_type = other RNA
tttgttaatctggaatatac

SEQ ID NO: 928 - RNA, 20 nt, synthetic construct
  source 1..20: mol_type = other RNA
ttgttaatctggaatataca

SEQ ID NO: 929 - RNA, 18 nt, synthetic construct
  source 1..18: mol_type = other RNA
ttttagcaaaatggaaac

SEQ ID NO: 930 - RNA, 19 nt, synthetic construct
  source 1..19: mol_type = other RNA
tttagcaaaatggaaacaa

SEQ ID NO: 931 - RNA, 18 nt, synthetic construct
  source 1..18: mol_type = other RNA
tttagcaaaatggaaaca

SEQ ID NO: 932 - RNA, 19 nt, synthetic construct
  source 1..19: mol_type = other RNA
ttgttaatctggaatatac

SEQ ID NO: 933 - RNA, 17 nt, synthetic construct
  source 1..17: mol_type = other RNA
tggaaacaaacagttgt

SEQ ID NO: 934 - RNA, 18 nt, synthetic construct
  source 1..18: mol_type = other RNA
tggaaacaaacagttgtg

SEQ ID NO: 935 - RNA, 17 nt, synthetic construct
  source 1..17: mol_type = other RNA
tgacgtgaattttatac

SEQ ID NO: 936 - RNA, 18 nt, synthetic construct
  source 1..18: mol_type = other RNA
agtgcctaaatataatct

(19) United States
(12) Patent Application Publication
(10) Pub. No.: US 2026/0103714 A1
(43) Pub. Date: Apr. 16, 2026

(54) ANTISENSE OLIGONUCLEOTIDES FOR THE TREATMENT OF CANAVAN DISEASE

(71) Applicant: Contera Pharma A/S, Hørsholm (DK)

(72) Inventors: Tue Fryland, Rønde (DK); Marianne Lerbech Jensen, Hørsholm (DK); Søren Vestergaard Rasmussen, Hørsholm (DK)

(21) Appl. No.: 19/116,332

(22) PCT Filed: Sep. 29, 2023
(86) PCT No.: PCT/EP2023/077175

§ 371 Date: Mar. 27, 2025

(30) Foreign Application Priority Data
Sep. 30, 2022 (EP) 22199007.0

(51) Int. Cl. C12N 15/113; A61P 25/28; G01N 33/50

(52) U.S. Cl. CPC C12N 15/1137; A61P 25/28; C12Y 203/01017; G01N 33/5023; C12N 2310/14

(57) ABSTRACT

The present invention relates to antisense oligonucleotides (oligomers) complementary to NAT8L pre-mRNA and mRNA and which are capable of inhibiting the expression of NAT8L. Inhibition of NAT8L expression is beneficial for the treatment of Canavan disease.

### FIELD OF THE INVENTION

The present invention relates to antisense oligonucleotides (oligomers) complementary to NAT8L (N-acetyltransferase 8 like) pre-mRNA and mRNA and which are capable of inhibiting the expression of NAT8L. Inhibition of NAT8L expression is beneficial for the treatment of Canavan disease.

### BACKGROUND

Canavan disease is a recessively inherited vacuolar leukodystrophy caused by ASPA mutations. ASPA encodes aspartoacylase, an oligodendroglial enzyme required for cleavage of the abundant brain amino acid N-acetyl-1-aspartate (NAA) to acetate and L-aspartate.

Canavan disease is a rare and fatal autosomal recessive degenerative disorder that causes progressive damage to nerve cells and pathology of the white matter in the brain. It is one of the most frequent degenerative brain diseases of infancy. Canavan disease belongs to a group of genetic diseases known as leukodystrophies, referring to defects in the development and growth of white matter. Early symptoms of Canavan disease such a hypotonia, macrocephaly, poor head control usually become apparent between the age of 3 and 6 months. The affected infants show delayed developmental milestones (e.g., sitting, standing) and usually never develop the ability to walk. The progressive nature of the disease results in loss of previously acquired abilities and additional symptoms such as seizures and mental retardation also become apparent during infancy. The prognosis for Canavan disease is poor and, in most cases, death occurs before the age of ten.

Canavan disease is caused by mutations in the in the ASPA gene, which encodes aspartoacyclase, an enzyme required conversion of the N-acetyl-1-aspartate (NAA) into acetate and L-aspartate. Currently, there are two prevailing hypotheses of the resulting pathological process. The ‘Oligodendroglial starvation’ hypothesis proposes that the lack of functional aspartoacyclase results in a deficiency of NAA-derived acetate required for the biosynthesis of myelin (white matter material) and as a result degeneration of myelin/white matter in patients. The ‘NAA toxicity’ hypothesis in contrary proposes, that the excessive NAA concentration in the brain of patients lacking aspartoacyclase, causes an impaired osmolar homeostasis and as result the astroglial and myelinic vacuolation, which is hallmark of Canavan disease (Pleasure et al., Neurochem Res. 2020 March; 45(3):561-565. doi: 10.1007/s11064-018-2693-6. Epub 2018 Dec. 8. Pathophysiology and Treatment of Canavan Disease.).

In support of the ‘NAA toxicity’ Maier et al., 2015 reported that in Aspa deficient mice (Asap^{nur7/nur7}, 40 Canavan disease model) also deficient for NAA synthase Nat8l (Aspa^{nur7/nur7}/Nat8l^{-/-}) showed normal myelin content and no axonal degeneration, despite having no detectable NAA. However, survival time was not improved (Maier et al., J Neurosci. 2015 Oct. 28; 35(43):14501-16. doi: 10.1523/JNEUROSCI.1056-15.2015. N-Acetylaspartate Synthase Deficiency Corrects the Myelin Phenotype in a Canavan Disease Mouse Model But Does Not Affect Survival Time). Moreover, mice with only one intact Nat8l allele (Aspa^{nur7/nur7}/Nat8l^{-/-}) accumulated less NAA and developed a less severe disease pathology showed phenotypic improvements and normal lifespan. Similar results have been reported by Sohn et al. (J Neurosci. 2017 Jan. 11; 37(2):413-421. doi: 10.1523/JNEUROSCI.2013-16.2016. Suppressing N-Acetyl-1-Aspartate Synthesis Prevents Loss of Neurons in a Murine Model of Canavan Leukodystrophy).

Subsequently, Bannerman et al., 2018 showed that knocking down Nat8l expression in Aspa^{nur7/nur7} using adeno-associated viral vector carrying short hairpin Nat8l inhibitory RNA reduced NAA levels, suppressed the development disease pathology, and improved enhanced motor performance (Bannerman et al., Brain Nat8l Knockdown Suppresses Spongiform Leukodystrophy in an Aspartoacylase-Deficient Canavan Disease Mouse Model Mol Ther. 2018 Mar. 7; 26(3):793-800. doi: 10.1016/j.ymthe.2018.01.002. Epub 2018 Jan. 10).

Furthermore, Hull et al., 2020 showed that antisense oligonucleotides reducing Nat8l expression in Aspa^{nur7/nur7} likewise reduced levels of NAA, reversed ataxia and reduced the cerebellar and thalamic vacuolation in young-adult Aspa^{nur7/nur7} mice (Ann Neurol. 2020 March; 87(3):480-485. doi: 10.1002/ana.25674. Epub 2020 Jan. 22. Antisense Oligonucleotide Reverses Leukodystrophy in Canavan Disease Mice).

Altogether these data points towards inhibition NAT8L and lowering of NAA levels as a potentially effective therapeutic concept for treating Canavan disease, a progressive and fatal neurodegenerative disease for which there are currently no effective treatments available.

There remains a need for means and methods for the efficient down-regulation of the NAT8L gene.

### BRIEF SUMMARY OF THE PRESENT INVENTION

The present invention provides an antisense oligonucleotide comprising a stretch of at least 10 nucleotides which is at least 90% complementary to a target sequence in a NAT8L (N-acetyltransferase 8 like) gene.

Preferably, the stretch of at least 10 nucleotides is at least 90% complementary to a target sequence selected from the group of target sequences consisting of the target sequences shown in Table B1 or D1 in the Examples section.

In an embodiment, the stretch of at least 10 nucleotides is at least 90% complementary to a target sequence selected from the group of target sequences consisting of SEQ ID NO: 4 to SEQ ID NO: 81 and SEQ ID NO: 882 to SEQ ID NO: 906.

In a preferred embodiment, the stretch of at least 10 nucleotides is at least 90% complementary to a target sequence selected from the group of target sequences consisting of SEQ ID NO: 30 to SEQ ID NO: 81. The sequences are shown in Table B1 in the Examples section.

In a further preferred embodiment, the stretch of at least 10 nucleotides is at least 90% complementary to a target sequence selected from the group of target sequences consisting of SEQ ID NO: 11 to SEQ ID NO: 29. The sequences are shown in Table B2 in the Examples section.

In another preferred embodiment, the stretch of at least 10 nucleotides is at least 90% complementary to a target sequence selected from the group of target sequences consisting of SEQ ID NO: 883, 892, 894, 895, 896, 897, 898 or 899. The sequences are shown in Table D2 in the Examples section.

In a particularly preferred embodiment, the stretch of at least 10 nucleotides is at least 90% complementary to a target sequence selected from the group of target sequences consisting of SEQ ID NO: 4 to SEQ ID NO: 10. The sequences are shown in Table B3 in the Examples section.

In another particularly preferred embodiment, the stretch of at least 10 nucleotides is at least 90% complementary to a target sequence selected from the group of target sequences consisting of SEQ ID NO: 883, 894, 895, 897, 898 and 899. The sequences are shown in Table D3 in the Examples section.

In an embodiment, the stretch of at least 10 nucleotides is at least 90% complementary to a target sequence as shown in SEQ ID NO: 4.

In an embodiment, the stretch of at least 10 nucleotides is at least 90% complementary to a target sequence as shown in SEQ ID NO: 5.

In an embodiment, the stretch of at least 10 nucleotides is at least 90% complementary to a target sequence as shown in SEQ ID NO: 6.

In an embodiment, the stretch of at least 10 nucleotides is at least 90% complementary to a target sequence as shown in SEQ ID NO: 7.

In an embodiment, the stretch of at least 10 nucleotides is at least 90% complementary to a target sequence as shown in SEQ ID NO: 8.

In an embodiment, the stretch of at least 10 nucleotides is at least 90% complementary to a target sequence as shown in SEQ ID NO: 9.

In an embodiment, the stretch of at least 10 nucleotides is at least 90% complementary to a target sequence as shown in SEQ ID NO: 10.

In an embodiment, the stretch of at least 10 nucleotides is at least 90% complementary to a target sequence as shown in SEQ ID NO: 883.

In an embodiment, the stretch of at least 10 nucleotides is at least 90% complementary to a target sequence as shown in SEQ ID NO: 894.

In an embodiment, the stretch of at least 10 nucleotides is at least 90% complementary to a target sequence as shown in SEQ ID NO: 895.

In an embodiment, the stretch of at least 10 nucleotides is at least 90% complementary to a target sequence as shown in SEQ ID NO: 897.

In an embodiment, the stretch of at least 10 nucleotides is at least 90% complementary to a target sequence as shown in SEQ ID NO: 898.

In an embodiment, the stretch of at least 10 nucleotides is at least 90% complementary to a target sequence as shown in SEQ ID NO: 899.

Preferably, the antisense oligonucleotide comprises a stretch of at least 12, or more preferably at least 14 nucleotides which is at least 90% complementary to a target sequence as set forth herein.

Also preferable, the target sequence comprises or consists of a nucleic acid sequence as shown in a sequence selected from the group consisting of SEQ ID NO: 83 to SEQ ID NO: 402 and SEQ ID NO: 907 to SEQ ID NO: 936.

In preferred embodiment, the stretch is 95%, in particular 100% complementary to the target sequence.

Preferably, the antisense oligonucleotide has a length of 12 to 30 nucleotides, more preferably, a length of 14 to 22 nucleotides, and most preferably a length of 16 to 20 nucleotides.

In some embodiments, the antisense oligonucleotide comprises or consists of a nucleic acid sequence as shown in SEQ ID NO: 403 to SEQ ID NO: 881, such as in SEQ ID NO: 403 to SEQ ID NO: 723, or in SEQ ID NO: 724 to SEQ ID NO: 881.

In some embodiments, the antisense oligonucleotide is an antisense compound as shown in Table A1 in FIG. 1, wherein
Adx represents 2′deoxyadenosine-3′-phosphorothioate
Aox represents 2′-O-methyladenosine-3′-phosphorothioate
Amx represents 2′-O-Methoxyethyladenosine-3′-phosphorothioate
Alx represents 2′-O-beta-D-oxy LNA adenosine-3′-phosphorothioate
Cdx represents 2′deoxycytidine-3′-phosphorothioate
Cox represents 2′-O-methylcytidine-3′-phosphorothioate
Cmx represents 2′-O-Methoxyethyl-5-methylcytidine-3′-phosphorothioate
Clx represents 2′-O-beta-D-oxy LNA-5-methylcytidine-3′-phosphorothioate
Edx represents 2′deoxy-5-methylcytidine-3′-phosphorothioate
Emx represents 2′-O-Methoxyethyl-5-methylcytidine-3′-phosphorothioate
Elx represents 2′-O-beta-D-oxy LNA-5-methylcytidine-3′-phosphorothioate
Gdx represents 2′deoxyguanosine-3′-phosphorothioate
Gox represents 2′-O-methylguanosine-3′-phosphorothioate
Gmx represents 2′-O-Methoxyethylguanosine-3′-phosphorothioate
Glx represents 2′-O-beta-D-oxy LNA guanosine-3′-phosphorothioate
Tdx represents 2′deoxythymidine-3′-phosphorothioate
Tmx represents 2′-O-Methoxyethylthymidine-3′-phosphorothioate
Tlx represents 2′-O-beta-D-oxy LNA thymidine-3′-phosphorothioate
Uox represents 2′-O-methyluridine-3′-phosphorothioate
Ado represents 2′deoxyadenosine-3′-phosphate
Aoo represents 2′-O-methyladenosine-3′-phosphate
Amo represents 2′-O-Methoxyethyladenosine-3′-phosphate
Alo represents 2′-O-beta-D-oxy LNA adenosine-3′-phosphate
Cdo represents 2′deoxycytidine-3′-phosphate
Coo represents 2′-O-methylcytidine-3′-phosphate
Cmo represents 2′-O-Methoxyethyl-5-methylcytidine-3′-phosphate
Elo represents 2′-O-beta-D-oxy LNA-5-methylcytidine-3′-phosphate
Edo represents 2′deoxy-5-methylcytidine-3′-phosphate
Emo represents 2′-O-Methoxyethyl-5-methylcytidine-3′-phosphate
Elo represents 2′-O-beta-D-oxy LNA-5-methylcytidine-3′-phosphate
Gdo represents 2′deoxyguanosine-3′-phosphate
Goo represents 2′-O-methylguanosine-3′-phosphate
Gmo represents 2′-O-Methoxyethylguanosine-3′-phosphate
Glo represents 2′-O-beta-D-oxy LNA guanosine-3′-phosphate
Tdo represents 2′deoxythymidine-3′-phosphate
Tmo represents 2′-O-Methoxyethylthymidine-3′-phosphate
Tlo represents 2′-O-beta-D-oxy LNA thymidine-3′-phosphate
Uoo represents 2′-O-methyluridine-3′-phosphate
Ad represents 2′deoxyadenosine-3′
Ao represents 2′-O-methyladenosine-3′
Am represents 2′-O-Methoxyethyladenosine-3′
Al represents 2′-O-beta-D-oxy LNA adenosine-3′
Cd represents 2′deoxycytidine-3′
Co represents 2′-O-methylcytidine-3′
Ed represents 2′deoxy-5-methylcytidine-3′
Em represents 2′-O-Methoxyethyl-5-methylcytidine-3′
El represents 2′-O-beta-D-oxy LNA-5-methylcytidine-3′
Gd represents 2′deoxyguanosine-3′
Go represents 2′-O-methylguanosine-3′
Gm represents 2′-O-Methoxyethylguanosine-3′
Gl represents 2′-O-beta-D-oxy LNA guanosine-3′
Td represents 2′deoxythymidine-3′
Tm represents 2′-O-Methoxyethylthymidine-3′,
Tl represents 2′-O-beta-D-oxy LNA thymidine-3′, and
Uo represents 2′-O-methyluridine-3′.

In some embodiments, the antisense oligonucleotide is an antisense compound as shown in Table C1 in FIG. 2.

In an embodiment, the antisense oligonucleotide is an antisense oligonucleotide with ASO ID 2_17, 2_35, 2_50, 2_51, 2_52, 6_8, 25_113, 29_2, 29_5, 29_6, 29_7, 29_10, 29_23, 29_24, 29_32, 29_34, 29_36, 29_54, 29_70, 29_72, 29_78, 29_79, 29_84, 29_85, 29_86, 29_100, 29_112, 29_121, 29_123, 29_124, 29_125, 29_130, 29_133, 29_138, 29_154, 34_38, 34_39, 34_46, 34_47, 37_15, 38_5, 38_10, 38_11, or 51_66 as shown in Table A1. The ASO compounds with these ASO IDs had the best effect on the down-regulation of the target gene (see Examples 1 and 2).

In preferred embodiment, the antisense oligonucleotide is an antisense oligonucleotide compound selected from the compounds shown in Table A3. Here, the compounds are provided in the so called HELM annotation format.

In another preferred embodiment, the antisense oligonucleotide is an antisense oligonucleotide compound selected from the compounds shown in Table C3. Here, the compounds are provided in the so called HELM annotation format. Thus, the antisense oligonucleotide is an preferably antisense oligonucleotide with ASO ID 2_17, 25_111, 25_113, 29_10, 29_124, 29_130, 29_138, 29_24, 29_34, 29_36, 29_5, 29_70, 29_78, 29_79, 29_84, 29_85, 29_86, 34_39, 34_46, 37_15, 38_10, 51_6, 66_117, 66_120, 66_134, 66_135, 66_123, 66_124, 66_130, 66_126, 66_127, 66_149, 66_189, 66_181, 66_182, 66_173, 66_153, 66_160, 66_185, 66_183, 66_177, 66_188, 66_140, 66_176, 66_174, 66_217, 66_220, 66_27, 66_36, 66_39, 66_408, 66_412, 66_430, 66_431, 66_456, 66_459, 66_47, 66_485, 66_492, 66_496, 66_545, 66_547, 66_567, 66_573, 66_584, 66_587, 66_576, 66_588, 66_63, 66_592, 66_593, 66_600, 66_64, 68_16, 54_1, 54_3, 54_2 or 66_544.

More preferably, the antisense oligonucleotide is an antisense oligonucleotide with ASO ID 2_17, 25_113, 29_10, 29_124, 29_70, 29_78, 29_79, 29_84, 29_85, 29_86, 34_39, 51_6, 66_120, 66_134, 66_135, 66_124, 66_130, 66_126, 66_127, 66_149, 66_181, 66_182, 66_173, 66_160, 66_185, 66_183, 66_177, 66_188, 66_140, 66_176, 66_174, 66_220, 66_27, 66_36, 66_39, 66_408, 66_430, 66_431, 66_456, 66_47, 66_485, 66_492, 66_496, 66_545, 66_547, 66_567, 66_573, 66_584, 66_587, 66_576, 66_588, 66_63, 66_592, 66_593, 66_600, 66_64, 54_3, 54_2, or 66_544.

Most preferably, the antisense oligonucleotide is an antisense oligonucleotide with ASO ID 2_17, 29_124, 29_79, 29_84, 29_85, 29_86, 66_134, 66_135, 66_124, 66_130, 66_126, 66_149, 66_181, 66_182, 66_173, 66_160, 66_185, 66_183, 66_177, 66_188, 66_140, 66_176, 66_174, 66_220, 66_27, 66_36, 66_39, 66_408, 66_47, 66_485, 66_496, 66_545, 66_547, 66_567, 66_573, 66_584, 66_587, 66_576, 66_588, 66_63, 66_592, 66_593, 66_600, 66_64, 543, 54_2 or 66_544.

The antisense oligonucleotide of the present invention shall be capable of reducing the amount of NAT8L (N-acetyltransferase 8 like) mRNA in a host cell expressing said NAT8L mRNA. Typically, the host cell is a mammalian cell, such as a primate cell. In a preferred embodiment, said host cell is human host cell.

Preferably, the antisense oligonucleotide is a chemically modified antisense oligonucleotide. A chemically modified antisense oligonucleotide typically comprises modifications of the phosphodiester backbone chemistry, nucleobase modifications and sugar modifications.

In a preferred embodiment, the chemically modified antisense oligonucleotide comprises at least one 2′ modified sugar or bicyclic sugar.

Alternatively or additionally, the chemically modified antisense oligonucleotide contains at least one modified nucleobase. For example, at least one modified nucleobase is 5-methylcytosine.

Alternatively or additionally, the chemically modified antisense oligonucleotide comprises at least one modified nucleoside selected from the group consisting of: 2′-O-Methoxyethyl-RNA, 2′-O-Methyl-RNA, 2′-Fluoro-RNA.

Further, the chemically modified antisense oligonucleotide may comprise at least one modified internucleoside linkage. In an embodiment, at least five, such as at least 10 internucleoside linkages are modified internucleoside linkages. In an embodiment, all internucleoside linkages are modified internucleoside linkages.

A “modified internucleoside linkage” as used herein, refers to an internucleoside linkage other than a phosphodiester linkage. Thus, the antisense oligonucleotide may comprise unmodified internucleoside linkages (i.e. phosphodiester linkages), modified internucleoside linkages, or a combination thereof.

Preferably, the modified linkage(s) is (are) selected from: a Phosphorothioate internucleoside linkage, a Phosphorodithioate internucleoside linkage, a Phophoroamidate internucleoside linkage, a methyl phosphonate internucleoside linkage, a phosphotriester internucleoside linkage, a boranophosphate internucleoside linkage and a phosphoryl guanidine internucleoside linkage. Moreover, internucleoside linkage can be stereodefined versions of said linkages.

More preferably, the at least one modified linkage is a phosphorothioate linkage. Most preferably, at least 50% of the internucleoside linkages, such as all internucleoside linkages, are phosphorothioate internucleoside linkages.

Preferably, the antisense oligonucleotide comprises at least one nucleoside with a modified sugar moiety, typically at least four nucleosides with a modified sugar moiety. (herein also referred to as sugar modified nucleosides).

In an embodiment, the antisense oligonucleotide comprises at least one, such as one, two, three, four or more LNA (locked nucleic acid) or MOE (2′-O-Methoxyethyl) nucleosides. For example, the LNA nucleoside is a beta-D-oxy LNA nucleoside.

Moreover, it is envisaged that the antisense oligonucleotide has a gapmer structure, i.e. is a gapmer.

The present invention further relates to a conjugate comprising the antisense oligonucleotide according to the present invention, wherein the said antisense oligonucleotide is covalently attached to a conjugate moiety.

The present invention further relates to pharmaceutical composition comprising the antisense oligonucleotide according to the present invention or the conjugate according to the present invention. In an embodiment, the composition further comprises diluents and carriers.

The present invention further relates to the antisense oligonucleotide according to the present invention, the conjugate according to the present invention, or the pharmaceutical composition according to the present invention for use in treating Canavan disease.

The present invention further relates to a method for treating Canavan disease, comprising administering to a subject suffering from Canavan disease a pharmaceutically effective amount of the antisense oligonucleotide according to the present invention, the conjugate according to the present invention, or the pharmaceutical composition according to the present invention for use in treating Canavan disease.

The present invention further relates to a method for identifying a candidate compound for the treatment of Canavan disease, comprising
a) providing an antisense oligonucleotide according to the present invention,
b) contacting a host cell expressing NAT8L mRNA with said antisense oligonucleotide,
c) determining the amount of NAT8L mRNA in the said host cell, and
d) identifying a candidate compound based on the results of step c).

### FIGURES

FIG. 1: Table A1 with information on antisense compounds tested in the studies underlying the present invention.

FIG. 2: Table C1 with information on antisense compounds tested in the studies underlying the present invention.

### DETAILED OVERVIEW ON THE PRESENT INVENTION

Inhibition of NAT8L (N-acetyltransferase 8 like) expression is considered as therapeutic concept for treating Canavan disease. In the studies underlying the present invention, target regions within the NAT8L pre-mRNA were identified which—when targeted by antisense oligonucleotides—allow for efficient downregulation of the human NAT8L pre-mRNA (or mRNA) in a host cell expressing said pre-mRNA or mRNA (see Tables 3, 6 and 8). Further, down-regulation of expression was observed in a neuronal cell line. The sequences of the target regions are shown in Tables B1, B2, B3 and D1. Thus, the invention provides antisense oligonucleotides which are capable of downregulating NAT8L. Preferably, the antisense oligonucleotides (ASOs) comprise a stretch of at least 10 nucleotides which is preferably 90%, more preferably, 95% and most preferably fully complementary (i.e. 100% complementary) to the target region (herein also referred to as target sequence). The antisense oligonucleotides of the present invention are candidates for the treatment of Canavan disease. Advantageously, compounds with a low neuronal toxicity were identified. Moreover, after administration of a compound of the present invention to a non-human primate no adverse side effects were observed.

Accordingly, the present invention relates to an antisense oligonucleotide comprising a stretch of at least 10 nucleotides which is at least 90% complementary to a target sequence in a NAT8L (N-acetyltransferase 8 like) gene.

The term “oligonucleotide” as used herein is well known in the art. As used herein, the term refers to a molecule of at least ten covalently linked nucleotides. Typically, the oligonucleotides as referred to herein are chemically synthesized, for example by solid-phase chemical synthesis. The oligonucleotides as referred to herein shall contain various chemical modifications which typically do occur in nature. For example, the antisense oligonucleotide may contain at least one 2′ modified sugar. In a preferred embodiment, the antisense oligonucleotides are gapmers. The oligonucleotides of the present invention are antisense oligonucleotides, and in particular single-stranded oligonucleotides. Accordingly, they shall be capable of binding the NAT8L gene, in particular to the NAT8L pre-mRNA, when expressed in a cell, thereby down-regulating the expression of NAT8L gene in the cell. In an embodiment, the cell is a human cell is a cell of the central nervous system (CNS). Typically, the cell is a brain cell.

The NAT8L (N-acetyltransferase 8 like) gene is well known the art. The NAT8L gene is typically the human NAT8L gene. Information on the gene, such as on the nucleic acid sequence, can be found in the known databases, for example, under NCBI Gene ID: 339983). Alternative names of the gene are FLJ37478, Hcml3, Shati or N-acetylaspartate synthetase gene)

The human NAT8L gene encodes a protein having N-acetylaspartate synthetase activity (EC 2.3.1.17). Accordingly, it catalyzes the synthesis of N-acetylaspartate acid (NAA) from L-aspartate and acetyl-CoA. The protein sequence can be assessed in the Uniprot database under the accession number Q8N9F0 (NAT8L_HUMAN). Typically, the human NAT8L protein has an amino acid sequence as shown in SEQ ID NO: 3 (which is encoded by a transcript having a sequence as shown in SEQ ID NO: 2).

The NAT8L protein is typically referred to as “N-acetyltransferase 8 like”. Alternative names are N-acetylaspartate synthetase, NAA Synthetase, or aspartate N-acetyltransferase).

Typically, the ASO of the present invention targets the human NAT8L pre-mRNA, i.e. downregulates expression of said pre-mRNA. The sequence of the human NAT8L pre-mRNA can be e.g. assessed in the Ensembl database under accession number in ENST00000423729.3. It is encoded by a region on human Chromosome 4: position 2,059,327-2,069,089 on the forward strand (Assembly GRCh38). The sequence of the human pre-mRNA has a sequence as shown in SEQ ID NO: 1. Within the cell, the pre-mRNA is further processed, i.e., by splicing, thereby generating a protein coding mRNA (herein also referred to as transcript). In some embodiments, the antisense oligonucleotide of the present invention may also target the human NAT8L mRNA (if the target region is located within an exon, either coding or in the 3′- or 5′UTR). For example, the antisense oligonucleotide of the present invention may target the human NAT8L mRNA having a sequence as shown in SEQ ID NO: 3. SEQ ID NO: 1 and 3 are RNA sequences. In the sequence listing, they are provided as DNA sequences. It is understood by the skilled person that the target RNA sequences have uracil (U) bases instead of thymidine bases (T). The same applies to the other target sequences as referred to herein (such as SEQ ID NO: 10 to SEQ ID NO: 402 or SEQ ID 882 to 936) which are provided as DNA sequences as well.

The human NAT8L pre-mRNA comprises three exons and two introns. An overview on the location of the introns, the exon, and the 3′ and 5′ UTR with the pre-mRNA sequence can be found in the following table.

Overview on UTR, exon and intron regions
in the human NAT8L pre-mRNA

| | Pos start in SEQ ID NO: 1 | Pos End in SEQ ID NO: 1 |
|---|---|---|
| 5′ UTR | 1 | 185 |
| Exon 1 | 186 | 560 |
| Intron 1 | 561 | 1670 |
| Exon 2 | 1671 | 1835 |
| Intron 2 | 1836 | 4432 |
| Exon 3 | 4433 | 4799 |
| 3′UTR | 4800 | 9763 |

As set forth above, the antisense oligonucleotides of the present invention shall be capable of down-regulating, i.e. reducing expression of the NAT8L mRNA in a cell that expresses said mRNA. Preferably, the expression is reduced in a call by antisense oligonucleotides of the present invention by least 30%, at least 40%, at least 50%, at least 60%, at least 70%, at least 80%, or at least 90% as compared to a control cell (i.e. an untreated control cell). How to assess whether the expression is reduced can be assessed by well-known methods, i.e. by measuring the expression level (i.e. the amount of the target mRNA) in ASO treated cells. In an embodiment, the down-regulation of the target gene is assessed as described in the Examples section. As control for down regulation untreated cells can be used. Down-regulating the expression of the NAT8L mRNA, typically, leads down-regulation of the NAT8L protein and thus to reduced levels of N-acetyl-L-Aspartate (NAA) as compared to a control. Down-regulation of the NAT8L protein can be assessed by e.g. assessing the N-acetylaspartate synthetase activity in cells treated with the ASO of the present invention by using well known enzymatic assays or by or quantifying the protein expression, such as by Western Blotting, mass spectrometry or ELISA. Preferably, the N-acetylaspartate synthetase activity is reduced in a cell by antisense oligonucleotides of the present invention by at least 30%, at least 40%, at least 50%, at least 60%, at least 70%, at least 80%, or at least 90% as compared to a control cell (i.e. an untreated control cell).

The above assessments can be done in vivo or in vitro. If they are done in vitro, they are typically done in human cells, such as in human cells used in the Examples section. In vivo, it is e.g. envisaged that a down-regulation of the target mRNA or protein, of at least 30%, such as at least 40% is achieved. For example, the down-regulation of the target mRNA or protein may be between 40% to 60%, or between 50% to 60% as compared to a control.

As set forth above, target sequences within the NAT8L pre-mRNA were identified which can be efficiently targeted with ASOs. In total, 77 of such target regions/sequences were identified. 52 of these target regions are shown in the following table. In the table, each identified target region was assigned a so called “Target ID” (Target ID 1 to 52). These IDs are used throughout the application. The terms “target region”, “target sequence” and “target nucleic acid” are used interchangeably herein.

Overview on locations of target regions identified
within the NAT8L pre-mRNA sequence (Part 1)

| Start in | End in | | |
|---|---|---|---|
| SEQ ID NO: 1 | SEQ ID NO 1 | Target ID | Location |
| 179 | 194 | 1 | Exon 1 (partially 5′ |
| | | | UTR and coding sequence) |
| 890 | 919 | 2 | Intron 1 |
| 937 | 960 | 3 | Intron 1 |
| 1508 | 1551 | 4 | Intron 1 |
| 1755 | 1770 | 5 | Exon 2 |
| 1800 | 1832 | 6 | Exon 2 |
| 2034 | 2049 | 7 | Intron 2 |
| 2314 | 2330 | 8 | Intron 2 |
| 2346 | 2362 | 9 | Intron 2 |
| 3032 | 3046 | 10 | Intron 2 |
| 3825 | 3844 | 11 | Intron 2 |
| 3912 | 3934 | 12 | Intron 2 |
| 3981 | 3996 | 13 | Intron 2 |
| 4143 | 4158 | 14 | Intron 2 |
| 4169 | 4192 | 15 | Intron 2 |
| 4227 | 4243 | 16 | Intron 2 |
| 4502 | 4516 | 17 | Exon 3 |
| 4582 | 4614 | 18 | Exon 3 |
| 4638 | 4651 | 19 | Exon 3 |
| 4666 | 4679 | 20 | Exon 3 |
| 4889 | 4908 | 21 | Exon 3 (3′UTR) |
| 4934 | 4949 | 22 | Exon 3 (3′UTR) |
| 5056 | 5073 | 23 | Exon 3 (3′UTR) |
| 5656 | 5671 | 24 | Exon 3 (3′UTR) |
| 5730 | 5805 | 25 | Exon 3 (3′UTR) |
| 6392 | 6414 | 26 | Exon 3 (3′UTR) |
| 6474 | 6497 | 27 | Exon 3 (3′UTR) |
| 6542 | 6559 | 28 | Exon 3 (3′UTR) |
| 6564 | 6605 | 29 | Exon 3 (3′UTR) |
| 6608 | 6631 | 30 | Exon 3 (3′UTR) |
| 6711 | 6726 | 31 | Exon 3 (3′UTR) |
| 6852 | 6874 | 32 | Exon 3 (3′UTR) |
| 7042 | 7056 | 33 | Exon 3 (3′UTR) |
| 7284 | 7340 | 34 | Exon 3 (3′UTR) |
| 7515 | 7534 | 35 | Exon 3 (3′UTR) |
| 8572 | 8588 | 36 | Exon 3 (3′UTR) |
| 8703 | 8731 | 37 | Exon 3 (3′UTR) |
| 8734 | 8763 | 38 | Exon 3 (3′UTR) |
| 8784 | 8844 | 39 | Exon 3 (3′UTR) |
| 8875 | 8914 | 40 | Exon 3 (3′UTR) |
| 8936 | 8956 | 41 | Exon 3 (3′UTR) |
| 8973 | 8991 | 42 | Exon 3 (3′UTR) |
| 9007 | 9027 | 43 | Exon 3 (3′UTR) |
| 9049 | 9087 | 44 | Exon 3 (3′UTR) |
| 9100 | 9128 | 45 | Exon 3 (3′UTR) |
| 9158 | 9175 | 46 | Exon 3 (3′UTR) |
| 9243 | 9266 | 47 | Exon 3 (3′UTR) |
| 9292 | 9313 | 48 | Exon 3 (3′UTR) |
| 9322 | 9360 | 49 | Exon 3 (3′UTR) |
| 9603 | 9619 | 50 | Exon 3 (3′UTR) |
| 9625 | 9641 | 51 | Exon 3 (3′UTR) |
| 9725 | 9762 | 52 | Exon 3 (3′UTR) |

Further information on the above target regions can be found in Table B1 in the Example 4 (e.g. the sequence of the target region or the SEQ ID NO). Tables B2 and B3 in Example 4 list preferred target regions. The target regions in Tables B2 and B3 may be present in the target regions shown in Table BSE, but may be shorter.

Further target regions are shown in the following table. Again, each identified target region was assigned a so called “Target ID” (Target ID 53 to 77). Some of the target regions shown in the following Table correspond to the target regions in the above table. For example, the target region with Target ID 53 is a subregion of the target region with Target ID 2, i.e. it is contained in this region. For more details, please see the column “Comments”.

Overview on locations of regions identified
within the NAT8L pre-mRNA sequence (Part 2)

| Start in | End in | | |
|---|---|---|---|
| SEQ ID NO: 1 | SEQ ID NO 1 | Target ID | Comments |
| 215 | 231 | 53 | |
| 894 | 917 | 54 | subregion of Target ID 2 |
| 940 | 957 | 55 | subregion of Target ID 3 |
| 1152 | 1168 | 56 | |
| 3043 | 3060 | 57 | expansion of Target ID 10 |
| 3535 | 3552 | 58 | |
| 3572 | 3589 | 59 | |
| 4228 | 4244 | 60 | expansion of Target ID 16 |
| 5735 | 5761 | 61 | subregion of Target ID 25 |
| 5768 | 5791 | 62 | subregion of Target ID 25 |
| 6187 | 6208 | 63 | |
| 6390 | 6410 | 64 | expansion of Target ID 26 |
| 6477 | 6504 | 65 | expansion of Target ID 27 |
| 6528 | 6597 | 66 | expansion of Target ID 29 |
| 7013 | 7036 | 67 | |
| 7291 | 7310 | 68 | subregion of Target ID 34 |
| 7507 | 7528 | 69 | expansion of Target ID 35 |
| 8722 | 8768 | 70 | expansion of Target ID 38 |
| 8810 | 8827 | 71 | subregion of Target ID 39 |
| 8883 | 8900 | 72 | subregion of Target ID 40 |
| 8938 | 8958 | 73 | expansion of Target ID 41 |
| 8966 | 8984 | 74 | expansion of Target ID 42 |
| 9154 | 9177 | 75 | expansion of Target ID 46 |
| 9329 | 9356 | 76 | subregion of Target ID 49 |
| 9743 | 9760 | 77 | subregion of Target ID 52 |

Further information on the above target regions can be found in Table D1 in the Examples section (e.g. the sequence of the target region or the SEQ ID NO). Tables D2 and D3 list preferred target sequences.

Preferably, the antisense oligonucleotide of the present invention is capable of binding (i.e. hybridizing) to a target region selected from a target region shown in the above table or in Table B1 in Example 5. Thus, the target sequence has a sequence selected from the group of target sequences consisting of SEQ ID NO: 30 to SEQ ID NO: 81.

More preferably, the antisense oligonucleotide is capable of binding (i.e. hybridizing) to a target region selected from a target region shown in Table B2. Accordingly, the target sequence has a sequence selected from the group of target sequences consisting of SEQ ID NO: 11 to SEQ ID NO: 29.

Most preferably, the antisense oligonucleotide is capable of binding (i.e. hybridizing, i.e. complementary) to a target region selected from a target region shown in Table B3. Accordingly, the target sequence has a sequence selected from the sequences consisting of SEQ ID NO: 4 to SEQ ID NO: 10.

Also preferably, the antisense oligonucleotide of the present invention is capable of binding (i.e. hybridizing) to a target region selected from a target region shown in the above table or in Table D1 in the Examples section. Thus, the target sequence has a sequence selected from the group of target sequences consisting of SEQ ID NO: 882 to SEQ ID NO: 906. More preferably, the antisense oligonucleotide is capable of binding (i.e. hybridizing) to a target region selected from a target region shown in Table D2. Accordingly, the target sequence has a sequence selected from the group of target sequences consisting of SEQ ID NO: 883, 892, 894, 895, 896, 897, 898 and 899. Most preferably, the antisense oligonucleotide is capable of binding (i.e. hybridizing) to a target region selected from a target region shown in Table D3. Accordingly, the target sequence has a sequence selected from the sequences consisting of SEQ ID NO: 883, 894, 895, 897, 898 and 899.

Accordingly, the antisense oligonucleotide typically comprises stretch of at least 10 nucleotides which is at least 90% complementary (such as 95% or 100%) to a target sequence selected from the group of target sequences consisting of SEQ ID NO: 4 to SEQ ID NO: 81 and SEQ ID NO: 882 to SEQ ID NO: 906.

In a preferred embodiment, the target sequence has a sequence as shown in SEQ ID NO: 4.

In another preferred embodiment, the target sequence has a sequence as shown in SEQ ID NO: 5.

In another preferred embodiment, the target sequence has a sequence as shown in SEQ ID NO: 6.

In another preferred, the target sequence has a sequence as shown in SEQ ID NO: 7.

In another preferred, the target sequence has a sequence as shown in SEQ ID NO: 8.

In another preferred, the target sequence has a sequence as shown in SEQ ID NO: 9.

In another preferred, embodiment, the target sequence has a sequence as shown in SEQ ID NO: 10.

In another preferred embodiment, the target sequence has a sequence as shown in SEQ ID: 894.

In another preferred embodiment, the target sequence has a sequence as shown in SEQ ID: 883.

In another preferred, the target sequence has a sequence as shown in SEQ ID NO: 895.

In another preferred, the target sequence has a sequence as shown in SEQ ID NO: 897.

In another preferred, the target sequence has a sequence as shown in SEQ ID NO: 898.

In another preferred, embodiment, the target sequence has a sequence as shown in SEQ ID NO: 899.

Further, the target sequence, preferably comprises a sequence selected from the group consisting of SEQ ID NO: 83 to SEQ ID NO: 402. Alternatively, the target sequence comprises a sequence selected from the group consisting of SEQ ID NO: 907 to SEQ ID NO: 936 (see Table D4). Also preferably, the target sequences consists of a sequence selected from the group consisting of SEQ ID NO: 83 to SEQ ID NO: 402, and SEQ ID NO: 907 to SEQ ID NO: 936.

As set forth above, the antisense oligonucleotides of the present invention are preferably single-stranded antisense oligonucleotides. Preferably, the antisense oligonucleotides of the present invention are not inhibitory RNAs. In particular, the antisense oligonucleotides of the present invention are not siRNAs or short-hairpin RNAs.

In order to bind to a target sequence as referred to herein, the antisense oligonucleotides of the invention shall comprise a “stretch of nucleotides” which is sufficient complementary to a target sequence as referred to herein. In an embodiment, the stretch of nucleotides is at least 90% complementary to a target sequence. In another embodiment, the stretch of nucleotides is at least 95% complementary to a target sequence. In particular preferred embodiment, the stretch of nucleotides is fully complementary (i.e. 100% complementary to the target sequence). The term “complementary” is well known in the art. The percentage of complementary is typically calculated by calculating the proportion of nucleotides (in %) within the stretch of oligonucleotides of the ASO of the present invention which are complementary to the target sequence within the NAT8L gene. A nucleotide present in the ASO of the present invention are considered as complementary if it forms a Watson-Crick base pair with the nucleotide present in the target RNA sequence. Watson Crick base pairs are guanine-cytosine; adenine-uracil, and adenine-thymine, i.e. G-C, A-U or A-T. As will be understood by the skilled person modified nucleotides have also the capacity to form such base pairs. For more information, see e.g. Table A2.

As will be understood by the skilled person, the “stretch of nucleotides” as referred to herein needs to have a certain length in order to allow for the binding of the oligonucleotide of the present invention to the target region. Preferably, the stretch of nucleotides has a length of at least 10 nucleotides, more preferably of at least 12 nucleotides and most preferably of at least 14 nucleotides. Further, the antisense oligonucleotide of the present invention may comprise further nucleotides—i.e. in addition to the stretch of nucleotides as referred to above, such as linker nucleotides. These further nucleotides may be complementary to the target sequence, or not.

In total, the antisense oligonucleotide of the present invention, preferably, has a length of 12 to 30 nucleotides, more preferably, of 14 to 22 nucleotides, and most preferably of 16 to 20 nucleotides. Accordingly, it is envisaged that the antisense oligonucleotide in not longer than 30 nucleotides. In some embodiments, the antisense oligonucleotide in not longer than 22 nucleotides or 20 nucleotides.

In a preferred embodiment, the antisense oligonucleotide comprises a nucleic acid sequence selected from SEQ ID NO: 403 to SEQ ID NO: 723. In another preferred embodiment, the antisense oligonucleotide consists of a nucleic acid sequence selected from SEQ ID NO: 403 to SEQ ID NO: 723.

In yet another preferred embodiment, the antisense oligonucleotide comprises a nucleic acid sequence selected from SEQ ID NO: 724 to 881. In another embodiment, the antisense oligonucleotide consists of a nucleic acid sequence selected from SEQ ID NO: 724 to 881.

In a particular preferred embodiment, the antisense oligonucleotide comprises or consists of a nucleic acid sequence selected from SEQ ID NO: 724 to 881. Preferably, the antisense oligonucleotide comprises a nucleic acid sequence selected from SEQ ID NO: 410, 517, 558, 580, 582, 584, 661, 557, 570, 573, 613, 614, 621, 630, 707, 725, 799, 801, 802, 803, 805, 785, 787, 819, 822, 823, 789, 828, 829, 830, 831, and 835. More preferably, the antisense oligonucleotide consists of a nucleic acid sequence selected from SEQ ID NO: 410, 517, 558, 580, 582, 584, 661, 557, 570, 573, 613, 614, 621, 630, 707, 725, 799, 801, 802, 803, 805, 785, 787, 819, 822, 823, 789, 828, 829, 830, 831, and 835.

Further, it is envisaged that the antisense oligonucleotide comprises, preferably, at least 10, more preferably at least 12, even more preferably at least 14 and most preferably, at least 15 consecutive nucleotides of the sequences selected from SEQ ID NO: 403 to 881. Moreover, it is envisaged that the antisense oligonucleotide comprises, preferably, at least 10, more preferably at least 12, even more preferably at least 14 and most preferably, at least 15 consecutive nucleotides of the compounds shown in Table A1 and C1.

In an embodiment, the antisense oligonucleotide comprises or, in particular, consists of a nucleic acid sequence as shown in SEQ ID NO: 410 (see e.g. compound with ASO ID 2_17).

In another embodiment, the antisense oligonucleotide comprises or, in particular, consists of a nucleic acid sequence as shown in SEQ ID NO: 413 (see e.g. compound with ASO ID 2_50, 2_52, or 2_35).

In another embodiment, the antisense oligonucleotide comprises or, in particular, consists of a nucleic acid sequence as shown in SEQ ID NO: 517 (see e.g. compound with ASO ID 25_111 or 25_113).

In another embodiment, the antisense oligonucleotide comprises or, in particular, consists of a nucleic acid sequence as shown in SEQ ID NO: 557 (see e.g. compound with ASO ID 29_5).

In another embodiment, the antisense oligonucleotide comprises or, in particular, consists of a nucleic acid sequence as shown in SEQ ID NO: 558 (see e.g. compound with ASO ID 29_10).

In another embodiment, the antisense oligonucleotide comprises or, in particular, consists of a nucleic acid sequence as shown in SEQ ID NO: 561 (see e.g. compound with ASO ID 29_34).

In another embodiment, the antisense oligonucleotide comprises or, in particular, consists of a nucleic acid sequence as shown in SEQ ID NO: 570 (see e.g. compound with ASO ID 29_70).

In another embodiment, the antisense oligonucleotide comprises or, in particular, consists of a nucleic acid sequence as shown in SEQ ID NO: 573 (see e.g. compound with ASO ID 29_78, 29_79, 29_84, 29_85 or 29_86).

In another embodiment, the antisense oligonucleotide comprises or, in particular, consists of a nucleic acid sequence as shown in SEQ ID NO: 580 (see e.g. compound with ASO ID 29_124 or 29_121).

In another embodiment, the antisense oligonucleotide comprises or, in particular, consists of a nucleic acid sequence as shown in SEQ ID NO: 582 (see e.g. compound with ASO ID 29_130).

In another embodiment, the antisense oligonucleotide comprises or, in particular, consists of a nucleic acid sequence as shown in SEQ ID NO: 584 (see e.g. compound with ASO ID 29_131).

In another embodiment, the antisense oligonucleotide comprises or, in particular, consists of a nucleic acid sequence as shown in SEQ ID NO: 613 (see e.g. compound with ASO ID 34_39).

In another embodiment, the antisense oligonucleotide comprises or, in particular, consists of a nucleic acid sequence as shown in SEQ ID NO: 614 (see e.g. compound with ASO ID 34_46).

In another embodiment, the antisense oligonucleotide comprises or, in particular, consists of a nucleic acid sequence as shown in SEQ ID NO: 621 (see e.g. compound with ASO ID 37_15).

In another embodiment, the antisense oligonucleotide comprises or, in particular, consists of a nucleic acid sequence as shown in SEQ ID NO: 630 (see e.g. compound with ASO ID 38_10).

In another embodiment, the antisense oligonucleotide comprises or, in particular, consists of a nucleic acid sequence as shown in SEQ ID NO: 707 (see e.g. compound with ASO ID 51_6).

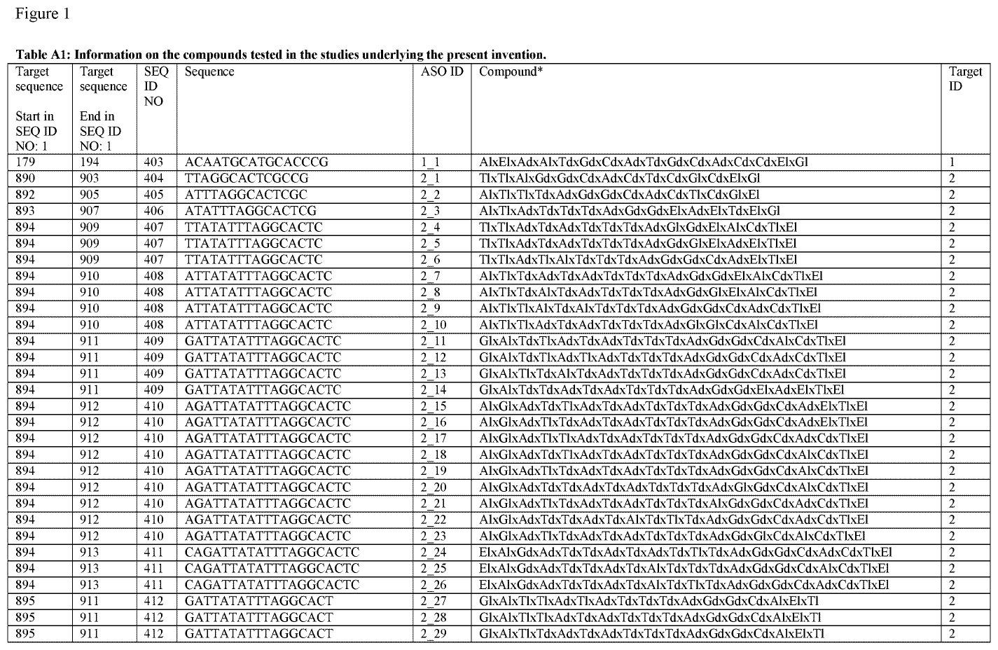

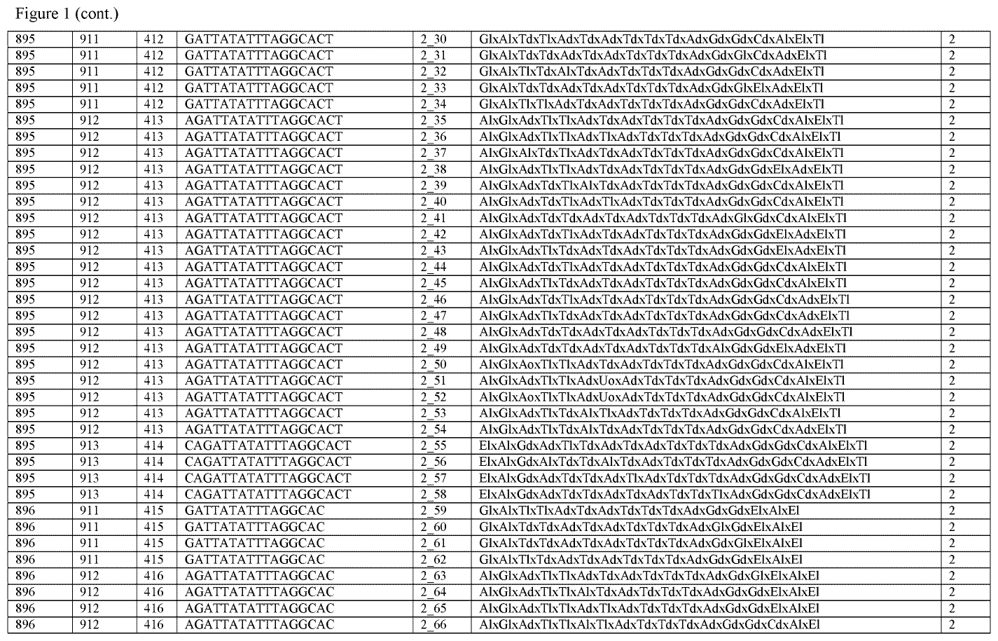

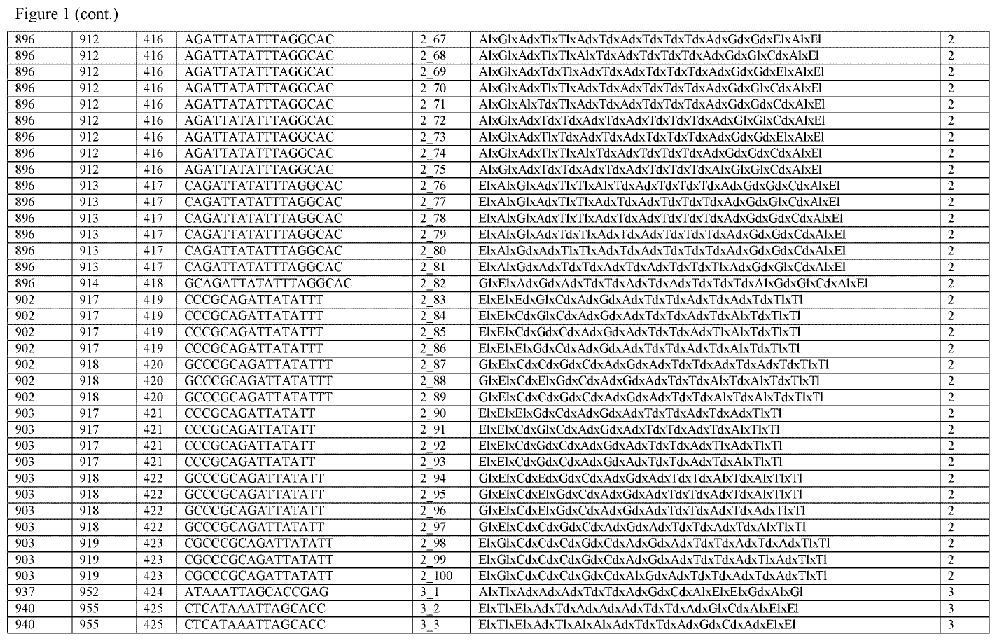

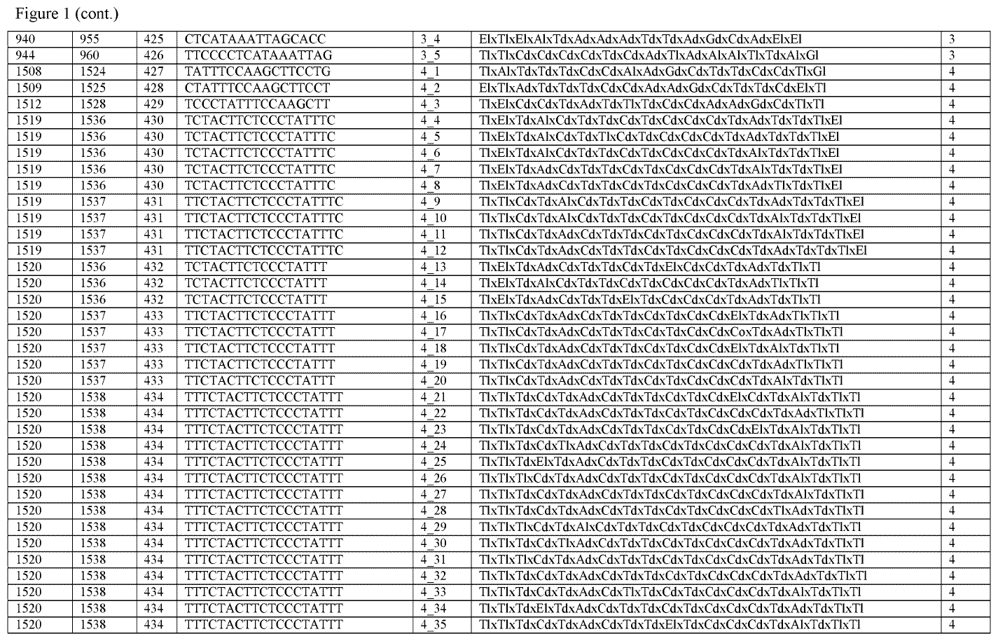

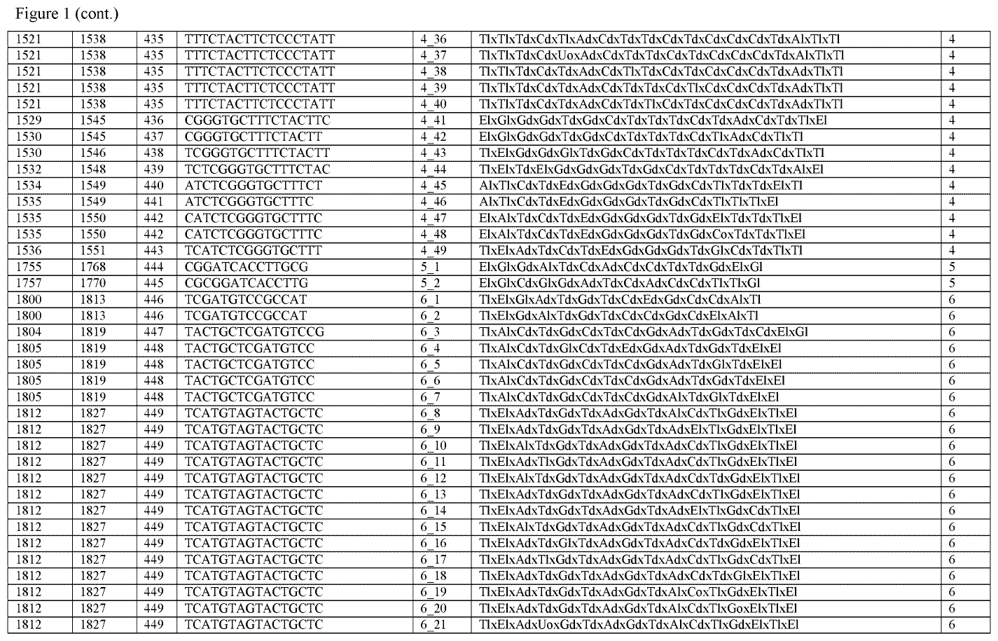

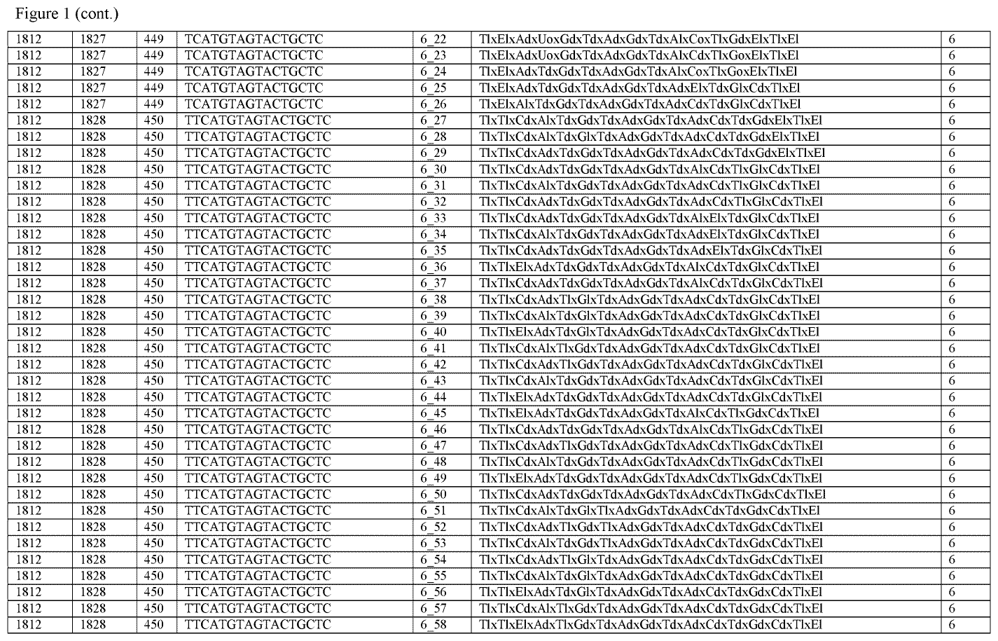

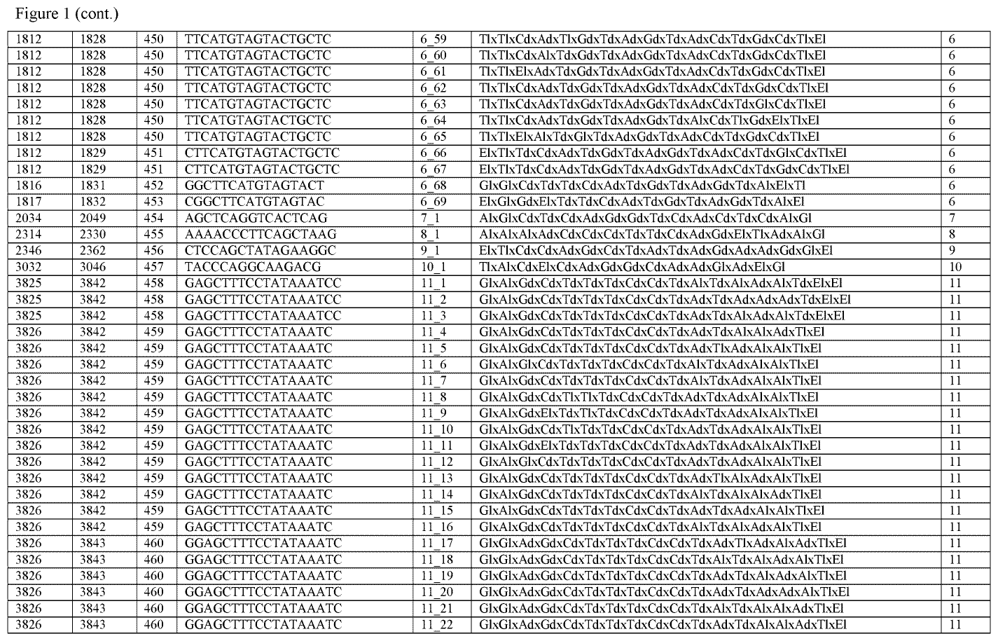

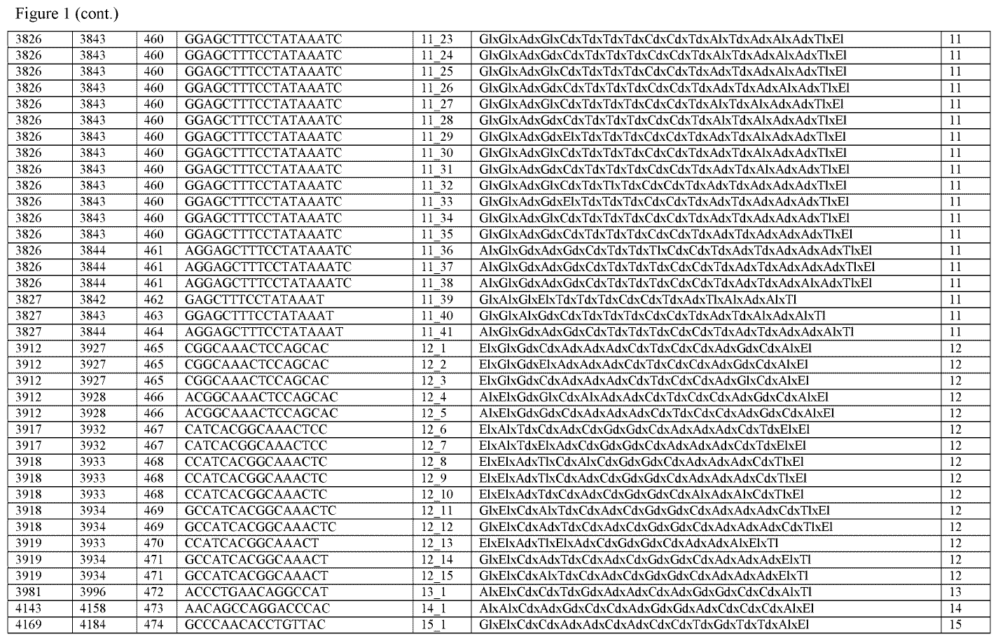

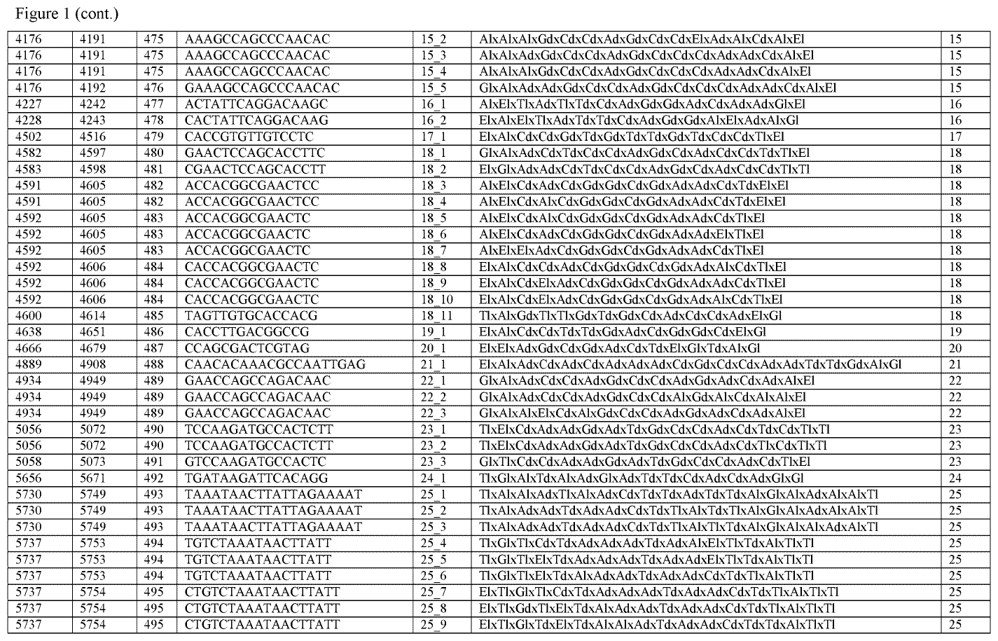

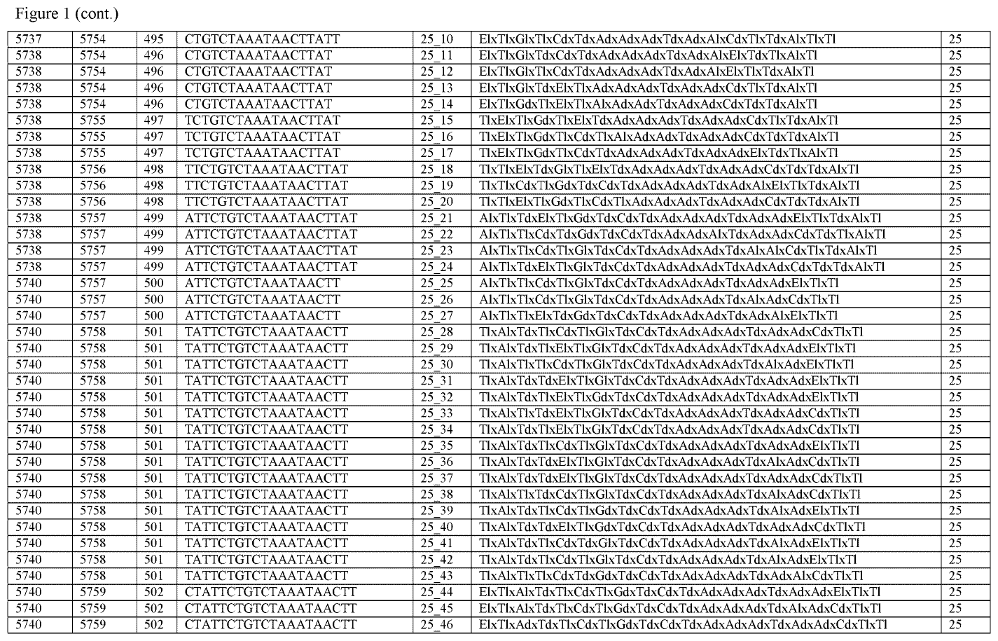

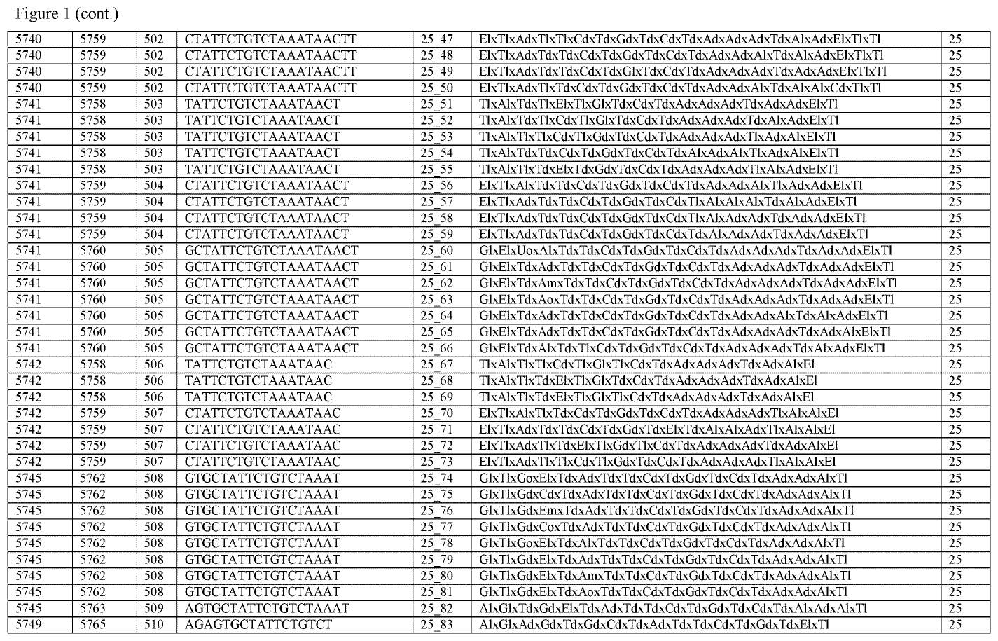

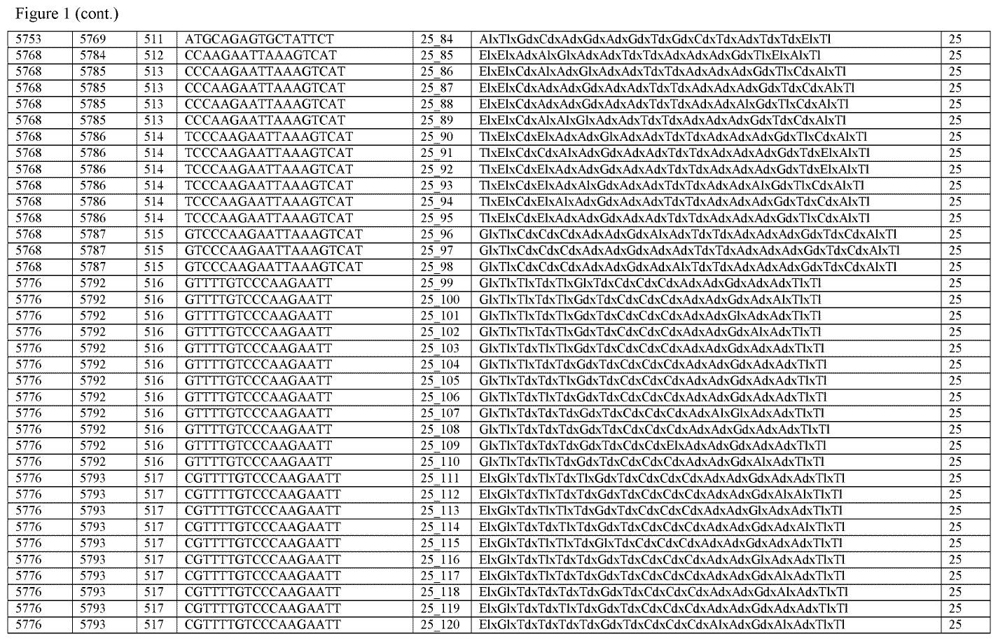

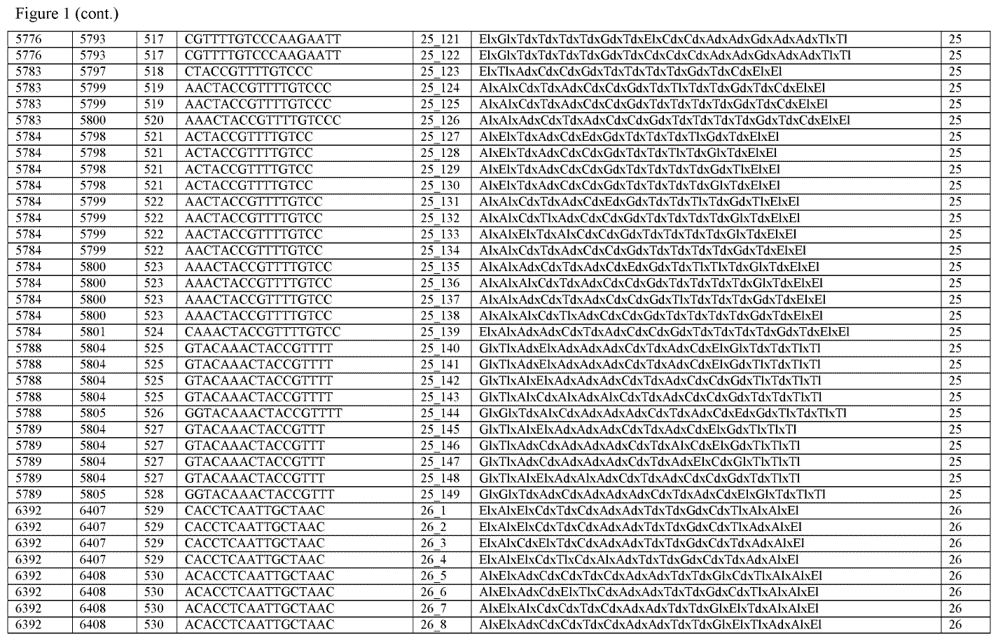

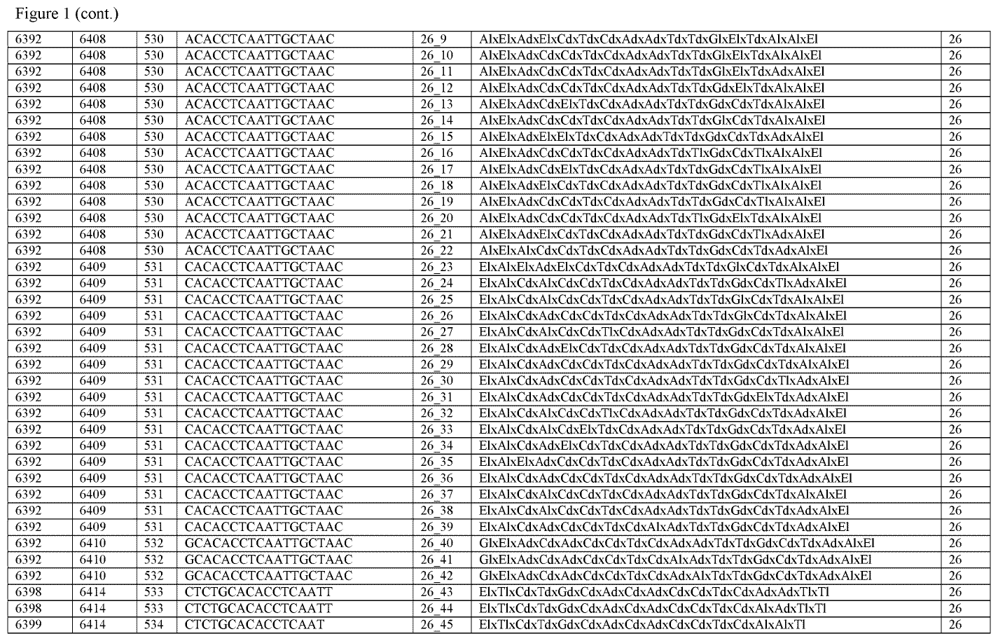

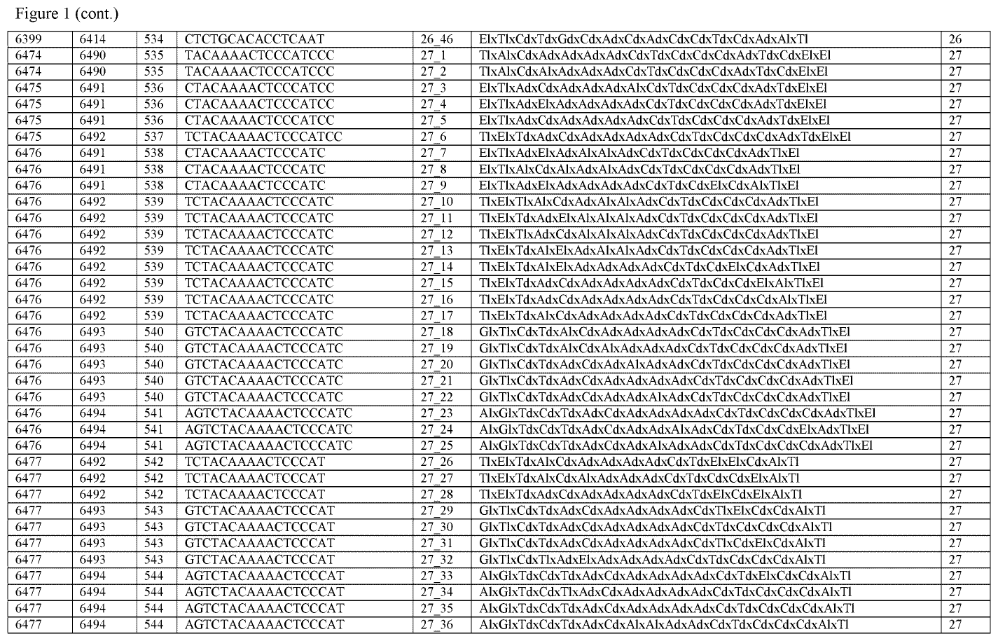

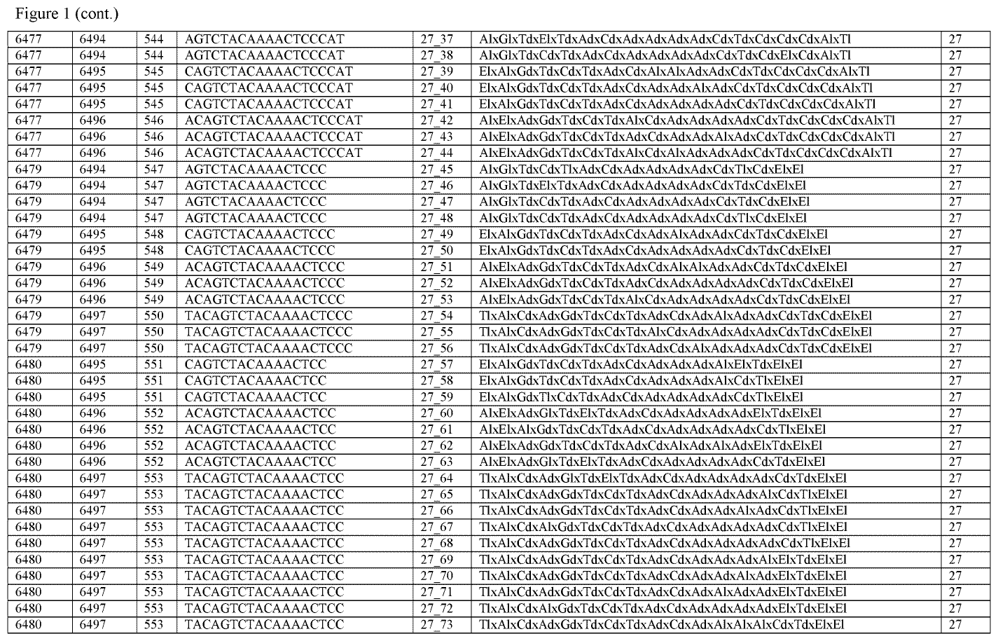

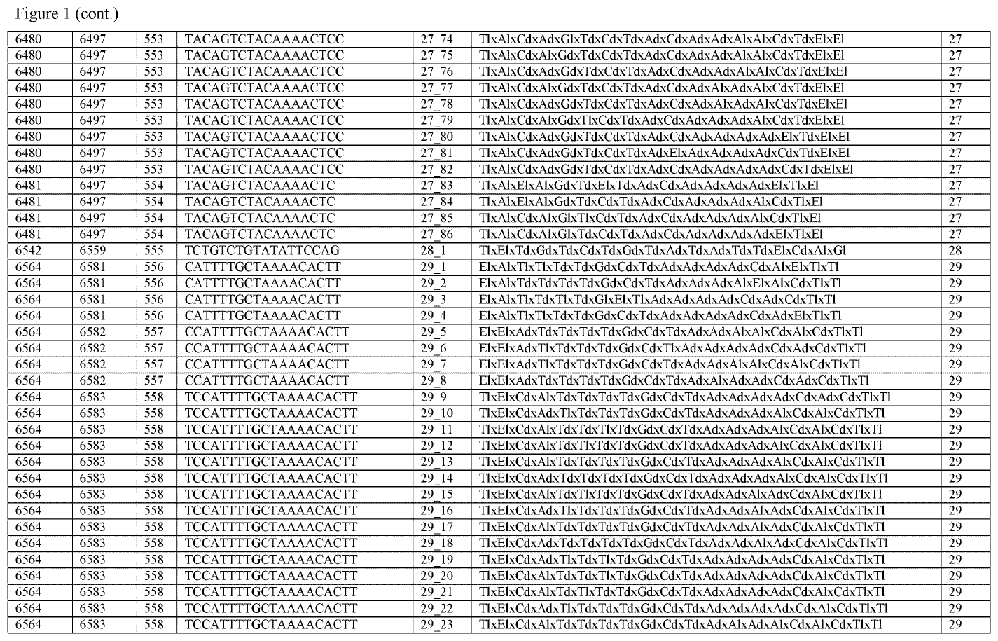

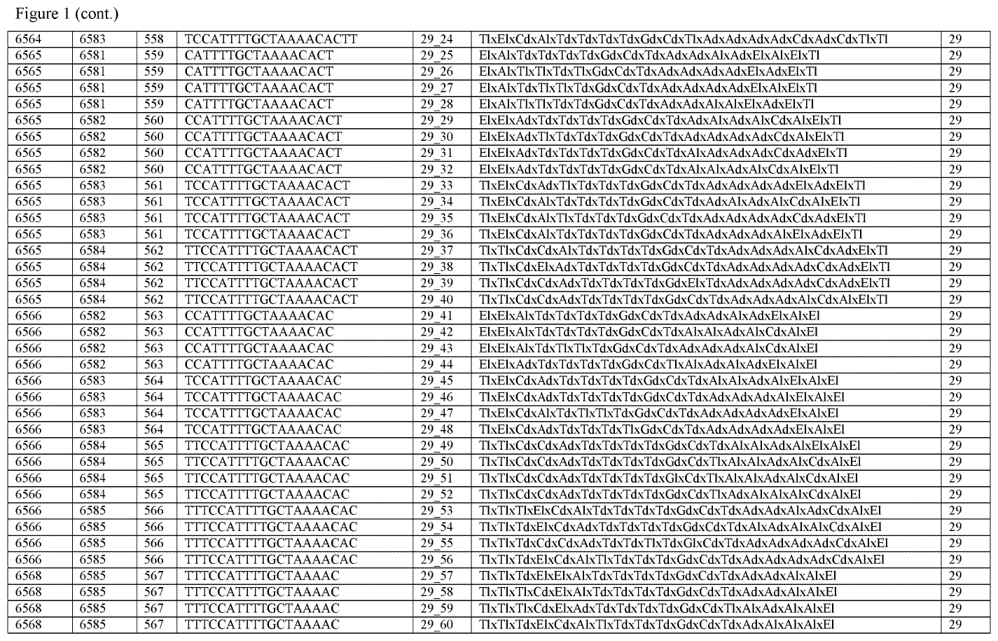

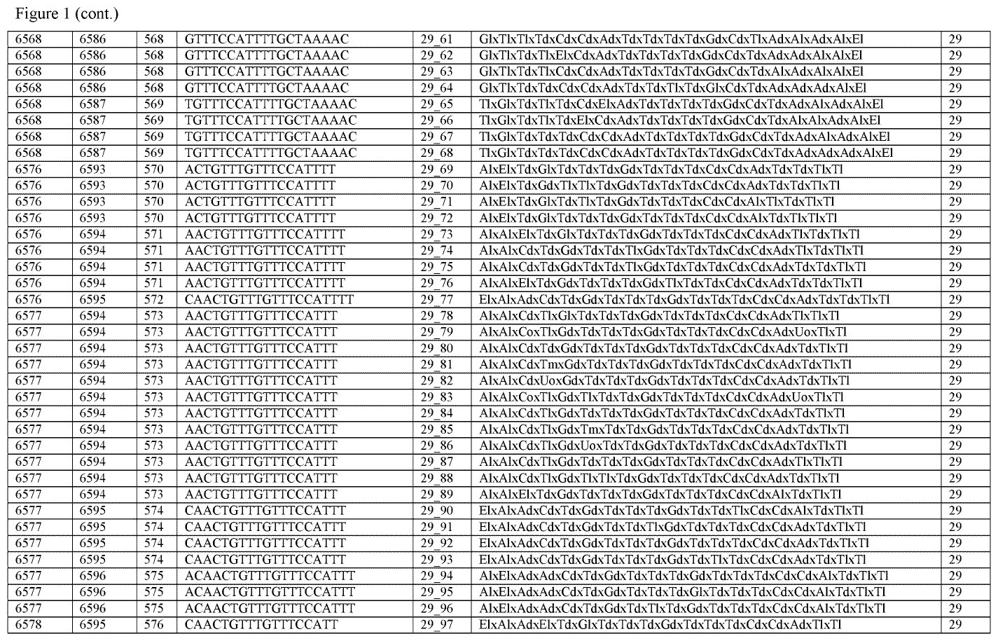

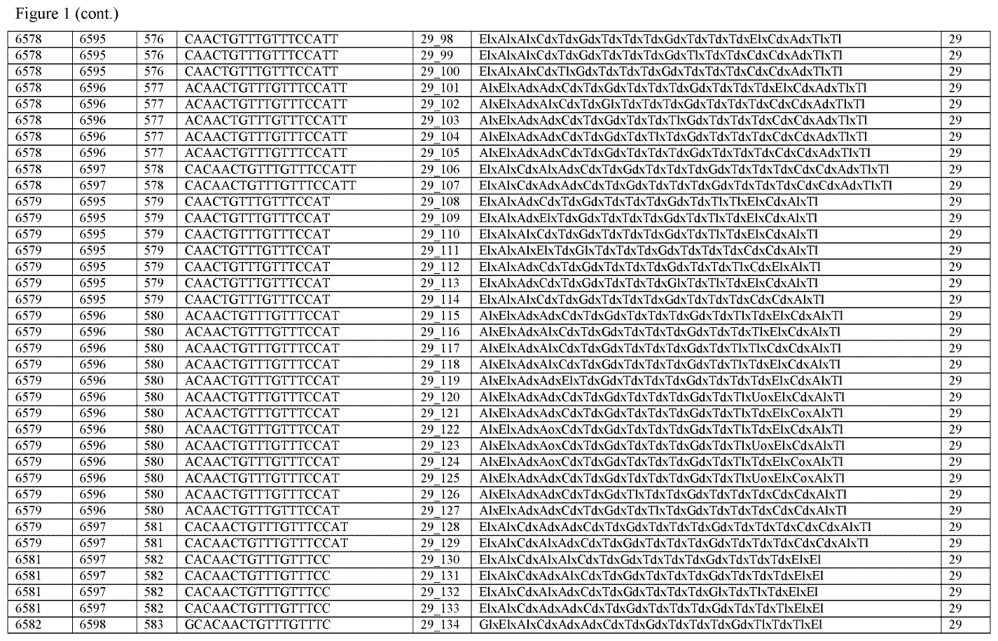

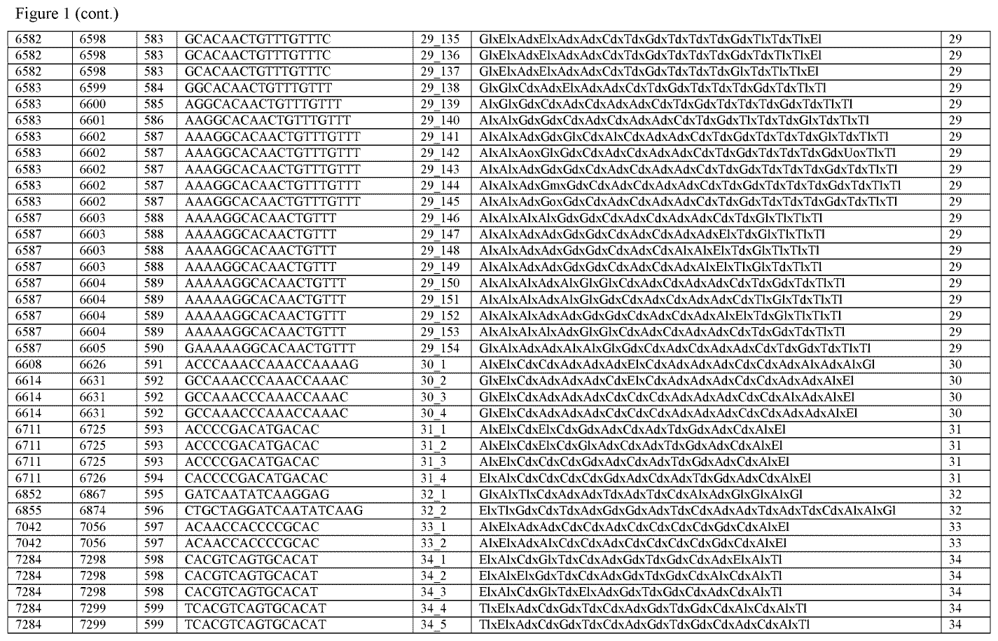

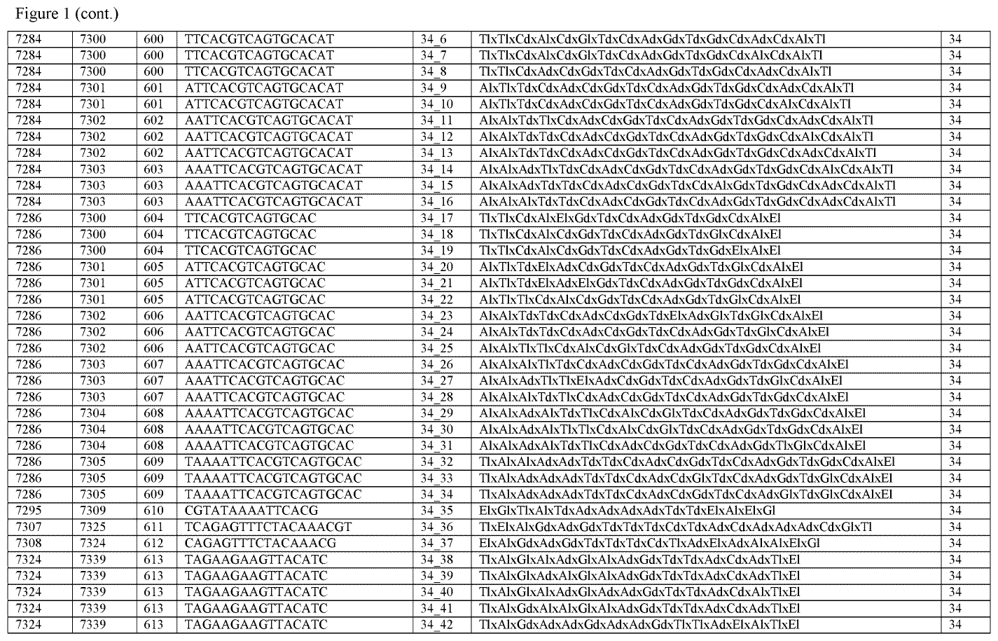

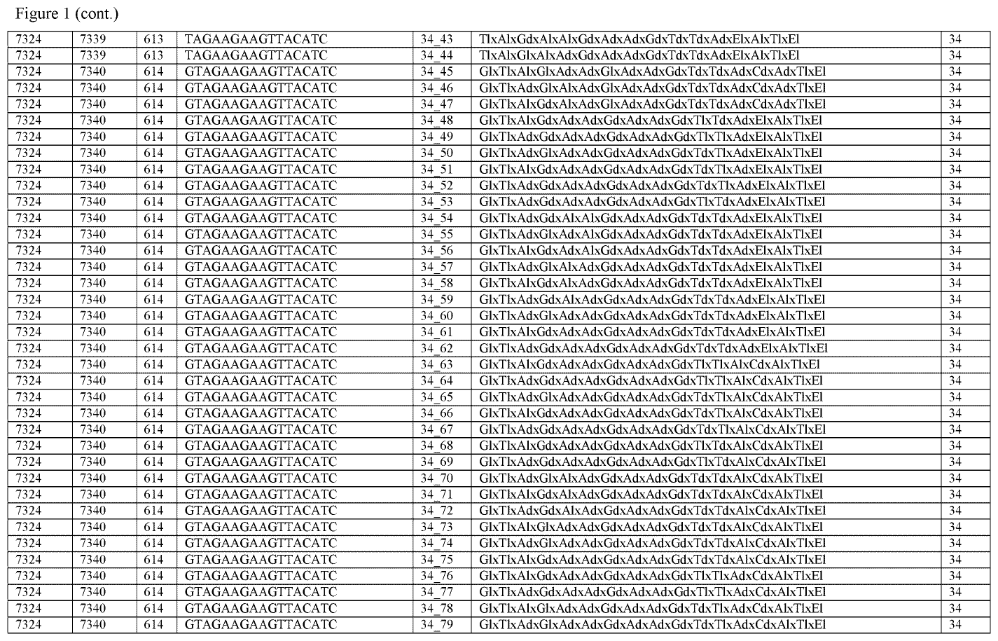

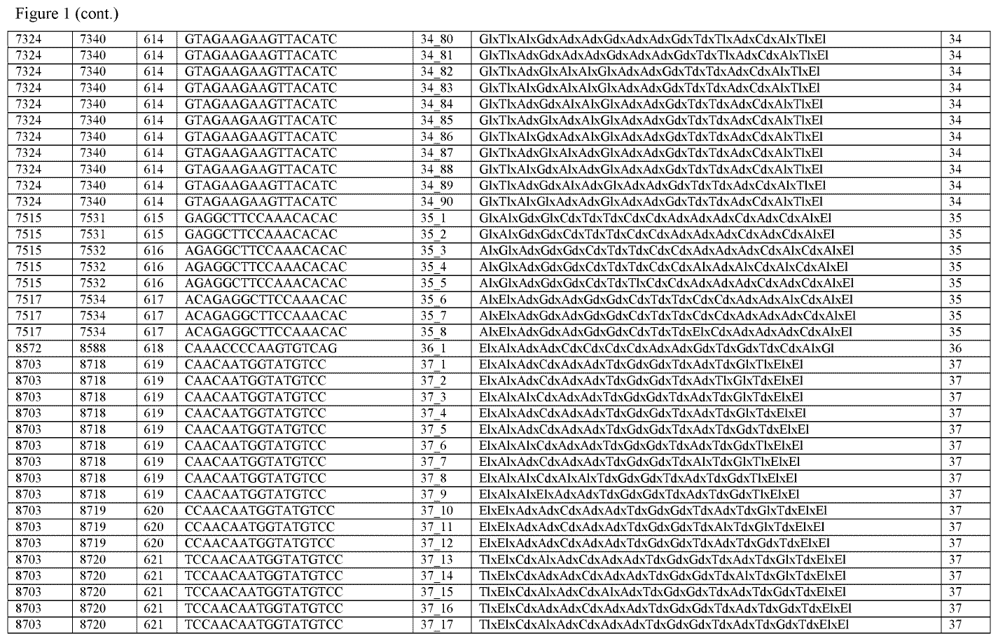

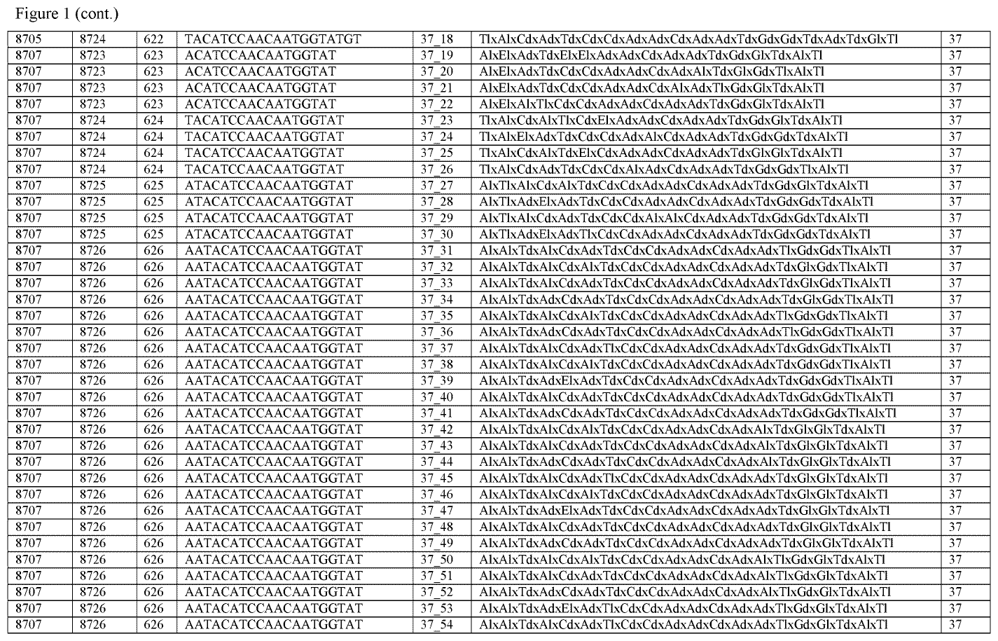

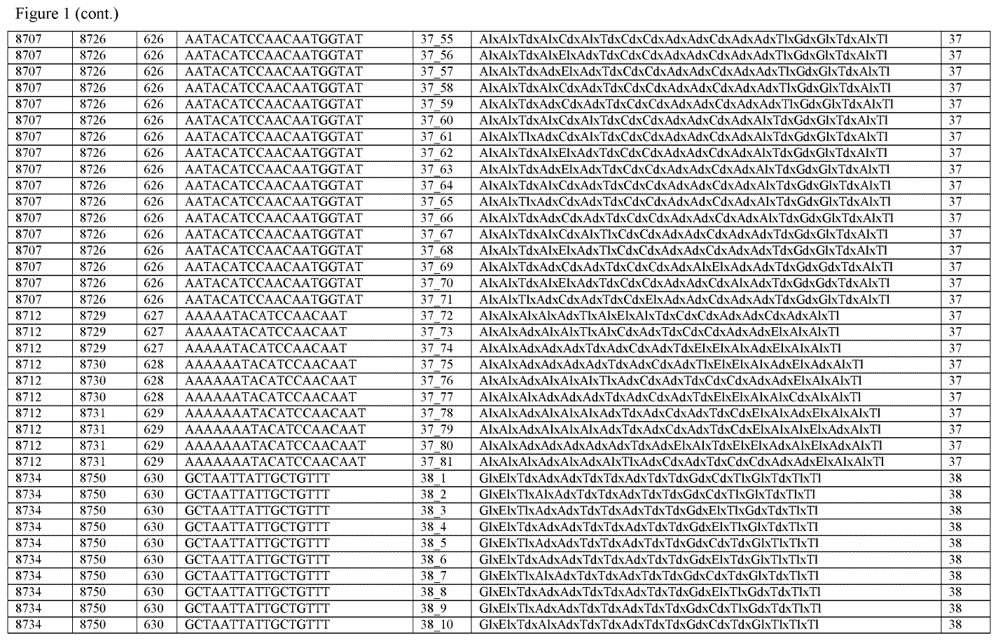

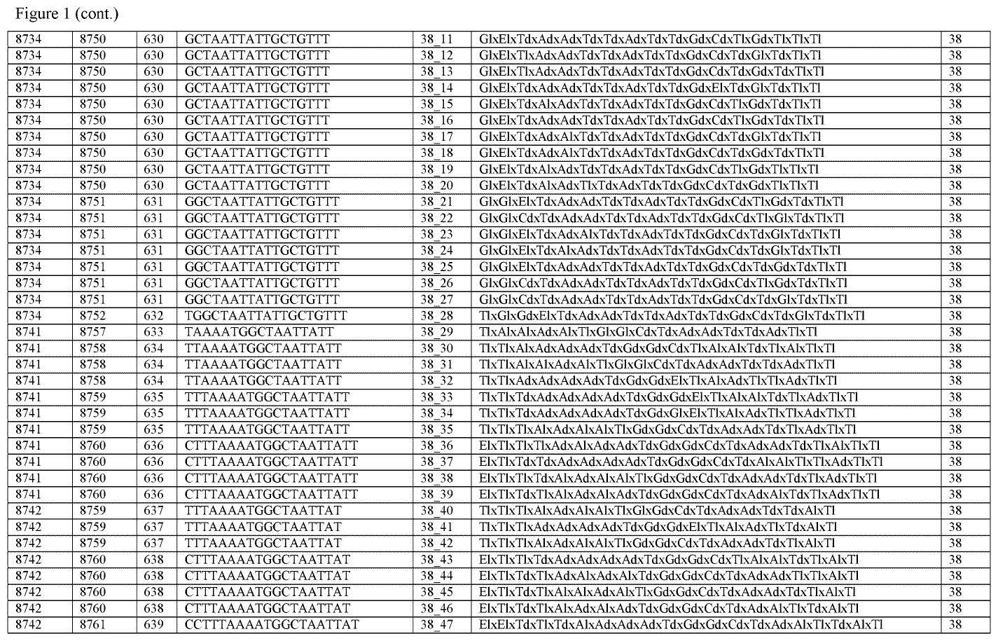

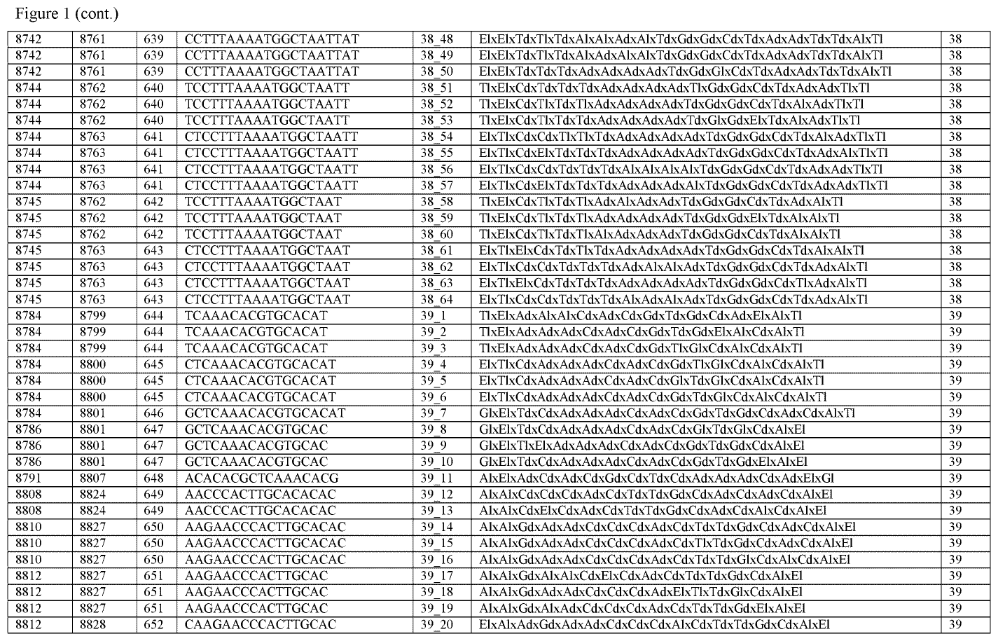

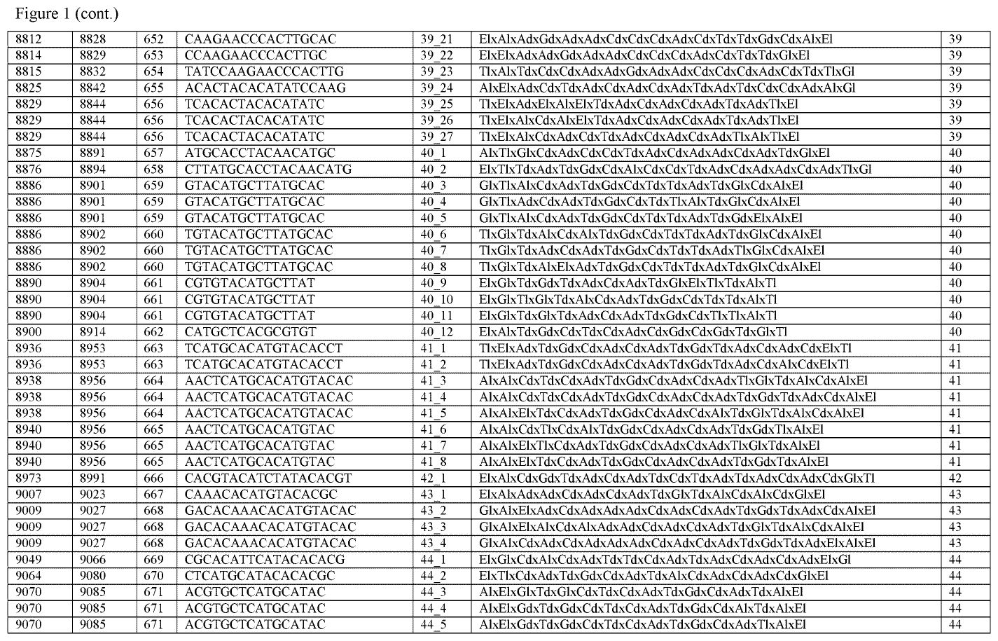

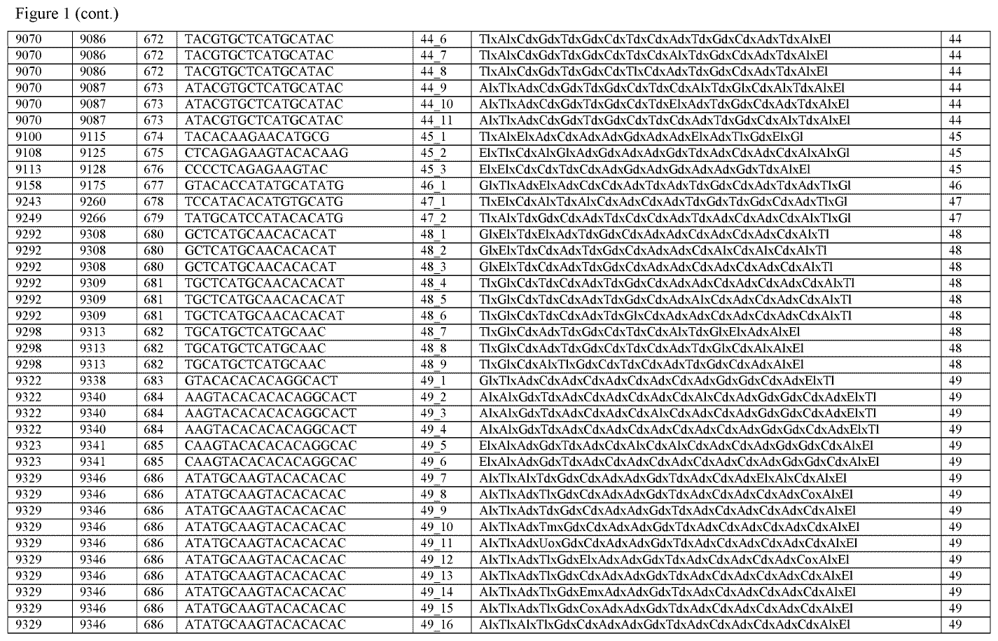

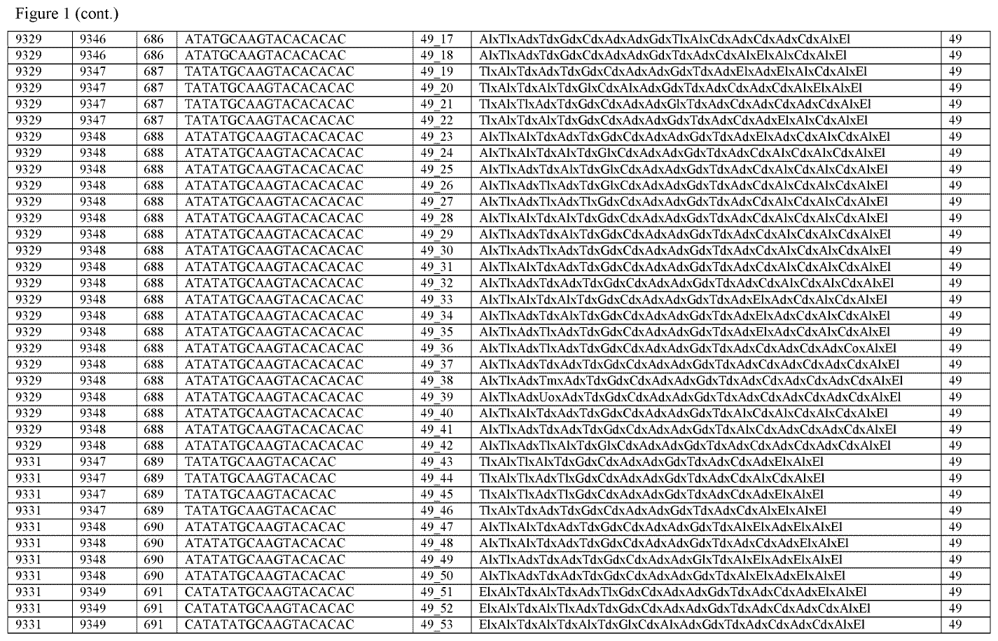

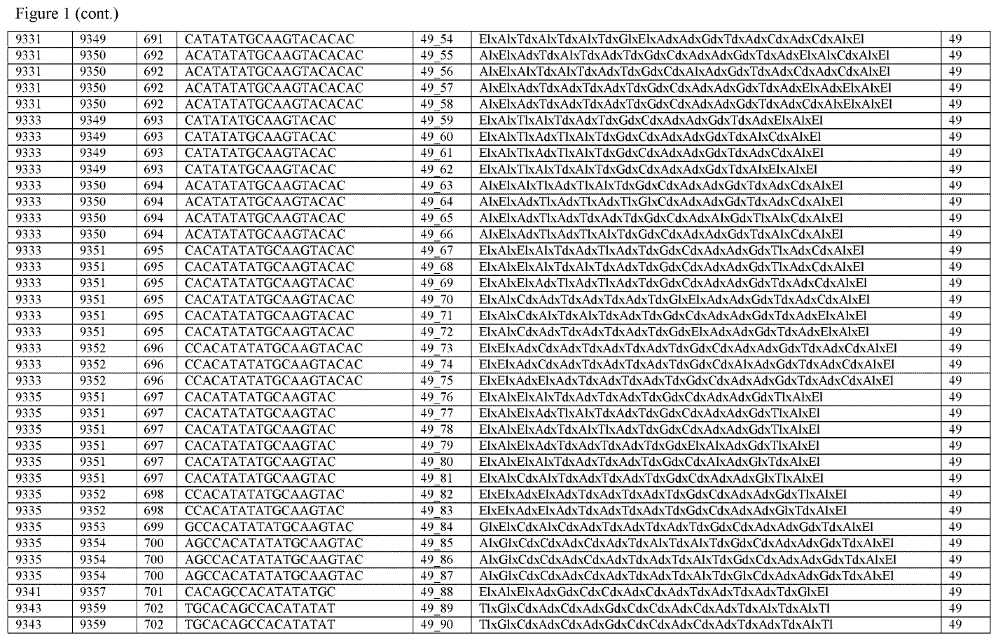

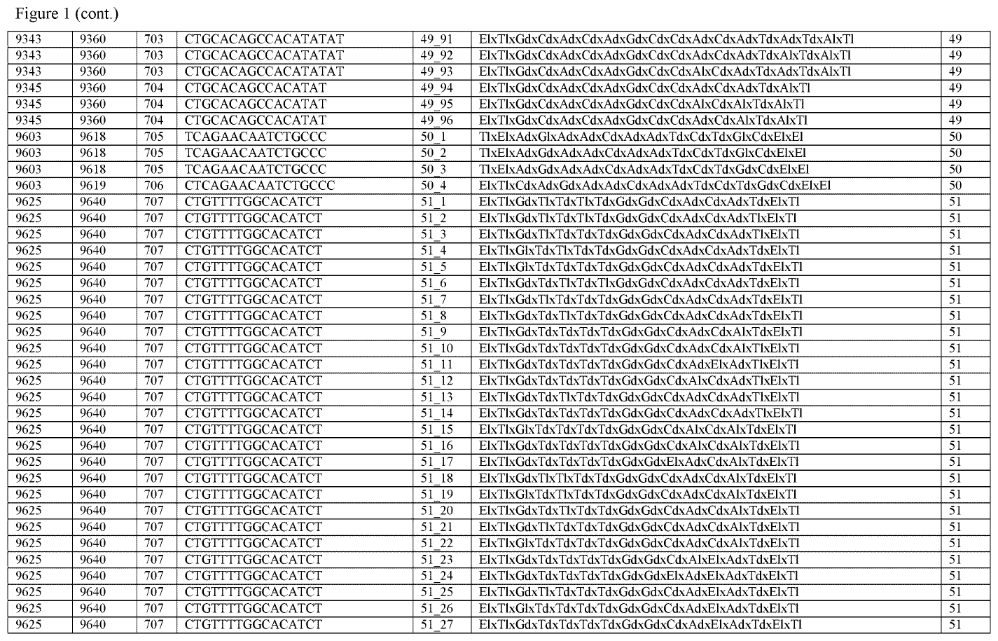

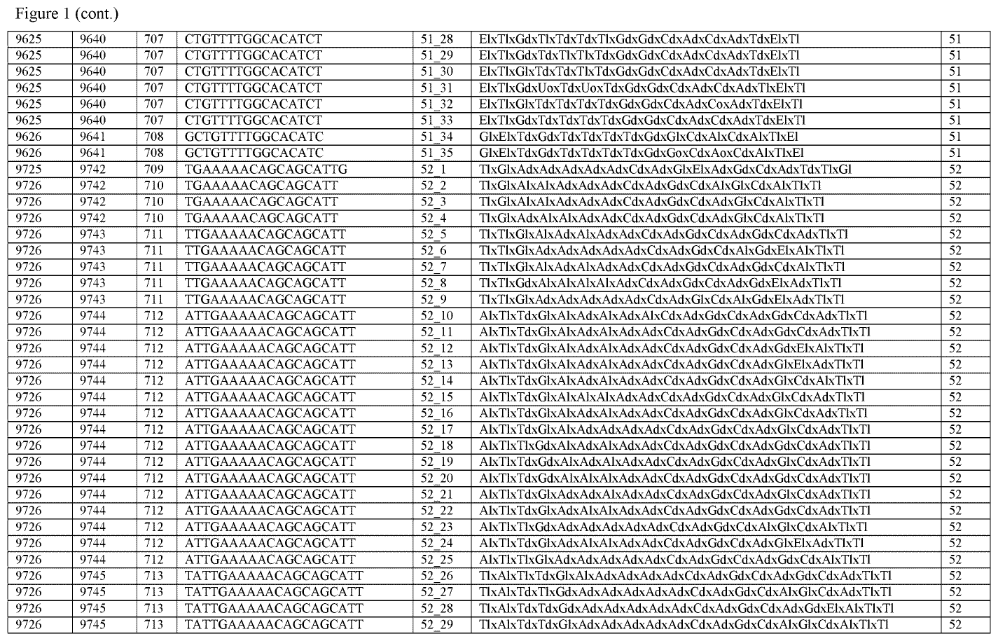

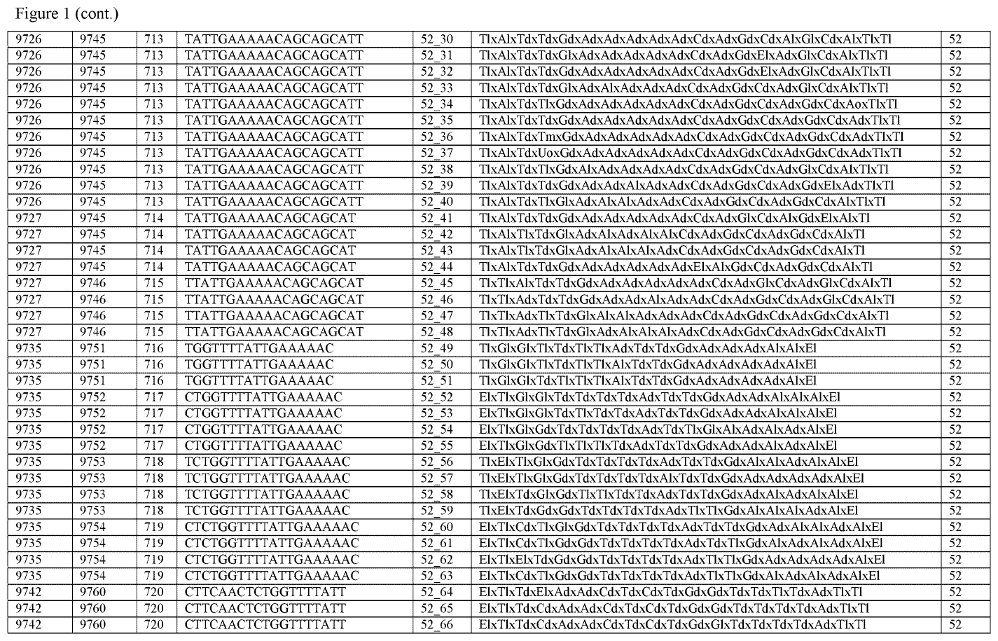

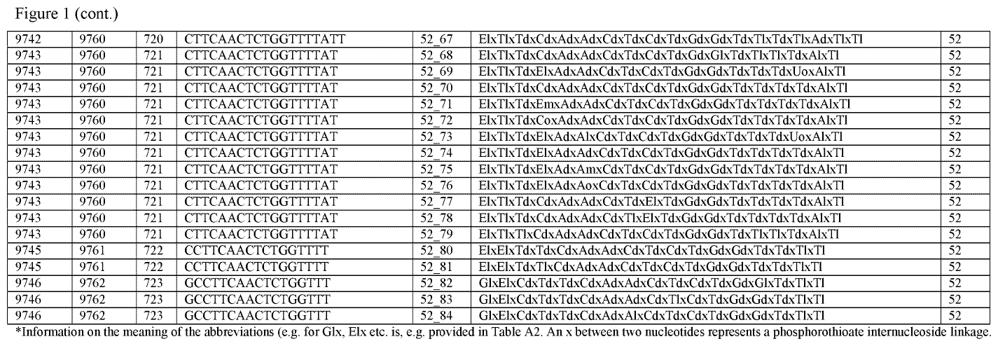

The sequences and compounds referred to above can be found in the Table A1 in FIG. 1.

Preferably, the antisense oligonucleotide of the present invention is a chemically modified antisense oligonucleotide. Accordingly, it does not occur in nature. As known by the skilled person a wide range of chemical modification can be incorporated into an oligonucleotide, such modification are e.g. reviewed in Crooke et al. which herewith is incorporated by reference in its entirety (Stanley T Crooke, Xue-Hai Liang, Brenda F Baker, Rosanne M Crooke. Review J Biol Chem. 2021 January-June; 296. antisense technology: A review). which herewith is incorporated by reference in its entirety. Preferably, at least one of the nucleotides (herein also referred to a monomer) present in the oligonucleotide comprises a chemical modification. More preferably, at least 30%, such as at least 50% of the nucleotides present in the oligonucleotide comprise a chemical modification. In some embodiments, all of the nucleotides comprise chemical modification. Modifications include modifications of the phosphodiester backbone chemistry (“backbone modifications”), nucleobase modifications and sugar modifications. Preferred chemical modifications are shown in Table A2 and C2, see in particular the column “Nucleotide”. The ASOs of the present invention may comprise such nucleotides.

Modifications of the phosphodiester backbone chemistry affect the linkage between the individual monomers of the ASO. Thus, the ASO of the present preferably comprises one or more internucleoside linkages other than a phosphodiester linkage. More preferably, the antisense oligonucleotide comprises at least five, such as at least ten modified internucleoside linkages. Most preferably, all internucleoside linkages are modified linkages.

Preferably, the antisense oligonucleotide comprises at least five, such as at least ten modified internucleoside linkages, such as Phosphorothioate internucleoside linkage. Most preferably, all internucleoside linkages are modified linkages. However, some linkages may be phosphodiester linkages, such as one, up to two, up to three or up to four, up to six, or up to eight phosphodiester linkages.

Preferably, the at least one modified internucleoside linkage is selected from the group consisting of: at least one Phosphorothioate internucleoside linkage, at least one Phosphorodithioate internucleoside linkage, at least one Phophoroamidate internucleoside linkage, at least one methyl phosphonate internucleoside linkage, at least one phosphotriester internucleoside linkage, at least one boranophosphate internucleoside linkage and at least one phosphoryl guanidine internucleoside linkage. Moreover, the at least one modified internucleoside linkage can be a stereodefined versions of said linkages.

In a preferred embodiment, the oligonucleotide comprises at least one phosphorodithioate internucleoside linkage.

In another preferred embodiment, the oligonucleotide comprises at least one phosphoryl guanidine internucleoside linkage. Most preferably, all internucleoside linkage are phosphoryl guanidine internucleoside linkages.

In particularly preferred embodiment, the oligonucleotide comprises at least one phosphorothioate internucleoside linkage. Most preferably, all internucleoside linkage are phosphorothioate linkages.

Further, it is envisaged that the backbone may be modified with Morpholino Phosphorodiamidate (PMO) and Peptide Nucleic Acid (PNA).

Modification applied to the sugar group could be acyclic modifications such as UNA (unlocked nucleic acid), FNA (Flexible nucleic acid), (S)- and (R)-GNA (glycol nucleic acid), D- and L-aTNA (threofuranosyl nucleic acids), SNA (Serinol nucleic acids), as described in further details in Bege & Borbas 2021 (Miklós Bege & Anikó Borbás Review Pharmaceuticals (Basel). 2022 Jul. 22; 15(8):909. doi: 10.3390/ph15080909. The Medicinal Chemistry of Artificial Nucleic Acids and Therapeutic).

In a preferred embodiment, the chemically modified antisense oligonucleotide comprises one or more modified nucleosides.

Preferably, the one or more modified nucleosides are sugar modified nucleosides, such as one, two, three, four or more sugar modified nucleosides. Typically, it comprises four sugar modified nucleosides. A sugar modified nucleoside is nucleoside with a modified sugar. In an embodiment, the one or more sugar modified nucleosides are 2′ sugar modified nucleosides, such as 2′O modified sugar nucleosides.

In particular, the 2′O modified sugar is, selected from the group consisting of 2′-O-Me, 2′MOE (2′-O-Methoxyethyl)), 2′-Npropyl, 2′-O-allyl, 2′F RNA, 2′-O-ethylamine.

In an embodiment, the 2′O modified sugar is 2′MOE (2′-O-Methoxyethyl). Thus, the modified nucleosides are 2′MOE nucleosides.

Moreover, the one or more modified nucleotides could be locked nucleic acids such as, beta-D-oxy-LNA, 2′,4′-constrained 2′-O-ethyl (cEt), such as R-cET and S-cEt, Beta-D-amino LNA, Beta-D-thio LNA, alpha-L-oxy LNA, ENA and other modifications as described in Wan & Seth 2016 (W Brad Wan, Punit P Seth, Review J Med Chem. 2016 Nov. 10; 59(21):9645-9667. The Medicinal Chemistry of Therapeutic Oligonucleotides). Thus, the oligonucleotide of the invention preferably comprises one more Locked Nucleic Acid Nucleosides (LNA nucleosides) which are well known 2′-modified nucleosides.

Preferably, the one or more modified nucleosides are (S)-6′-methyl-beta-D-oxy-LNA (ScET) LNA nucleosides. More preferably, the one or more modified nucleosides are beta-D-oxy-LNA nucleosides,

Nucleobase modification include, but are not limited to, 5-methyl-cytosine, pseudo-uridine, 5-Methyluridine, 8-Oxoguanine, 2-thio-thymine, Diaminopurine, abasic nucleosides and others as also described in Brad&Seth 2016 and Robert et al., 2020 (Thomas C Roberts, Robert Langer, Matthew J A Wood. Review Nat Rev Drug Discov. 2020 October; 19(10):673-694. Advances in oligonucleotide drug delivery)

Alternatively or additionally, the chemically modified antisense oligonucleotide contains at least one modified nucleobase. For example, the at least one modified nucleobase is 5-methylcytosine. Also, the ASO may comprise at least pseudouridine, or at least one 8-oxoguanine as modified nucleobase.

Alternatively or additionally, the chemically modified antisense oligonucleotide comprises at least one modified nucleoside selected from the group consisting of: 2-O-Methoxyethyl-RNA, 2′-O-Methyl-RNA, 2′-Fluoro-RNA.

In a preferred embodiment, the antisense oligonucleotide of the present invention has a gapmer structure, i.e. is a gapmer. Gapmers are well known in the art. The term refers to (single stranded) DNA antisense oligonucleotide structures with RNA-like segments on both sides (flanking regions). Gapmers bind to the target sequence and down-regulate target gene expression through the induction of RNase H cleavage.

Suitable gapmer designs are well known in the art and are e.g. reviewed in Crooke et al. which herewith is incorporated by reference in its entirety (Stanley T Crooke, Xue-Hai Liang, Brenda F Baker, Rosanne M Crooke. Review J Biol Chem. 2021 January-June; 296. Antisense technology: A review).

Preferably, the gapmer is a LNA gapmer in which the flanking regions comprise LNA nucleosides, such as D-oxy LNA nucleosides. However, the gapmer may also comprise 2′O-Methoxyethyl (MOE) nucleosides in the flanking regions. Such gapmers are frequently referred to as MOE gapmers.

In a preferred embodiment, the antisense oligonucleotide of the present invention has a gapmer structure and at least one modified internucleoside linkage. In a preferred embodiment, the antisense oligonucleotide of the present invention has a gapmer structure and at least 10 modified internucleoside linkages. In another preferred embodiment, the antisense oligonucleotide of the present invention has a gapmer structure and all linkages are modified internucleoside linkages. The modified linkages are described herein above. In an embodiment, the modified linkages are phosphorothioate internucleoside linkages. In another embodiment, the linkages are phosphorodithioates linkages.

In an embodiment, the antisense oligonucleotide of the present invention is a compound selected from the compounds shown in Table A1 (see column “Compound”), wherein
Adx represents 2′deoxyadenosine-3′-phosphorothioate
Aox represents 2′-O-methyladenosine-3′-phosphorothioate
Amx represents 2′-O-Methoxyethyladenosine-3′-phosphorothioate
Alx represents 2′-O-beta-D-oxy LNA adenosine-3′-phosphorothioate
Cdx represents 2′deoxycytidine-3′-phosphorothioate
Cox represents 2′-O-methylcytidine-3′-phosphorothioate
Edx represents 2′deoxy-5-methylcytidine-3′-phosphorothioate
Emx represents 2′-O-Methoxyethyl-5-methylcytidine-3′-phosphorothioate
Elx represents 2′-O-beta-D-oxy LNA-5-methylcytidine-3′-phosphorothioate
Gdx represents 2′deoxyguanosine-3′-phosphorothioate
Gox represents 2′-O-methylguanosine-3′-phosphorothioate
Gmx represents 2′-O-Methoxyethylguanosine-3′-phosphorothioate
Glx represents 2′-O-beta-D-oxy LNA guanosine-3′-phosphorothioate
Tdx represents 2′deoxythymidine-3′-phosphorothioate
Tmx represents 2′-O-Methoxyethylthymidine-3′-phosphorothioate
Tlx represents 2′-O-beta-D-oxy LNA thymidine-3′-phosphorothioate
Uo represents 2′-O-methyluridine-3′
Ad represents 2′deoxyadenosine-3′
Ao represents 2′-O-methyladenosine-3′
Am represents 2′-O-Methoxyethyladenosine-3′
Al represents 2′-O-beta-D-oxy LNA adenosine-3′
Cd represents 2′deoxycytidine-3′
Co represents 2′-O-methylcytidine-3′
Ed represents 2′deoxy-5-methylcytidine-3′
Em represents 2′-O-Methoxyethyl-5-methylcytidine-3′
El represents 2′-O-beta-D-oxy LNA-5-methylcytidine-3′
Gd represents 2′deoxyguanosine-3′
Go represents 2′-O-methylguanosine-3′
Gm represents 2′-O-Methoxyethylguanosine-3′
Gl represents 2′-O-beta-D-oxy LNA guanosine-3′
Td represents 2′deoxythymidine-3′
Tm represents 2′-O-Methoxyethylthymidine-3′,
Tl represents 2′-O-beta-D-oxy LNA thymidine-3′, and
Uo represents 2′-O-methyluridine-3.′

All internucleoside linkages present in the compounds shown in Table A1 are phosphorothioate linkages.

In Table A1, a three letter code or two letter code was used in order to describe the modified nucleotides that are present in the oligonucleotide compounds. Additionally, each of the compounds in Table A1 contains a two letter code in the 3′ end, which does not contain the phosphorothioate group. The base and sugar groups of the two letter code is otherwise identical to the three letter. Table A2 provides a translation of the two or three letter codes to their chemical names.

TABLE A2
Overview on modified nucleotides

| | | | Complementary |
|---|---|---|---|
| | | | nucleotide in the target |
| | | | pre-mRNA (Watson- |
| Abbreviation | Nucleotide | Sugar | Crick-base pairing) |
| Adx | 2′deoxyadenosine-3′-phosphorothioate | DNA | U (“T” in SEQ ID NO: 1) |
| Aox | 2′-O-methyladenosine-3′-phosphorothioate | 2'Ome | U (“T” in SEQ ID NO: 1) |
| Amx | 2′-O-Methoxyethyladenosine-3′-phosphorothioate | MOE | U (“T” in SEQ ID NO: 1) |
| Alx | 2′-O-beta-D-oxy LNA adenosine-3′- | LNA | U (“T” in SEQ ID NO: 1) |
| | phosphorothioate | | |
| Cdx | 2′deoxycytidine-3′-phosphorothioate | DNA | G |
| Cox | 2′-O-methylcytidine-3′-phosphorothioate | 2'Ome | G |
| Edx | 2′deoxy-5-methylcytidine-3′-phosphorothioate | DNA | G |
| Emx | 2′-O-Methoxyethy1-5-methylcytidine-3′- | MOE | G |
| | phosphorothioate | | |
| Elx | 2′-O-beta-D-oxy LNA-5-methylcytidine-3′- | LNA | G |
| | phosphorothioate | | |
| Gdx | 2′deoxy guanosine-3′-phosphorothioate | DNA | C |
| Gox | 2′-O-methylguanosine-3′-phosphorothioate | 2'Ome | C |
| Gmx | 2′-O-Methoxyethylguanosine-3′-phosphorothioate | MOE | C |
| Glx | 2′-O-beta-D-oxy LNA guanosine-3′- | LNA | C |
| | phosphorothioate | | |
| Tdx | 2′deoxythymidine-3′-phosphorothioate | DNA | A |
| Tmx | 2′-O-Methoxyethylthymidine-3′-phosphorothioate | MOE | A |
| Tlx | 2′-O-beta-D-oxy LNA thymidine-3′- | LNA | A |
| | phosphorothioate | | |
| Uo | 2′-O-methyluridine-3′ | 2′Ome | A |
| Ad | 2′deoxyadenosine-3′ | DNA | U (“T” in SEQ ID NO: 1) |
| Ao | 2′-O-methyladenosine-3′ | 2′Ome | U (“T” in SEQ ID NO: 1) |
| Am | 2′-O-Methoxyethyladenosine-3′ | MOE | U (“T” in SEQ ID NO: 1) |
| Al | 2′-O-beta-D-oxy LNA adenosine-3′ | LNA | U (“T” in SEQ ID NO: 1) |
| Cd | 2′deoxycytidine-3′ | DNA | G |
| Co | 2′-O-methylcytidine-3′ | 2′Ome | G |
| Ed | 2′deoxy-5-methylcytidine-3′ | DNA | G |
| Em | 2′-O-Methoxyethy1-5-methylcytidine-3′ | MOE | G |
| El | 2′-O-beta-D-oxy LNA -5-methylcytidine-3′ | LNA | G |
| Gd | 2′deoxy guanosine-3′ | DNA | C |
| Go | 2′-O-methylguanosine-3′ | 2′Ome | C |
| Gm | 2′-O-Methoxyethylguanosine-3′ | MOE | C |
| Gl | 2′-O-beta-D-oxy LNA guanosine-3′ | LNA | C |
| Td | 2′deoxythymidine-3′ | DNA | A |
| Tm | 2′-O-Methoxyethylthymidine-3′ | MOE | A |
| Tl | 2′-O-beta-D-oxy LNA thymidine-3′ | LNA | A |
| Uo | 2′-O-methyluridine-3′ | 2′Ome | A |

Complex Macromolecules, such antisense oligonucleotides according to the present invention with non-natural chemical modifications, can be also depicted in the HELM format (HELM: “Hierarchical Editing Language for Macromolecules”). The HELM format is described in Zhang T, Li H, Xi H, Stanton R V, Rotstein S H. HELM: a hierarchical notation language for complex biomolecule structure representation. J Chem Inf Model. 2012 Oct. 22; 52(10):2796-806. doi: 10.1021/ci3001925. Epub 2012 Sep. 26. PMID: 22947017. The document is herewith incorporated by reference in its entirety.

Table A3 shows selected compounds of the present invention in HELM Annotation Format. In a preferred embodiment, the antisense oligonucleotide is a compound as shown in Table A3.

TABLE A3
Exemplary compounds of the present invention - HELM Annotation Format

| | | Compound in HELM Annotation |
|---|---|---|
| ASO ID | SEQ ID | Written 5′ to 3′. |
| 2 _17 | 410 | [LR](A)[sP].[LR](G)[sP].[dR](A)[P].[LR](T)[sP |
| | | ].[LR](T)[sP].[dR](A)[sP].[dR](T)[sP].[dR](A) |
| | | [sP].[dR](T)[sP].[dR](T)[sP].[dR](T)[sP].[dR] |
| | | (A)[sP].[dR](G)[sP].[dR](G)[sP].[dR](C)[sP].[ |
| | | dR](A)[sP].[dR](C)[sP].[LR](T)[sP].[LR]([5meC |
| | | ]) |
| 2_50 | 413 | [LR](A)[sP].[LR](G)[sP].[mR](A)[sP].[LR](T)[s |
| | | P].[LR](T)[sP].[dR](A)[sP].[dR](T)[sP].[dR](A |
| | | )[sP].[dR](T)[sP].[dR](T)[sP].[dR](T)[sP].[dR |
| | | ](A)[sP].[dR](G)[sP].[dR](G)[sP].[dR](C)[sP]. |
| | | [LR](A)[sP].[LR]([5meC])[sP].[LR](T) |
| 2_52 | 413 | [LR](A)[sP].[LR](G)[sP].[mR](A)[sP].[LR](T)[s |
| | | P].[LR](T)[sP].[dR](A)[sP].[mR](U)[sP].[dR](A |
| | | )[sP].[dR](T)[sP].[dR](T)[sP].[dR](T)[sP].[dR |
| | | ](A)[sP].[dR](G)[sP].[dR](G)[sP].[dR](C)[sP]. |
| | | [LR](A)[sP].[LR]([5meC])[sP].[LR](T) |
| 2_35 | 413 | [LR](A)[sP].[LR](G)[sP].[dR](A)[sP].[LR](T)[s |
| | | P].[LR](T)[sP].[dR](A)[sP].[dR](T)[sP].[dR](A |
| | | )[sP].[dR](T)[sP].[dR](T)[sP].[dR](T)[sP].[dR |
| | | ](A)[sP].[dR](G)[sP].[dR](G)[sP].[dR](C)[sP]. |
| | | [LR](A)[sP].[LR]([5meC])[sP].[LR](T) |
| 25_111 | 517 | [LR]([5meC])[sP].[LR](G)[sP].[dR](T)[sP].[LR] |
| | | (T)[sP].[dR](T)[sP].[LR](T)[sP].[dR](G)[sP].[ |
| | | dR](T)[sP].[dR](C)[sP].[dR](C)[sP].[dR](C)[sP |
| | | ].[dR](A)[sP].[dR](A)[sP].[dR](G)[sP].[dR](A) |
| | | [sP].[dR](A)[sP].[LR](T)[sP].[LR](T) |
| 25_113 | 517 | [LR]([5meC])[sP].[LR](G)[sP].[dR](T)[sP].[LR] |
| | | (T)[sP].[LR](T)[sP].[dR](T)[sP].[dR](G)[sP].[ |
| | | dR](T)[sP].[dR](C)[sP].[dR](C)[sP].[dR](C)[sP |
| | | ].[dR](A)[sP].[dR](A)[sP].[LR](G)[sP].[dR](A) |
| | | [sP].[dR](A)[sP].[LR](T)[sP].[LR](T) |
| 29_5 | 557 | [LR]([5meC])[sP].[LR]([5meC])[sP].[dR](A)[sP] |
| | | .[dR](T)[sP].[dR](T)[sP].[dR](T)[sP].[dR](T)[ |
| | | sP].[dR](G)[sP].[dR](C)[P].[dR](T)[sP].[dR](A |
| | | )[sP].[dR](A)[sP].[LR](A)[sP].[LR](A)[sP].[dR |
| | | ](C)[sP].[LR](A)[sP].[dR](C)[sP].[LR](T)[sP]. |
| | | [LR](T) |
| 29_10 | 558 | [LR](T)[sP].[LR]([5meC])[sP].[dR](C)[sP].[dR] |
| | | (A)[sP].[LR](T)[sP].[dR](T)[sP].[dR](T)[sP].[ |
| | | dR](T)[sP].[dR](G)[sP].[dR](C)[sP].[dR](T)[sP |
| | | ].[dR](A)[sP].[dR](A)[sP].[dR](A)[sP].[LR](A) |
| | | [sP].[dR](C)[sP].[LR](A)[sP].[dR](C)[sP].[LR] |
| | | (T)[sP].[LR](T) |
| 29_34 | 561 | [LR](T)[sP].[LR]([5meC])[sP].[dR](C)[sP].[LR] |
| | | (A)[sP].[dR](T)[sP].[dR](T)[sP].[dR](T)[sP].[ |
| | | dR](T)[sP].[dR](G)[sP].[dR](C)[sP].[dR](T)[sP |
| | | ].[dR](A)[sP].[LR](A)[sP].[dR](A)[sP].[LR](A) |
| | | [sP].[dR](C)[sP].[LR](A)[sP].[LR]([5meC])[sP] |
| | | .[LR](T) |
| 29_70 | 570 | [LR](A)[sP].[LR]([5meC])[sP].[dR](T)[sP].[dR] |
| | | (G)[sP].[LR](T)[sP].[LR](T)[sP].[dR](T)[sP].[ |
| | | dR](G)[sP].[dR](T)[sP].[dR](T)[sP].[dR](T)[sP |
| | | ].[dR](C)[sP].[dR](C)[sP].[dR](A)[sP].[dR](T) |
| | | [sP].[dR](T)[sP].[LR](T)[sP].[LR](T) |
| 29_78 | 573 | [LR](A)[sP].[LR](A)[sP].[dR](C)[sP].[LR](T)[s |
| | | P].[LR](G)[sP].[dR](T)[sP].[dR](T)[sP].[dR](T |
| | | )[sP].[dR](G)[sP].[dR](T)[sP].[dR](T)[sP].[dR |
| | | ](T)[sP].[dR](C)[sP].[dR](C)[sP].[dR](A)[sP]. |
| | | [LR](T)[sP].[LR](T)[sP].[LR](T) |
| 29_79 | 573 | [LR](A)[sP].[LR](A)[P].[mR](C)[sP].[LR](T)[sP |
| | | ].[dR](G)[sP].[dR](T)[sP].[dR](T)[sP].[dR](T) |
| | | [sP].[dR](G)[sP].[dR](T)[sP].[dR](T)[sP].[dR] |
| | | (T)[sP].[dR](C)[sP].[dR](C)[sP].[dR](A)[sP].[ |
| | | mR](U)[sP].[LR](T)[sP].[LR](T) |
| 29_84 | 573 | [LR](A)[sP].[LR](A)[sP].[dR](C)[sP].[LR](T)[s |
| | | P].[dR](G)[sP].[dR](T)[sP].[dR](T)[sP].[dR](T |
| | | )[sP].[dR](G)[sP].[dR](T)[sP].[dR](T)[sP].[dR |
| | | ](T)[sP].[dR](C)[sP].[dR](C)[sP].[dR](A)[sP]. |
| | | [dR](T)[sP].[LR](T)[sP].[LR](T) |
| 29_85 | 573 | [LR](A)[sP].[LR](A)[sP].[dR](C)[sP].[LR](T)[s |
| | | P].[dR](G)[sP].[MOE](T)[sP].[dR](T)[sP].[dR]( |
| | | T)[sP].[dR](G)[sP].[dR](T)[sP].[dR](T)[sP].[d |
| | | R](T)[sP].[dR](C)[sP].[dR](C)[sP].[dR](A)[sP] |
| | | .[dR](T)[sP].[LR](T)[sP].[LR](T) |
| 29_86 | 573 | [LR](A)[sP].[LR](A)[sP].[dR](C)[sP].[LR](T)[s |
| | | P].[dR](G)[sP].[mR](U)[sP].[dR](T)[sP].[dR](T |
| | | )[sP].[dR](G)[sP].[dR](T)[sP].[dR](T)[sP].[dR |
| | | ](T)[sP].[dR](C)[sP].[dR](C)[sP].[dR](A)[sP]. |
| | | [dR](T)[sP].[LR](T)[sP].[LR](T) |
| 29_124 | 580 | [LR](A)[sP].[LR]([5meC])[sP].[dR](A)[sP].[mR] |
| | | (A)[sP].[dR](C)[sP].[dR](T)[sP].[dR](G)[sP].[ |
| | | dR](T)[sP].[dR](T)[sP].[dR](T)[sP].[dR](G)[sP |
| | | ].[dR](T)[sP].[LR](T)[sP].[dR](T)[sP].[LR]([5 |
| | | meC])[sP].[mR](C)[sP].[LR](A)[sP].[LR](T) |
| 29_121 | 580 | [LR](A)[P].[LR]([5meC])[sP].[dR](A)[sP].[dR]( |
| | | A)[sP].[dR](C)[sP].[dR](T)[sP].[dR](G)[sP].[d |
| | | R](T)[sP].[dR](T)[sP].[dR](T)[sP].[dR](G)[sP] |
| | | .[dR](T)[sP].[LR](T)[sP].[dR](T)[sP].[LR]([5m |
| | | eC])[sP].[mR](C)[sP].[LR](A)[sP].[LR](T) |
| 29_130 | 582 | [LR]([5meC])[sP].[LR](A)[sP].[dR](C)[sP].[LR] |
| | | (A)[sP].[LR](A)[sP].[dR](C)[sP].[dR](T)[sP].[ |
| | | dR](G)[sP].[dR](T)[sP].[dR](T)[sP].[dR](T)[sP |
| | | ].[dR](G)[sP].[dR](T)[sP].[dR](T)[sP].[dR](T) |
| | | [sP].[LR]([5meC])[sP].[LR]([5meC]) |
| 29_138 | 584 | [LR](G)[sP].[LR](G)[sP].[dR](C)[sP].[dR](A)[s |
| | | P].[LR]([5meC])[sP].[dR](A)[sP].[dR](A)[sP].[ |
| | | dR](C)[sP].[dR](T)[sP].[dR](G)[sP].[dR](T)[sP |
| | | ].[dR](T)[sP].[dR](T)[sP].[dR](G)[sP].[dR](T) |
| | | [sP].[LR](T)[sP].[LR](T) |
| 34_39 | 613 | [LR](T)[sP].[LR](A)[sP].[LR](G)[sP].[dR](A)[s |
| | | P].[LR](A)[sP].[LR](G)[sP].[LR](A)[sP].[dR](A |
| | | )[sP].[dR](G)[sP].[dR](T)[sP].[dR](T)[sP].[dR |
| | | ](A)[sP].[dR](C)[sP].[dR](A)[sP].[LR](T)[sP]. |
| | | [LR]([5meC]) |
| 34_46 | 614 | [LR](G)[sP].[LR](T)[sP].[dR](A)[sP].[LR](G)[s |
| | | P].[LR](A)[sP].[dR](A)[sP].[LR](G)[sP].[dR](A |
| | | )[sP].[dR](A)[sP].[dR](G)[sP].[dR](T)[sP].[dR |
| | | ](T)[sP].[dR](A)[sP].[dR](C)[sP].[dR](A)[sP]. |
| | | [LR](T)[sP].[LR]([5meC]) |
| 37_15 | 621 | [LR](T)[sP].[LR]([5meC])[sP].[dR](C)[sP].[LR] |
| | | (A)[sP].[dR](A)[sP].[dR](C)[sP].[LR](A)[sP].[ |
| | | dR](A)[sP].[dR](T)[sP].[dR](G)[sP].[dR](G)[sP |
| | | ].[dR](T)[sP].[dR](A)[sP].[dR](T)[sP].[dR](G) |
| | | [sP].[dR](T)[sP].[LR]([5meC])[sP].[LR]([5meC] |
| | | ) |
| 38_10 | 630 | [LR](G)[sP].[LR]([5meC])[sP].[dR](T)[sP].[LR] |
| | | (A)[sP].[dR](A)[sP].[dR](T)[sP].[dR](T)[sP].[ |
| | | dR](A)[sP].[dR](T)[sP].[dR](T)[sP].[dR](G)[sP |
| | | ].[dR](C)[sP].[dR](T)[sP].[LR](G)[sP].[LR](T) |
| | | [sP].[LR](T)[sP].[LR](T) |
| 51_6 | 707 | [LR]([5meC])[sP].[LR](T)[sP].[dR](G)[sP].[dR] |
| | | (T)[sP].[LR](T)[sP].[dR](T)[sP].[LR](T)[sP].[ |
| | | dR](G)[sP].[dR](G)[sP].[dR](C)[sP].[dR](A)[sP |
| | | ].[dR](C)[sP].[dR](A)[sP].[dR](T)[sP].[LR]([5 |
| | | meC])[sP].[LR](T) |

Helm Annotation Key for the compounds in Table A3:
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC] is a beta-D-oxy-LNA 5-methyl cytosine nucleoside,
[dR](G) is a DNA guanine nucleoside,
[dR](T) is a DNA thymine nucleoside,
[dR](A) is a DNA adenine nucleoside,
[dR](C) is a DNA cytosine nucleoside,
[mR](G) is a 2′-O-methyl RNA guanine nucleoside,
[mR](U) is a 2′-O-methyl RNA uracil nucleoside,
[mR](A) is a 2′-O-methyl RNA adenine nucleoside,
[mR](C) is a 2′-O-methyl RNA cytosine nucleoside,
[sP] is a phosphorothioate internucleoside linkage.

For more details, see also the Helm Annotation Key for Table C3 which also applies to Table A3 (and vice versa).

Preferably, the compound selected from Table A1 is a compound which resulted in an efficient down-regulation of the target gene in the studies described in Example 1 and/or Example 2.

The results for the experiments in Example 1 are shown in Table 3. In an embodiment, the compound is selected from the compounds resulting in an expression level of NAT8L of 40% or less than 40%, such as of 30% or less than 30% in relation to PBS treated control cells. Information on the expression level can be found in the column “NAT8L PBS norm (A549 High conc)” in Table 3.

The results for the experiments in Example 2 are shown in Table 6. In an embodiment, the compound is selected from the compounds resulting in an expression level of NAT8L of 50% or less than 50%, such as of 40% or less than 40% in relation to PBS treated control cells. Information on the expression level can be found in the column “NAT8L PBS norm (HEK293 Low conc)” in Table 6.

In an embodiment, the antisense oligonucleotide is an antisense oligonucleotide with ASO ID 2_17, 2_35, 2_50, 2_51, 2_52, 68, 25_113, 29_2, 295, 29_6, 29_7, 29_10, 29_23, 29_24, 29_32, 29_34, 29_36, 29_54, 29_70, 29_72, 29_78, 29_79, 29_84, 29_85, 29_86, 29_100, 29_112, 29_121, 29_123, 29_124, 29_125, 29_130, 29_133, 29_138, 29_154, 34_38, 34_39, 34_46, 34_47, 37_15, 385, 38_10, 38_11, 51_66 as shown in Table A1 in the Examples section.

In preferred embodiment, the antisense oligonucleotide is a compound selected from the compounds shown in Table A3.

In an embodiment, the antisense oligonucleotide is the compound with ASO ID 2_17.

In another embodiment, the antisense oligonucleotide is the compound with ASO ID 2_50.

In another embodiment, the antisense oligonucleotide is the compound with ASO ID 2_52.

In another embodiment, the antisense oligonucleotide is the compound with ASO ID 2_35.

In another embodiment, the antisense oligonucleotide is the compound with ASO ID 25_111.

In another embodiment, the antisense oligonucleotide is the compound with ASO ID 25_113.

In another embodiment, the antisense oligonucleotide is the compound with ASO ID 29_5.

In another embodiment, the antisense oligonucleotide is the compound with ASO ID 29_10.

In another embodiment, the antisense oligonucleotide is the compound with ASO ID 29_34.

In another embodiment, the antisense oligonucleotide is the compound with ASO ID 29_70.

In another embodiment, the antisense oligonucleotide is the compound with ASO ID 29_78.

In another embodiment, the antisense oligonucleotide is the compound with ASO ID 29_79.

In another embodiment, the antisense oligonucleotide is the compound with ASO ID 29_84.

In another embodiment, the antisense oligonucleotide is the compound with ASO ID 29_85.

In another embodiment, the antisense oligonucleotide is the compound with ASO ID 29_86.

In another embodiment, the antisense oligonucleotide is the compound with ASO ID 29_124.

In another embodiment, the antisense oligonucleotide is the compound with ASO ID 29_121.

In another embodiment, the antisense oligonucleotide is the compound with ASO ID 29_130.

In another embodiment, the antisense oligonucleotide is the compound with ASO ID 29_138.

In another embodiment, the antisense oligonucleotide is the compound with ASO ID 34_39.

In another embodiment, the antisense oligonucleotide is the compound with ASO ID 34_46.

In another embodiment, the antisense oligonucleotide is the compound with ASO ID 37_15.

In another embodiment, the antisense oligonucleotide is the compound with ASO ID 38_10.

In another embodiment, the antisense oligonucleotide is the compound with ASO ID 51_6.

In another embodiment, the antisense oligonucleotide of the present invention is a compound selected from the compounds shown in Table C1 in FIG. 1 (see column “Compound”).

In Table C1, a three letter code or two letter code was used in order to describe the modified nucleotides that are present in the oligonucleotide compounds. Additionally, each of the compounds in Table C1 contains a two letter code in the 3′ end, which does not contain the phosphorothioate group or phosphate. The base and sugar groups of the two letter code is otherwise identical to the three letter. Table C2 provides a translation of the two or three letter codes to their chemical names. The annotations, typically, also apply to the three or two letter codes of the compounds in Table A and vice versa. An x between two nucleotides represents a phosphorothioate internucleoside linkage. An o between two nucleotides represents a phosphodiester linkage.

TABLE C2
Overview on modified nucleotides

| Three- | | Two | |
|---|---|---|---|
| letter | | letter | |
| code | Nucleotide | code | Nucleotide |
| Adx | 2′deoxyadenosine-3′-phosphorothioate | Ad | 2′deoxyadenosine |
| Aox | 2′-O-methyladenosine-3′-phosphorothioate | Ao | 2′-O-methyladenosine-3′- |
| | | | phosphorothioate |
| Amx | 2′-O-Methoxyethyladenosine-3′- | Am | 2′-O-Methoxyethyladenosine |
| | phosphorothioate | | |
| Alx | 2′-O-beta-D-oxy LNA adenosine-3′- | Al | 2′-O-beta-D-oxy LNA adenosine |
| | phosphorothioate | | |
| Cdx | 2′deoxycytidine-3′-phosphorothioate | Cd | 2′deoxycytidine |
| Cox | 2′-O-methylcytidine-3′-phosphorothioate | Co | 2′-O-methylcytidine-3′- |
| | | | phosphorothioate |
| Edx | 2′deoxy-5-methylcytidine-3′- | Ed | 2′deoxy-5-methylcytidine |
| | phosphorothioate | | |
| Emx | 2′-O-Methoxyethyl-5-methylcytidine-3′- | Em | 2′-O-Methoxyethyl-5-methylcytidine |
| | phosphorothioate | | |
| Elx | 2′-O-beta-D-oxy LNA -5-methylcytidine-3′- | El | 2′-O-beta-D-oxy LNA-5- |
| | phosphorothioate | | methylcytidine |
| Gdx | 2′deoxyguanosine-3′-phosphorothioate | Gd | 2′deoxyguanosine |
| Gox | 2′-O-methylguanosine-3′-phosphorothioate | Go | 2′-O-methylguanosine-3′- |
| | | | phosphorothioate |
| Gmx | 2′-O-Methoxyethylguanosine-3′- | Gm | 2′-O-Methoxyethylguanosine |
| | phosphorothioate | | |
| Glx | 2′-O-beta-D-oxy LNA guanosine-3′- | Gl | 2′-O-beta-D-oxy LNA guanosine |
| | phosphorothioate | | |
| Tdx | 2′deoxythymidine-3′-phosphorothioate | Td | 2′deoxythymidine |
| Tmx | 2′-O-Methoxyethylthymidine-3′- | Tm | 2′-O-Methoxyethylthymidine |
| | phosphorothioate | | |
| Tlx | 2′-O-beta-D-oxy LNA thymidine-3′- | Tl | 2′-O-beta-D-oxy LNA thymidine |
| | phosphorothioate | | |
| Uox | 2′-O-methyluridine-3′-phosphorothioate | Uo | 2′-O-methyluridine-3′- |
| | | | phosphorothioate |
| Ado | 2′deoxyadenosine-3′-phosphate | | |
| Aoo | 2′-O-methyladenosine-3′-phosphate | | |
| Amo | 2′-O-Methoxyethyladenosine-3′-phosphate | | |
| Alo | 2′-O-beta-D-oxy LNA adenosine-3′- | | |
| | phosphate | | |
| Cdo | 2′deoxycytidine-3′-phosphate | | |
| Coo | 2′-O-methylcytidine-3′-phosphate | | |
| Edo | 2′deoxy-5-methylcytidine-3′-phosphate | | |
| Emo | 2′-O-Methoxyethyl-5-methylcytidine-3′- | | |
| | phosphate | | |
| Elo | 2′-O-beta-D-oxy LNA-5-methylcytidine-3′- | | |
| | phosphate | | |
| Gdo | 2′deoxyguanosine-3′-phosphate | | |
| Goo | 2′-O-methylguanosine-3′-phosphate | | |
| Gmo | 2′-O-Methoxyethylguanosine-3′-phosphate | | |
| Glo | 2′-O-beta-D-oxy LNA guanosine-3′- | | |
| | phosphate | | |
| Tdo | 2′deoxythymidine-3′-phosphate | | |
| Tmo | 2′-O-Methoxyethylthymidine-3′-phosphate | | |
| Tlo | 2′-O-beta-D-oxy LNA thymidine-3′- | | |
| | phosphate | | |
| Uoo | 2′-O-methyluridine-3′-phosphate | | |

Table C3 shows selected compounds of the present invention in HELM Annotation Format. In a preferred embodiment, the antisense oligonucleotide is a compound as shown in Table C3.

TABLE C3
Exemplary compounds of the present invention - HELM Annotation Format

| | SEQ | |
|---|---|---|
| ASO | ID | Compound in HELM Annotation |
| ID | NO | Written 5′ to 3′ |
| 217 | 410 | [LR](A)[sP].[LR](G)[sP].[dR](A)[sP].[LR](T)[sP].[LR](T) |
| | | [sP].[dR](A)[sP].[dR](T)[sP].[dR](A)[sP].[dR](T)[sP].[d |
| | | R](T)[sP].[dR](T)[sP].[dR](A)[sP].[dR](G)[sP].[dR](G)[s |
| | | P].[dR](C)[sP].[dR](A)[sP].[dR](C)[sP].[LR](T)[P].[LR]( |
| | | [5meC])} |
| 25_111 | 517 | [LR]([5meC])[sP].[LR](G)[sP].[dR](T)[sP].[LR](T)[sP].[d |
| | | R](T)[sP].[LR](T)[sP][dR](G)[sP].[dR](T)[sP].[dR](C)[sP |
| | | ].[dR](C)[sP].[dR](C)[sP].[dR](A)[sP].[dR](A)[sP].[dR]( |
| | | G)[sP].[dR](A)[sP].[dR](A)[sP].[LR](T)[P].[LR](T)} |
| 25_113 | 517 | [LR]([5meC])[sP].[LR](G)[sP].[dR](T)[sP].[LR](T)[sP].[L |
| | | R](T)[sP].[dR](T)[sP][dR](G)[sP].[dR](T)[sP].[dR](C)[sP |
| | | ].[dR](C)[sP].[dR](C)[sP].[dR](A)[sP].[dR](A)[sP].[LR]( |
| | | G)[sP].[dR](A)[sP].[dR](A)[sP].[LR](T)[sP].[LR](T)} |
| 29_10 | 558 | [LR](T)[sP].[LR]([5meC])[sP].[dR](C)[sP].[dR](A)[sP].[L |
| | | R](T)[sP].[dR](T)[sP][dR](T)[sP].[dR](T)[sP].[dR](G)[sP |
| | | ].[dR](C)[sP].[dR](T)[sP].[dR](A)[sP].[dR](A)[sP].[dR]( |
| | | A)[sP].[LR](A)[sP].[dR](C)[sP].[LR](A)[sP].[dR](C)[sP]. |
| | | [LR](T)[sP].[LR](T)} |
| 29_124 | 580 | [LR](A)[sP].[LR]([5meC])[sP].[dR](A)[sP].[mR](A)[sP].[d |
| | | R](C)[sP].[dR](T)[sP].[dR](G)[sP].[dR](T)[sP].[dR](T)[s |
| | | P].[dR](T)[sP].[dR](G)[sP].[dR](T)[sP].[LR](T)[sP].[dR] |
| | | (T)[sP].[LR]([5meC])[sP].[mR](C)[sP].[LR](A)[sP].[LR](T |
| | | )} |
| 29_130 | 582 | [LR]([5meC])[sP].[LR](A)[sP].[dR](C)[sP].[LR](A)[sP].[L |
| | | R](A)[sP].[dR](C)[sP].[dR](T)[sP].[dR](G)[sP].[dR](T)[s |
| | | P].[dR](T)[sP].[dR](T)[sP].[dR](G)[sP].[dR](T)[sP].[dR] |
| | | (T)[sP].[dR](T)[sP].[LR]([5meC])[sP].[LR]([5meC])} |
| 29_138 | 584 | [LR](G)[sP].[LR](G)[sP].[dR](C)[sP].[dR](A)[sP].[LR]([5 |
| | | meC])[sP].[dR](A)[sP].[dR](A)[sP].[dR](C)[sP].[dR](T)[s |
| | | P].[dR](G)[sP].[dR](T)[sP].[dR](T)[sP].[dR](T)[sP].[LR] |
| | | (T)[sP].[LR](T)} |
| 29_24 | 558 | [LR](T)[sP].[LR]([5meC])[sP].[dR](C)[sP].[LR](A)[sP].[d |
| | | R](T)[sP].[dR](T)[sP].[dR](T)[sP].[dR](T)[sP].[dR](G)[s |
| | | P].[dR](C)[sP].[LR](T)[sP].[dR](A)[sP].[dR](A)[sP].[dR] |
| | | (A)[sP].[dR](A)[sP].[dR](C)[sP].[dR](A)[sP].[dR](C)[sP] |
| | | .[LR](T)[sP].[LR](T)} |
| 29_34 | 661 | [LR](T)[sP].[LR]([5meC])[sP].[dR](C)[sP].[LR](A)[sP].[d |
| | | R](T)[sP].[dR](T)[sP].[dR](T)[sP].[dR](T)[sP].[dR](G)[s |
| | | P].[dR](C)[sP].[dR](T)[sP].[dR](A)[sP].[LR](A)[sP].[dR] |
| | | (A)[sP].[LR](A)[sP].[dR](C)[sP].[LR](A)[sP].[LR]([5meC] |
| | | )[sP].[LR](T)} |
| 29_36 | 661 | [LR](T)[sP].[LR]([5meC])[sP].[dR](C)[sP].[LR](A)[sP].[d |
| | | R](T)[sP].[dR](T)[sP].[dR](T)[sP].[dR](T)[sP].[dR](G)[s |
| | | P].[dR](C)[sP].[dR](T)[sP].[dR](A)[sP].[dR](A)[sP].[dR] |
| | | (A)[sP].[LR](A)[sP].[LR]([5meC])[sP].[dR](A)[sP].[LR]([ |
| | | 5meC])[sP].[LR](T)} |
| 29_5 | 557 | [LR]([5meC])[sP].[LR]([5meC])[sP].[dR](A)[sP].[dR](T)[s |
| | | P].[dR](T)[sP].[dR](T)[sP].[dR](T)[sP].[dR](G)[sP].[dR] |
| | | (C)[sP].[dR](T)[sP].[dR](A)[sP].[dR](A)[sP].[LR](A)[sP] |
| | | .[LR](A)[sP].[dR](C)[sP].[LR](A)[sP].[dR](C)[sP].[LR](T |
| | | )[sP].[LR](T)} |
| 29_70 | 570 | [LR](A)[sP].[LR]([5meC])[sP].[dR](T)[sP].[dR](G)[sP].[L |
| | | R](T)[sP].[LR](T)[sP].[dR](T)[sP].[dR](G)[sP].[dR](T)[s |
| | | P].[dR](T)[sP].[dR](T)[sP].[dR](C)[sP].[dR](C)[sP].[dR] |
| | | (A)[sP].[dR](T)[sP].[dR](T)[sP].[LR](T)[sP].[LR](T)} |
| 29_78 | 573 | [LR](A)[sP].[LR](A)[sP].[dR](C)[sP].[LR](T)[sP].[LR](G) |
| | | [sP].[dR](T)[sP].[dR](T)[sP].[dR](T)[sP].[dR](G)[sP].[d |
| | | R](T)[sP].[dR](T)[sP].[dR](T)[sP].[dR](C)[sP].[dR](C)[s |
| | | P].[dR](A)[sP].[LR](T)[sP].[LR](T)[sP].[LR](T)} |
| 29_79 | 573 | [LR](A)[sP].[LR](A)[sP].[mR](C)[sP].[LR](T)[sP].[dR](G) |
| | | [sP].[dR](T)[sP].[dR](T)[sP].[dR](T)[sP].[dR](G)[sP].[d |
| | | R](T)[sP].[dR](T)[sP].[dR](T)[sP].[dR](C)[sP].[dR](C)[s |
| | | P].[dR](A)[sP].[mR](U)[sP].[LR](T)[sP].[LR](T)} |
| 29_84 | 573 | [LR](A)[sP].[LR](A)[sP].[dR](C)[sP].[LR](T)[sP].[dR](G) |
| | | [sP].[dR](T)[sP].[dR](T)[sP].[dR](T)[sP].[dR](G)[sP].[d |
| | | R](T)[sP].[dR](T)[sP].[dR](T)[sP].[dR](C)[sP].[dR](C)[s |
| | | P].[dR](A)[sP].[dR](T)[sP].[LR](T)[sP].[LR](T)} |
| 29_85 | 573 | [LR](A)[sP].[LR](A)[sP].[dR](C)[sP].[LR](T)[sP].[dR](G) |
| | | [sP].[MOE](T)[sP].[dR](T)[sP].[dR](T)[sP].[dR](G)[sP].[ |
| | | dR](T)[sP].[dR](T)[sP].[dR](T)[sP].[dR](C)[sP].[dR](C)[ |
| | | sP].[dR](A)[sP].[dR](T)[sP].[LR](T)[sP].[LR](T)} |
| 29_86 | 573 | [LR](A)[sP].[LR](A)[sP].[dR](C)[sP].[LR](T)[sP].[dR](G) |
| | | [sP].[mR](U)[sP].[dR](T)[sP].[dR](T)[sP].[dR](G)[sP].[d |
| | | R](T)[sP].[dR](T)[sP].[dR](T)[sP].[dR](C)[sP].[dR](C)[s |
| | | P].[dR](A)[sP].[dR](T)[sP].[LR](T)[sP].[LR](T)} |
| 34_39 | 613 | [LR](T)[sP].[LR](A)[sP].[LR](G)[sP].[dR](A)[sP].[LR](A) |
| | | [sP].[LR](G)[sP].[LR](A)[sP].[dR](A)[sP].[dR](G)[sP].[d |
| | | R](T)[sP].[dR](T)[sP].[dR](A)[sP].[dR](C)[SP].[dR](A)[s |
| | | P].[LR](T)[sP].[LR]([5meC])} |
| 34_46 | 614 | [LR](G)[sP].[LR](T)[sP].[dR](A)[sP].[LR](G)[sP].[LR](A) |
| | | [sP].[dR](A)[sP].[LR](G)[sP].[dR](A)[sP].[dR](A)[sP].[d |
| | | R](G)[sP].[dR](T)[sP].[dR](T)[sP].[dR](A)[sP].[dR](C)[s |
| | | P].[dR](A)[sP].[LR](T)[sP].[LR]([5meC])} |
| 37_15 | 62 | [LR](T)[sP].[LR]([5meC])[sP].[dR](C)[sP].[LR](A)[sP].[d |
| | | R](A)[sP].[dR](C)[sP].[LR](A)[sP].[dR](A)[sP].[dR](T)[s |
| | | P].[dR](G)[sP].[dR](G)[sP].[dR](T)[sP].[dR](A)[sP].[dR] |
| | | (T)[sP].[dR](G)[sP].[dR](T)[sP].[LR]([5meC])[sP].[LR]([ |
| | | 5meC])} |
| 38_10 | 630 | [LR](G)[sP].[LR]([5meC])[sP].[dR](T)[sP].[LR](A)[sP].[d |
| | | R](A)[sP].[dR](T)[sP].[dR](T)[sP].[dR](A)[sP].[dR](T)[s |
| | | P].[dR](T)[sP].[dR](G)[sP].[dR](C)[sP].[dR](T)[sP].[LR] |
| | | (G)[sP].[LR](T)[sP].[LR](T)[sP].[LR](T)} |
| 51_6 | 707 | [LR]([5meC])[sP].[LR](T)[sP].[dR](G)[sP].[dR](T)[sP].[L |
| | | R](T)[sP].[dR](T)[sP].[LR](T)[sP].[dR](G)[sP].[dR](G)[s |
| | | P].[dR](C)[sP].[dR](A)[sP].[dR](C)[sP].[dR](A)[sP].[dR] |
| | | (T)[sP].[LR]([5meC])[sP].[LR](T)} |
| 54_1 | 725 | [LR](A)P.[LR](G)[sP].[mR](A)[sP].[LR](T)[P].[LR](T)[sP] |
| | | .[dR](A)[sP].[mR](U)[sP].[dR](A)[sP].[dR](T)[sP].[dR](T |
| | | )[sP].[dR](T)[sP].[dR](A)[sP].[dR](G)[sP].[dR](G)[sP].[ |
| | | dR](C)[sP].[LR](A)P.[LR]([5meC])[sP].[LR](T)} |
| 54_2 | 725 | [LR](A)[sP].[LR](G)[sP].[mR](A)[sP].[LR](T)[sP].[LR](T) |
| | | [sP].[dR](A)[sP].[dR](T)[sP].[dR](A)[sP].[dR](T)[sP].[d |
| | | R](T)[sP].[dR](T)[sP].[dR](A)[sP].[dR](G)[sP].[dR](G)[s |
| | | P].[dR](C)[sP].[LR](A)[sP].[LR]([5meC])P.[LR](T)} |
| 54_3 | 725 | [LR](A)P.[LR](G)[P].[dR](A)[sP].[LR](T)[sP].[LR](T)[P]. |
| | | [dR](A)[sP].[dR](T)[sP].[dR](A)[sP].[dR](T)[sP].[dR](T) |
| | | [sP].[dR](T)[sP].[dR](A)[sP].[dR](G)[sP].[dR](G)[sP].[d |
| | | R](C)[sP].[LR](A)P.[LR]([5meC])[sP].[LR](T)} |
| 66_117 | 799 | [LR]([5meC])[sP].[LR](T)[sP].[dR](G)[sP].[LR](T)[sP].[d |
| | | R](A)[sP].[dR](T)[sP][dR](A)[sP].[dR](T)[sP].[dR](T)[sP |
| | | ].[dR](C)[sP].[dR](C)[sP].[dR](A)[sP].[dR](G)[sP].[dR]( |
| | | A)[sP].[LR](T)[sP].[LR](T)[sP].[LR](A)} |
| 66_120 | 799 | [LR]([5meC])[sP].[LR](T)[P].[LR](G)[sP].[LR](T)[sP].[dR |
| | | ](A)[sP].[dR](T)[sP].[dR](A)[sP].[dR](T)[sP].[dR](T)[sP |
| | | ].[dR](C)[sP].[dR](C)[sP].[dR](A)[sP].[dR](G)[sP].[dR]( |
| | | A)[sP].[dR](T)[sP].[LR](T)[sP].[LR](A)} |
| 66_123 | 801 | [LR](T)[sP].[LR]([5meC])[sP].[dR](T)[sP].[dR](G)[sP].[L |
| | | R](T)[sP].[dR](C)[sP].[dR](T)[sP].[dR](G)[sP].[dR](T)[s |
| | | P].[dR](A)[sP].[dR](T)[sP].[dR](A)[sP].[dR](T)[sP].[dR] |
| | | (T)[sP].[dR](C)[sP].[LR]([5meC])[sP].[LR](A)} |
| 66_124 | 801 | [LR](T)[sP].[LR]([5meC])[sP].[mR](U)[sP].[dR](G)[sP].[M |
| | | OE](T)[sP].[dR](C)[sP].[dR](T)[sP].[dR](G)[sP].[dR](T)[ |
| | | sP].[dR](A)[sP].[dR](T)[sP].[dR](A)[sP].[dR](T)[sP].[dR |
| | | ](T)[sP].[dR](C)[sP].[MOE]([5meC])[sP].[LR](A)} |
| 66_124 | 801 | [LR](T)[sP].[LR]([5meC])[sP].[mR](U)[sP].[dR](G)[sP].[M |
| | | OE](T)[sP].[dR](C)[sP].[dR](T)[sP].[dR](G)[sP].[dR](T)[ |
| | | sP].[dR](A)[sP].[dR](T)[sP].[dR](A)[sP].[dR](T)[sP].[dR |
| | | ](T)[sP].[dR](C)[sP].[MOE]([5meC])[sP].[LR](A)} |
| 66_126 | 801 | [LR](T)[sP].[LR]([5meC])[sP].[dR](T)[sP].[mR](G)[sP].[L |
| | | R](T)[sP].[dR](C)[sP].[dR](T)[sP].[dR](G)[sP].[dR](T)[s |
| | | P].[dR](A)[sP].[dR](T)[sP].[dR](A)[sP].[dR](T)[sP].[dR] |
| | | (T)[sP].[dR](C)[sP].[LR]([5meC])[sP].[LR](A)} |
| 66_127 | 801 | [LR](T)[sP].[LR]([5meC])[P].[mR](U)[sP].[dR](G)[sP].[LR |
| | | ](T)[sP].[dR](C)[sP].[dR](T)[sP].[dR](G)[sP].[dR](T)[sP |
| | | ].[dR](A)[sP].[dR](T)[sP].[dR](A)[sP].[dR](T)[sP].[dR]( |
| | | T)[sP].[dR](C)[sP].[LR]([5meC])[sP].[LR](A)} |
| 66_130 | 801 | [LR](T)[sP].[MOE]([5meC])[sP].[MOE](T)[sP].[dR](G)[sP]. |
| | | [MOE](T)[sP].[dR](C)[sP].[dR](T)[sP].[dR](G)[sP].[dR](T |
| | | )[sP].[dR](A)[sP].[dR](T)[sP].[dR](A)[sP].[dR](T)[sP].[ |
| | | dR](T)[sP].[MOE](C)P.[LR]([5meC])[sP].[LR](A)} |
| 66_134 | 801 | [LR](T)[sP].[LR]([5meC])[sP].[dR](T)[sP].[dR](G)[sP].[L |
| | | R](T)[sP].[dR](C)[sP].[dR](T)[sP].[dR](G)[sP].[dR](T)[s |
| | | P].[dR](A)[sP].[dR](T)[sP].[dR](A)[sP].[dR](T)[sP].[dR] |
| | | (T)[sP].[dR](C)[sP].[MOE]([5meC])[sP].[LR](A)} |
| 66_135 | 801 | [LR](T)[sP].[MOE]([5meC])[sP].[MOE](T)[sP].[dR](G)[sP]. |
| | | [MOE](T)[sP].[dR](C)[sP].[dR](T)[sP].[dR](G)[sP].[dR](T |
| | | )[sP].[dR](A)[sP].[dR](T)[sP].[dR](A)[sP].[dR](T)[sP].[ |
| | | dR](T)[sP].[MOE](C)[sP].[LR]([5meC])[sP].[LR](A)} |
| 66_140 | 802 | [LR](A)[sP].[LR](A)[sP].[dR](C)[P].[LR](A)P.[MOE]([5meC |
| | | ])[sP].[dR](T)[sP].[dR](T)[sP].[dR](T)[sP].[dR](A)[sP]. |
| | | [dR]([5meC])[sP].[dR](G)[sP].[dR](T)[sP].[dR](C)[sP].[M |
| | | OE](T)[sP].[LR](G)[sP].[dR](T)[sP].[MOE]([5meC])[sP].[L |
| | | R](T)} |
| 66_149 | 802 | [LR](A)[sP].[LR](A)[sP].[dR](C)[P].[LR](A)[sP].[dR](C)[ |
| | | sP].[dR](T)[sP].[dR](T)[sP].[dR](T)[sP].[dR](A)[sP].[dR |
| | | ](C)[sP].[dR](G)[sP].[dR](T)[sP].[dR](C)[sP].[dR](T)[sP |
| | | ].[LR](G)[sP].[dR](T)[sP].[LR]([5meC])[sP].[LR](T)} |
| 66_153 | 802 | [LR](A)[sP].[LR](A)[sP].[dR](C)[sP].[LR](A)[sP].[MOE]([ |
| | | 5meC])[sP].[dR](T)[sP].[dR](T)[sP].[dR](T)[sP].[dR](A)[ |
| | | sP].[dR](C)[sP].[dR](G)[sP].[dR](T)[sP].[dR](C)[sP].[MO |
| | | E](T)[sP].[LR](G)[sP].[dR](T)[sP].[MOE]([5meC])[sP].[LR |
| | | ](T)} |
| 66_160 | 803 | [LR](A)[sP].[LR]([5meC])P.[LR](A)[sP].[dR](C)[sP].[dR]( |
| | | T)[sP].[dR](T)[sP].[dR](T)[sP].[dR](A)[sP].[dR]([5meC]) |
| | | [sP].[dR](G)[sP].[dR](T)[sP].[dR](C)[sP].[dR](T)[sP].[d |
| | | R](G)[sP].[dR](T)[sP].[LR]([5meC])[sP].[LR](T)} |
| 66_173 | 802 | [LR](A)[sP].[LR](A)[sP].[dR](C)[sP].[MOE](A)[sP].[MOE]( |
| | | [5meC])[sP].[dR](T)[sP].[dR](T)[sP].[dR](T)[sP].[dR](A) |
| | | [sP].[dR]([5meC])[sP].[dR](G)[sP].[dR](T)[sP].[dR](C)[s |
| | | P].[MOE](T)P.[LR](G)[sP].[mR](U)[sP].[MOE]([5meC])[sP]. |
| | | [LR](T)} |
| 66_174 | 803 | [LR](A)[sP].[LR]([5meC])[sP].[MOE](A)[sP].[dR](C)[sP].[ |
| | | dR](T)[sP].[dR](T)[sP].[dR](T)[sP].[dR](A)[sP].[dR]([5m |
| | | eC])[sP].[dR](G)[sP].[dR](T)[sP].[dR](C)[SP].[dR](T)[sP |
| | | ].[MOE](G)[sP].[MOE](T)[sP].[LR]([5meC])[sP].[LR](T)} |
| 66_176 | 803 | [LR](A)[sP].[LR]([5meC])[sP].[LR](A)[sP].[dR](C)[P].[mR |
| | | ](U)[P].[dR](T)[sP].[dR](T)[sP].[dR](A)[sP].[dR]([5meC] |
| | | )[sP].[dR](G)[sP].[dR](T)[sP].[dR](C)[sP].[dR](T)[sP].[ |
| | | dR](G)[sP].[dR](T)[sP].[LR]([5meC])[sP].[LR](T)} |
| 66_177 | 802 | [LR](A)[sP].[LR](A)[sP].[dR](C)[sP].[LR](A)[sP].[MOE]([ |
| | | 5meC])[sP].[dR](T)[sP].[dR](T)[sP].[dR](T)[sP].[dR](A)[ |
| | | sP].[dR]([5meC])[sP].[dR](G)[sP].[dR](T)[sP].[dR](C)[sP |
| | | ].[MOE](T)[sP].[LR](G)[sP].[mR](U)[P].[MOE]([5meC])[sP] |
| | | .[LR](T)} |
| 66_181 | 802 | [LR](A)[sP].[MOE](A)[sP].[MOE](C)[sP].[LR](A)[sP].[dR]( |
| | | C)[sP].[dR](T)[sP].[dR](T)[sP].[dR](T)[sP].[dR](A)[sP]. |
| | | [dR]([5meC])[sP].[dR](G)[sP].[dR](T)[sP].[dR](C)[sP].[M |
| | | OE](T)[P].[MOE](G)[sP].[MOE](T)[P].[LR]([5meC])[sP].[LR |
| | | ](T)} |
| 66_182 | 802 | [LR](A)[sP].[LR](A)[sP].[dR](C)[sP].[LR](A)[sP].[MOE]([ |
| | | 5meC])[sP].[dR](T)[sP].[dR](T)[sP].[dR](T)[sP].[dR](A)[ |
| | | sP].[dR]([5meC])[sP].[dR](G)[sP].[dR](T)[sP].[dR](C)[sP |
| | | ].[MOE](T)[sP].[LR](G)[P].[MOE](T)[sP].[MOE]([5meC])[sP |
| | | ].[LR](T)} |
| 66_183 | 802 | [LR](A)[sP].[LR](A)[sP].[dR](C)[sP].[LR](A)[sP].[MOE]([ |
| | | 5meC])[sP].[dR](T)[sP].[dR](T)[sP].[dR](T)[sP].[dR](A)[ |
| | | sP].[dR]([5meC])[sP].[dR](G)[sP].[dR](T)[sP].[dR](C)[sP |
| | | ].[MOE](T)P.[LR](G)[sP].[mR](U)[P].[MOE]([5meC])[sP].[L |
| | | R](T)} |
| 66_185 | 802 | [LR](A)[sP].[LR](A)[sP].[dR](C)[sP].[LR](A)P.[MOE]([5me |
| | | C])[sP].[dR](T)[SP].[dR](T)[sP].[dR](T)[sP].[dR](A)[sP] |
| | | .[dR]([5meC])[sP].[dR](G)[sP].[dR](T)[sP].[dR](C)[sP].[ |
| | | MOE](T)P.[LR](G)[sP].[mR](U)[sP].[MOE]([5meC])[sP].[LR] |
| | | (T)} |
| 66_188 | 802 | [LR](A)[sP].[LR](A)[P].[dR](C)[sP].[LR](A)[sP].[dR](C)[ |
| | | P].[dR](T)[sP].[dR](T)[sP].[dR](T)[P].[dR](A)[sP].[dR]( |
| | | [5meC])[sP].[dR](G)[sP].[dR](T)[sP].[dR](C)[sP].[dR](T) |
| | | [sP].[LR](G)[sP].[mR](U)[sP].[LR]([5meC])[sP].[LR](T)} |
| 66_189 | 802 | [LR](A)[sP].[LR]([5meC])[sP].[LR](A)[P].[dR](C)[sP].[dR |
| | | ](T)[sP].[dR](T)[sP].[dR](T)[sP].[dR](A)[sP].[dR](C)[sP |
| | | ].[dR](G)[sP].[dR](T)[sP].[dR](C)[sP].[dR](T)[sP].[dR]( |
| | | G)[sP].[dR](T)[sP].[LR]([5meC])[sP].[LR](T)} |
| 66_217 | 805 | [LR]([5meC])[sP].[LR](T)[sP].[dR](A)[sP].[LR](A)[sP].[L |
| | | R](A)[sP].[dR](A)[SP].[dR](C)[sP].[dR](A)[sP].[dR](C)[s |
| | | P].[dR](T)[sP].[dR](T)[sP].[dR](T)[sP].[dR](A)[sP].[dR] |
| | | (C)[sP].[dR](G)[sP].[LR](T)[sP].[LR]([5meC])[sP].[LR](T |
| | | )} |
| 66_220 | 805 | [LR]([5meC])[sP].[LR](T)[sP].[dR](A)[sP].[LR](A)[sP].[L |
| | | R](A)[sP].[dR](A)[sP].[mR](C)[sP].[dR](A)[sP].[dR](C)[s |
| | | P].[dR](T)[sP].[dR](T)[sP].[dR](T)[sP].[dR](A)[sP].[dR] |
| | | ([5meC])[sP].[dR](G)[sP].[LR](T)[sP].[LR]([5meC])[sP].[ |
| | | LR](T)} |
| 66_27 | 785 | [LR](G)[sP].[LR](T)[sP].[dR](A)[sP].[LR](T)[sP].[LR](A) |
| | | [sP].[dR](T)[sP].[dR](T)[sP].[dR](C)[sP].[dR](C)[sP].[d |
| | | R](A)[sP].[dR](G)[sP].[dR](A)[sP].[dR](T)[sP].[dR](T)[s |
| | | P].[dR](A)[sP].[dR](A)[sP].[dR](C)[sP].[LR](A)[P].[LR]( |
| | | A)[sP].[LR](A)} |
| 66_36 | 787 | [LR](T)[sP].[LR](G)[sP].[dR](T)[sP].[LR](A)[sP].[dR](T) |
| | | [sP].[dR](A)[sP].[dR](T)[sP].[dR](T)[sP].[LR]([5meC])[s |
| | | P].[LR]([5meC])[sP].[dR](A)[sP].[dR](G)[sP].[dR](A)[sP] |
| | | .[dR](T)[sP].[dR](T)[sP].[dR](A)[sP].[dR](A)[sP].[dR](C |
| | | )[sP].[LR](A)[sP].[LR](A)} |
| 66_39 | 787 | [LR](T)[P].[LR](G)[sP].[dR](T)[sP].[LR](A)[sP].[dR](T)[ |
| | | sP].[dR](A)[sP].[mR](U)[sP].[dR](T)[sP].[LR]([5meC])[sP |
| | | ].[LR]([5meC])[sP].[dR](A)[sP].[dR](G)[sP].[dR](A)[sP]. |
| | | [dR](T)[sP].[dR](T)[sP].[dR](A)[sP].[dR](A)[sP].[dR](C) |
| | | [P].[LR](A)[sP].[LR](A)} |
| 66_408 | 819 | [LR](G)[sP].[LR](T)[sP].[dR](T)[sP].[dR](T)[sP].[LR]([5 |
| | | meC])[sP].[dR](C)[sP].[dR](A)[sP].[dR](T)[sP].[dR](T)[s |
| | | P].[dR](T)[sP].[dR](T)[sP].[dR](G)[sP].[dR](C)[sP].[dR] |
| | | (T)[sP].[LR](A)[sP].[dR](A)[sP].[LR](A)[sP].[LR](A)} |
| 66_412 | 819 | [LR](G)[sP].[LR](T)[sP].[LR](T)[sP].[LR](T)[sP].[dR](C) |
| | | [sP].[dR](C)[sP].[dR](A)[sP].[dR](T)[sP].[dR](T)[sP].[d |
| | | R](T)[sP].[dR](T)[sP].[dR](G)[sP].[dR](C)[sP].[dR](T)[s |
| | | P].[dR](A)[sP].[dR](A)[sP].[LR](A)[sP].[LR](A)} |
| 66_430 | 822 | [LR](T)[sP].[LR](T)[sP].[dR](G)[sP].[dR](T)[sP].[MOE](T |
| | | )[sP].[LR](T)[sP].[dR](C)[sP].[dR](C)[sP].[dR](A)[sP].[ |
| | | dR](T)[sP].[dR](T)[sP].[dR](T)[sP].[dR](T)[sP].[dR](G)[ |
| | | sP].[dR](C)[sP].[MOE](T)[sP].[dR](A)[sP].[MOE](A)[sP].[ |
| | | LR](A)} |
| 66_431 | 822 | [LR](T)[sP].[LR](T)[sP].[MOE](G)[sP].[MOE](T)[sP].[LR]( |
| | | T)[sP].[MOE](T)[sP].[dR](C)[sP].[dR](C)[sP].[dR](A)[sP] |
| | | .[dR](T)[sP].[dR](T)[sP].[dR](T)[sP].[dR](T)[sP].[dR](G |
| | | )[sP].[dR](C)[sP].[MOE](T)[P].[MOE](A)[sP].[LR](A)[sP]. |
| | | [LR](A)} |
| 66_456 | 823 | [LR](T)[sP].[LR](G)[sP].[dR](T)[sP].[dR](T)[sP].[dR](T) |
| | | [sP].[LR]([5meC])[sP].[dR](C)[P].[dR](A)[sP].[dR](T)[sP |
| | | ].[dR](T)[sP].[dR](T)[sP].[dR](T)[sP].[dR](G)[sP].[dR]( |
| | | C)[sP].[dR](T)[sP].[dR](A)[sP].[LR](A)[sP].[LR](A)} |
| 66_459 | 822 | [LR](T)[sP].[LR](T)[sP].[dR](G)[sP].[dR](T)[sP].[LR](T) |
| | | [P].[LR](T)[sP].[dR](C)[sP].[dR](C)[sP].[dR](A)[sP].[dR |
| | | ](T)[sP].[dR](T)[sP].[dR](T)[sP].[dR](T)[sP].[dR](G)[sP |
| | | ].[dR](C)[sP].[LR](T)[sP].[dR](A)[sP].[LR](A)[sP].[LR]( |
| | | A)} |
| 66_47 | 789 | [LR](G)[sP].[LR](T)[sP].[dR](A)[sP].[LR](T)[P].[dR](A)[ |
| | | sP].[dR](T)[sP].[dR](T)[sP].[dR](C)[sP].[dR](C)[sP].[dR |
| | | ](A)[sP].[LR](G)[sP].[dR](A)[sP].[dR](T)[sP].[dR](T)[sP |
| | | ].[dR](A)[sP].[dR](A)[sP].[dR](C)[sP].[LR](A)[sP].[LR]( |
| | | A)} |
| 66_485 | 828 | [LR](A)[sP].[MOE](A)[sP].[MOE](C)P.[LR](T)[sP].[dR](G)[ |
| | | sP].[MOE](T)[sP].[dR](T)[sP].[dR](T)[sP].[dR](G)[sP].[d |
| | | R](T)[sP].[dR](T)[sP].[dR](T)[sP].[dR](C)[sP].[dR](C)[s |
| | | P].[MOE](A)[sP].[dR](T)[sP].[LR](T)[sP].[LR](T)} |
| 66_492 | 828 | [LR](A)P.[LR](A)[sP].[dR](C)[sP].[LR](T)[sP].[dR](G)[sP |
| | | ].[dR](T)[sP].[dR](T)[sP].[dR](T)[sP].[dR](G)[sP].[dR]( |
| | | T)[sP].[dR](T)[sP].[dR](T)[sP].[dR](C)[sP].[dR](C)[sP]. |
| | | [dR](A)[sP].[dR](T)[sP].[LR](T)P.[LR](T)} |
| 66_496 | 828 | [LR](A)[sP].[LR](A)[sP].[dR](C)[sP].[LR](T)[sP].[dR](G) |
| | | [sP].[mR](U)[sP].[dR](T)[sP].[dR](T)[sP].[dR](G)[sP].[d |
| | | R](T)[sP].[dR](T)[sP].[dR](T)[sP].[dR](C)[sP].[dR](C)[s |
| | | P].[dR](A)[sP].[mR](U)[sP].[LR](T)[sP].[LR](T)} |
| 66_544 | 829 | [LR](A)P.[LR]([5meC])[sP].[dR](A)[sP].[dR](A)[sP].[dR]( |
| | | C)[sP].[dR](T)[sP].[dR](G)[sP].[dR](T)[sP].[dR](T)[sP]. |
| | | [dR](T)[sP].[dR](G)[sP].[dR](T)[sP].[LR](T)[sP].[dR](T) |
| | | [sP].[LR]([5meC])[sP].[mR](C)[sP].[LR](A)[sP].[LR](T)} |
| 66_545 | 829 | [LR](A)P.[LR]([5meC])[sP].[dR](A)[sP].[mR](A)[sP].[dR]( |
| | | C)[sP].[dR](T)[sP].[dR](G)[sP].[dR](T)[sP].[dR](T)[sP]. |
| | | [dR](T)[sP].[dR](G)[sP].[dR](T)[sP].[LR](T)[sP].[dR](T) |
| | | [sP].[LR]([5meC])[sP].[mR](C)[sP].[LR](A)P.[LR](T)} |
| 66_547 | 829 | [LR](A)P.[LR]([5meC])[sP].[dR](A)[sP].[mR](A)[sP].[dR]( |
| | | C)[sP].[dR](T)[sP].[dR](G)[sP].[dR](T)[sP].[dR](T)[sP]. |
| | | [dR](T)[sP].[dR](G)[sP].[dR](T)[sP].[LR](T)[sP].[mR](U) |
| | | [sP].[LR]([5meC])[sP].[dR](C)[sP].[LR](A)P.[LR](T)} |
| 66_567 | 829 | [LR](A)[sP].[LR]([5meC])[sP].[dR](A)[sP].[mR](A)[sP].[d |
| | | R](C)[sP].[dR](T)[sP].[dR](G)[sP].[dR](T)[sP].[dR](T)[s |
| | | P].[dR](T)[sP].[dR](G)[sP].[dR](T)[sP].[MOE](T)[sP].[mR |
| | | ](U)[sP].[LR]([5meC])[sP].[mR](C)[sP].[LR](A)[sP].[LR]( |
| | | T)} |
| 66_573 | 829 | [LR](A)[sP].[MOE]([5meC])[P].[dR](A)[sP].[mR](A)[sP].[d |
| | | R](C)[sP].[dR](T)[sP].[dR](G)[P].[dR](T)[sP].[dR](T)[sP |
| | | ].[dR](T)[sP].[dR](G)[sP].[dR](T)[sP].[LR](T)[sP].[mR]( |
| | | U)[sP].[MOE]([5meC])[sP].[mR](C)[sP].[MOE](A)[sP].[LR]( |
| | | T)} |
| 66_576 | 830 | [LR](A)[sP].[LR]([5meC])[sP].[LR](A)[sP].[LR](A)[sP].[d |
| | | R](C)[sP].[dR](T)[sP].[dR](G)[sP].[dR](T)[sP].[dR](T)[s |
| | | P].[dR](T)[sP].[dR](G)[sP].[dR](T)[sP].[dR](T)[sP].[dR] |
| | | (T)[sP].[dR](C)[sP].[LR]([5meC])[sP].[LR](A)} |
| 66_584 | 830 | [LR](A)[sP].[LR]([5meC])[sP].[LR](A)[sP].[LR](A)[sP].[d |
| | | R](C)[sP].[mR](U)[sP].[dR](G)[sP].[dR](T)[sP].[dR](T)[s |
| | | P].[dR](T)[sP].[dR](G)[sP].[dR](T)[sP].[dR](T)[sP].[dR] |
| | | (T)[sP].[dR](C)[sP].[LR]([5meC])[sP].[LR](A)} |
| 66_587 | 830 | [LR](A)[sP].[LR]([5meC])[sP].[MOE](A)P.[MOE](A)[P].[dR] |
| | | (C)[sP].[dR](T)[sP].[dR](G)[sP].[dR](T)[sP].[dR](T)[sP] |
| | | .[dR](T)[sP].[dR](G)[sP].[dR](T)[sP].[dR](T)[sP].[MOE]( |
| | | T)[sP].[MOE](C)[sP].[LR]([5meC])[sP].[LR](A)} |
| 66_588 | 830 | [LR]([5meC])[sP].[LR](A)[sP].[dR](C)[sP].[LR](A)[sP].[d |
| | | R](A)[sP].[dR](C)[sP].[dR](T)[sP].[dR](G)[sP].[dR](T)[s |
| | | P].[dR](T)[sP].[dR](T)[sP].[dR](G)[sP].[dR](T)[sP].[dR] |
| | | (T)[sP].[dR](T)[sP].[dR](C)[sP].[LR]([5meC])[sP].[LR](A |
| | | )} |
| 66_592 | 830 | [LR](A)[sP].[LR]([5meC])[sP].[MOE](A)[sP].[MOE](A)[sP]. |
| | | [dR](C)[sP].[dR](T)[sP].[dR](G)[sP].[dR](T)[sP].[dR](T) |
| | | [sP].[dR](T)[sP].[dR](G)[sP].[dR](T)[sP][dR](T)[sP].[MO |
| | | E](T)[P].[MOE](C)[sP].[LR]([5meC])[sP].[LR](A)} |
| 66_593 | 831 | [LR]([5meC])[sP].[LR](A)[sP].[dR](C)[sP].[LR](A)[sP].[d |
| | | R](A)[sP].[mR](C)[sP].[dR](T)[sP].[dR](G)[sP].[dR](T)[s |
| | | P].[dR](T)[sP].[dR](T)[sP].[dR](G)[sP].[dR](T)[sP].[dR] |
| | | (T)[sP].[dR](T)[sP].[dR](C)[sP].[LR]([5meC])[sP].[LR](A |
| | | )} |
| 66_600 | 83 | [LR]([5meC])[sP].[MOE](A)[sP].[dR](C)[sP].[LR](A)[sP].[ |
| | | dR](A)[sP].[mR](C)[sP].[dR](T)[sP].[dR](G)[sP].[dR](T)[ |
| | | sP].[dR](T)[sP].[dR](T)[sP].[dR](G)[sP].[dR](T)[sP].[dR |
| | | ](T)[sP].[dR](T)[sP].[dR](C)[sP].[LR]([5meC])[sP].[LR]( |
| | | A)} |
| 66_63 | 787 | [LR](T)[sP].[MOE](G)[sP].[dR](T)[sP].[LR](A)[sP].[dR](T |
| | | )[sP].[mR](A)[sP].[dR](T)[sP].[dR](T)[sP].[MOE]([5meC]) |
| | | P.[LR]([5meC])[sP].[dR](A)[sP].[dR](G)[sP].[dR](A)[sP]. |
| | | [dR](T)[sP].[dR](T)[sP].[dR](A)[sP].[dR](A)[sP].[dR](C) |
| | | [sP].[LR](A)[sP].[LR](A)} |
| 66_64 | 787 | [LR](T)[sP].[MOE](G)[sP].[dR](T)[sP].[MOE](A)[sP].[dR]( |
| | | T)[sP].[dR](A)[sP].[dR](T)[sP].[dR](T)[sP].[LR]([5meC]) |
| | | P.[LR]([5meC])[sP].[dR](A)[P].[dR](G)[sP].[dR](A)[sP].[ |
| | | dR](T)[sP].[dR](T)[sP].[dR](A)[sP].[dR](A)[sP].[dR](C)[ |
| | | sP].[LR](A)[sP].[LR](A)} |
| 68_16 | 835 | [LR](G)[sP].[LR](T)[sP].[dR](A)[sP].[LR](T)[sP].[LR](A) |
| | | [sP].[dR](A)[sP].[dR](A)[sP].[dR](A)[sP].[dR](T)[sP].[d |
| | | R](T)[sP].[dR](C)[sP].[dR](A)[sP].[dR](C)[sP].[LR](G)[s |
| | | P].[LR](T)[sP].[LR]([5meC])[sP].[LR](A)} |

Helm Annotation Key for the compounds in Table C3:
[LR](G) is a beta-D-oxy-LNA guanine nucleoside,
[LR](T) is a beta-D-oxy-LNA thymine nucleoside,
[LR](A) is a beta-D-oxy-LNA adenine nucleoside,
[LR]([5meC]) is a beta-D-oxy-LNA 5-methyl cytosine nucleoside,
[MOE](G) is a 2′-O-Methoxyethyl guanine nucleoside,
[MOE](T) is a 2′-O-Methoxyethyl thymine nucleoside,
[MOE](A) is a 2′-O-Methoxyethyl adenine nucleoside,
[MOE]([5meC]) is a 2′-O-Methoxyethyl-5-methyl cytosine nucleoside,

The number prior to the underline indicates the target ID of the antisense compound. Thus, ASO ID 66_117 binds to a target sequence with Target ID No 66. The compound might also bind to other target IDs of the present invention (as some target IDs overlap, see e.g. Table D1).

Preferably, the compound selected from Table C1 is a compound which resulted in an efficient down-regulation of the target gene in the studies described in Example 5. The results for the experiments are shown in Table 8. Preferably, the compound is selected from the compounds resulting in an expression level of NAT8L of 50% or less than 50% in relation to PBS treated control cells. Preferably, the compound is selected from the compounds resulting in an expression level of NAT8L of 40% or less than 40% in relation to PBS treated control cells.

Preferably, the antisense oligonucleotide (or composition) of the present invention shall be administered to the CNS, in particular to the brain. Accordingly, the ASO is delivered to CNS through intrathecal injection—as it is e.g. the current state of art for similar ASO e.g. Nusinersen/Spinraza. Thus, the ASO of the present invention or the pharmaceutical composition is, preferably, administered intrathecally. In addition to delivery to the CNS, the ASO can be administered by subcutaneous or intravenous administration either with or with or without a conjugate in order to reach the peripheral nervous system, which is also negatively affected by the loss of ASPA expression.

The present invention further relates to a conjugate comprising the antisense oligonucleotide of the present invention and a conjugate moiety. Preferably, the conjugate moiety is covalently bound to the antisense oligonucleotide, e.g. via one or more linker nucleotides, such as one, two, three or four linker nucleotides (or more). The linker may be cleaved after administration to the patient.

Preferably, the antisense oligonucleotide of the present invention shall be delivered or administered to the CNS, in particular to the brain. Accordingly, it is envisaged that the conjugate moiety is a moiety that allows the crossing of the conjugate of the blood brain barrier. For example, the moiety can be and antibody or antigen-binding fragment thereof targeting the transferrin receptor.

The antisense oligonucleotides of the present invention can be administered/delivered ‘unassisted’ in saline solution. However, distribution to certain tissues and uptake in cells can be enhanced by conjugates and formulation techniques. Conjugation to ASOs could be, peptides, antibodies and aptamers binding to receptors on target cells or proteins mediating transcytosis e.g. the transferrin receptor. Antisense oligos can also be conjugated to naturally occurring ligands or modifications hereof as exemplified by GalNac conjugation binding with high affinity to asialoglycoprotein receptor 1 (ASGR1, ASPGR) and Alpha-tocopherol conjugation and interaction with transfer protein Alfa-TTP. This could also be small molecules generated through medicinal chemistry with high affinity for known receptors and transporter. Moreover, it could be conjugations to long chained fatty acids that modify the hydrophobicity and protein binding properties of the ASOs, but also could function through their capacity to bind to lipoprotein particles and hence function through the endogenous mechanism for lipid transport and uptake.

In addition to conjugation, tissues delivery and cellular uptake of ASOs of the present invention can be enhanced through formulation with nanocarriers, that facilitates crossing of biological barriers such as cellular membranes. Various types of nanocarriers have been described with with favorable properties for delivery of nucleic acids e.g. lipid nanoparticles (LNPs) as used for BioNTech mRNA vaccines, LNPs functionalizes with peptides, pegylated lipids, cationic lipids, exomes (lipid bilayers) both artificial and natural exosomes such as milk exosomes and spherical nucleic acids and others as described in further details in Roberts et al., 2020 (Thomas C Roberts, Robert Langer, Matthew J A Wood. Review Nat Rev Drug Discov. 2020 October; 19(10):673-694. Advances in oligonucleotide drug delivery).

The present invention further relates to a pharmaceutical composition comprising the antisense oligonucleotide of the present invention or the conjugate of the present invention.

Typically, a pharmaceutical composition comprises the antisense oligonucleotide or the conjugate of the present invention together with a pharmaceutically acceptable carrier and/or, in particular, a pharmaceutically acceptable excipient. The term “pharmaceutically acceptable”, as used herein, refers to the non-toxicity of a material which, in certain exemplary embodiments, does not interact with the action of the oligonucleotide or the conjugate present in the pharmaceutical composition.

The term “carrier”, as used herein, refers to an organic or inorganic component, of a natural or synthetic nature, in which the active component is combined in order to facilitate, enhance or enable application.

The term “excipient”, as used herein, is intended to include all substances which may be present in a pharmaceutical composition and which are not active ingredients, such as salts, binders (e.g., lactose, dextrose, sucrose, trehalose, sorbitol, mannitol), fillers, lubricants, thickeners, surface active agents, preservatives, emulsifiers or buffer substances.

The form of the pharmaceutical composition, the route of administration, the dosage and the regimen naturally depend upon the condition to be treated, the severity of the illness, the age, weight, and gender of the patient, etc.

In an embodiment, the pharmaceutical composition can be formulated for intrathecal administration. Thus, the antisense oligonucleotide or conjugate of the present invention is preferably administered by intrathecal administration a route of administration for drugs via an injection into the spinal canal. Thereby, it reaches the cerebrospinal fluid and the brain.

The present invention further relates to the antisense oligonucleotide according to the present invention, the conjugate according to the present invention, or the pharmaceutical composition according to the present invention for use in medicine.

Accordingly, the present invention relates to the antisense oligonucleotide according to the present invention, the conjugate according to the present invention, or the pharmaceutical composition according to the present invention for use treating Canavan disease.

Further, the present invention relates to the use of the antisense oligonucleotide according to the present invention, the conjugate according to the present invention, or the pharmaceutical composition according to the present invention for the manufacture of a medicament for treating Canavan disease. Canavan disease, also referred to as Canavan-van Bogaert-Bertrand disease is a degenerative disease that is associated with a progressive damage to nerve cells and loss of white matter in the brain. The disease is inherited in an autosomal recessive manner. It is caused by mutations the ASPA gene which codes for the enzyme aspartoacylase. Decreased aspartoacylase activity prevents the normal breakdown of N-acetyl aspartate.

Accordingly, the present invention relates to the antisense oligonucleotide according to the present invention, the conjugate according to the present invention, or the pharmaceutical composition according to the present invention for use treating Canavan disease.

Further, the present invention relates to the use of the antisense oligonucleotide according to the present invention, the conjugate according to the present invention, or the pharmaceutical composition according to the present invention for the manufacture of a medicament for treating Canavan disease.

Further, the present invention relates to a method of treating Canavan disease, comprising administering pharmaceutically effective amount of the antisense oligonucleotide according to the present invention, the conjugate according to the present invention, or the pharmaceutical composition according to the present invention to a subject suffering from Canavan disease.

The term “treating” or “treatment”, as used herein, refers to the administration of a compound or composition or a combination of compounds or compositions to a subject in order to: ameliorate Canavan disease. Thus, the term encompasses both the amelioration of one or more symptoms of the Canavan disease or prevention of the worsening of one or more symptoms, i.e. prophylaxis. The amelioration of symptoms also includes the reduction of one or more symptoms. Thus, the term, preferably, refers to the reduction of one or more symptoms of the disease. In other words, the treatment is typically a disease modifying treatment that reduces one or more symptoms of Canavan disease. In an embodiment, the development of disease pathology is inhibited. It will therefore be recognized that treatment as referred to herein may, in some embodiments, be prophylactic or disease modifying. It is to be understood that the treatment does not allow a complete cure of Canavan disease.

Canavan disease is known to decrease the life expectancy. Thus, the term “treatment” also includes increasing the life expectancy of a subject (as compared to an untreated subject).

In accordance with the present invention, the subject to be treated is a subject suffering from Canavan disease. However, the patient might not yet show symptoms of Canavan disease at the time of the treatment. Typically, the subject shows symptoms of Canavan disease. Symptoms of Canavan disease are well known in the art and include (but are not limited to) one or more of lack of motor development, macrocephaly, lack of head control and abnormal muscle tone. Preferably, the subject has been diagnosed through genetic testing to suffer from Canavan disease. Typically, the disease is diagnosed at the infant age. Also typically, the diagnosis involves genetic testing and/or the detection of increased levels of N-acetylaspartic acid (NAA) in the urine.

The terms “subject” and “patient” are used interchangeably herein. The “subject” or “patient” may be a vertebrate. The term includes both humans and other animals, particularly mammals, and other organisms. In some embodiments, the subject is a mammal. In some embodiments, the subject is a primate. Preferably, the subject is a human subject suffering from Canavan disease.

The present invention further relates to a method for identifying a candidate compound for the treatment of Canavan disease, comprising
a) providing an antisense oligonucleotide according to the present invention,
b) contacting a host cell expressing NAT8L mRNA with said antisense oligonucleotide,
c) determining the amount of NAT8L mRNA in the said host cell, and
d) identifying a candidate compound based on the results of step c).

The antisense oligonucleotide is preferably the antisense oligonucleotide of the present invention. Accordingly, it shall comprise a stretch of at least 10 nucleotides which is at least 90% complementary to a target sequence in the human NAT8L gene (i.e. mRNA or premRNA). The definitions provided herein above preferably apply mutatis mutandis.

Preferably, said method is an in vitro method. In step a) of the above method of the present invention an antisense oligonucleotide of the present invention is provided. Preferably, the antisense oligonucleotides are complementary to a target region as set forth herein elsewhere. In step b) the antisense oligonucleotide shall be contacted with a host cell. Said host cell shall express the NAT8L gene.

### EMBODIMENTS OF THE PRESENT INVENTION

In the following, preferred embodiments of the present invention are provided. The definitions and explanations made herein above apply mutatis mutandis.
1. An antisense oligonucleotide comprising a stretch of at least 10 nucleotides which is at least 90% complementary to a target sequence in the human NAT8L (N-acetyltransferase 8 like) gene.
2. The antisense oligonucleotide of embodiment 1, wherein the stretch of at least 10 nucleotides is at least 90% complementary to a target sequence selected from the group of target sequences consisting of SEQ ID NO: 4 to SEQ ID NO: 81.
3. The antisense oligonucleotide of embodiment 1 or 2, wherein the stretch of at least 10 nucleotides is at least 90% complementary to a target sequence selected from the group of target sequences consisting of SEQ ID NO: 4 to SEQ ID NO: 29.
4. The antisense oligonucleotide of any one of embodiments 1 to 3, wherein the stretch of at least 10 nucleotides is at least 90% complementary to a target sequence selected from the group of target sequences consisting of: SEQ ID NO: 4 to SEQ ID NO: 10.
5. The antisense oligonucleotide of any one of embodiments 1 to 4, comprising a stretch of at least 12, or at least 14 nucleotides which is at least 90% complementary to said target sequence.
6. The antisense oligonucleotide of any one of embodiments 1 to 5, wherein said stretch is 100% complementary to said target sequence.
7. The antisense oligonucleotide of any one of embodiments 1 to 6, wherein the antisense oligonucleotide has a length of 12 to 30 nucleotides.
8. The antisense oligonucleotide of embodiment 7, wherein the antisense oligonucleotide has a length of 14 to 22 nucleotides, such as a length of 16 to 20 nucleotides.
9. The antisense oligonucleotide of any one of embodiments 1 to 8, wherein the antisense oligonucleotide comprises or consists of a nucleic acid sequence as shown in SEQ ID NO: 403 to SEQ ID NO: 723.
10. The antisense oligonucleotide of any one of embodiments 1 to 9, wherein the antisense oligonucleotide is capable of reducing the amount of NAT8L (N-acetyltransferase 8 like) mRNA in a host cell expressing said NAT8L mRNA.
11. The antisense oligonucleotide embodiment 10, wherein the target cell is a human cell, such as a cell of the CNS.
12. The antisense oligonucleotide of any one of embodiments 1 to 11, wherein the antisense oligonucleotide is a chemically modified antisense oligonucleotide.
13. The antisense oligonucleotide of embodiment 12, wherein the chemically modified antisense oligonucleotide contains one or more modified nucleosides.
14. The antisense oligonucleotide of embodiment 13, wherein the one or more modified nucleosides, is a sugar modified nucleoside, such as a 2′ sugar modified nucleoside.
15. The antisense oligonucleotide of any one of embodiments 11 to 14, wherein the chemically modified antisense oligonucleotide contains at least one modified nucleobase.
16. The antisense oligonucleotide of embodiment 13, wherein the at least one modified nucleobase is 5-methylcytosine.
17. The antisense oligonucleotide of any one of embodiments 11 to 14, wherein the chemically modified antisense oligonucleotide comprises at least one modified nucleoside selected from the group consisting of: 2′-O-Methoxyethyl-RNA, 2′-O-Methyl-RNA, 2′-Fluoro-RNA.
18. The antisense oligonucleotide of any one of embodiments 1 to 17, wherein the antisense oligonucleotide comprises at least one modified internucleoside linkage, such as at least one Phosphorothioate internucleoside linkage, at least one Phosphorodithioate internucleoside linkage, at least one Phophoroamidate internucleoside linkage, at least one methyl phosphonate internucleoside linkage, at least one phosphotriester internucleoside linkage, at least one boranophosphate internucleoside linkage or at least one phosphoryl guanidine internucleoside linkage.
19. The antisense oligonucleotide of embodiment 18, wherein all internucleoside linkages are modified internucleoside linkages, such as Phosphorothioate internucleoside linkages.
20. The antisense oligonucleotide of any one of embodiments 1 to 19, wherein the antisense oligonucleotide comprises one or more modified nucleosides being LNA (locked nucleic acid) nucleosides,
21. The antisense oligonucleotide of embodiment 20, wherein the LNA nucleoside(s) is (are) a beta-D-oxy LNA nucleosides.
22. The antisense oligonucleotide of any one of embodiments 1 to 21, wherein the antisense oligonucleotide has a gapmer structure.
23. The antisense oligonucleotide of any one of embodiments 1 to 22, wherein the antisense oligonucleotide is a compound shown in Table A1, such as a compound as shown in Table A3.
24. A conjugate comprising the antisense oligonucleotide of any one of embodiments 1 to 23, wherein a conjugate moiety is covalently bound to the antisense oligonucleotide.
25. A pharmaceutical composition comprising the antisense oligonucleotide of any one of embodiments 1 to 23 or the conjugate of embodiment 24.
26. The antisense oligonucleotide of any one of embodiments 1 to 23, the conjugate of embodiment 24, or the pharmaceutical composition of embodiment 25 for use in treating Canavan disease.
27. A method for identifying a candidate compound for the treatment of Canavan disease, comprising
   a) providing an antisense oligonucleotide as defined in any one of embodiments 1 to 23,
   b) contacting a host cell expressing NAT8L mRNA with said antisense oligonucleotide,
   c) determining the amount of NAT8L mRNA in the said host cell, and
   d) identifying a candidate compound based on the results of step c).

All patents, patent applications, and publications or public disclosures referred to or cited herein are incorporated by reference in their entirety.

The invention will be further described with reference to the examples described herein; however, it is to be understood that the invention is not limited to such examples.

### EXAMPLES

### Oligonucleotide Synthesis

Oligonucleotide synthesis is generally known in the art and can be acquired from multiple different providers. In the listed in vitro experiments (example 1, 2, 3, 5, 6 and 7) all oligonucleotides were acquired from Biosearch Technologies (Lystrup, Denmark). In brief, after cleavage from the solid support the oligonucleotides are cartridge purified using Ammonium acetate; Dissolved to 750 μM in PBS and purity determined by LC/MS (≥80%).

In the listed in vivo experiments (example 9 and 10) the oligonucleotides were acquired from WuXi AppTec (China). After cleavage from the solid support the oligonucleotides were HPLC-purified to >90% purity (LC-TOF/MS) and lyophylized as sodium salt followed by formulation in sterile 0.9% sodiumchloride. Endotoxin test were performed (Kinetic turbidimetric or chromogenic LAL assay) to ensure endotoxin levels<1.0 EU/mg.

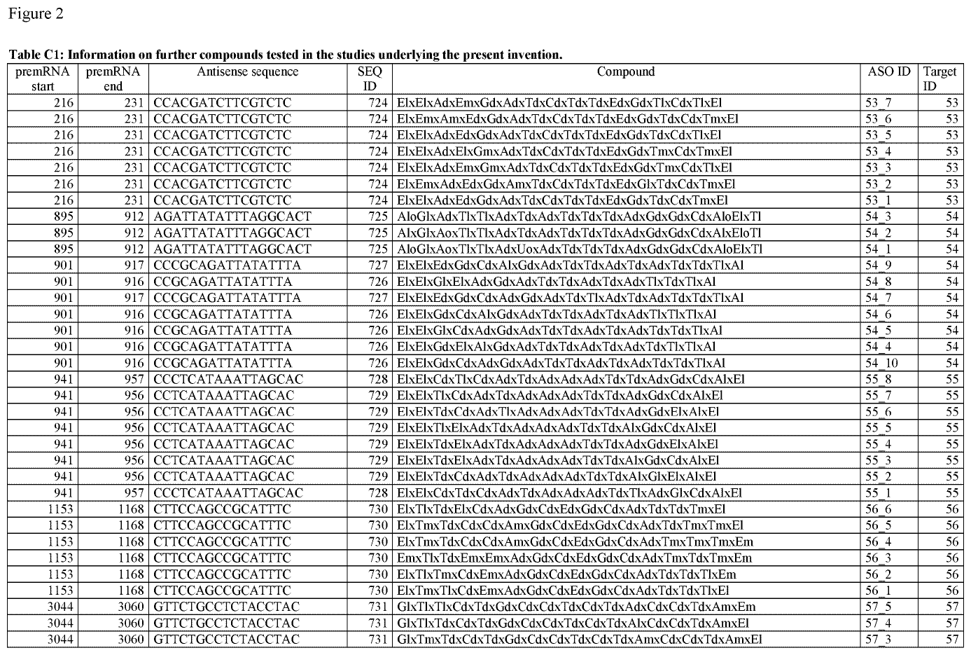

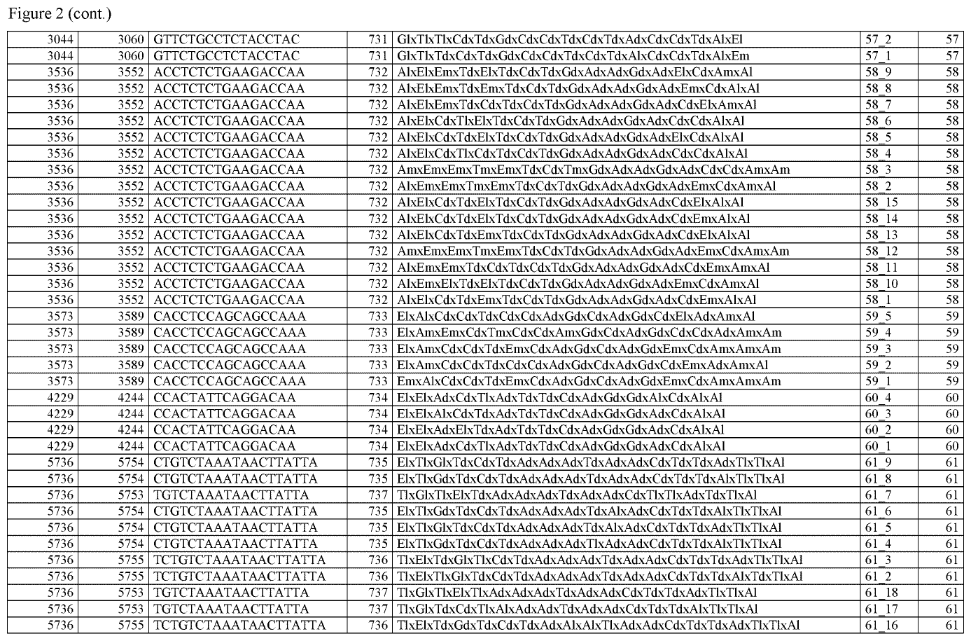

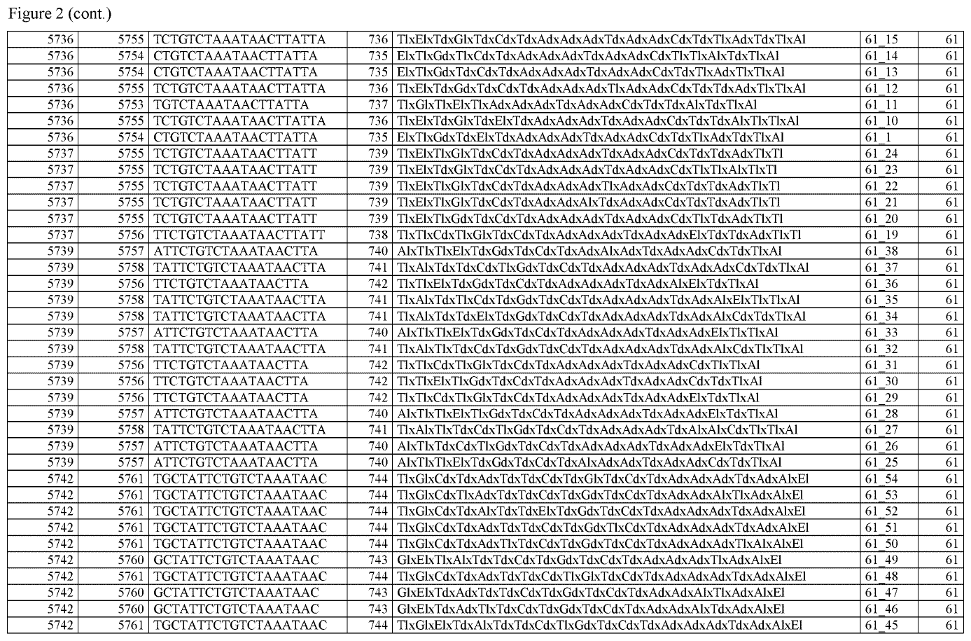

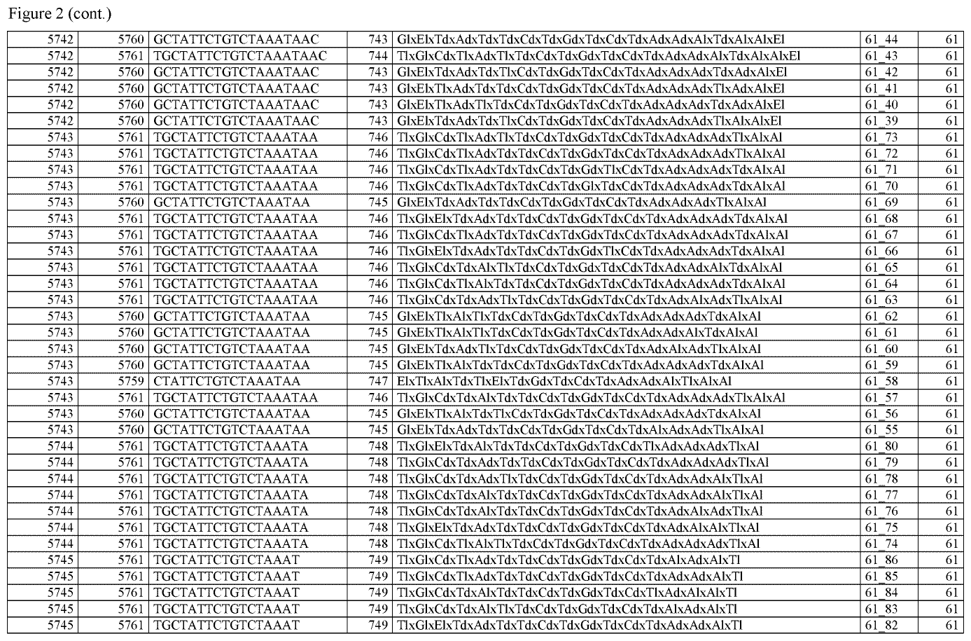

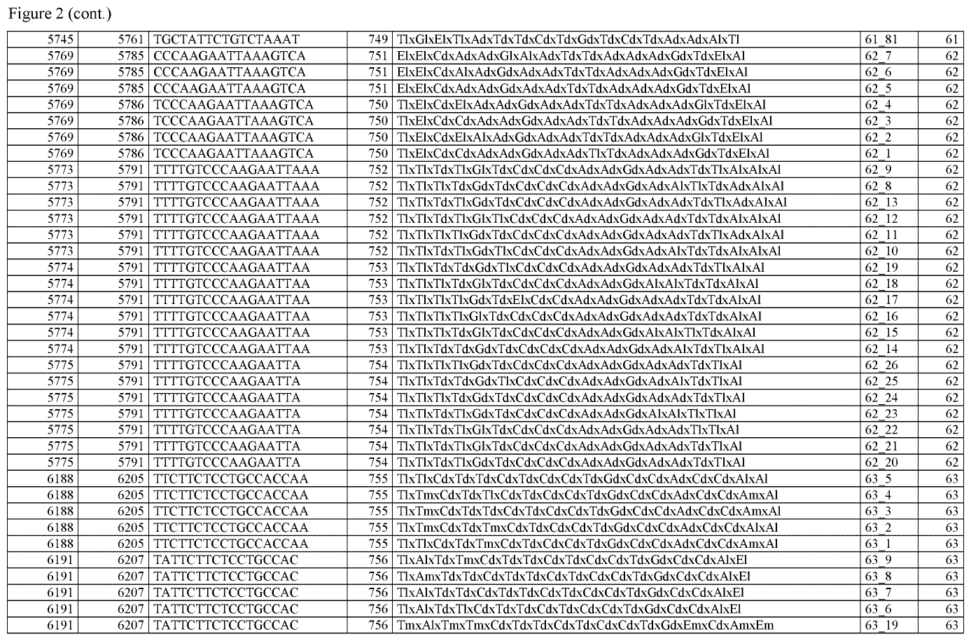

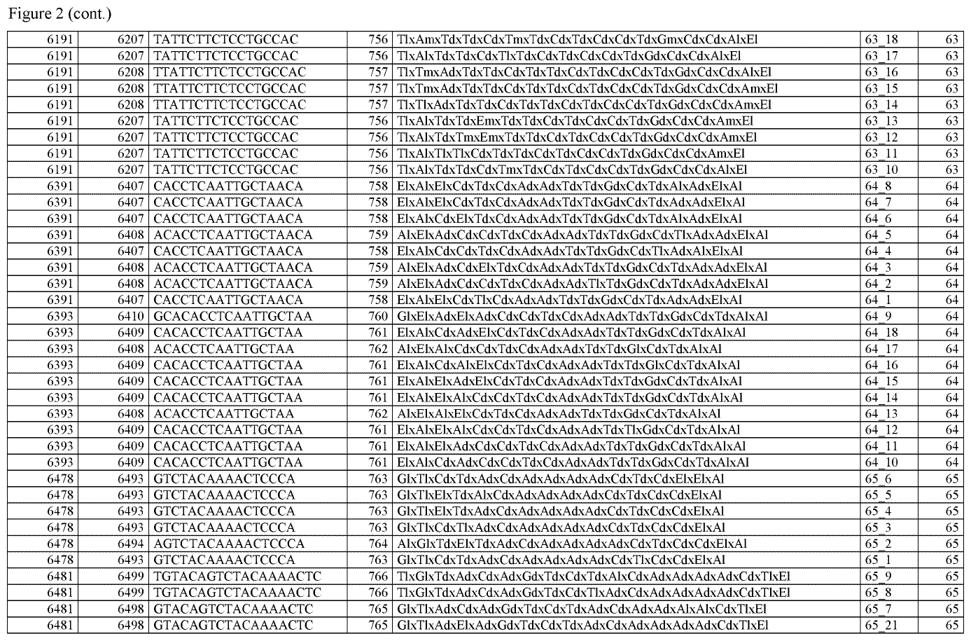

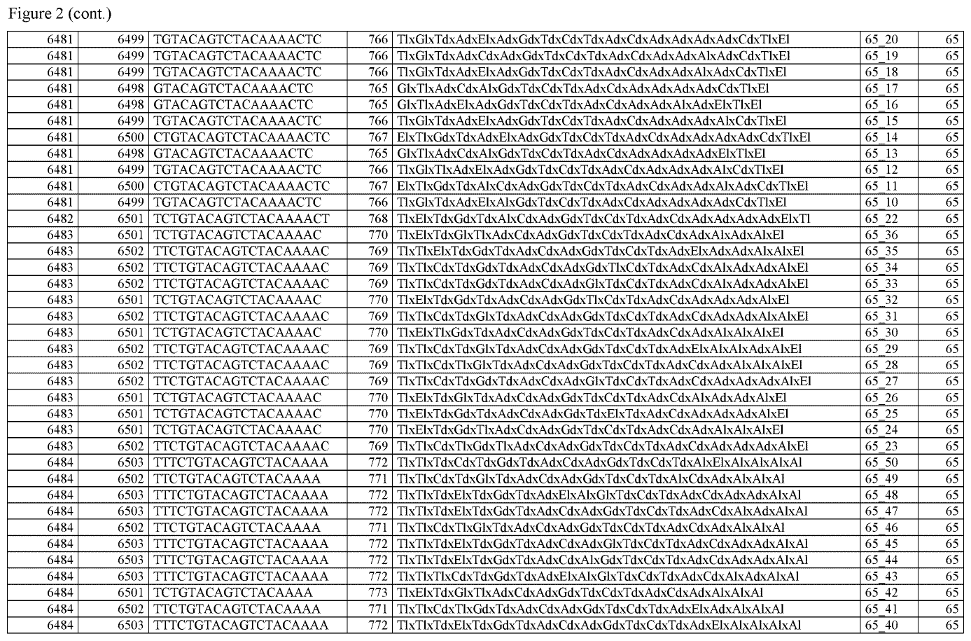

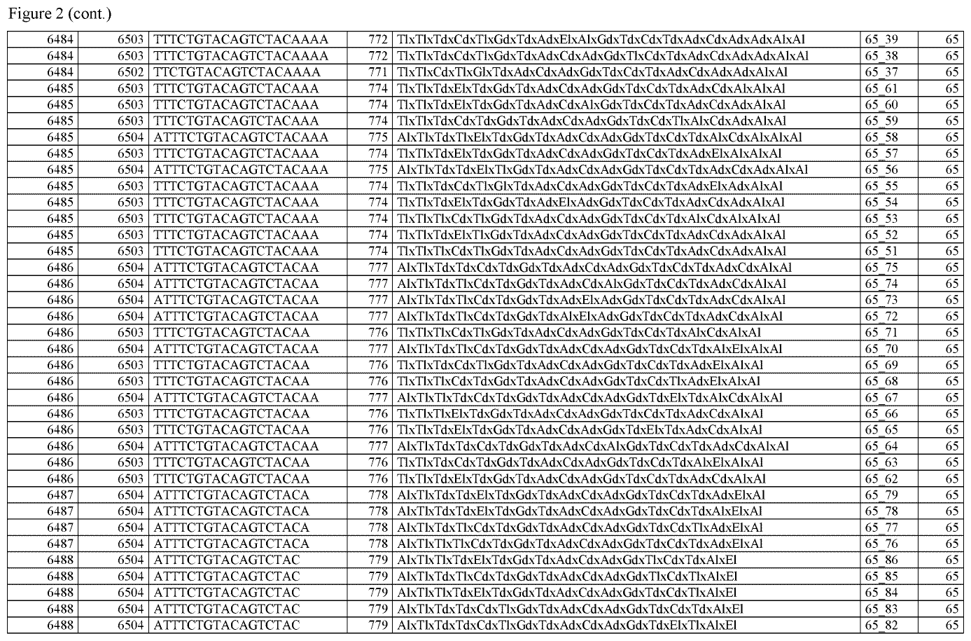

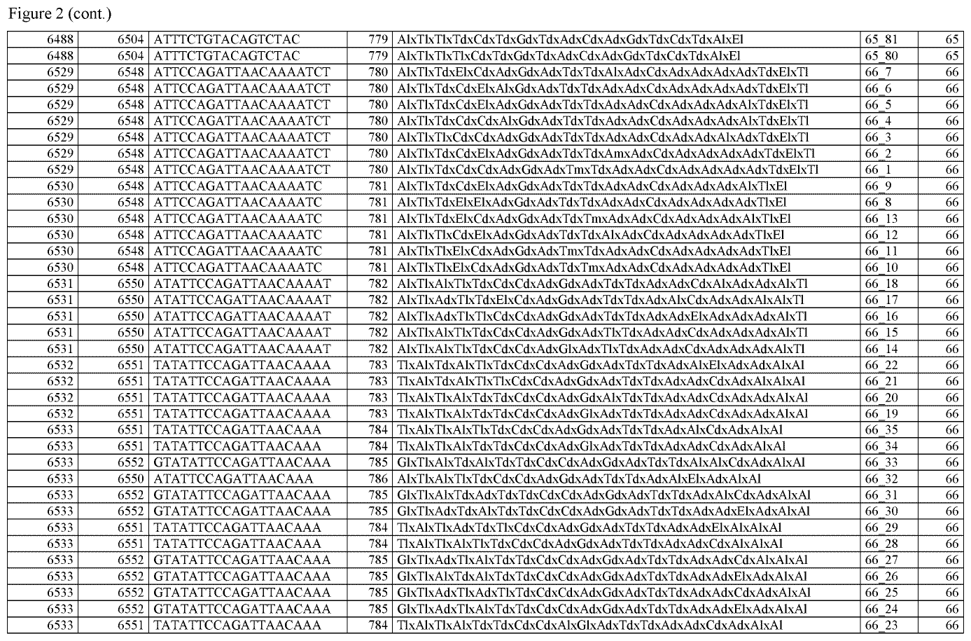

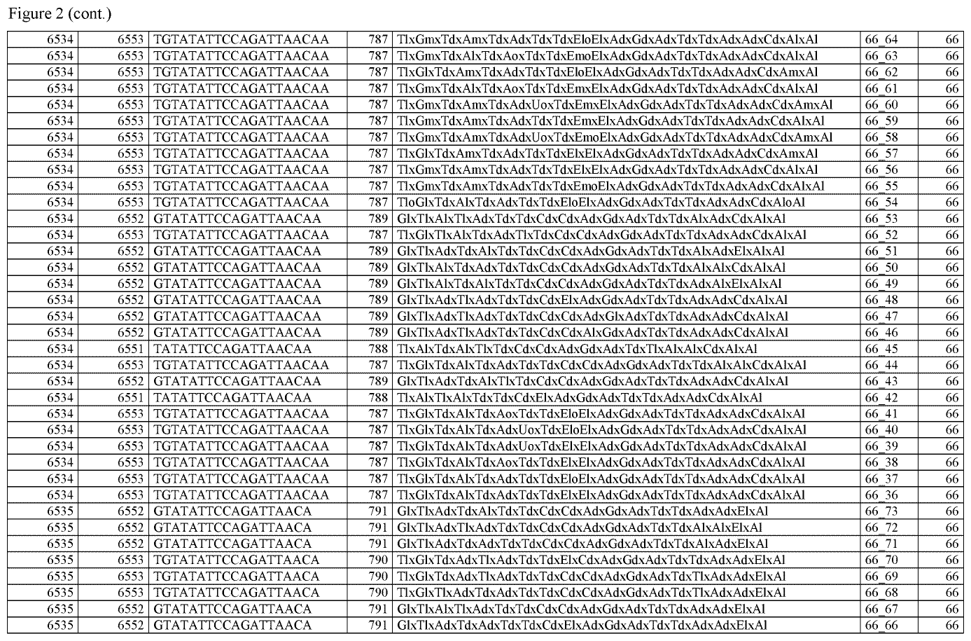

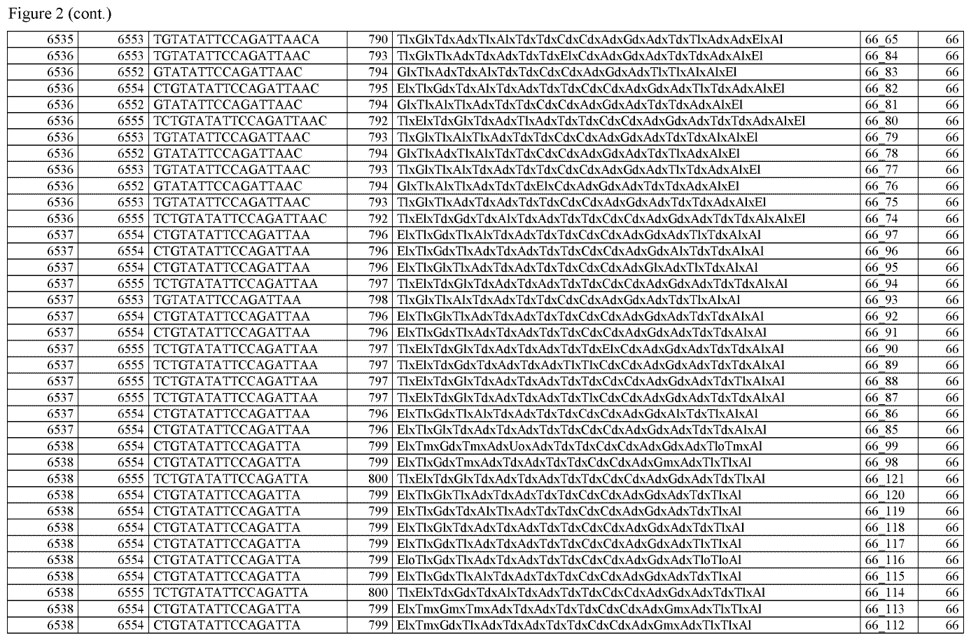

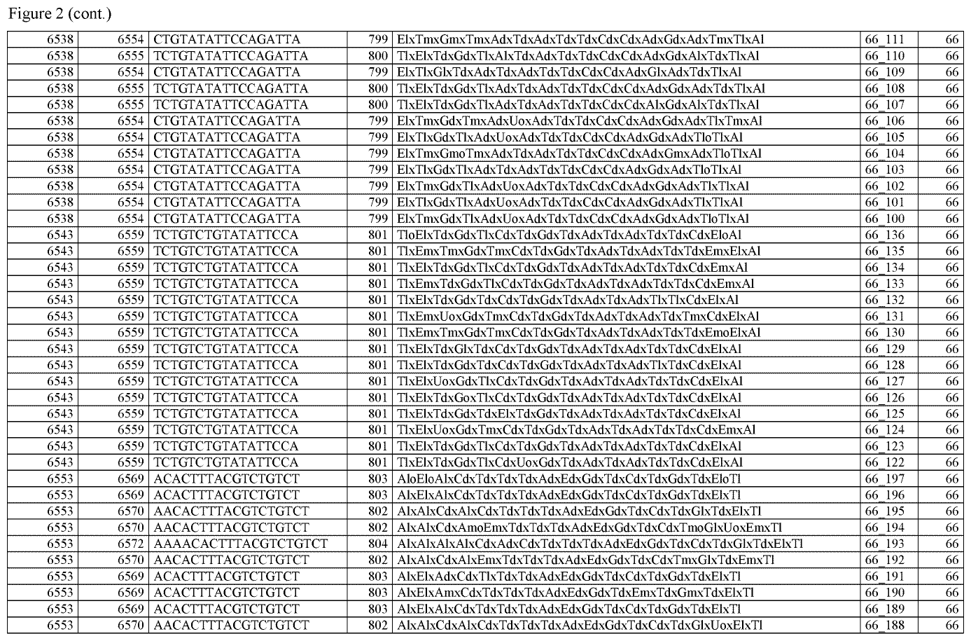

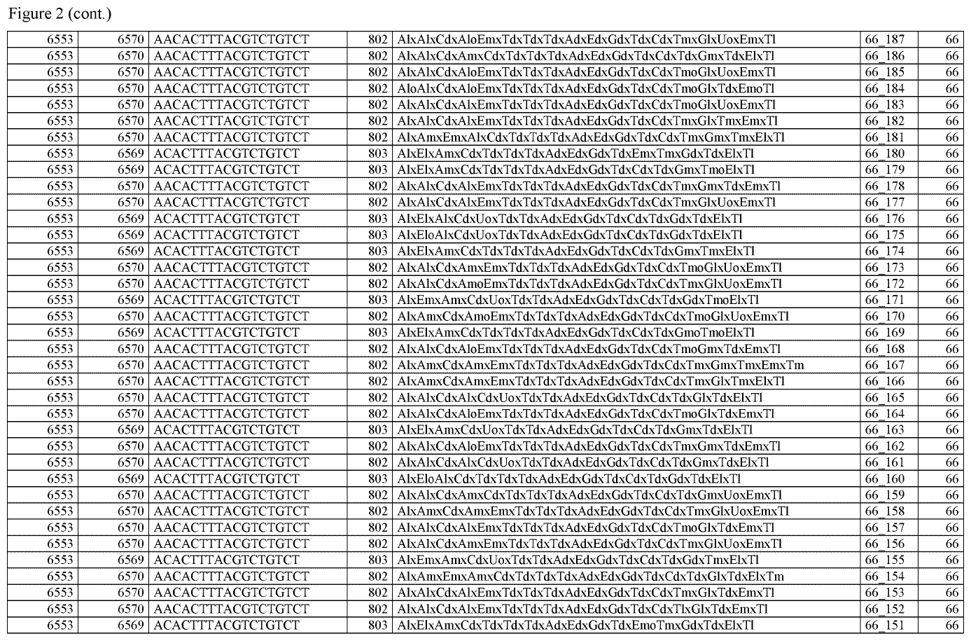

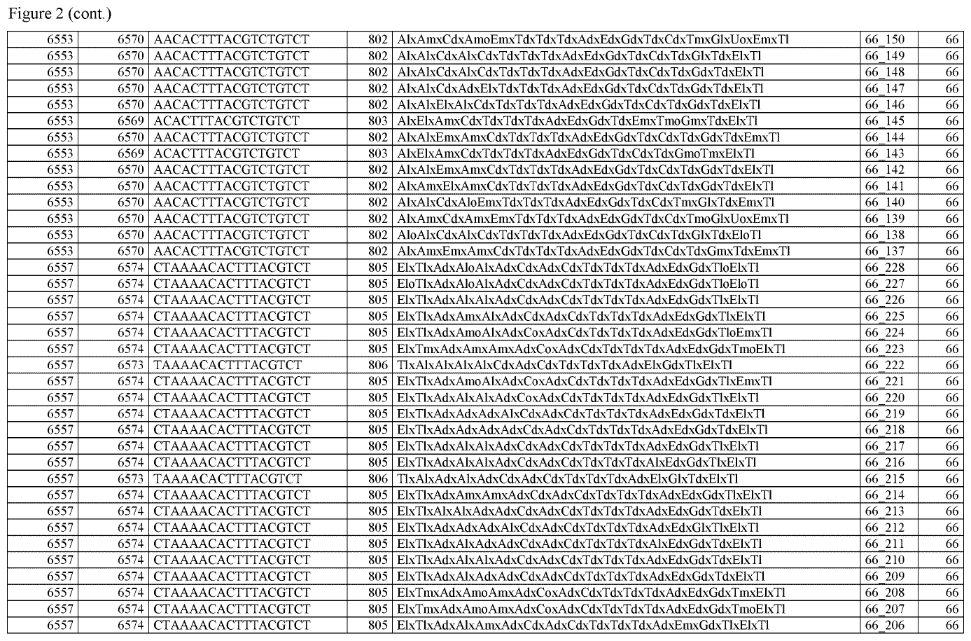

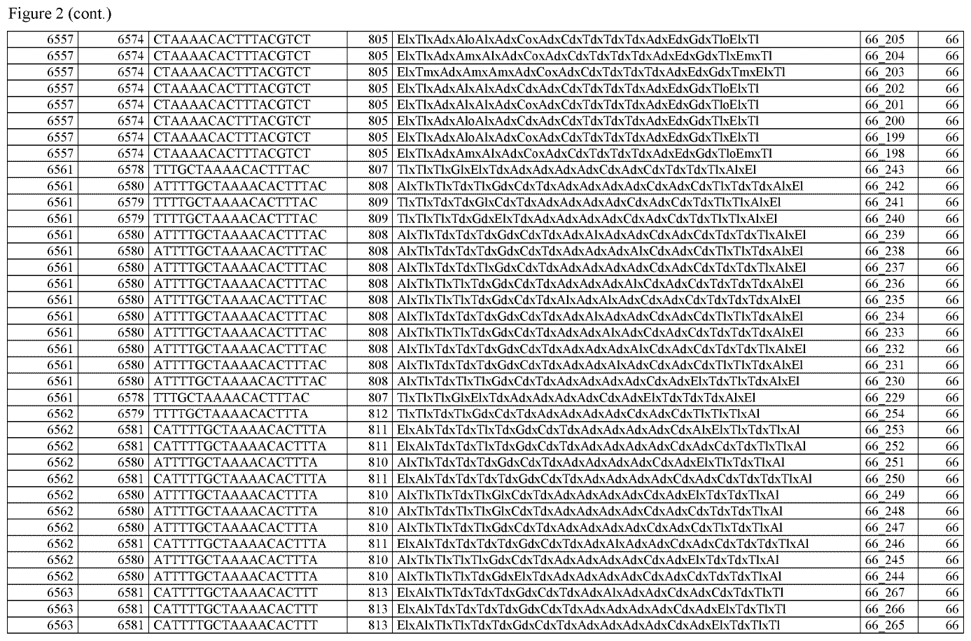

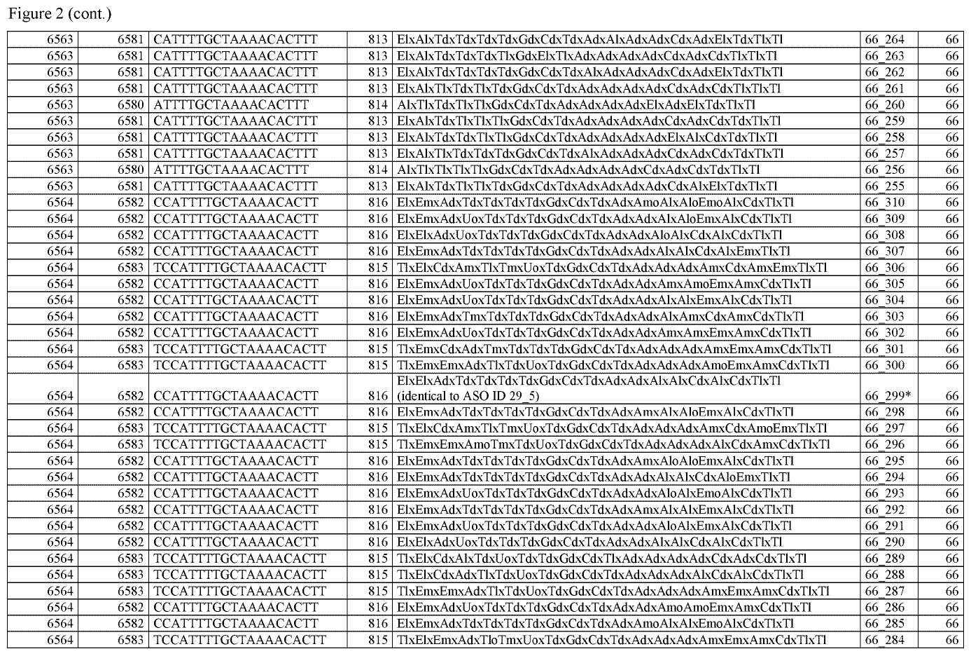

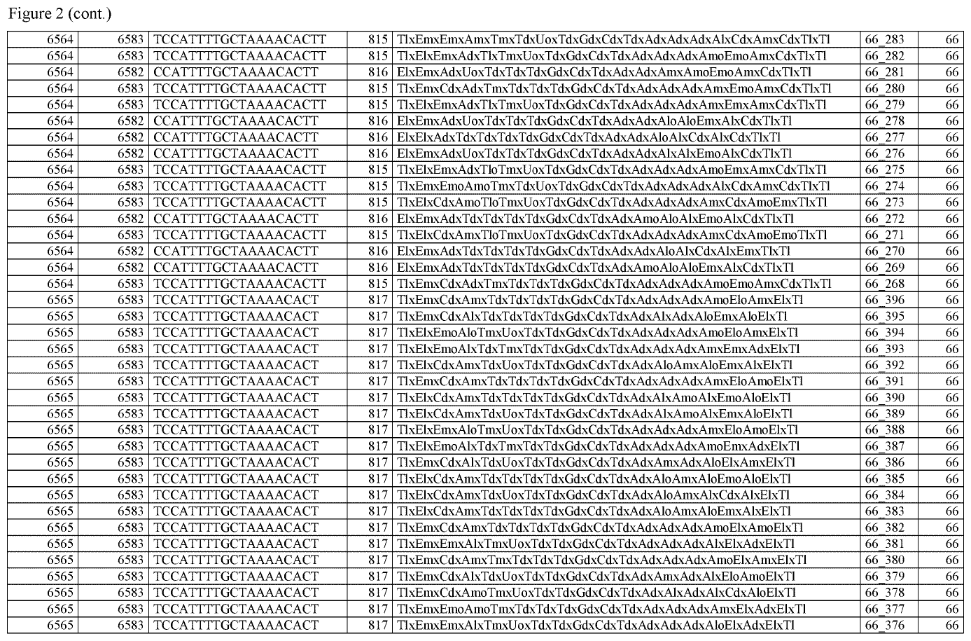

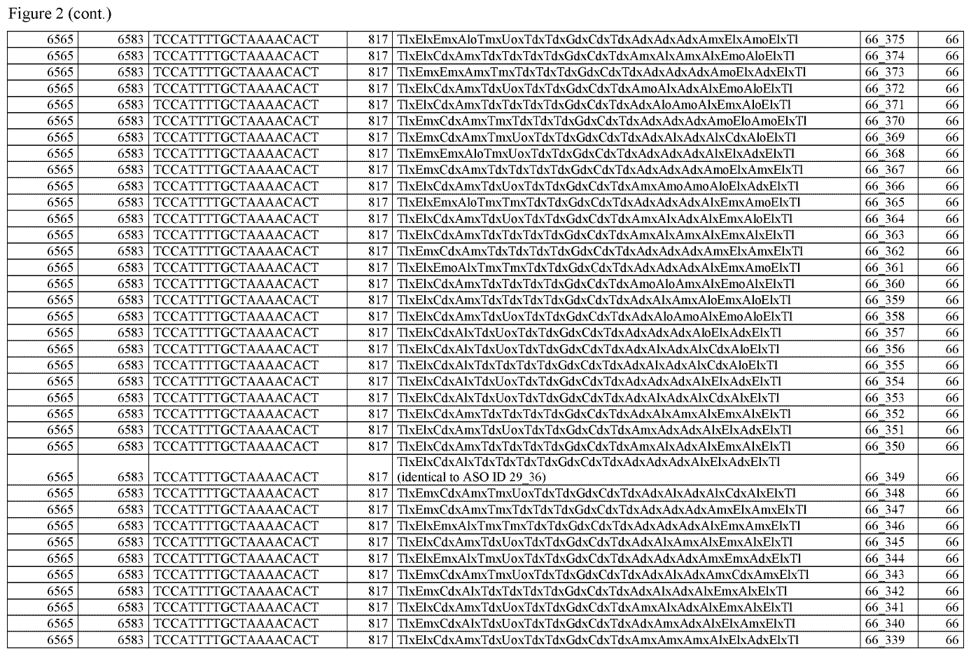

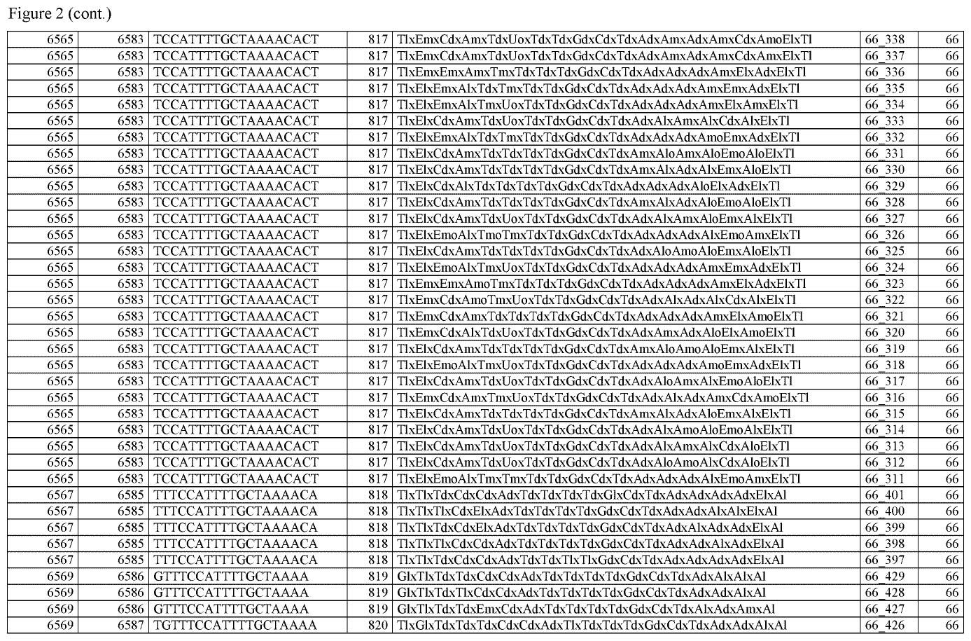

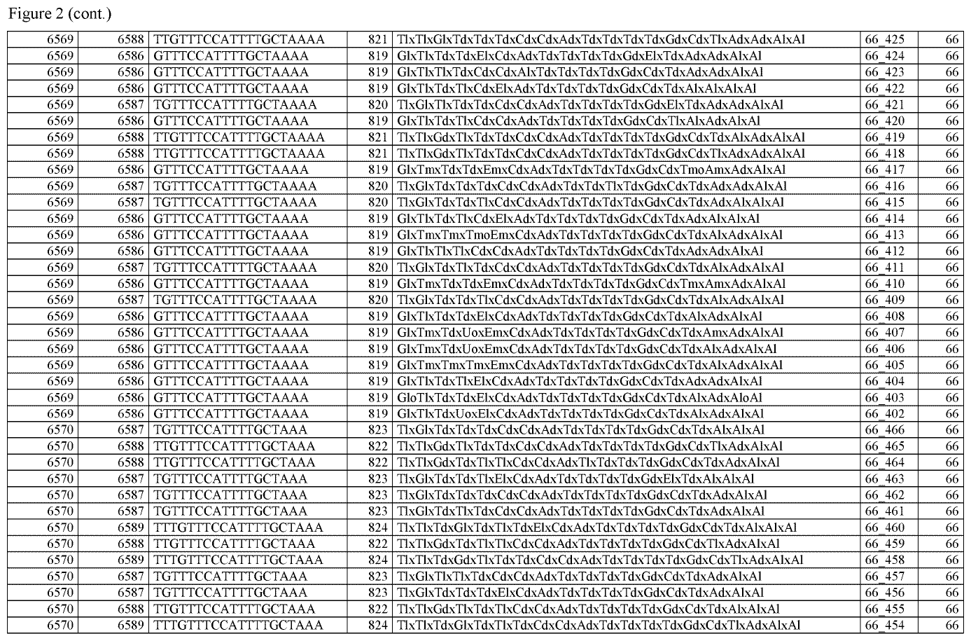

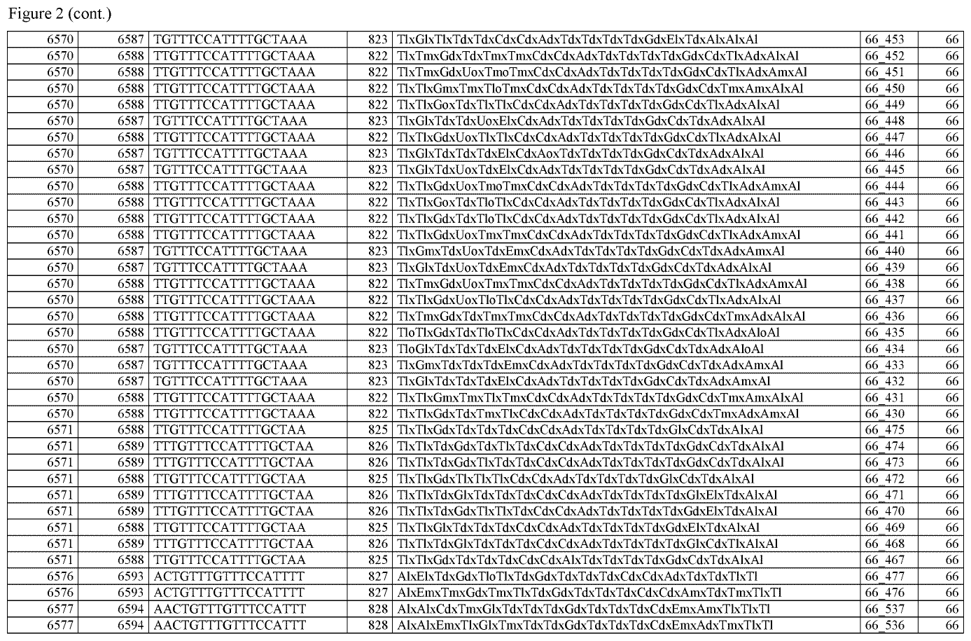

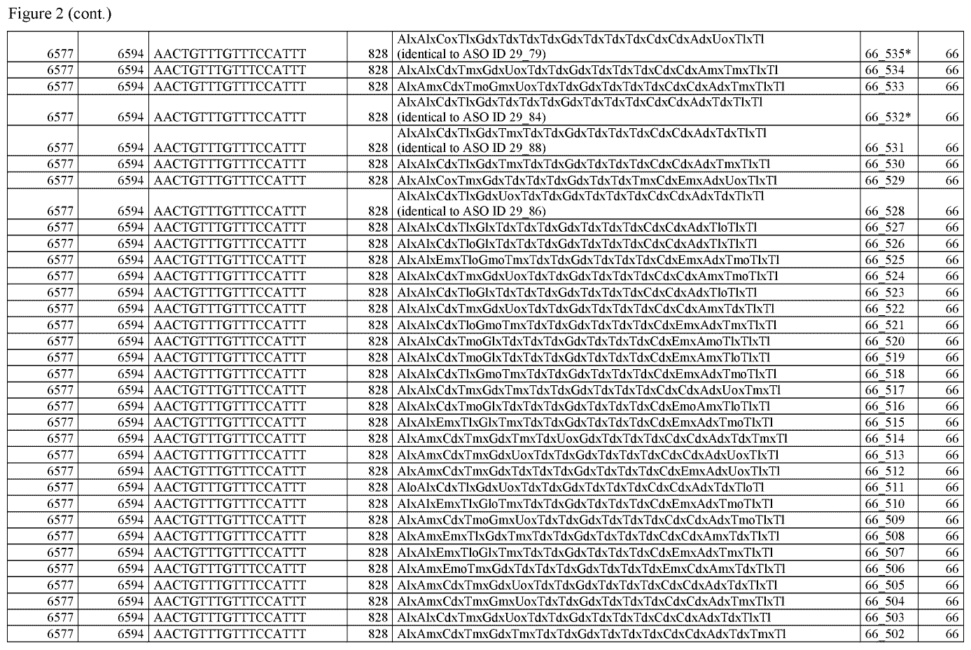

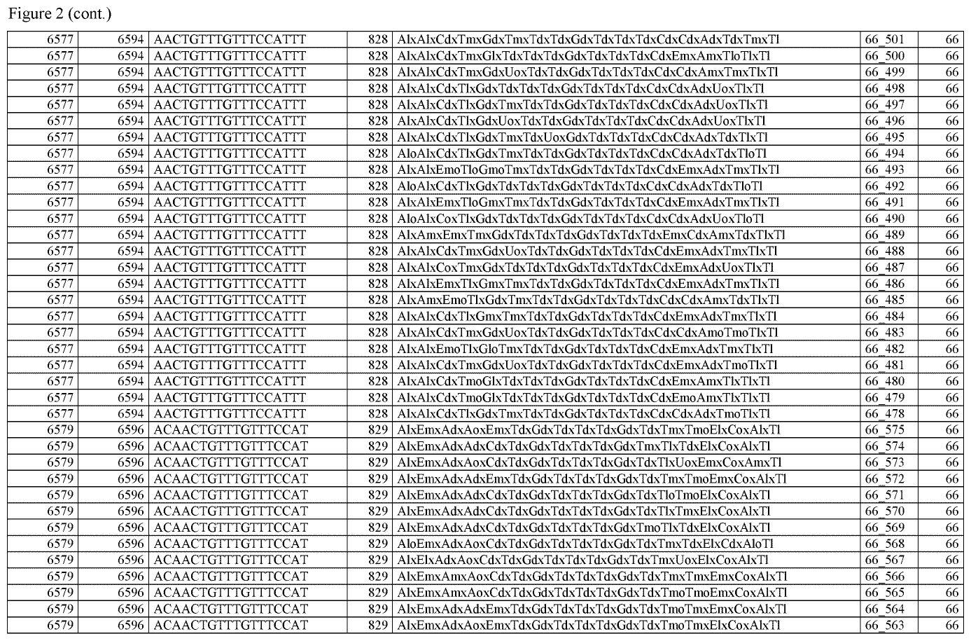

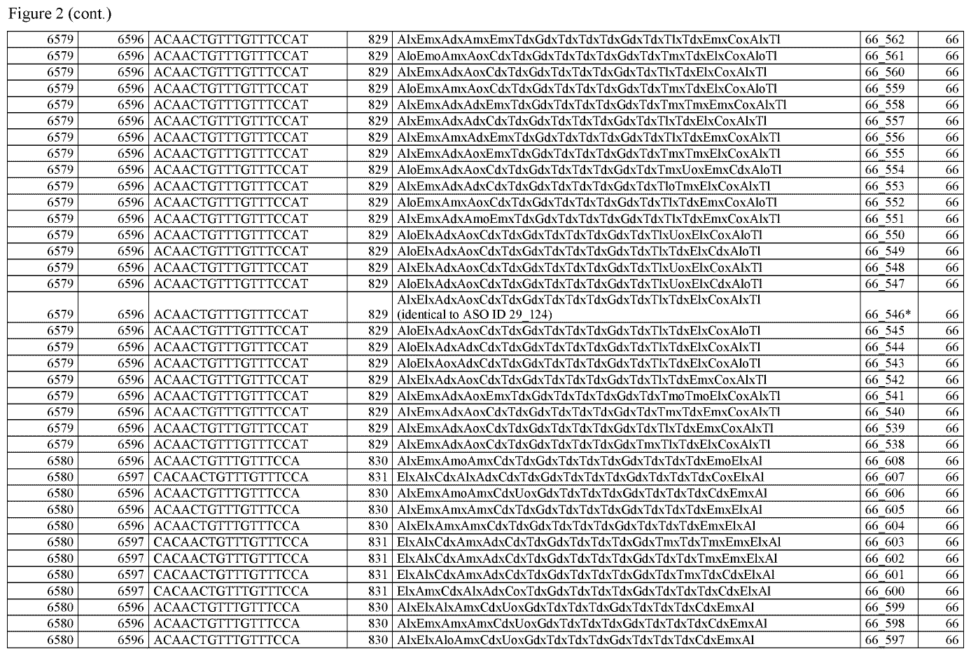

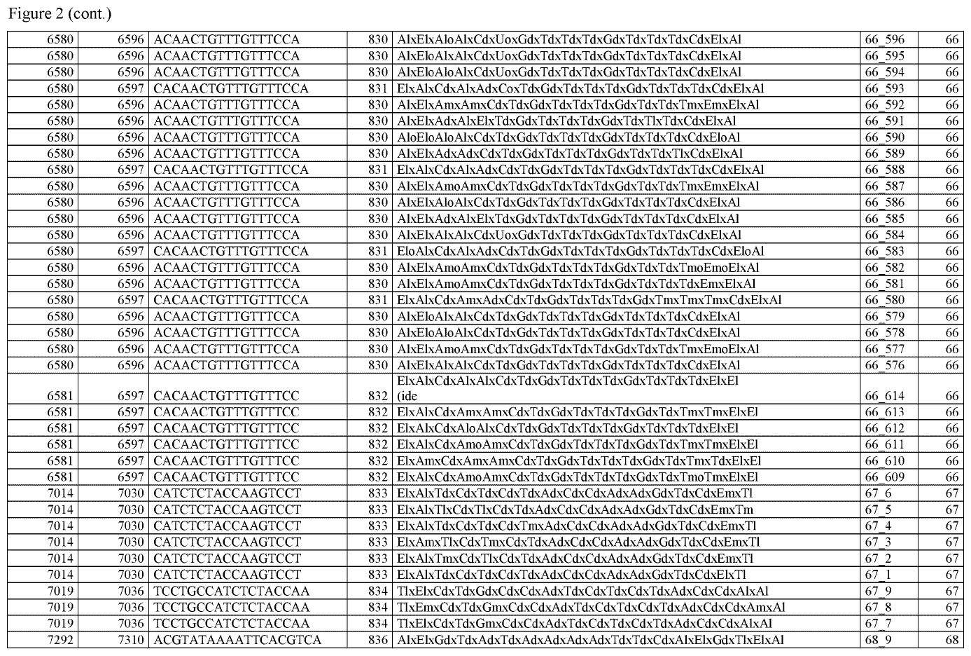

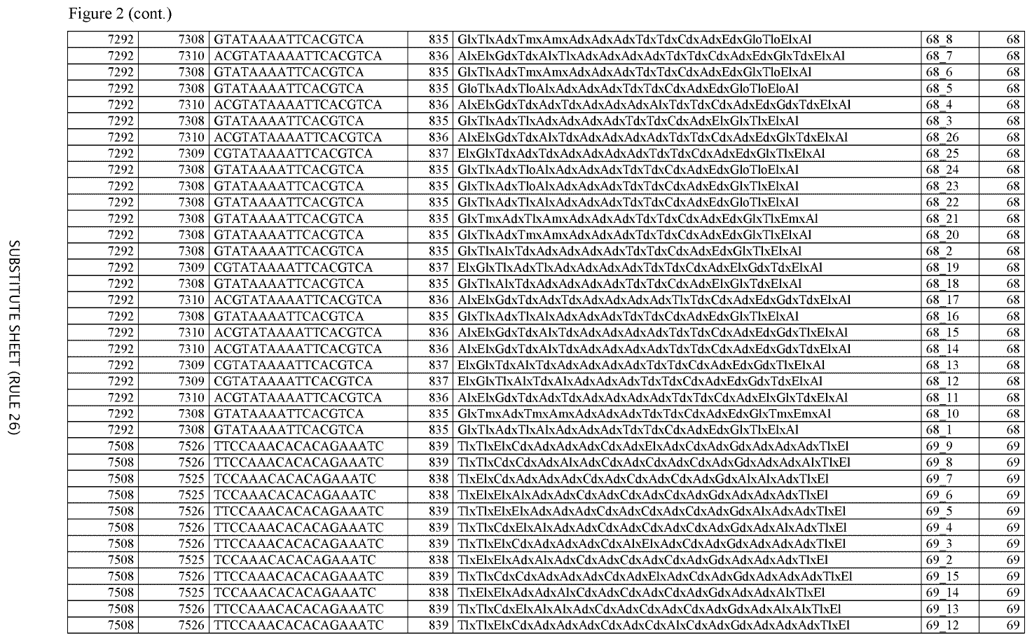

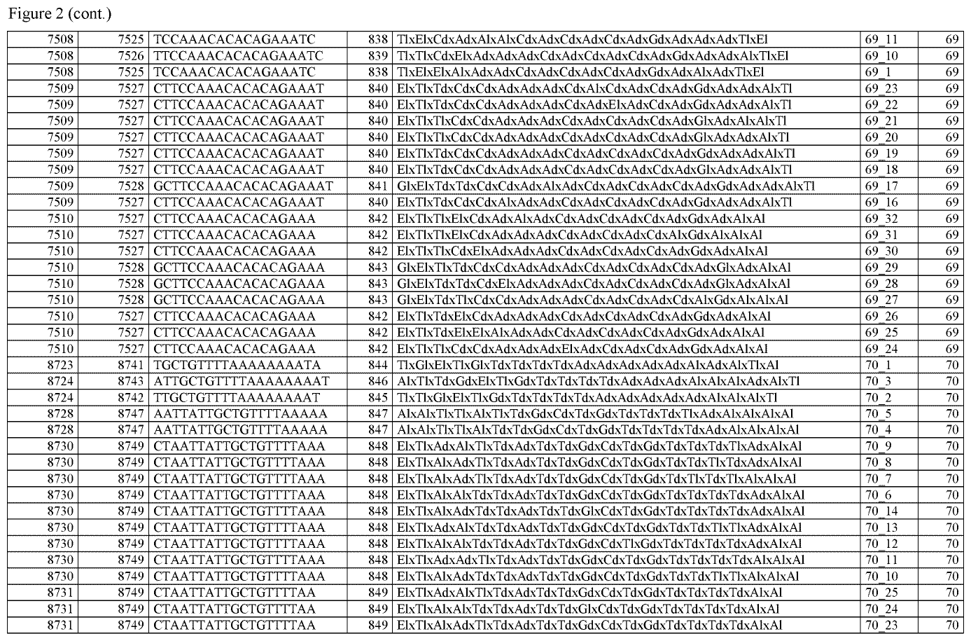

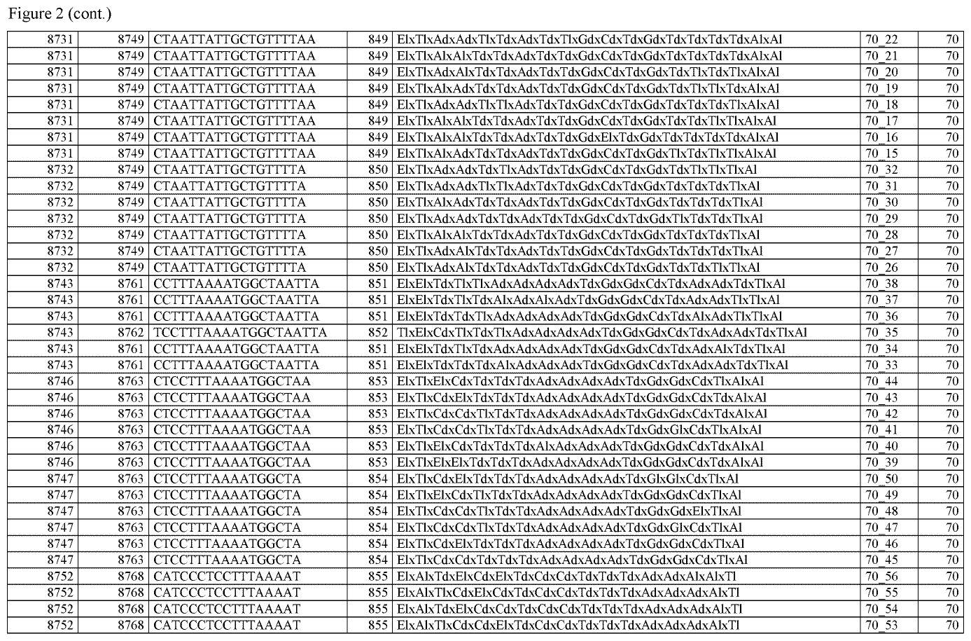

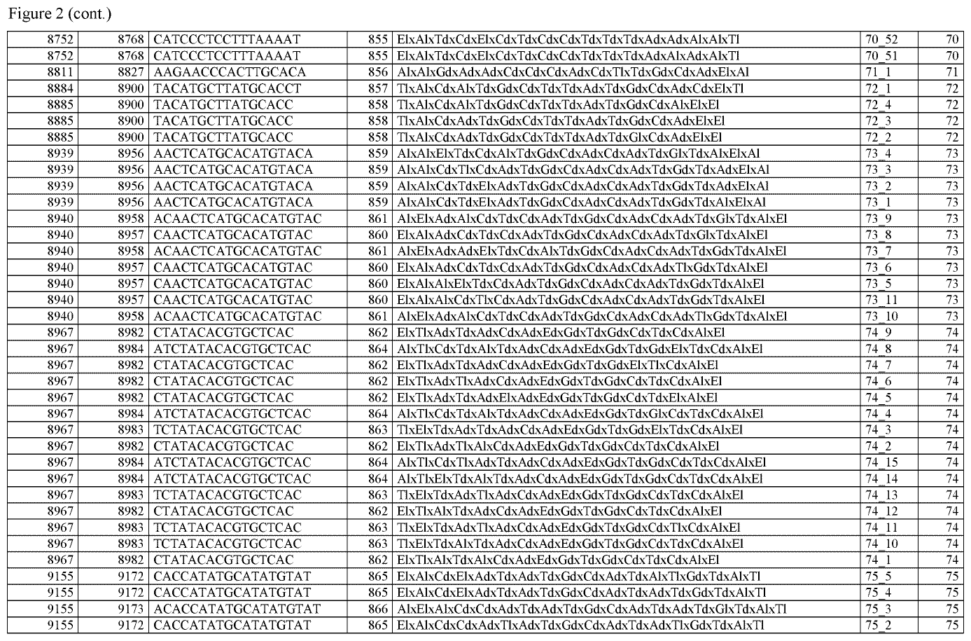

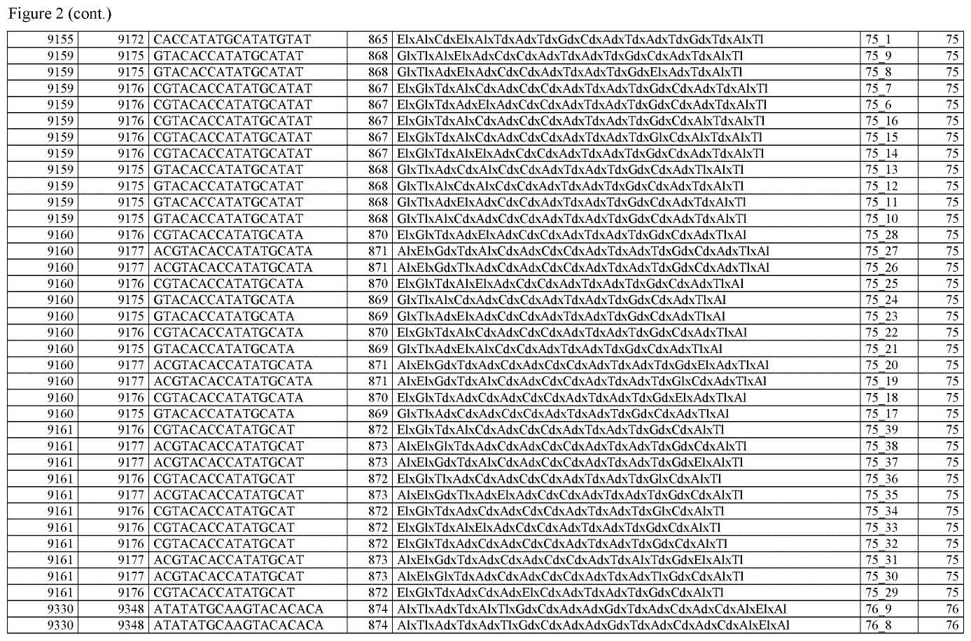

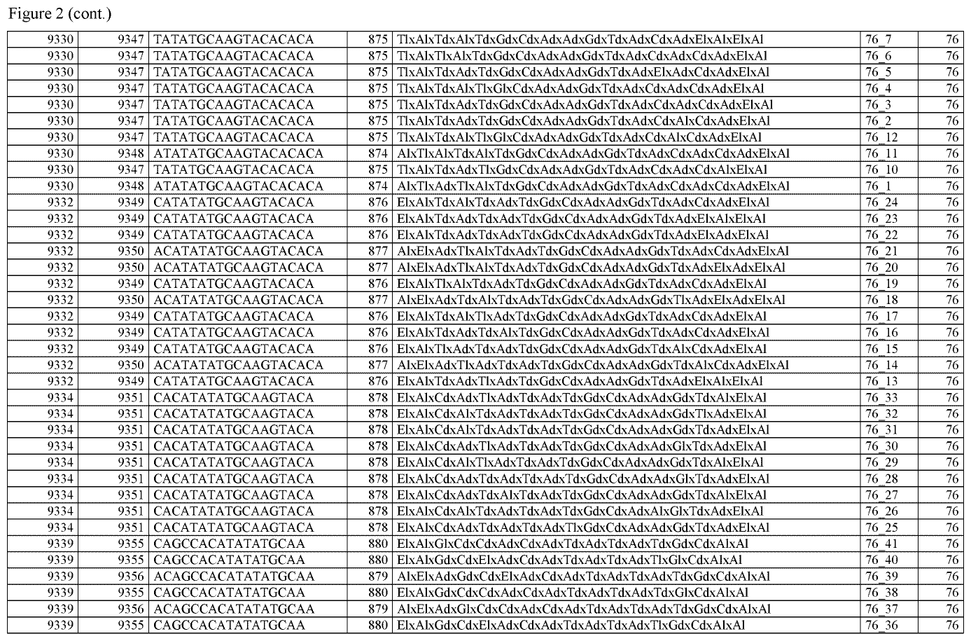

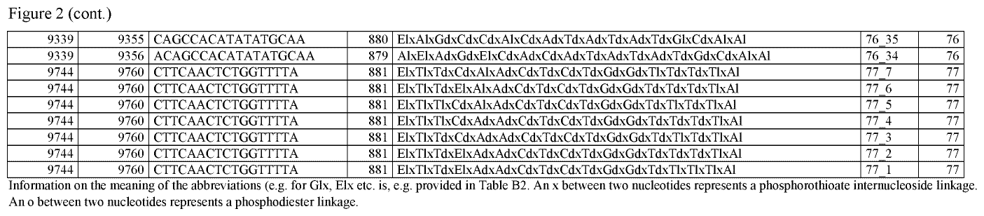

The produced compounds are shown in Table A1 and Table C1 (see FIGS. 1 and 2). All compounds shown in Table A1 were tested in A549 cells (see Example 1) and/or in HEK293 cells (see Example 2 and example 4). All compounds shown in Table C1 were tested in HEK293 cells (see Example 5). Some of the ASO compounds which led to an efficient downregulation of NAT8L were further tested for potency in HEK cells and iPSC derived neurons as well as for in vivo acute tolerability in mice and for pharmacodynamics and tolerability in non-human primates,

### Example 1: Testing In Vitro Efficacy of Antisense Oligonucleotides Targeting NAT8L in A549 Cells at Single Test Concentration

In order to interrogate the efficacy of the designed and synthesized ASOs a cell-based screening assay was developed using cells with an endogenous expression of NAT8L premRNA. In that optimization process various cell densities and compound incubation periods and concentrations were tested before reaching the assay conditions as described below.

The A549 cells were maintained and expanded as recommended by the supplier (ECACC, acquired from Merck, 86012804-1VL). The cells were grown to 70-80% confluency, the cells are then trypsinized and resuspended in growth media. Viable cells are counted using trypan blue and a Countess 3 automatic cell counter. The appropriate number of cells are diluted in complete growth media, mixed by gentle pipetting, added to reagent reservoirs, and distributed into 96-well plates using a multichannel pipette in a total volume of 195 μl/well. Sterile PBS is added to the moats of the 96-well culture plates (Nunc™ Edge™ 96-Well, Nunclon Delta-Treated, Flat-Bottom Microplates) to reduce evaporation and potential plate effect. After 24 h of incubation the NAT8L ASOs (Table 3, see columns “ASO ID”, the compounds are described in Table A1), are added directly to the growth media from a 20-Fold stock dilution in PBS reaching a final ASO concentration of 10 μM. Table 1 summarizes the most important parameters relating to the cellular work.

TABLE 1
Information on cell line A549

| | | | Seeding | Incubation | Incubation |
|---|---|---|---|---|---|
| | | Culturing | density | prior to ASO | time with |
| Cell Line | Provider | conditions | (cells/well) | addition | ASO |
| A549 | ATCC | 5% CO₂ and | 10,000 cells/ | 24 h | 48 h |
| | | 95% humidity | well in 195 | | |
| | | at 37° C. | μl of media: | | |
| | | | Ham's F12K | | |
| | | | with 10% | | |
| | | | FBS and 2 mM | | |
| | | | Glutamine | | |

In addition to the ASOs designed to target NAT8L each plate also included 7 PBS controls (5 μL) positive a positive control ASOs (N=2, pr. plate) targeting the ATXN3 gene, moreover 30 non-targeting gapmer ASOs were used as negative controls distributed across randomly across all screening plates to monitor possible false positives.

After 48 hours, cells were harvested by gently aspirating and RNA was extracted using the Macherey-Nagel NucleoSpin 96 RNA Kit, according to the manufactures instructions and eluted in 75 μl of water.

Before qPCR based expression analysis 10 μl of the RNA containing eluate is transferred to a new plate and diluted 10-fold in RNAse free water, then heat chocked at 90° C. for 40 s and placed on ice. The diluted and heat shocked RNA is used as input template for the qPCR, using qScript™ XLT One-Step RT-qPCR ToughMix® (cat #95134-500) from QuantaBio and qPCR assays from Integrated DNA technologies (IDT) listed in Table 2.

TABLE 2
assay used for screening NAT8L targeting ASOs.

| Gene of | | | | Manufacture/ | Flurophor |
|---|---|---|---|---|---|
| Interest | | | | assay ID | Endogene |
| (GOI) | manufacture/ | Probe | Endogen | Endogen | normalizer |
| assay | Assay ID - GOI | GOI | normalizer | normalizer | assay |
| NAT8L | Integrated DNA | HEX with | ATXN3 | Integrated DNA | FAM with |
| | technologies - (IDT)/ | ZEN/IBFQ | | technologies - (IDT)/ | ZEN/IBFQ |
| | Hs.PT.58.39061553 | double | | Hs.PT.58.39355049 | double |
| | | quencher | | | quencher |

The QPCR reaction was run in 384 wells using a QuantStudio 7 Flex (applied biosystems by Thermo Fisher Scientific. Quantities of NAT8L mRNA was calculated applying the ddCT method and using the median of all the PBS treated wells within the same plate as the ‘untreated control’.

The expression level of NAT8L following ASO treatment (Table 3) is thus shown as percent of the PBS-treated wells.

Altogether the screen identified a number of different ASOs with high level of KD (such as more than 70%) targeting various regions of the NAT8L premRNA. These sequences and their target sequences are of particular interest for the further optimization of our compounds.

TABLE 3
Expression level of NAT8L in relation
to PBS treated control cells (in %)

| | NAT8L PBS norm |
|---|---|
| ASO ID | (A549 High conc) |
| 1_1 | 41.8 |
| 2_1 | 134.5 |
| 2_2 | 74 |
| 2_3 | 61.2 |
| 2_4 | 86.5 |
| 2_5 | 54.7 |
| 2_6 | 17.1 |
| 2_8 | 32.1 |
| 2_9 | 29.2 |
| 2_10 | 78.9 |
| 2_12 | 13.1 |
| 2_13 | 25.6 |
| 2_14 | 41 |
| 2_21 | 24.6 |
| 2_22 | 48.1 |
| 2_23 | 47.4 |
| 2_24 | 52.8 |
| 2_25 | 36.9 |
| 2_26 | 28.1 |
| 2_32 | 59.3 |
| 2_33 | 53.4 |
| 2_34 | 22.5 |
| 2_35 | 8.4 |
| 2_53 | 17.6 |
| 2_54 | 26.5 |
| 2_57 | 45.3 |
| 2_58 | 50.9 |
| 2_60 | 47.4 |
| 2_61 | 61.5 |
| 2_62 | 62.5 |
| 2_73 | 25.1 |
| 2_74 | 15.8 |
| 2_75 | 69.7 |
| 2_84 | 96.7 |
| 2_85 | 54.7 |
| 2_86 | 88.3 |
| 2_87 | 82.6 |
| 2_88 | 62.1 |
| 2_89 | 78.4 |
| 2_91 | 71.3 |
| 2_92 | 90.3 |
| 2_93 | 97.1 |
| 2_95 | 125 |
| 2_96 | 82.6 |
| 2_97 | 96.5 |
| 2_98 | 77.9 |
| 2_99 | 99.5 |
| 2_100 | 64.4 |
| 3_1 | 58.7 |
| 3_2 | 44.3 |
| 3_3 | 46.3 |
| 3_4 | 37.2 |
| 3_5 | 80.3 |
| 4_1 | 83.8 |
| 4_3 | 83.2 |
| 4_4 | 28.7 |
| 4_8 | 64.4 |
| 4_13 | 77.7 |
| 4_14 | 38.7 |
| 4_15 | 56.1 |
| 4_18 | 77 |
| 4_19 | 59.5 |
| 4_20 | 71.7 |
| 4_33 | 103.4 |
| 4_34 | 77.2 |
| 4_35 | 91.2 |
| 4_38 | 78.6 |
| 4_39 | 64.5 |
| 4_40 | 84.7 |
| 5_1 | 60.4 |
| 5_2 | 69 |
| 6_2 | 29.4 |
| 6_3 | 62.5 |
| 6_5 | 56.7 |
| 6_6 | 81.3 |
| 6_7 | 40.3 |
| 6_8 | 11.5 |
| 6_25 | 53.4 |
| 6_26 | 69.5 |
| 6_63 | 91.3 |
| 6_64 | 30.8 |
| 6_65 | 63.4 |
| 6_66 | 66.1 |
| 6_67 | 65 |
| 7_1 | 69.8 |
| 8_1 | 81.5 |
| 9_1 | 87.5 |
| 10_1 | 88.9 |
| 11_1 | 24.7 |
| 11_2 | 52.6 |
| 11_3 | 59.9 |
| 11_14 | 54.4 |
| 11_15 | 60.8 |
| 11_16 | 91.7 |
| 11_36 | 93.9 |
| 11_37 | 79.2 |
| 11_38 | 79.9 |
| 11_39 | 66.1 |
| 11_41 | 70.5 |
| 12_1 | 126 |
| 12_2 | 32.7 |
| 12_3 | 42.3 |
| 12_4 | 73.1 |
| 12_5 | 77.4 |
| 12_6 | 43.2 |
| 12_7 | 114 |
| 12_8 | 84.6 |
| 12_9 | 70.2 |
| 12_10 | 125.8 |
| 12_11 | 105.2 |
| 12_12 | 98.5 |
| 12_13 | 82.8 |
| 12_14 | 103.5 |
| 12_15 | 76.3 |
| 13_1 | 94.2 |
| 14_1 | 83.2 |
| 15_1 | 77.4 |
| 15_2 | 44.3 |
| 15_3 | 75.2 |
| 15_4 | 46.2 |
| 15_5 | 117.1 |
| 16_1 | 73.9 |
| 16_2 | 68.6 |
| 17_1 | 41.6 |
| 18_1 | 86.4 |
| 18_2 | 81.3 |
| 18_3 | 83.8 |
| 18_4 | 110.5 |
| 18_5 | 105.6 |
| 18_6 | 92 |
| 18_7 | 100 |
| 18_8 | 47.9 |
| 18_9 | 74.6 |
| 18_10 | 76.4 |
| 18_11 | 36.8 |
| 19_1 | 78.4 |
| 20_1 | 58.6 |
| 21_1 | 101.8 |
| 22_1 | 111.3 |
| 22_2 | 48.3 |
| 22_3 | 58.1 |
| 23_1 | 82.5 |
| 23_2 | 109 |
| 23_3 | 74.4 |
| 24_1 | 9 |
| 25_1 | 75.4 |
| 25_2 | 33 |
| 25_3 | 38.4 |
| 25_4 | 73.1 |
| 25_5 | 73.7 |
| 25_6 | 25.1 |
| 25_8 | 61.6 |
| 25_9 | 38.3 |
| 25_10 | 27.8 |
| 25_12 | 30.2 |
| 25_13 | 65.5 |
| 25_14 | 62.9 |
| 25_15 | 64.4 |
| 25_16 | 71.9 |
| 25_17 | 75 |
| 25_18 | 66.8 |
| 25_19 | 59.9 |
| 25_20 | 52.7 |
| 25_22 | 77.4 |
| 25_23 | 54.2 |
| 25_24 | 56.6 |
| 25_28 | 24 |
| 25_42 | 56.2 |
| 25_43 | 85.8 |
| 25_48 | 33.8 |
| 25_49 | 40 |
| 25_50 | 46.4 |
| 25_53 | 96.3 |
| 25_54 | 57.4 |
| 25_55 | 72.6 |
| 25_57 | 37.6 |
| 25_58 | 73.8 |
| 25_59 | 62.6 |
| 25_64 | 39.1 |
| 25_65 | 46.3 |
| 25_66 | 24.8 |
| 25_67 | 89.9 |
| 25_68 | 47.2 |
| 25_69 | 48.9 |
| 25_71 | 25.9 |
| 25_72 | 40.4 |
| 25_73 | 56 |
| 25_87 | 85.1 |
| 25_88 | 79.8 |
| 25_89 | 48.8 |
| 25_93 | 77.6 |
| 25_94 | 47.9 |
| 25_95 | 111.1 |
| 25_96 | 52.1 |
| 25_97 | 78 |
| 25_98 | 59.7 |
| 25_108 | 63 |
| 25_109 | 43.6 |
| 25_110 | 39.1 |
| 25_111 | 22 |
| 25_121 | 41 |
| 25_122 | 68.3 |
| 25_123 | 32.3 |
| 25_124 | 40.6 |
| 25_125 | 31 |
| 25_126 | 40.7 |
| 25_128 | 51 |
| 25_129 | 32.3 |
| 25_130 | 28.5 |
| 25_132 | 49.6 |
| 25_133 | 42.7 |
| 25_134 | 66.2 |
| 25_136 | 29.6 |
| 25_137 | 69.7 |
| 25_138 | 51 |
| 25_139 | 59.5 |
| 25_141 | 99.7 |
| 25_142 | 77.8 |
| 25_143 | 65.1 |
| 25_146 | 30.1 |
| 25_147 | 39.8 |
| 25_148 | 74.6 |
| 26_2 | 73.6 |
| 26_3 | 63.5 |
| 26_4 | 57.7 |
| 26_5 | 27.8 |
| 26_21 | 42.1 |
| 26_22 | 53.9 |
| 26_37 | 60.7 |
| 26_38 | 50 |
| 26_39 | 73 |
| 26_40 | 56.1 |
| 26_41 | 64.8 |
| 26_42 | 59.9 |
| 26_43 | 55.4 |
| 26_44 | 68.2 |
| 26_45 | 47.8 |
| 26_46 | 82.2 |
| 27_1 | 61.2 |
| 27_2 | 107.4 |
| 27_3 | 65.4 |
| 27_4 | 59.9 |
| 27_5 | 53.2 |
| 27_6 | 101.2 |
| 27_7 | 28.5 |
| 27_8 | 41.5 |
| 27_9 | 106.3 |
| 27_15 | 75.1 |
| 27_16 | 60.6 |
| 27_17 | 57.3 |
| 27_20 | 85.8 |
| 27_21 | 66.3 |
| 27_22 | 72 |
| 27_23 | 81.8 |
| 27_24 | 41.4 |
| 27_25 | 99.5 |
| 27_26 | 47.3 |
| 27_27 | 79.4 |
| 27_28 | 77.2 |
| 27_30 | 82.6 |
| 27_31 | 67.9 |
| 27_32 | 60.9 |
| 27_36 | 81.1 |
| 27_37 | 68.6 |
| 27_38 | 59.3 |
| 27_39 | 33 |
| 27_40 | 109.8 |
| 27_41 | 72.2 |
| 27_42 | 67 |
| 27_43 | 61.2 |
| 27_44 | 41.7 |
| 27_46 | 45.9 |
| 27_47 | 47 |
| 27_48 | 53.8 |
| 27_49 | 46.7 |
| 27_50 | 58.6 |
| 27_51 | 42.1 |
| 27_52 | 70.4 |
| 27_53 | 55.3 |
| 27_54 | 94.2 |
| 27_55 | 66.1 |
| 27_56 | 68.8 |
| 27_57 | 73.2 |
| 27_58 | 110 |
| 27_59 | 42.2 |
| 27_61 | 42.4 |
| 27_62 | 52.4 |
| 27_63 | 49.3 |
| 27_80 | 40.9 |
| 27_81 | 83.1 |
| 27_82 | 78.7 |
| 27_84 | 44.7 |
| 27_85 | 65.4 |
| 27_86 | 86.5 |
| 28_1 | 8.6 |
| 29_2 | 12.5 |
| 29_3 | 44.1 |
| 29_4 | 16.5 |
| 29_6 | 8 |
| 29_7 | 10.9 |
| 29_8 | 25.6 |
| 29_9 | 18.5 |
| 29_23 | 8.9 |
| 29_24 | 6.8 |
| 29_26 | 40.3 |
| 29_27 | 15.2 |
| 29_28 | 20.7 |
| 29_30 | 14.7 |
| 29_31 | 34.1 |
| 29_32 | 12.3 |
| 29_34 | 8.7 |
| 29_35 | 21.6 |
| 29_36 | 7.2 |
| 29_38 | 18.9 |
| 29_39 | 50.7 |
| 29_40 | 25 |
| 29_42 | 16.9 |
| 29_43 | 31.2 |
| 29_44 | 23.5 |
| 29_46 | 19.8 |
| 29_47 | 20.5 |
| 29_48 | 18 |
| 29_50 | 25.7 |
| 29_51 | 39.4 |
| 29_52 | 14 |
| 29_54 | 12.8 |
| 29_55 | 47 |
| 29_56 | 21 |
| 29_58 | 22.8 |
| 29_59 | 53.8 |
| 29_60 | 42 |
| 29_62 | 19.5 |
| 29_63 | 30.8 |
| 29_64 | 32.4 |
| 29_66 | 25.6 |
| 29_67 | 46.7 |
| 29_68 | 75.6 |
| 29_69 | 25.6 |
| 29_71 | 15.5 |
| 29_72 | 14 |
| 29_74 | 30.4 |
| 29_75 | 26.3 |
| 29_76 | 46.8 |
| 29_77 | 64.3 |
| 29_87 | 14.3 |
| 29_88 | 31.9 |
| 29_89 | 19.7 |
| 29_91 | 26.7 |
| 29_92 | 60.7 |
| 29_93 | 44.1 |
| 29_94 | 40.7 |
| 29_95 | 26.1 |
| 29_96 | 38.7 |
| 29_97 | 17.6 |
| 29_99 | 40.2 |
| 29_100 | 11.8 |
| 29_103 | 26 |
| 29_104 | 59.8 |
| 29_105 | 31 |
| 29_106 | 24.9 |
| 29_107 | 51.5 |
| 29_112 | 10.4 |
| 29_113 | 32.6 |
| 29_114 | 29.7 |
| 29_126 | 32.3 |
| 29_127 | 31.1 |
| 29_128 | 90 |
| 29_129 | 23.6 |
| 29_130 | 5.5 |
| 29_132 | 38 |
| 29_133 | 9 |
| 29_135 | 8.1 |
| 29_136 | 14.1 |
| 29_137 | 35.8 |
| 29_147 | 45.2 |
| 29_148 | 51.8 |
| 29_149 | 48.1 |
| 29_151 | 65.2 |
| 29_152 | 53.1 |
| 29_153 | 26.5 |
| 30_1 | 89.7 |
| 30_2 | 48.6 |
| 30_3 | 78.8 |
| 30_4 | 94.6 |
| 31_1 | 63.4 |
| 31_2 | 78.7 |
| 31_3 | 99.7 |
| 31_4 | 66.6 |
| 32_1 | 52.5 |
| 32_2 | 80.8 |
| 33_1 | 97.9 |
| 33_2 | 68.5 |
| 34_1 | 53.2 |
| 34_2 | 63.1 |
| 34_3 | 87.5 |
| 34_4 | 80.6 |
| 34_5 | 61.9 |
| 34_6 | 49.4 |
| 34_7 | 52.5 |
| 34_8 | 56 |
| 34_9 | 59.8 |
| 34_10 | 97.4 |
| 34_11 | 93.8 |
| 34_12 | 34 |
| 34_13 | 81.4 |
| 34_14 | 43 |
| 34_15 | 69.1 |
| 34_16 | 71.2 |
| 34_17 | 58.8 |
| 34_18 | 72.8 |
| 34_19 | 67.8 |
| 34_20 | 79.6 |
| 34_21 | 82.7 |
| 34_22 | 65 |
| 34_23 | 81.7 |
| 34_24 | 34.6 |
| 34_25 | 44.9 |
| 34_26 | 96.1 |
| 34_27 | 66 |
| 34_28 | 53 |
| 34_29 | 38.2 |
| 34_30 | 45.3 |
| 34_31 | 99.3 |
| 34_32 | 38.8 |
| 34_33 | 60 |
| 34_34 | 72.2 |
| 34_35 | 71.6 |
| 34_36 | 31.4 |
| 34_37 | 50.9 |
| 34_38 | 8.4 |
| 34_43 | 60.9 |
| 34_44 | 23.8 |
| 35_1 | 39.8 |
| 35_2 | 52.8 |
| 35_3 | 56.1 |
| 35_4 | 45.6 |
| 35_5 | 54.2 |
| 35_6 | 64.6 |
| 35_7 | 72.3 |
| 35_8 | 104.1 |
| 36_1 | 65.1 |
| 37_7 | 51.2 |
| 37_8 | 19.2 |
| 37_9 | 21.7 |
| 37_10 | 43 |
| 37_11 | 29.8 |
| 37_12 | 43.3 |
| 37_13 | 12.1 |
| 37_16 | 56.8 |
| 37_17 | 27.7 |
| 37_18 | 76.5 |
| 37_20 | 54.6 |
| 37_21 | 61.9 |
| 37_22 | 32.2 |
| 37_24 | 64 |
| 37_25 | 99.3 |
| 37_26 | 90.5 |
| 37_28 | 66.7 |
| 37_29 | 68.3 |
| 37_30 | 67.2 |
| 37_69 | 93.8 |
| 37_70 | 71.1 |
| 37_71 | 72.1 |
| 37_72 | 73.1 |
| 37_73 | 105.2 |
| 37_74 | 51.3 |
| 37_75 | 68.9 |
| 37_76 | 55.6 |
| 37_77 | 79.9 |
| 37_79 | 65.3 |
| 37_80 | 77.9 |
| 37_81 | 63 |
| 38_1 | 19.1 |
| 38_19 | 24.3 |
| 38_20 | 26.3 |
| 38_29 | 67.5 |
| 38_30 | 93.5 |
| 38_31 | 82.2 |
| 38_32 | 68.4 |
| 38_33 | 52.1 |
| 38_34 | 104.4 |
| 38_35 | 85.4 |
| 38_37 | 64.3 |
| 38_38 | 55.6 |
| 38_39 | 73.3 |
| 38_40 | 122.3 |
| 38_41 | 102 |
| 38_42 | 92.1 |
| 38_44 | 60.9 |
| 38_45 | 60.1 |
| 38_46 | 71.3 |
| 38_48 | 51.1 |
| 38_49 | 47.9 |
| 38_50 | 72.1 |
| 38_51 | 47.5 |
| 38_52 | 56.2 |
| 38_53 | 101.5 |
| 38_55 | 45.2 |
| 38_56 | 45.6 |
| 38_57 | 73.7 |
| 38_58 | 84 |
| 38_59 | 57.6 |
| 38_60 | 45.8 |
| 38_62 | 40.2 |
| 38_63 | 44.2 |
| 38_64 | 44.6 |
| 39_1 | 51.3 |
| 39_2 | 75.5 |
| 39_3 | 62.7 |
| 39_4 | 74.8 |
| 39_5 | 99.4 |
| 39_6 | 101 |
| 39_7 | 100.6 |
| 39_8 | 96.1 |
| 39_9 | 76.1 |
| 39_10 | 54.6 |
| 39_11 | 61 |
| 39_12 | 76.8 |
| 39_13 | 73.6 |
| 39_14 | 59.4 |
| 39_15 | 103.4 |
| 39_16 | 81.4 |
| 39_17 | 48.3 |
| 39_18 | 68 |
| 39_19 | 63.1 |
| 39_20 | 60.8 |
| 39_21 | 58.4 |
| 39_22 | 107.5 |
| 39_23 | 125.1 |
| 39_24 | 106.1 |
| 39_25 | 71.2 |
| 39_26 | 111.9 |
| 39_27 | 75.9 |
| 40_1 | 50.6 |
| 40_2 | 78.5 |
| 40_3 | 56.2 |
| 40_4 | 82.7 |
| 40_5 | 55.5 |
| 40_6 | 81.1 |
| 40_7 | 76.7 |
| 40_8 | 99.6 |
| 40_9 | 74.6 |
| 40_10 | 83.3 |
| 40_11 | 45.9 |
| 40_12 | 99.5 |
| 41_1 | 99.4 |
| 41_2 | 70.4 |
| 41_3 | 79.6 |
| 41_4 | 93.2 |
| 41_5 | 50.8 |
| 41_6 | 66.6 |
| 41_7 | 71.9 |
| 41_8 | 55.8 |
| 42_1 | 62.6 |
| 43_1 | 63.8 |
| 43_2 | 82.7 |
| 43_3 | 68.2 |
| 43_4 | 77.1 |
| 44_1 | 103.8 |
| 44_2 | 106 |
| 44_3 | 52.5 |
| 44_4 | 105.5 |
| 44_5 | 80.3 |
| 44_6 | 101.6 |
| 44_7 | 88.5 |
| 44_8 | 87 |
| 44_9 | 97.8 |
| 44_10 | 93.4 |
| 44_11 | 112.6 |
| 45_1 | 91.1 |
| 45_2 | 74.8 |
| 45_3 | 72.1 |
| 46_1 | 89 |
| 47_1 | 88.7 |
| 47_2 | 97.9 |
| 48_1 | 88.4 |
| 48_2 | 122.4 |
| 48_3 | 82.1 |
| 48_4 | 73.3 |
| 48_5 | 104.8 |
| 48_6 | 96.6 |
| 48_7 | 58.6 |
| 48_8 | 98.8 |
| 48_9 | 89.5 |
| 49_1 | 60.4 |
| 49_2 | 74.2 |
| 49_3 | 91.8 |
| 49_4 | 67.5 |
| 49_5 | 58 |
| 49_6 | 74.1 |
| 49_16 | 56.5 |
| 49_17 | 35.3 |
| 49_18 | 68.6 |
| 49_20 | 79 |
| 49_21 | 52.8 |
| 49_22 | 88.1 |
| 49_40 | 67.7 |
| 49_41 | 100.6 |
| 49_42 | 103.7 |
| 49_44 | 83.7 |
| 49_45 | 70.5 |
| 49_46 | 85.3 |
| 49_48 | 120.4 |
| 49_49 | 103.8 |
| 49_50 | 70.3 |
| 49_52 | 54.1 |
| 49_53 | 68.6 |
| 49_54 | 76.7 |
| 49_56 | 72.5 |
| 49_57 | 90.4 |
| 49_58 | 68 |
| 49_60 | 108.6 |
| 49_61 | 109.9 |
| 49_62 | 76.6 |
| 49_64 | 77.7 |
| 49_65 | 82.4 |
| 49_66 | 90 |
| 49_70 | 56.9 |
| 49_71 | 65.4 |
| 49_72 | 64.9 |
| 49_73 | 47.9 |
| 49_74 | 67.1 |
| 49_75 | 44.4 |
| 49_76 | 25.4 |
| 49_80 | 76.7 |
| 49_81 | 78.3 |
| 49_85 | 86 |
| 49_86 | 48.7 |
| 49_87 | 57.8 |
| 49_88 | 61.9 |
| 49_89 | 80.3 |
| 49_90 | 58.9 |
| 49_91 | 84.2 |
| 49_92 | 64.5 |
| 49_93 | 105.1 |
| 49_94 | 77.8 |
| 49_95 | 79.1 |
| 49_96 | 77 |
| 50_1 | 101.4 |
| 50_2 | 71.3 |
| 50_3 | 80.3 |
| 50_4 | 46.7 |
| 51_1 | 27.3 |
| 51_33 | 44.5 |
| 52_1 | 53.5 |
| 52_2 | 61.7 |
| 52_3 | 63.7 |
| 52_4 | 34.8 |
| 52_7 | 39.2 |
| 52_8 | 40.3 |
| 52_9 | 66.8 |
| 52_10 | 16.1 |
| 52_24 | 41.5 |
| 52_25 | 60.9 |
| 52_38 | 44.1 |
| 52_39 | 82.7 |
| 52_40 | 29.3 |
| 52_42 | 72 |
| 52_43 | 45 |
| 52_44 | 115.3 |
| 52_46 | 86.4 |
| 52_47 | 21.9 |
| 52_48 | 43 |
| 52_49 | 66.3 |
| 52_50 | 104.6 |
| 52_51 | 89 |
| 52_53 | 127.9 |
| 52_54 | 23.6 |
| 52_55 | 65.9 |
| 52_57 | 47.3 |
| 52_58 | 64.2 |
| 52_59 | 35.8 |
| 52_61 | 38.4 |
| 52_62 | 45.4 |
| 52_63 | 53.6 |
| 52_65 | 77 |
| 52_66 | 39 |
| 52_67 | 52 |
| 52_77 | 73.8 |
| 52_78 | 49.2 |
| 52_79 | 48.9 |
| 52_80 | 48.7 |
| 52_81 | 35 |
| 52_82 | 39.4 |
| 52_83 | 23.4 |
| 52_84 | 33.7 |

### Example 2 Testing In Vitro Efficacy of Antisense Oligonucleotides Targeting NAT8L in HEK Cells at Single Test Concentration

Based on screening results from example 1, more ASOs were designed and synthesized. The majority of these were designed to target in or vicinity of NAT8L sequences that had been identified in example 1 as being target sequences where high efficacy ASOs could be generated. To better identify the most potent and efficacious compounds lower concentration of compounds were used (1 μM) than in the first screen (10 μM) in example 1. Moreover, a new cell-based screening assay was developed in HEK293 cells as described below.

The HEK293 cells were maintained and expanded as recommended by the supplier. The cells were grown to 70-80% confluency, the cells are then trypsinized and resuspended in growth media. Viable cells are counted using trypan blue and Vi-CELL automatic cell counter (Beckman Coulter). The appropriate number of cells are diluted in complete growth media, mixed by gentle pipetting, added to reagent reservoirs, and distributed into 96-well plates using a multichannel pipette in a total volume of 190 μl/well. Sterile PBS is added to the moats of the 96-well culture plates (Nunc™ Edge™ 96-Well, Nunclon Delta-Treated, Flat-Bottom Microplates) to reduce evaporation and potential plate effect. After 24 h of incubation the NAT8L ASOs (Table 6, see columns “ASO ID”), are added directly to the growth media from a 20-Fold stock dilution in PBS reaching a final ASO concentration of 1 μM. Table 4 summarizes the most important parameters relating to the cellular work.

TABLE 4
Information on HEK293 cells

| | | | Seeding | Incubation | Incubation |
|---|---|---|---|---|---|
| | | Culturing | density | prior to ASO | time with |
| Cell Line | Provider | conditions | (cells/well) | addition | ASO |
| HEK293 | ATCC | 5% CO₂ and | 8,000 cells/ | 24 h | 120 h |
| | | 95% humidity | well in 190 | | |
| | | at 37° C. | μl of media: | | |
| | | | DMEM + | | |
| | | | 10% FBS + | | |
| | | | Sodium | | |
| | | | pyruvate | | |

In addition to the ASOs designed to target NAT8L each plate also included 10 PBS controls (10 μL), 4 NAT8L positive control ASOs and two ASO controls targeting the ATXN3 gene. After 120 hours, cells were harvested by gently aspirating and RNA was extracted using the Macherey-Nagel NucleoSpin 96 RNA Kit, according to the manufactures instructions and eluted in 75 μl of water.

qPCR experiments were carried out as described in Example 1. Quantities of NAT8L mRNA was calculated applying the ddCT method and using the median of all the PBS treated wells within the same plate as the ‘untreated control’. The expression level of NAT8L following ASO treatment (Table 6) is thus shown as percent of the PBS-treated wells.

Altogether, the data shows that additional high efficacy compounds can be generated in previous identified target sites (Example 1) and in vicinity of these target sites.

TABLE 6
Expression level of NAT8L in relation
to PBS treated control cells (in %)

| | NAT8L PBS norm |
|---|---|
| ASO ID | (HEK293 Low conc |
| 2_7 | 74.8 |
| 2_11 | 47.2 |
| 2_15 | 40.6 |
| 2_16 | 43.8 |
| 2_17 | 23.6 |
| 2_18 | 50.1 |
| 2_19 | 47.2 |
| 2_20 | 55.1 |
| 2_27 | 31.7 |
| 2_28 | 43.7 |
| 2_29 | 73.5 |
| 2_30 | 46.2 |
| 2_31 | 69.1 |
| 2_35 | 29.6 |
| 2_36 | 31.3 |
| 2_37 | 37.9 |
| 2_38 | 33.7 |
| 2_39 | 43.6 |
| 2_40 | 37.9 |
| 2_41 | 50.6 |
| 2_42 | 52.6 |
| 2_43 | 46.7 |
| 2_44 | 46.2 |
| 2_45 | 54.1 |
| 2_46 | 56.4 |
| 2_47 | 59.5 |
| 2_48 | 83.4 |
| 2_49 | 55.1 |
| 2_50 | 26.1 |
| 2_51 | 36.3 |
| 2_52 | 33.9 |
| 2_55 | 45.4 |
| 2_56 | 37.5 |
| 2_59 | 53.2 |
| 2_63 | 74.5 |
| 2_64 | 26.8 |
| 2_65 | 42 |
| 2_66 | 40.6 |
| 2_67 | 44.2 |
| 2_68 | 69.6 |
| 2_69 | 63.1 |
| 2_70 | 66.1 |
| 2_71 | 52 |
| 2_72 | 70.6 |
| 2_76 | 42.7 |
| 2_77 | 75.9 |
| 2_78 | 33.3 |
| 2_79 | 33 |
| 2_80 | 43.2 |
| 2_81 | 59.7 |
| 2_82 | 60.4 |
| 2_83 | 97.3 |
| 2_90 | 86.3 |
| 2_94 | 83 |
| 4_2 | 72.3 |
| 4_4 | 45.1 |
| 4_5 | 41.6 |
| 4_6 | 52.2 |
| 4_7 | 70.6 |
| 4_9 | 68.6 |
| 4_10 | 54.7 |
| 4_11 | 84.8 |
| 4_12 | 70.2 |
| 4_16 | 66.4 |
| 4_17 | 82.1 |
| 4_21 | 87.5 |
| 4_22 | 84.7 |
| 4_23 | 96.2 |
| 4_24 | 71.9 |
| 4_25 | 70.9 |
| 4_26 | 75.9 |
| 4_27 | 90.4 |
| 4_28 | 80.5 |
| 4_29 | 45.7 |
| 4_30 | 72.4 |
| 4_31 | 54.5 |
| 4_32 | 91.4 |
| 4_36 | 78.2 |
| 4_37 | 96.7 |
| 4_41 | 45.7 |
| 4_42 | 52.9 |
| 4_43 | 49.5 |
| 4_44 | 40.2 |
| 4_45 | 61.1 |
| 4_46 | 52.2 |
| 4_47 | 68.4 |
| 4_48 | 82.4 |
| 4_49 | 55.5 |
| 6_1 | 68.1 |
| 6_4 | 92.7 |
| 6_8 | 78.4 |
| 6_9 | 84.3 |
| 6_10 | 72.3 |
| 6_11 | 74.4 |
| 6_12 | 85.3 |
| 6_13 | 84.4 |
| 6_14 | 79.6 |
| 6_15 | 75.6 |
| 6_16 | 79.1 |
| 6_17 | 76.8 |
| 6_18 | 53.6 |
| 6_19 | 91.3 |
| 6_20 | 89.7 |
| 6_21 | 88.5 |
| 6_22 | 92.3 |
| 6_23 | 95.4 |
| 6_24 | 91.2 |
| 6_27 | 72.7 |
| 6_28 | 86.3 |
| 6_29 | 82.1 |
| 6_30 | 97.8 |
| 6_31 | 72.2 |
| 6_32 | 81.2 |
| 6_33 | 91.5 |
| 6_34 | 70.5 |
| 6_35 | 90.1 |
| 6_36 | 81.3 |
| 6_37 | 77.5 |
| 6_38 | 75.4 |
| 6_39 | 69.8 |
| 6_40 | 85.6 |
| 6_41 | 77 |
| 6_42 | 76.5 |
| 6_43 | 75.5 |
| 6_44 | 72.5 |
| 6_45 | 52.7 |
| 6_46 | 70.3 |
| 6_47 | 85.6 |
| 6_48 | 69.5 |
| 6_49 | 61 |
| 6_50 | 79.3 |
| 6_51 | 78.6 |
| 6_52 | 73.7 |
| 6_53 | 80.9 |
| 6_54 | 74.9 |
| 6_55 | 82.1 |
| 6_56 | 81.8 |
| 6_57 | 67.6 |
| 6_58 | 80.1 |
| 6_59 | 74 |
| 6_60 | 73.1 |
| 6_61 | 72.6 |
| 6_62 | 71.6 |
| 6_68 | 71.3 |
| 6_69 | 83.7 |
| 11_1 | 67.8 |
| 11_4 | 69.4 |
| 11_5 | 75.6 |
| 11_6 | 98.1 |
| 11_7 | 83.8 |
| 11_8 | 78 |
| 11_9 | 90.5 |
| 11_10 | 78.3 |
| 11_11 | 87.5 |
| 11_12 | 68.1 |
| 11_13 | 62.2 |
| 11_17 | 57.5 |
| 11_18 | 68.4 |
| 11_19 | 57 |
| 11_20 | 63.8 |
| 11_21 | 78.6 |
| 11_22 | 57.5 |
| 11_23 | 84.2 |
| 11_24 | 78.8 |
| 11_25 | 71.8 |
| 11_26 | 63.2 |
| 11_27 | 91.5 |
| 11_28 | 80.4 |
| 11_29 | 73.9 |
| 11_30 | 70.3 |
| 11_31 | 60.5 |
| 11_32 | 83 |
| 11_33 | 87.1 |
| 11_34 | 76.9 |
| 11_35 | 59.5 |
| 11_40 | 71.5 |
| 25_7 | 52.7 |
| 25_11 | 83.3 |
| 25_21 | 58.8 |
| 25_25 | 62.9 |
| 25_26 | 73.2 |
| 25_27 | 64.2 |
| 25_28 | 71.6 |
| 25_29 | 52.8 |
| 25_30 | 73.6 |
| 25_31 | 46.6 |
| 25_32 | 59.8 |
| 25_33 | 71.8 |
| 25_34 | 67.9 |
| 25_35 | 70.6 |
| 25_36 | 61.3 |
| 25_37 | 59.2 |
| 25_38 | 70.7 |
| 25_39 | 69.3 |
| 25_40 | 61.4 |
| 25_41 | 66.1 |
| 25_44 | 51.3 |
| 25_45 | 56.8 |
| 25_46 | 44.4 |
| 25_47 | 49.9 |
| 25_51 | 69.1 |
| 25_52 | 83.3 |
| 25_56 | 70.3 |
| 25_60 | 43.2 |
| 25_61 | 72 |
| 25_62 | 67.4 |
| 25_63 | 66.8 |
| 25_70 | 58.3 |
| 25_74 | 51.4 |
| 25_75 | 52.1 |
| 25_76 | 53.1 |
| 25_77 | 48.7 |
| 25_78 | 60.8 |
| 25_79 | 61.2 |
| 25_80 | 66.4 |
| 25_81 | 53.2 |
| 25_82 | 50.4 |
| 25_83 | 58.9 |
| 25_84 | 48.5 |
| 25_85 | 58.3 |
| 25_86 | 74 |
| 25_90 | 75.7 |
| 25_91 | 62.6 |
| 25_92 | 65.2 |
| 25_99 | 52.2 |
| 25_100 | 42.9 |
| 25_101 | 53.6 |
| 25_102 | 45.3 |
| 25_103 | 37.6 |
| 25_104 | 41 |
| 25_105 | 41 |
| 25_106 | 42.1 |
| 25_107 | 46.6 |
| 25_111 | 31.2 |
| 25_112 | 53.6 |
| 25_113 | 29.3 |
| 25_114 | 40.6 |
| 25_115 | 37 |
| 25_116 | 38.2 |
| 25_117 | 31.1 |
| 25_118 | 57.4 |
| 25_119 | 51.8 |
| 25_120 | 44.1 |
| 25_127 | 71 |
| 25_131 | 63.2 |
| 25_135 | 43.4 |
| 25_140 | 60.7 |
| 25_144 | 57.5 |
| 25_145 | 84.9 |
| 25_149 | 62.7 |
| 26_1 | 54.2 |
| 26_5 | 71 |
| 26_6 | 67.9 |
| 26_7 | 63.3 |
| 26_8 | 49.2 |
| 26_9 | 57.7 |
| 26_10 | 59.5 |
| 26_11 | 62 |
| 26_12 | 69.3 |
| 26_13 | 58.2 |
| 26_14 | 73.4 |
| 26_15 | 77.7 |
| 26_16 | 54.5 |
| 26_17 | 56.9 |
| 26_18 | 55.7 |
| 26_19 | 58.6 |
| 26_20 | 69.8 |
| 26_23 | 59.8 |
| 26_24 | 52.1 |
| 26_25 | 67.1 |
| 26_26 | 37.1 |
| 26_27 | 57.1 |
| 26_28 | 64.4 |
| 26_29 | 68.2 |
| 26_30 | 59.4 |
| 26_31 | 77.4 |
| 26_32 | 47 |
| 26_33 | 57.8 |
| 26_34 | 66.7 |
| 26_35 | 56.2 |
| 26_36 | 72.6 |
| 27_7 | 45.4 |
| 27_10 | 46.3 |
| 27_11 | 49.5 |
| 27_12 | 53 |
| 27_13 | 45.4 |
| 27_14 | 69.7 |
| 27_18 | 67.6 |
| 27_19 | 48.6 |
| 27_29 | 64.8 |
| 27_33 | 73.1 |
| 27_34 | 64.9 |
| 27_35 | 90.8 |
| 27_45 | 63.4 |
| 27_60 | 64.7 |
| 27_64 | 56.5 |
| 27_65 | 88 |
| 27_66 | 65.3 |
| 27_67 | 53.8 |
| 27_68 | 82.1 |
| 27_69 | 81.7 |
| 27_70 | 76 |
| 27_71 | 68.7 |
| 27_72 | 61 |
| 27_73 | 89.9 |
| 27_74 | 75 |
| 27_75 | 60.3 |
| 27_76 | 70.3 |
| 27_77 | 53.7 |
| 27_78 | 80.1 |
| 27_79 | 47.4 |
| 27_83 | 65.2 |
| 29_1 | 57.8 |
| 29_5 | 28.7 |
| 29_9 | 35.1 |
| 29_10 | 12.2 |
| 29_11 | 38.9 |
| 29_12 | 43.2 |
| 29_13 | 38.3 |
| 29_14 | 39.3 |
| 29_15 | 42.7 |
| 29_16 | 40.4 |
| 29_17 | 30.5 |
| 29_18 | 33 |
| 29_19 | 47.2 |
| 29_20 | 33.6 |
| 29_21 | 55.5 |
| 29_22 | 50.8 |
| 29_25 | 39.2 |
| 29_29 | 35.8 |
| 29_33 | 40.5 |
| 29_37 | 51.5 |
| 29_41 | 36.6 |
| 29_45 | 47 |
| 29_49 | 34.5 |
| 29_53 | 47.2 |
| 29_57 | 44.1 |
| 29_61 | 59.1 |
| 29_65 | 32.1 |
| 29_69 | 37.3 |
| 29_70 | 17.4 |
| 29_73 | 49.6 |
| 29_78 | 22.9 |
| 29_79 | 17.4 |
| 29_80 | 74.8 |
| 29_81 | 45.6 |
| 29_82 | 43.3 |
| 29_83 | 27.2 |
| 29_84 | 25.4 |
| 29_85 | 23.4 |
| 29_86 | 21.6 |
| 29_90 | 38.2 |
| 29_97 | 34.5 |
| 29_98 | 28.1 |
| 29_101 | 47.4 |
| 29_102 | 28 |
| 29_108 | 33.5 |
| 29_109 | 31.1 |
| 29_110 | 45.7 |
| 29_111 | 50.7 |
| 29_115 | 39.7 |
| 29_116 | 32.2 |
| 29_117 | 43.5 |
| 29_118 | 28.8 |
| 29_119 | 33.7 |
| 29_120 | 44.2 |
| 29_121 | 24.1 |
| 29_122 | 38.6 |
| 29_123 | 24.4 |
| 29_124 | 24.6 |
| 29_125 | 24.3 |
| 29_130 | 13.4 |
| 29_131 | 48.5 |
| 29_134 | 45.6 |
| 29_138 | 24 |
| 29_139 | 42.7 |
| 29_140 | 38.3 |
| 29_141 | 53.9 |
| 29_142 | 42.5 |
| 29_143 | 63.7 |
| 29_144 | 55.3 |
| 29_145 | 63.6 |
| 29_146 | 37 |
| 29_150 | 49.4 |
| 29_154 | 26.3 |
| 34_38 | 32.8 |
| 34_39 | 21.6 |
| 34_40 | 75.1 |
| 34_41 | 31.5 |
| 34_42 | 91.5 |
| 34_45 | 37.8 |
| 34_46 | 24.9 |
| 34_47 | 28.6 |
| 34_48 | 62.6 |
| 34_49 | 68.1 |
| 34_50 | 74.8 |
| 34_51 | 80.6 |
| 34_52 | 70.9 |
| 34_53 | 58.7 |
| 34_54 | 52.4 |
| 34_55 | 58 |
| 34_56 | 65.2 |
| 34_57 | 62.3 |
| 34_58 | 53.6 |
| 34_59 | 57.3 |
| 34_60 | 66.1 |
| 34_61 | 59.3 |
| 34_62 | 60 |
| 34_63 | 83.6 |
| 34_64 | 70.3 |
| 34_65 | 74.9 |
| 34_66 | 74.8 |
| 34_67 | 59.3 |
| 34_68 | 91.6 |
| 34_69 | 49.8 |
| 34_70 | 35 |
| 34_71 | 66.7 |
| 34_72 | 64.2 |
| 34_73 | 66.1 |
| 34_74 | 61.7 |
| 34_75 | 70 |
| 34_76 | 78 |
| 34_77 | 65.1 |
| 34_78 | 79 |
| 34_79 | 82.5 |
| 34_80 | 67.6 |
| 34_81 | 63.5 |
| 34_82 | 68.1 |
| 34_83 | 61.3 |
| 34_84 | 48.7 |
| 34_85 | 75.6 |
| 34_86 | 36.3 |
| 34_87 | 58.4 |
| 34_88 | 58.1 |
| 34_89 | 52.2 |
| 34_90 | 53.1 |
| 37_1 | 39.7 |
| 37_2 | 56.8 |
| 37_3 | 50.9 |
| 37_4 | 51.7 |
| 37_5 | 68 |
| 37_6 | 49.4 |
| 37_13 | 38.6 |
| 37_14 | 46.9 |
| 37_15 | 28.4 |
| 37_19 | 69.1 |
| 37_23 | 79.8 |
| 37_27 | 85.7 |
| 37_31 | 79.7 |
| 37_32 | 74.5 |
| 37_33 | 61.4 |
| 37_34 | 61.1 |
| 37_35 | 59.2 |
| 37_36 | 63.7 |
| 37_37 | 67.3 |
| 37_38 | 70.1 |
| 37_39 | 69.7 |
| 37_40 | 65 |
| 37_41 | 77.9 |
| 37_42 | 75.9 |
| 37_43 | 57.1 |
| 37_44 | 66.3 |
| 37_45 | 54.1 |
| 37_46 | 61.7 |
| 37_47 | 56.5 |
| 37_48 | 59.1 |
| 37_49 | 60.5 |
| 37_50 | 64.7 |
| 37_51 | 63.2 |
| 37_52 | 62.1 |
| 37_53 | 63.7 |
| 37_54 | 66.5 |
| 37_55 | 54.6 |
| 37_56 | 60.3 |
| 37_57 | 64.9 |
| 37_58 | 69.8 |
| 37_59 | 63.9 |
| 37_60 | 67.7 |
| 37_61 | 73.8 |
| 37_62 | 74.5 |
| 37_63 | 78.2 |
| 37_64 | 70 |
| 37_65 | 79.2 |
| 37_66 | 75.3 |
| 37_67 | 69 |
| 37_68 | 59.9 |
| 37_78 | 62.9 |
| 38_1 | 44.2 |
| 38_2 | 46.8 |
| 38_3 | 64.5 |
| 38_4 | 65.1 |
| 38_5 | 27.3 |
| 38_6 | 45.5 |
| 38_7 | 32.8 |
| 38_8 | 62.9 |
| 38_9 | 35.8 |
| 38_10 | 24.6 |
| 38_11 | 29.6 |
| 38_12 | 51.6 |
| 38_13 | 44.4 |
| 38_14 | 56.3 |
| 38_15 | 36.7 |
| 38_16 | 46.1 |
| 38_17 | 36.1 |
| 38_18 | 56.7 |
| 38_21 | 40.7 |
| 38_22 | 36.1 |
| 38_23 | 33.3 |
| 38_24 | 33.6 |
| 38_25 | 31.3 |
| 38_26 | 31.5 |
| 38_27 | 32.6 |
| 38_28 | 54.6 |
| 38_36 | 60.7 |
| 38_43 | 83.3 |
| 38_47 | 68.5 |
| 38_54 | 65.9 |
| 38_61 | 60.7 |
| 49_7 | 76.5 |
| 49_8 | 93.8 |
| 49_9 | 98 |
| 49_10 | 83.6 |
| 49_11 | 88.4 |
| 49_12 | 67.4 |
| 49_13 | 73.3 |
| 49_14 | 83.3 |
| 49_15 | 69.8 |
| 49_19 | 93.1 |
| 49_23 | 85 |
| 49_24 | 87.6 |
| 49_25 | 90.1 |
| 49_26 | 85.9 |
| 49_27 | 89.3 |
| 49_28 | 73.3 |
| 49_29 | 84 |
| 49_30 | 81.1 |
| 49_31 | 83.2 |
| 49_32 | 97.6 |
| 49_33 | 99.6 |
| 49_34 | 72 |
| 49_35 | 80.6 |
| 49_36 | 84 |
| 49_37 | 100.7 |
| 49_38 | 95.3 |
| 49_39 | 101.8 |
| 49_43 | 72.6 |
| 49_47 | 86.4 |
| 49_51 | 94.7 |
| 49_55 | 80.6 |
| 49_59 | 83 |
| 49_63 | 81 |
| 49_67 | 64.5 |
| 49_68 | 77.9 |
| 49_69 | 54.1 |
| 49_76 | 82.1 |
| 49_77 | 70.6 |
| 49_78 | 65.2 |
| 49_79 | 82.7 |
| 49_82 | 63.8 |
| 49_83 | 62.9 |
| 49_84 | 63.5 |
| 51_1 | 35.7 |
| 51_2 | 31.9 |
| 51_3 | 45 |
| 51_4 | 32.6 |
| 51_5 | 44 |
| 51_6 | 31.3 |
| 51_7 | 36.8 |
| 51_8 | 40.1 |
| 51_9 | 44.4 |
| 51_10 | 36.1 |
| 51_11 | 45.6 |
| 51_12 | 38.9 |
| 51_13 | 43.5 |
| 51_14 | 42.3 |
| 51_15 | 60.5 |
| 51_16 | 57.1 |
| 51_17 | 77.1 |
| 51_18 | 40 |
| 51_19 | 43.4 |
| 51_20 | 43.2 |
| 51_21 | 43.6 |
| 51_22 | 53.5 |
| 51_23 | 53.8 |
| 51_24 | 76.5 |
| 51_25 | 49.1 |
| 51_26 | 46.2 |
| 51_27 | 50.7 |
| 51_28 | 32.2 |
| 51_29 | 38.6 |
| 51_30 | 36.2 |
| 51_31 | 49.6 |
| 51_32 | 51.6 |
| 51_34 | 94.1 |
| 51_35 | 73.6 |
| 52_5 | 43.7 |
| 52_6 | 59.7 |
| 52_10 | 40.8 |
| 52_11 | 42.7 |
| 52_12 | 47.3 |
| 49_45 | 70.5 |
| 49_46 | 85.3 |
| 49_48 | 120.4 |
| 49_49 | 103.8 |
| 49_50 | 70.3 |
| 49_52 | 54.1 |
| 49_53 | 68.6 |
| 49_54 | 76.7 |
| 49_56 | 72.5 |
| 49_57 | 90.4 |
| 49_58 | 68 |
| 49_60 | 108.6 |
| 49_61 | 109.9 |
| 49_62 | 76.6 |
| 49_64 | 77.7 |
| 49_65 | 82.4 |
| 49_66 | 90 |
| 49_70 | 56.9 |
| 49_71 | 65.4 |
| 49_72 | 64.9 |
| 49_73 | 47.9 |
| 49_74 | 67.1 |
| 49_75 | 44.4 |
| 49_76 | 25.4 |
| 49_80 | 76.7 |
| 49_81 | 78.3 |
| 49_85 | 86 |
| 49_86 | 48.7 |
| 49_87 | 57.8 |
| 49_88 | 61.9 |
| 49_89 | 80.3 |
| 49_90 | 58.9 |
| 49_91 | 84.2 |
| 49_92 | 64.5 |
| 49_93 | 105.1 |
| 49_94 | 77.8 |
| 49_95 | 79.1 |
| 49_96 | 77 |
| 50_1 | 101.4 |
| 50_2 | 71.3 |
| 50_3 | 80.3 |
| 50_4 | 46.7 |
| 51_1 | 27.3 |
| 51_33 | 44.5 |
| 52_1 | 53.5 |
| 52_2 | 61.7 |
| 52_3 | 63.7 |
| 52_4 | 34.8 |
| 52_7 | 39.2 |
| 52_8 | 40.3 |
| 52_9 | 66.8 |
| 52_10 | 16.1 |
| 52_24 | 41.5 |
| 52_25 | 60.9 |
| 52_38 | 44.1 |
| 52_39 | 82.7 |
| 52_40 | 29.3 |
| 52_42 | 72 |
| 52_43 | 45 |
| 52_44 | 115.3 |
| 52_46 | 86.4 |
| 52_47 | 21.9 |
| 52_48 | 43 |
| 52_49 | 66.3 |
| 52_50 | 104.6 |
| 52_51 | 89 |
| 52_53 | 127.9 |
| 52_54 | 23.6 |
| 52_55 | 65.9 |
| 52_57 | 47.3 |
| 52_58 | 64.2 |
| 52_59 | 35.8 |
| 52_61 | 38.4 |
| 52_62 | 45.4 |
| 52_63 | 53.6 |
| 52_65 | 77 |
| 52_66 | 39 |
| 52_67 | 52 |
| 52_77 | 73.8 |
| 52_78 | 49.2 |
| 52_79 | 48.9 |
| 52_80 | 48.7 |
| 52_81 | 35 |
| 52_82 | 39.4 |
| 52_83 | 23.4 |
| 52_84 | 33.7 |

### Example 3: Determination of IC50 Values of NAT8L Targeting ASOs in HEK Cells

To validate the hits identified in single concentration screens (Example 1 and 2) and to rank compounds on their potency a concentration response experiments were subsequently carried out for compounds showing high level of knock down.

Using the same method as describe in example 2 but incubating the cells with different concentrations of ASO (0.01; 0.0316; 1; 0.316; 1.0; 3.16; 10; 31.6 M) allowed the generation of concentration response curves and IC50 values.

Concentration response curves were generated using the GraphPad prism software version9 using the “log(inhibitor) vs. response—Variable slope (four parameters)” fit with bottom constrained to >0 and top=100. IC50 values are shown in Table 7.

TABLE 7
IC50 level of selected compounds

| ASO ID | IC50 value [M] |
|---|---|
| 2_17 | 3.113E−07 |
| 2_35 | 2.84E−07 |
| 2_50 | 2.526E−07 |
| 2_51 | 3.812E−07 |
| 2_52 | 2.729E−07 |
| 6_8 | 2.457E−07 |
| 25_113 | 2.789E−07 |
| 29_2 | 8.119E−07 |
| 29_5 | 2.756E−07 |
| 29_6 | 3.305E−07 |
| 29_7 | 5.679E−07 |
| 29_10 | 5.352E−07 |
| 29_23 | 4.00E−07 |
| 29_24 | 2.68E−07 |
| 29_32 | 3.398E−07 |
| 29_34 | 3.343E−07 |
| 29_36 | 2.553E−07 |
| 29_54 | 1.977E−07 |
| 29_70 | 3.13E−07 |
| 29_72 | 3.601E−07 |
| 29_78 | 1.221E−07 |
| 29_79 | 1.204E−07 |
| 29_84 | 9.804E−08 |
| 29_85 | 1.443E−07 |
| 29_86 | 1.818E−07 |
| 29_100 | 2.303E−07 |
| 29_112 | 2.781E−07 |
| 29_121 | 9.105E−08 |
| 29_123 | 1.26E−07 |
| 29_124 | 9.52E−08 |
| 29_125 | 1.635E−07 |
| 29_130 | 1.75E−07 |
| 29_133 | 3.702E−07 |
| 29_138 | 3.571E−07 |
| 29_154 | 2.059E−07 |
| 34_38 | 5.06E−07 |
| 34_39 | 1.537E−07 |
| 34_46 | 1.63E−07 |
| 34_47 | 1.323E−07 |
| 37_15 | 9.094E−08 |
| 38_5 | 1.256E−07 |
| 38_10 | 1.713E−07 |
| 38_11 | 1.232E−07 |
| 51_6 | 1.224E−07 |

In conclusion, the concentration response experiments validated the hits identified from single concentration screen in example 1 and 2 and showed that highly potent compounds have been identified.

### Example 4: Overview on Identified Target Regions within the NAT8L Pre-mRNA Sequence (SEQ ID NO: 1) which Allow for Efficiently Downregulating NAT8L

Target sequences that were found in the studies underlying the present invention are shown in Table B1.

TABLE B1
Target sequences

| Start in | End in | SEQ | | |
|---|---|---|---|---|
| SEQ ID | SEQ ID | ID | | Target |
| NO: 1 | NO 1 | NO | Target Sequence | ID |
| 179 | 194 | 30 | CGGGTGCATGCATTGT | 1 |
| 890 | 919 | 31 | CGGCGAGTGCCTAAATATAATCTGCGGGCG | 2 |
| 937 | 960 | 32 | CTCGGTGCTAATTTATGAGGGGAA | 3 |
| 1508 | 1551 | 33 | CAGGAAGCTTGGAAATAGGGAGAAGTAGAAAG | 4 |
| | | | CACCCGAGATGA | |
| 1755 | 1770 | 34 | CGCAAGGTGATCCGCG | 5 |
| 1800 | 1832 | 35 | ATGGCGGACATCGAGCAGTACTACATGAAGCC | 6 |
| | | | G | |
| 2034 | 2049 | 36 | CTGAGTGACCTGAGCT | 7 |
| 2314 | 2330 | 37 | CTTAGCTGAAGGGTTTT | 8 |
| 2346 | 2362 | 38 | GCCTTCTATAGCTGGAG | 9 |
| 3032 | 3046 | 39 | CGTCTTGCCTGGGTA | 10 |
| 3825 | 3844 | 40 | GGATTTATAGGAAAGCTCCT | 11 |
| 3912 | 3934 | 41 | GTGCTGGAGTTTGCCGTGATGGC | 12 |
| 3981 | 3996 | 42 | ATGGCCTGTTCAGGGT | 13 |
| 4143 | 4158 | 43 | GTGGGTCCTGGCTGTT | 14 |
| 4169 | 4192 | 44 | GTAACAGGTGTTGGGCTGGCTTTC | 15 |
| 4227 | 4243 | 45 | GCTTGTCCTGAATAGTG | 16 |
| 4502 | 4516 | 46 | GAGGACAACACGGTG | 17 |
| 4582 | 4614 | 47 | GAAGGTGCTGGAGTTCGCCGTGGTGCACAACT | 18 |
| | | | A | |
| 4638 | 4651 | 48 | CGGCCGTCAAGGTG | 19 |
| 4666 | 4679 | 49 | CTACGAGTCGCTGG | 20 |
| 4889 | 4908 | 50 | CTCAATTGGCGTTTGTGTTG | 21 |
| 4934 | 4949 | 51 | GTTGTCTGGCTGGTTC | 22 |
| 5056 | 5073 | 52 | AAGAGTGGCATCTTGGAC | 23 |
| 5656 | 5671 | 53 | CCTGTGAATCTTATCA | 24 |
| 5730 | 5805 | 54 | ATTTTCTAATAAGTTATTTAGACAGAATAGCAC | 25 |
| | | | TCTGCATGACTTTAATTCTTGGGACAAAACGGT | |
| | | | AGTTTGTACC | |
| 6392 | 6414 | 55 | GTTAGCAATTGAGGTGTGCAGAG | 26 |
| 6474 | 6497 | 56 | GGGATGGGAGTTTTGTAGACTGTA | 27 |
| 6542 | 6559 | 57 | CTGGAATATACAGACAGA | 28 |
| 6564 | 6605 | 58 | AAGTGTTTTAGCAAAATGGAAACAAACAGTTG | 29 |
| | | | TGCCTTTTTC | |
| 6608 | 6631 | 59 | CTTTTGGTTTGGTTTGGGTTTGGC | 30 |
| 6711 | 6726 | 60 | GTGTCATGTCGGGGTG | 31 |
| 6852 | 6874 | 61 | CTCCTTGATATTGATCCTAGCAG | 32 |
| 7042 | 7056 | 62 | GTGCGGGGTGGTTGT | 33 |
| 7284 | 7340 | 63 | ATGTGCACTGACGTGAATTTTATACGTTTGTAG | 34 |
| | | | AAACTCTGATGTAACTTCTTCTAC | |
| 7515 | 7534 | 64 | GTGTGTTTGGAAGCCTCTGT | 35 |
| 8572 | 8588 | 65 | CTGACACTTGGGGTTTG | 36 |
| 8703 | 8731 | 66 | GGACATACCATTGTTGGATGTATTTTTTT | 37 |
| 8734 | 8763 | 67 | AAACAGCAATAATTAGCCATTTTAAAGGAG | 38 |
| 8784 | 8844 | 68 | ATGTGCACGTGTTTGAGCGTGTGTGTGTGTGCA | 39 |
| | | | AGTGGGTTCTTGGATATGTGTAGTGTGA | |
| 8875 | 8914 | 69 | GCATGTTGTAGGTGCATAAGCATGTACACGCGT | 40 |
| | | | GAGCATG | |
| 8936 | 8956 | 70 | AGGTGTACATGTGCATGAGTT | 41 |
| 8973 | 8991 | 71 | ACGTGTATAGATGTACGTG | 42 |
| 9007 | 9027 | 72 | GCGTGTACATGTGTTTGTGTC | 43 |
| 9049 | 9087 | 73 | CGTGTGTATGAATGTGCGTGTGTATGCATGAGC | 44 |
| | | | ACGTAT | |
| 9100 | 9128 | 74 | CGCATGTTCTTGTGTACTTCTCTGAGGGG | 45 |
| 9158 | 9175 | 75 | CATATGCATATGGTGTAC | 46 |
| 9243 | 9266 | 76 | CATGCACATGTGTATGGATGCATA | 47 |
| 9292 | 9313 | 77 | ATGTGTGTTGCATGAGCATGCA | 48 |
| 9322 | 9360 | 78 | AGTGCCTGTGTGTGTACTTGCATATATGTGGCT | 49 |
| | | | GTGCAG | |
| 9603 | 9619 | 79 | GGGCAGATTGTTCTGAG | 50 |
| 9625 | 9641 | 80 | AGATGTGCCAAAACAGC | 51 |
| 9725 | 9762 | 81 | CAATGCTGCTGTTTTTCAATAAAACCAGAGTTG | 52 |
| | | | AAGGC | |

Targeting sequences as shown in Table B2 allowed for a very efficient down-regulation of the target gene.

TABLE B2
Target sequences

| Start | End | | | |
|---|---|---|---|---|
| in | in | | | |
| SEQ | SEQ | SEQ | | Tar- |
| ID | ID | ID | | get |
| NO: 1 | NO 1 | NO | Target Sequence | ID |
| 894 | 913 | 11 | GAGTGCCTAAATATAATCTG | 2 |
| 1519 | 1548 | 12 | GAAATAGGGAGAAGTAGAAAGCA | 4 |
| | | | CCCGAGA | |
| 1800 | 1828 | 13 | ATGGCGGACATCGAGCAGTACTAC | 6 |
| | | | ATGAA | |
| 3825 | 3842 | 14 | GGATTTATAGGAAAGCTC | 11 |
| 3912 | 3927 | 15 | GTGCTGGAGTTTGCCG | 12 |
| 5656 | 5671 | 16 | CCTGTGAATCTTATCA | 24 |
| 5730 | 5760 | 17 | ATTTTCTAATAAGTTATTTAGACAGA | 25 |
| | | | ATAGC | |
| 5776 | 5804 | 18 | AATTCTTGGGACAAAACGGTAGTT | 25 |
| | | | TGTAC | |
| 6392 | 6409 | 19 | GTTAGCAATTGAGGTGTG | 26 |
| 6476 | 6495 | 20 | GATGGGAGTTTTGTAGACTG | 27 |
| 6542 | 6559 | 21 | CTGGAATATACAGACAGA | 28 |
| 6564 | 6605 | 22 | AAGTGTTTTAGCAAAATGGAAACAAAC | 29 |
| | | | AGTTGTGCCTTTTTC | |
| 7284 | 7302 | 23 | ATGTGCACTGACGTGAATT | 34 |
| 7307 | 7340 | 24 | ACGTTTGTAGAAACTCTGATGTAAC | 34 |
| | | | TTCTTCTAC | |
| 8703 | 8723 | 25 | GGACATACCATTGTTGGATGT | 37 |
| 8734 | 8751 | 26 | AAACAGCAATAATTAGCC | 38 |
| 9335 | 9351 | 27 | GTACTTGCATATATGTG | 49 |
| 9625 | 9640 | 28 | AGATGTGCCAAAACAG | 51 |
| 9726 | 9762 | 29 | AATGCTGCTGTTTTTCAATAAAAC | 52 |
| | | | CAGAGTTGAAGGC | |

The best results were obtained when ASOs were used that are complementary to target sequences shown in Table B3.

TABLE B3
Target sequences

| Start | End | | | |
|---|---|---|---|---|
| in | in | | | |
| SEQ | SEQ | SEQ | | |
| ID | ID | ID | | Target |
| NO: 1 | NO 1 | NO | Target Sequence | ID |
| 895 | 912 | 4 | AGTGCCTAAATATAATCT | 2 |
| 5776 | 5793 | 5 | AATTCTTGGGACAAAACG | 25 |
| 6564 | 6599 | 6 | AAGTGTTTTAGCAAAATG | 29 |
| | | | GAAACAAACAGTTGTGCC | |
| 7324 | 7340 | 7 | GATGTAACTTCTTCTAC | 34 |
| 8703 | 8720 | 8 | GGACATACCATTGTTGGA | 37 |
| 8734 | 8750 | 9 | AAACAGCAATAATTAGC | 38 |
| 9625 | 9640 | 10 | AGATGTGCCAAAACAG | 51 |

### Example 5. Testing In Vitro Efficacy of Antisense Oligonucleotides Targeting NAT8 in HEK Cells

Further ASOs were designed and synthesized. The newly synthesized are shown in Table C1. The in vitro efficacy of antisense oligonucleotides was tested as described in Example 2. The results are shown in Table 8 which shows the expression level of NAT8L following ASO treatment as percent of the PBS-treated wells.

Altogether, the data shows that additional high efficacy compounds can be generated in previous identified target sites and in vicinity of these target sites. Further, high efficacy compounds were generated for new target sequences (e.g. target sequences with Targed ID 53, 56, 58, 59, 63, and 67, see Table D1 in Example 10.

TABLE 8
The table shows the expression level of NAT8L relative to untreated
control (UTC %). The header of each column refers to the cell
line (e.g. HEK293, A549), then the experiment number of each
independent experiment (e.g. 1, 2 and 3 and 4), and the concentration
of the ASO in (e.g. 1 μM and 5 μM).

| ASO ID | |
|---|---|
| | % UTC(HEK293_1_5 uM) |
| 54_4 | 95 |
| 54_5 | 92.9 |
| 54_6 | 113.1 |
| 54_7 | 87.9 |
| 54_8 | 119 |
| 54_9 | 100 |
| 54_10 | 127.9 |
| 55_1 | 58 |
| 55_2 | 77.5 |
| 55_3 | 108.3 |
| 55_4 | 116 |
| 55_5 | 109.4 |
| 55_6 | 86.3 |
| 55_7 | 88.2 |
| 55_8 | 88.6 |
| 60_1 | 96.5 |
| 60_2 | 97.7 |
| 60_3 | 93.7 |
| 60_4 | 92.2 |
| 61_1 | 78 |
| 61_2 | 63.5 |
| 61_3 | 57.2 |
| 61_4 | 74.1 |
| 61_5 | 88.5 |
| 61_6 | 66.5 |
| 61_7 | 53.9 |
| 61_8 | 87.7 |
| 61_9 | 88.7 |
| 61_10 | 63.9 |
| 61_11 | 78.9 |
| 61_12 | 78.8 |
| 61_13 | 77.5 |
| 61_14 | 68 |
| 61_15 | 68 |
| 61_16 | 70.4 |
| 61_17 | 91.5 |
| 61_18 | 72 |
| 61_19 | 73.6 |
| 61_20 | 72.3 |
| 61_21 | 64.8 |
| 61_22 | 64.1 |
| 61_23 | 88 |
| 61_24 | 86.9 |
| 61_25 | 64.3 |
| 61_26 | 98.4 |
| 61_27 | 87.9 |
| 61_28 | 93.2 |
| 61_29 | 91.5 |
| 61_30 | 98.4 |
| 61_31 | 75.6 |
| 61_32 | 100.2 |
| 61_33 | 73.1 |
| 61_34 | 101.5 |
| 61_35 | 66.2 |
| 61_36 | 90.4 |
| 61_37 | 103.1 |
| 61_38 | 57.5 |
| 61_39 | 59 |
| 61_40 | 71.7 |
| 61_41 | 63.4 |
| 61_42 | 84.1 |
| 61_43 | 102.1 |
| 61_44 | 69.1 |
| 61_45 | 82.4 |
| 61_46 | 104.2 |
| 61_47 | 74 |
| 61_48 | 91 |
| 61_49 | 50.2 |
| 61_50 | 75.8 |
| 61_51 | 108.5 |
| 61_52 | 82.1 |
| 61_53 | 72.6 |
| 61_54 | 66.7 |
| 61_55 | 67.7 |
| 61_56 | 81.3 |
| 61_57 | 75.5 |
| 61_58 | 99.6 |
| 61_59 | 65.1 |
| 61_60 | 74 |
| 61_61 | 86.4 |
| 61_62 | 67.6 |
| 61_63 | 92.6 |
| 61_64 | 78.7 |
| 61_65 | 93.3 |
| 61_66 | 101.4 |
| 61_67 | 93.5 |
| 61_68 | 102.8 |
| 61_69 | 79.7 |
| 61_70 | 81.3 |
| 61_71 | 75.9 |
| 61_72 | 79.2 |
| 61_73 | 94.1 |
| 61_74 | 68.1 |
| 61_75 | 67.5 |
| 61_76 | 79.8 |
| 61_77 | 67.2 |
| 61_78 | 109.3 |
| 61_79 | 97.6 |
| 61_80 | 70.5 |
| 61_81 | 83.4 |
| 61_82 | 71 |
| 61_83 | 76.1 |
| 61_84 | 106.1 |
| 61_85 | 68.8 |
| 61_86 | 92.2 |
| 62_1 | 86.2 |
| 62_2 | 49 |
| 62_3 | 87.1 |
| 62_4 | 69.4 |
| 62_5 | 101.9 |
| 62_6 | 60.1 |
| 62_7 | 56.2 |
| 62_8 | 82.8 |
| 62_9 | 78 |
| 62_10 | 83 |
| 62_11 | 74.6 |
| 62_12 | 75.2 |
| 62_13 | 83.8 |
| 62_14 | 92.7 |
| 62_15 | 83.7 |
| 62_16 | 84.3 |
| 62_17 | 67.9 |
| 62_18 | 83.3 |
| 62_19 | 94.5 |
| 62_20 | 66.4 |
| 62_21 | 63 |
| 62_22 | 79.2 |
| 62_23 | 91.2 |
| 62_24 | 85 |
| 62_25 | 102.6 |
| 62_26 | 61.4 |
| 64_1 | 68.2 |
| 64_2 | 85.8 |
| 64_3 | 82.8 |
| 64_4 | 75.4 |
| 64_5 | 86.5 |
| 64_6 | 73.2 |
| 64_7 | 68.7 |
| 64_8 | 80.4 |
| 64_9 | 75.5 |
| 64_10 | 95.6 |
| 64_11 | 64 |
| 64_12 | 68 |
| 64_13 | 82.8 |
| 64_14 | 57.7 |
| 64_15 | 67.4 |
| 64_16 | 86.7 |
| 64_17 | 81.6 |
| 64_18 | 79.1 |
| 65_1 | 70.4 |
| 65_2 | 75.8 |
| 65_3 | 55.3 |
| 65_4 | 67.2 |
| 65_5 | 72.2 |
| 65_6 | 73.3 |
| 65_7 | 81.7 |
| 65_8 | 88.2 |
| 65_9 | 87.8 |
| 65_10 | 87.4 |
| 65_11 | 92.9 |
| 65_12 | 90.9 |
| 65_13 | 82.6 |
| 65_14 | 102.1 |
| 65_15 | 76.2 |
| 65_16 | 87.8 |
| 65_17 | 64.2 |
| 65_18 | 81.5 |
| 65_19 | 93.2 |
| 65_20 | 70.2 |
| 65_21 | 79.3 |
| 65_22 | 100.5 |
| 65_23 | 90 |
| 65_24 | 85.6 |
| 65_25 | 74.6 |
| 65_26 | 72.7 |
| 65_27 | 71.5 |
| 65_28 | 56.6 |
| 65_29 | 81.4 |
| 65_30 | 68.4 |
| 65_31 | 72.3 |
| 65_32 | 90.3 |
| 65_33 | 80.1 |
| 65_34 | 78.3 |
| 65_35 | 57.3 |
| 65_36 | 92.9 |
| 65_37 | 64.1 |
| 65_38 | 99.6 |
| 65_39 | 105.2 |
| 65_40 | 77.4 |
| 65_41 | 102.2 |
| 65_42 | 95.3 |
| 65_43 | 83.9 |
| 65_44 | 63.8 |
| 65_45 | 72.7 |
| 65_46 | 74.5 |
| 65_47 | 64 |
| 65_48 | 85.1 |
| 65_49 | 59.8 |
| 65_50 | 64.9 |
| 65_51 | 69.8 |
| 65_52 | 60.8 |
| 65_53 | 77.5 |
| 65_54 | 72.3 |
| 65_55 | 85.2 |
| 65_56 | 74.5 |
| 65_57 | 80.1 |
| 65_58 | 60.7 |
| 65_59 | 97.8 |
| 65_60 | 71.9 |
| 65_61 | 80.7 |
| 65_62 | 51.6 |
| 65_63 | 94.4 |
| 65_64 | 85.8 |
| 65_65 | 46 |
| 65_66 | 44.6 |
| 65_67 | 32.2 |
| 65_68 | 61.9 |
| 65_69 | 102.8 |
| 65_70 | 66.3 |
| 65_71 | 60.3 |
| 65_72 | 73.2 |
| 65_73 | 70.1 |
| 65_74 | 73.1 |
| 65_75 | 88.5 |
| 65_76 | 58.5 |
| 65_77 | 64.7 |
| 65_78 | 61.1 |
| 65_79 | 83.9 |
| 65_80 | 49.6 |
| 65_81 | 79.9 |
| 65_82 | 52.7 |
| 65_83 | 78.3 |
| 65_84 | 31.3 |
| 65_85 | 33.3 |
| 65_86 | 48.7 |
| 66_14 | 78.8 |
| 66_15 | 87.8 |
| 66_16 | 80.5 |
| 66_17 | 96.6 |
| 66_18 | 102 |
| 66_19 | 74.3 |
| 66_20 | 88 |
| 66_21 | 70.5 |
| 66_22 | 58.5 |
| 66_23 | 52.6 |
| 66_24 | 44.3 |
| 66_25 | 45.2 |
| 66_26 | 44.1 |
| 66_27 | 28.6 |
| 66_28 | 68.8 |
| 66_29 | 71.7 |
| 66_30 | 65.5 |
| 66_31 | 73.9 |
| 66_32 | 80.3 |
| 66_33 | 64.7 |
| 66_34 | 68.7 |
| 66_35 | 83.3 |
| 66_36 | 18.2 |
| 66_42 | 50.2 |
| 66_43 | 60.4 |
| 66_44 | 71.4 |
| 66_45 | 71.9 |
| 66_46 | 28.2 |
| 66_47 | 26.1 |
| 66_48 | 22 |
| 66_49 | 70.4 |
| 66_50 | 38.3 |
| 66_51 | 54 |
| 66_52 | 71 |
| 66_53 | 49.6 |
| 66_65 | 61.1 |
| 66_66 | 41.5 |
| 66_67 | 46.4 |
| 66_68 | 54 |
| 66_69 | 54.1 |
| 66_70 | 41.2 |
| 66_71 | 62.2 |
| 66_72 | 82.1 |
| 66_73 | 53.7 |
| 66_74 | 74.1 |
| 66_75 | 55.7 |
| 66_76 | 66.8 |
| 66_77 | 70.6 |
| 66_78 | 40.2 |
| 66_79 | 57.5 |
| 66_80 | 47.8 |
| 66_81 | 41.4 |
| 66_82 | 71.8 |
| 66_83 | 59.3 |
| 66_84 | 41.3 |
| 66_85 | 84.8 |
| 66_86 | 65 |
| 66_87 | 59.5 |
| 66_88 | 76.5 |
| 66_89 | 61.2 |
| 66_90 | 84.8 |
| 66_91 | 42.9 |
| 66_92 | 30.6 |
| 66_93 | 58.8 |
| 66_94 | 83.6 |
| 66_95 | 70.8 |
| 66_96 | 67.4 |
| 66_97 | 31.1 |
| 66_107 | 80.9 |
| 66_108 | 72.5 |
| 66_109 | 57.1 |
| 66_110 | 54 |
| 66_114 | 71.7 |
| 66_115 | 33.4 |
| 66_117 | 26.1 |
| 66_118 | 61.9 |
| 66_119 | 40.1 |
| 66_120 | 19.9 |
| 66_121 | 62.9 |
| 66_123 | 18.2 |
| 66_125 | 38.4 |
| 66_128 | 44.8 |
| 66_129 | 43.5 |
| 66_132 | 12.5 |
| 66_189 | 26.4 |
| 66_191 | 38.5 |
| 66_193 | 30.2 |
| 66_209 | 52.2 |
| 66_210 | 36.3 |
| 66_211 | 49.7 |
| 66_212 | 37.9 |
| 66_213 | 34.3 |
| 66_215 | 51.8 |
| 66_216 | 45.9 |
| 66_217 | 20.9 |
| 66_218 | 85.1 |
| 66_219 | 62.6 |
| 66_222 | 83.3 |
| 66_229 | 84.7 |
| 66_230 | 49 |
| 66_231 | 63.5 |
| 66_232 | 100.4 |
| 66_233 | 31.7 |
| 66_234 | 53.5 |
| 66_235 | 71.4 |
| 66_236 | 56.4 |
| 66_237 | 73.4 |
| 66_238 | 54.4 |
| 66_239 | 60.4 |
| 66_240 | 68.1 |
| 66_241 | 45.9 |
| 66_242 | 55.1 |
| 66_243 | 49 |
| 66_244 | 66.6 |
| 66_245 | 45.3 |
| 66_246 | 102.4 |
| 66_247 | 37.8 |
| 66_248 | 39.6 |
| 66_249 | 58.9 |
| 66_250 | 93.5 |
| 66_251 | 96.1 |
| 66_252 | 84.2 |
| 66_253 | 47.9 |
| 66_254 | 59.3 |
| 66_255 | 45.2 |
| 66_256 | 46.4 |
| 66_257 | 61.7 |
| 66_258 | 62.5 |
| 66_259 | 41.5 |
| 66_260 | 68.6 |
| 66_261 | 54.6 |
| 66_262 | 70.2 |
| 66_263 | 38.1 |
| 66_264 | 71 |
| 66_265 | 65.6 |
| 66_266 | 96.6 |
| 66_267 | 86.1 |
| 66_268 | 85.2 |
| 66_269 | 58.9 |
| 66_270 | 43.1 |
| 66_271 | 65.3 |
| 66_272 | 56.7 |
| 66_273 | 74.6 |
| 66_274 | 73.4 |
| 66_275 | 67.9 |
| 66_276 | 71.4 |
| 66_277 | 32.5 |
| 66_278 | 77.7 |
| 66_279 | 75 |
| 66_280 | 81.6 |
| 66_281 | 89.8 |
| 66_282 | 73 |
| 66_283 | 85.3 |
| 66_284 | 78 |
| 66_285 | 64.4 |
| 66_286 | 87.5 |
| 66_287 | 85.3 |
| 66_288 | 65.4 |
| 66_289 | 47.7 |
| 66_290 | 56.7 |
| 66_291 | 64.5 |
| 66_292 | 56.3 |
| 66_293 | 67.2 |
| 66_294 | 40.6 |
| 66_295 | 60.6 |
| 66_296 | 71.7 |
| 66_297 | 57.8 |
| 66_298 | 67 |
| 66_300 | 79.8 |
| 66_301 | 81.5 |
| 66_302 | 86.5 |
| 66_303 | 93 |
| 66_304 | 62.5 |
| 66_305 | 81.2 |
| 66_306 | 66.1 |
| 66_307 | 47.4 |
| 66_308 | 36.9 |
| 66_309 | 78.8 |
| 66_310 | 53 |
| 66_311 | 80.9 |
| 66_312 | 67.8 |
| 66_313 | 70.2 |
| 66_314 | 72 |
| 66_315 | 55.8 |
| 66_316 | 79.2 |
| 66_317 | 72.7 |
| 66_318 | 65.5 |
| 66_319 | 55.5 |
| 66_320 | 70.3 |
| 66_321 | 60.9 |
| 66_322 | 75.6 |
| 66_323 | 48.9 |
| 66_324 | 67.4 |
| 66_325 | 45.4 |
| 66_326 | 76.2 |
| 66_327 | 70.5 |
| 66_328 | 63.6 |
| 66_329 | 31.1 |
| 66_330 | 50.4 |
| 66_331 | 69.9 |
| 66_332 | 59 |
| 66_333 | 89.1 |
| 66_334 | 42.7 |
| 66_335 | 59.2 |
| 66_336 | 56.7 |
| 66_337 | 98.3 |
| 66_338 | 86.3 |
| 66_339 | 83.9 |
| 66_340 | 62.4 |
| 66_341 | 81.8 |
| 66_342 | 62.5 |
| 66_343 | 92.4 |
| 66_344 | 38.6 |
| 66_345 | 69.8 |
| 66_346 | 59.2 |
| 66_347 | 59.5 |
| 66_348 | 71.1 |
| 66_350 | 54.2 |
| 66_351 | 82.7 |
| 66_352 | 42.4 |
| 66_353 | 70.3 |
| 66_354 | 55.7 |
| 66_355 | 32.6 |
| 66_356 | 73.5 |
| 66_357 | 47.8 |
| 66_358 | 86.8 |
| 66_359 | 44.8 |
| 66_360 | 44.3 |
| 66_361 | 69.5 |
| 66_362 | 58.1 |
| 66_363 | 45.7 |
| 66_364 | 78.3 |
| 66_365 | 75.5 |
| 66_366 | 82.3 |
| 66_367 | 62.9 |
| 66_368 | 56.2 |
| 66_369 | 87.8 |
| 66_370 | 72.7 |
| 66_371 | 51.6 |
| 66_372 | 92.6 |
| 66_373 | 70.7 |
| 66_374 | 66.8 |
| 66_375 | 65.2 |
| 66_376 | 66 |
| 66_377 | 67.7 |
| 66_378 | 83.2 |
| 66_379 | 74.6 |
| 66_380 | 79.2 |
| 66_381 | 52 |
| 66_382 | 63 |
| 66_383 | 56.9 |
| 66_384 | 77.1 |
| 66_385 | 60.4 |
| 66_386 | 79.6 |
| 66_387 | 56 |
| 66_388 | 45.1 |
| 66_389 | 66.6 |
| 66_390 | 50.6 |
| 66_391 | 66.2 |
| 66_392 | 74.1 |
| 66_393 | 62.5 |
| 66_394 | 65.4 |
| 66_395 | 64 |
| 66_396 | 70.3 |
| 66_397 | 60.7 |
| 66_398 | 47.2 |
| 66_399 | 54.1 |
| 66_400 | 42 |
| 66_401 | 88.8 |
| 66_404 | 44 |
| 66_408 | 28 |
| 66_409 | 42.9 |
| 66_411 | 38.4 |
| 66_412 | 29.5 |
| 66_414 | 40.6 |
| 66_415 | 45.8 |
| 66_416 | 80.9 |
| 66_418 | 60.8 |
| 66_419 | 38 |
| 66_420 | 70.6 |
| 66_421 | 87.8 |
| 66_422 | 42.3 |
| 66_423 | 65.7 |
| 66_424 | 88.9 |
| 66_425 | 53.4 |
| 66_426 | 91.3 |
| 66_428 | 38.8 |
| 66_429 | 78.7 |
| 66_453 | 60 |
| 66_454 | 70.9 |
| 66_455 | 29.2 |
| 66_456 | 23.5 |
| 66_457 | 39.6 |
| 66_458 | 55.5 |
| 66_459 | 32 |
| 66_460 | 57.8 |
| 66_461 | 46.3 |
| 66_462 | 92.9 |
| 66_463 | 53.4 |
| 66_464 | 38.2 |
| 66_465 | 39.8 |
| 66_466 | 87.5 |
| 66_467 | 36.8 |
| 66_468 | 53.2 |
| 66_469 | 53.5 |
| 66_470 | 70.9 |
| 66_471 | 61.5 |
| 66_472 | 89 |
| 66_473 | 64 |
| 66_474 | 69.8 |
| 66_475 | 38.8 |
| 66_476 | 91.3 |
| 66_477 | 22.2 |
| 66_478 | 21.6 |
| 66_479 | 50.7 |
| 66_480 | 38.5 |
| 66_481 | 72.2 |
| 66_482 | 83.9 |
| 66_484 | 72.7 |
| 66_486 | 89.1 |
| 66_487 | 53.8 |
| 66_488 | 76.2 |
| 66_489 | 74.7 |
| 66_491 | 80 |
| 66_493 | 87.1 |
| 66_499 | 65.8 |
| 66_500 | 49.3 |
| 66_506 | 75.7 |
| 66_507 | 81.8 |
| 66_510 | 92.6 |
| 66_515 | 79.6 |
| 66_516 | 42.5 |
| 66_518 | 67.9 |
| 66_519 | 41.1 |
| 66_520 | 38.3 |
| 66_521 | 75.3 |
| 66_522 | 78.7 |
| 66_523 | 31.3 |
| 66_525 | 87 |
| 66_526 | 32.5 |
| 66_527 | 25.8 |
| 66_529 | 53.8 |
| 66_530 | 23.1 |
| 66_536 | 87 |
| 66_537 | 33.3 |
| 66_538 | 62.2 |
| 66_541 | 20.8 |
| 66_551 | 56 |
| 66_553 | 36.2 |
| 66_555 | 34 |
| 66_556 | 65.7 |
| 66_557 | 29.9 |
| 66_558 | 61.8 |
| 66_560 | 29.7 |
| 66_562 | 48.6 |
| 66_563 | 31.6 |
| 66_564 | 41.5 |
| 66_565 | 49.3 |
| 66_566 | 59.5 |
| 66_569 | 51.5 |
| 66_570 | 29.7 |
| 66_571 | 38.8 |
| 66_572 | 51.6 |
| 66_574 | 37 |
| 66_575 | 39 |
| 66_576 | 27.4 |
| 66_585 | 32.3 |
| 66_588 | 22.5 |
| 66_589 | 18 |
| 66_591 | 26 |
| 66_609 | 25.9 |
| 66_610 | 56 |
| 66_611 | 34 |
| 66_612 | 35.6 |
| 66_613 | 25.3 |
| 68_2 | 59.6 |
| 68_3 | 74.5 |
| 68_4 | 70.2 |
| 68_7 | 81.3 |
| 68_9 | 92 |
| 68_11 | 104.5 |
| 68_12 | 62.3 |
| 68_13 | 49.4 |
| 68_14 | 77.7 |
| 68_15 | 67.9 |
| 68_16 | 27.6 |
| 68_17 | 60.3 |
| 68_18 | 70.3 |
| 68_19 | 80.7 |
| 68_25 | 62.4 |
| 68_26 | 84.5 |
| 69_1 | 67.5 |
| 69_2 | 69.3 |
| 69_3 | 87.2 |
| 69_4 | 83 |
| 69_5 | 84.2 |
| 69_6 | 70.1 |
| 69_7 | 87.8 |
| 69_8 | 89.9 |
| 69_9 | 81.8 |
| 69_10 | 84.5 |
| 69_11 | 77.4 |
| 69_12 | 90.5 |
| 69_13 | 86.1 |
| 69_14 | 85 |
| 69_15 | 81.5 |
| 69_16 | 92.5 |
| 69_17 | 78.1 |
| 69_18 | 106.3 |
| 69_19 | 84.5 |
| 69_20 | 79 |
| 69_21 | 92.3 |
| 69_22 | 91.3 |
| 69_23 | 94.5 |
| 69_24 | 45.9 |
| 69_25 | 76.9 |
| 69_26 | 87.7 |
| 69_27 | 69.2 |
| 69_28 | 35 |
| 69_29 | 39.5 |
| 69_30 | 44.4 |
| 69_31 | 82.9 |
| 69_32 | 86.6 |
| 70_1 | 46.2 |
| 70_2 | 90.7 |
| 70_3 | 42.6 |
| 70_4 | 42.7 |
| 70_5 | 45.2 |
| 70_6 | 60 |
| 70_7 | 89.7 |
| 70_8 | 106.6 |
| 70_9 | 99.5 |
| 70_10 | 116.1 |
| 70_11 | 71.7 |
| 70_12 | 89.8 |
| 70_13 | 47.7 |
| 70_14 | 34.4 |
| 70_15 | 63.2 |
| 70_16 | 69.6 |
| 70_17 | 70.9 |
| 70_18 | 91.1 |
| 70_19 | 69.1 |
| 70_20 | 75.5 |
| 70_21 | 103.6 |
| 70_22 | 82.6 |
| 70_23 | 119.7 |
| 70_24 | 94.2 |
| 70_25 | 112.2 |
| 70_26 | 70.5 |
| 70_27 | 75.9 |
| 70_28 | 73.5 |
| 70_29 | 76.5 |
| 70_30 | 90.3 |
| 70_31 | 80.1 |
| 70_32 | 44.5 |
| 70_33 | 98 |
| 70_34 | 85.1 |
| 70_35 | 91 |
| 70_36 | 89 |
| 70_37 | 85 |
| 70_38 | 86.2 |
| 70_39 | 82.8 |
| 70_40 | 87.6 |
| 70_41 | 92.8 |
| 70_42 | 89.3 |
| 70_43 | 80.5 |
| 70_44 | 74.4 |
| 70_45 | 77.3 |
| 70_46 | 75.1 |
| 70_47 | 79.8 |
| 70_48 | 84.5 |
| 70_49 | 83.6 |
| 70_50 | 96 |
| 70_51 | 108 |
| 70_52 | 102 |
| 70_53 | 97.2 |
| 70_54 | 105.6 |
| 70_55 | 108.1 |
| 70_56 | 107.4 |
| 71_1 | 105.9 |
| 72_1 | 85.9 |
| 72_2 | 104.6 |
| 72_3 | 99.7 |
| 72_4 | 72.2 |
| 73_1 | 102.6 |
| 73_2 | 103 |
| 73_3 | 100 |
| 73_4 | 108.4 |
| 73_5 | 83.2 |
| 73_6 | 88.4 |
| 73_7 | 100.1 |
| 73_8 | 90.5 |
| 73_9 | 89.2 |
| 73_10 | 94.6 |
| 73_11 | 93.6 |
| 74_1 | 91.2 |
| 74_2 | 68.1 |
| 74_3 | 97.2 |
| 74_4 | 96.9 |
| 74_5 | 68.8 |
| 74_6 | 88.4 |
| 74_7 | 95.9 |
| 74_8 | 93.5 |
| 74_9 | 100.4 |
| 74_10 | 79.1 |
| 74_11 | 89.5 |
| 74_12 | 101.4 |
| 74_13 | 67 |
| 74_14 | 71.4 |
| 74_15 | 79 |
| 75_1 | 88.7 |
| 75_2 | 89.7 |
| 75_3 | 92.9 |
| 75_4 | 96.1 |
| 75_5 | 92.4 |
| 75_6 | 56.1 |
| 75_7 | 50.9 |
| 75_8 | 93 |
| 75_9 | 53.2 |
| 75_10 | 53.3 |
| 75_11 | 50.5 |
| 75_12 | 55.1 |
| 75_13 | 53.2 |
| 75_14 | 49.5 |
| 75_15 | 53.5 |
| 75_16 | 47.4 |
| 75_17 | 105.7 |
| 75_18 | 99.5 |
| 75_19 | 44.3 |
| 75_20 | 91.4 |
| 75_21 | 107.9 |
| 75_22 | 103.1 |
| 75_23 | 85.4 |
| 75_24 | 90.1 |
| 75_25 | 99.4 |
| 75_26 | 112 |
| 75_27 | 115.4 |
| 75_28 | 96 |
| 75_29 | 92.8 |
| 75_30 | 90.2 |
| 75_31 | 83.5 |
| 75_32 | 79.5 |
| 75_33 | 67.4 |
| 75_34 | 89.7 |
| 75_35 | 95.1 |
| 75_36 | 73.5 |
| 75_37 | 97.3 |
| 75_38 | 100.3 |
| 75_39 | 89.2 |
| 76_1 | 83.5 |
| 76_2 | 102.3 |
| 76_3 | 105.3 |
| 76_4 | 82.5 |
| 76_5 | 90.7 |
| 76_6 | 97.2 |
| 76_7 | 99.3 |
| 76_8 | 85.4 |
| 76_9 | 98.1 |
| 76_10 | 96 |
| 76_11 | 113.2 |
| 76_12 | 87.4 |
| 76_13 | 78 |
| 76_14 | 86.2 |
| 76_15 | 98 |
| 76_16 | 101.3 |
| 76_17 | 80.5 |
| 76_18 | 83 |
| 76_19 | 83.3 |
| 76_20 | 90.6 |
| 76_21 | 80.4 |
| 76_22 | 99.3 |
| 76_23 | 83.2 |
| 76_24 | 86 |
| 76_25 | 99.4 |
| 76_26 | 97.2 |
| 76_27 | 91.4 |
| 76_28 | 84. |
| 76_29 | 81.5 |
| 76_30 | 87.9 |
| 76_31 | 91.6 |
| 76_32 | 78.8 |
| 76_33 | 98.8 |
| 76_34 | 82.6 |
| 76_35 | 110.2 |
| 76_36 | 91.8 |
| 76_37 | 85 |
| 76_38 | 79.8 |
| 76_39 | 59.3 |
| 76_40 | 91.3 |
| 76_41 | 69.8 |
| 77_1 | 58.5 |
| 77_2 | 59.1 |
| 77_3 | 72 |
| 77_4 | 53.3 |
| 77_5 | 70.8 |
| 77_6 | 70.9 |
| 77_7 | 68.9 |
| | % UTC(HEK293_2_1 uM) |
| 66_36 | 57 |
| 66_37 | 41 |
| 66_38 | 64.7 |
| 66_39 | 53.1 |
| 66_40 | 43.6 |
| 66_41 | 65.8 |
| 66_54 | 98.4 |
| 66_55 | 94.8 |
| 66_56 | 65.1 |
| 66_57 | 41.8 |
| 66_58 | 94.2 |
| 66_59 | 71.4 |
| 66_60 | 113.4 |
| 66_61 | 118.1 |
| 66_62 | 73.6 |
| 66_63 | 80.8 |
| 66_64 | 74.6 |
| 66_98 | 99.1 |
| 66_99 | 82.5 |
| 66_100 | 84.5 |
| 66_101 | 79.9 |
| 66_102 | 102.1 |
| 66_103 | 36.5 |
| 66_104 | 94.5 |
| 66_105 | 46 |
| 66_106 | 106.9 |
| 66_111 | 73.6 |
| 66_112 | 80.8 |
| 66_113 | 70.4 |
| 66_116 | 102.1 |
| 66_117 | 55.6 |
| 66_122 | 73.9 |
| 66_123 | 63.3 |
| 66_124 | 53.6 |
| 66_126 | 52.7 |
| 66_127 | 25.9 |
| 66_130 | 65.5 |
| 66_131 | 65 |
| 66_133 | 48.8 |
| 66_134 | 60.8 |
| 66_135 | 60.4 |
| 66_136 | 84.7 |
| 66_138 | 76.9 |
| 66_139 | 80.7 |
| 66_140 | 48.9 |
| 66_143 | 54.7 |
| 66_145 | 68.9 |
| 66_149 | 42 |
| 66_150 | 69.6 |
| 66_151 | 82.7 |
| 66_153 | 41.8 |
| 66_155 | 101.5 |
| 66_156 | 63 |
| 66_157 | 38.7 |
| 66_158 | 63.2 |
| 66_159 | 53.5 |
| 66_160 | 52.2 |
| 66_161 | 65.8 |
| 66_162 | 78.6 |
| 66_163 | 60.5 |
| 66_164 | 59.2 |
| 66_165 | 48.2 |
| 66_166 | 51.8 |
| 66_168 | 61.9 |
| 66_169 | 71.1 |
| 66_170 | 100.4 |
| 66_171 | 98.9 |
| 66_172 | 70.3 |
| 66_173 | 58.7 |
| 66_174 | 50.8 |
| 66_175 | 63.5 |
| 66_176 | 66.8 |
| 66_177 | 41.1 |
| 66_178 | 54.7 |
| 66_179 | 94.3 |
| 66_180 | 72.8 |
| 66_181 | 53.3 |
| 66_182 | 55.4 |
| 66_183 | 48.4 |
| 66_184 | 83.4 |
| 66_185 | 44 |
| 66_186 | 99.8 |
| 66_187 | 50.9 |
| 66_188 | 24.4 |
| 66_189 | 57.2 |
| 66_190 | 47.9 |
| 66_192 | 69.2 |
| 66_194 | 58.5 |
| 66_195 | 68 |
| 66_196 | 72.1 |
| 66_197 | 77 |
| 66_198 | 98.1 |
| 66_199 | 73.2 |
| 66_200 | 68 |
| 66_201 | 79.6 |
| 66_202 | 66.2 |
| 66_203 | 101 |
| 66_204 | 87.7 |
| 66_205 | 47.1 |
| 66_206 | 40.2 |
| 66_207 | 107.2 |
| 66_208 | 43.8 |
| 66_214 | 55 |
| 66_217 | 49.7 |
| 66_220 | 59.8 |
| 66_221 | 94.9 |
| 66_223 | 65.3 |
| 66_224 | 57.1 |
| 66_225 | 59.2 |
| 66_226 | 81.1 |
| 66_227 | 104.8 |
| 66_228 | 60.6 |
| 66_402 | 43.5 |
| 66_403 | 94.5 |
| 66_405 | 50.8 |
| 66_406 | 103.6 |
| 66_407 | 89.8 |
| 66_408 | 51.6 |
| 66_410 | 70.5 |
| 66_413 | 76 |
| 66_417 | 88.5 |
| 66_427 | 43.5 |
| 66_430 | 64.3 |
| 66_431 | 78.9 |
| 66_432 | 80.5 |
| 66_433 | 94.9 |
| 66_434 | 71.4 |
| 66_435 | 68.7 |
| 66_436 | 66.4 |
| 66_437 | 67.4 |
| 66_438 | 85.5 |
| 66_439 | 103.7 |
| 66_440 | 113.6 |
| 66_441 | 77.3 |
| 66_442 | 44.1 |
| 66_443 | 92.2 |
| 66_444 | 67.4 |
| 66_445 | 57.1 |
| 66_446 | 67 |
| 66_447 | 71.8 |
| 66_448 | 37.3 |
| 66_449 | 68.1 |
| 66_450 | 72.7 |
| 66_451 | 96.3 |
| 66_452 | 93.5 |
| 66_456 | 59.7 |
| 66_459 | 61.2 |
| 66_483 | 108.6 |
| 66_485 | 81.8 |
| 66_490 | 91.9 |
| 66_492 | 94.4 |
| 66_494 | 91.1 |
| 66_495 | 61.3 |
| 66_496 | 51.9 |
| 66_497 | 59.1 |
| 66_498 | 27 |
| 66_501 | 84.4 |
| 66_502 | 91.6 |
| 66_503 | 53 |
| 66_504 | 76 |
| 66_505 | 83.6 |
| 66_508 | 56.2 |
| 66_509 | 77.9 |
| 66_511 | 87.6 |
| 66_512 | 96.3 |
| 66_513 | 106.9 |
| 66_514 | 91.4 |
| 66_517 | 75.2 |
| 66_524 | 80.4 |
| 66_528 | 62 |
| 66_531 | 53.2 |
| 66_532 | 48.8 |
| 66_533 | 73.3 |
| 66_534 | 62.1 |
| 66_535 | 45.9 |
| 66_539 | 81 |
| 66_540 | 84.9 |
| 66_542 | 47.7 |
| 66_543 | 65.7 |
| 66_545 | 52.7 |
| 66_546 | 53.9 |
| 66_547 | 58.7 |
| 66_548 | 36 |
| 66_549 | 62.9 |
| 66_550 | 57.7 |
| 66_552 | 78.1 |
| 66_554 | 128.3 |
| 66_559 | 83.7 |
| 66_561 | 64.8 |
| 66_567 | 29.3 |
| 66_568 | 111 |
| 66_573 | 67.5 |
| 66_576 | 59.9 |
| 66_577 | 56.6 |
| 66_578 | 85.3 |
| 66_579 | 60.4 |
| 66_580 | 80.5 |
| 66_581 | 65.1 |
| 66_582 | 43.9 |
| 66_583 | 100.6 |
| 66_584 | 42.7 |
| 66_586 | 39.8 |
| 66_587 | 60.1 |
| 66_588 | 52.2 |
| 66_590 | 105.1 |
| 66_592 | 47.8 |
| 66_593 | 38.9 |
| 66_594 | 83.4 |
| 66_595 | 59.4 |
| 66_596 | 62.4 |
| 66_597 | 60.1 |
| 66_598 | 88.2 |
| 66_599 | 97.6 |
| 66_600 | 65.1 |
| 66_601 | 87.9 |
| 66_602 | 79.2 |
| 66_603 | 99.9 |
| 66_604 | 46.5 |
| 66_605 | 96 |
| 66_606 | 76.9 |
| 66_607 | 62.8 |
| 66_608 | 69.9 |
| 68_1 | 88.4 |
| 68_5 | 117.1 |
| 68_6 | 93.7 |
| 68_8 | 69.2 |
| 68_10 | 103.2 |
| 68_20 | 89.5 |
| 68_21 | 98.7 |
| 68_22 | 51.3 |
| 68_23 | 62.2 |
| 68_24 | 50.9 |
| | % UTC(HEK293_3_5 uM) |
| 66_36 | 28.7 |
| 66_37 | 59.3 |
| 66_38 | 50.8 |
| 66_39 | 34.1 |
| 66_40 | 44.8 |
| 66_41 | 50.9 |
| 66_54 | 72.8 |
| 66_55 | 90.9 |
| 66_56 | 70.2 |
| 66_57 | 41.8 |
| 66_58 | 87.8 |
| 66_59 | 82.5 |
| 66_60 | 81.9 |
| 66_61 | 59.7 |
| 66_62 | 53.1 |
| 66_63 | 35.1 |
| 66_64 | 41.3 |
| 66_98 | 86.2 |
| 66_99 | 81.2 |
| 66_100 | 75.9 |
| 66_101 | 50.2 |
| 66_102 | 71.1 |
| 66_103 | 47.5 |
| 66_104 | 82.8 |
| 66_105 | 44.7 |
| 66_106 | 92 |
| 66_111 | 66.6 |
| 66_112 | 55.5 |
| 66_113 | 69.7 |
| 66_116 | 77.4 |
| 66_117 | 27.1 |
| 66_122 | 56.4 |
| 66_123 | 26.7 |
| 66_124 | 32.5 |
| 66_126 | 26.7 |
| 66_127 | 26.3 |
| 66_130 | 41.9 |
| 66_131 | 42.6 |
| 66_133 | 64 |
| 66_134 | 37.4 |
| 66_135 | 36.8 |
| 66_136 | 72.5 |
| 66_138 | 57.7 |
| 66_139 | 55.6 |
| 66_140 | 30.5 |
| 66_143 | 42.4 |
| 66_145 | 47.9 |
| 66_149 | 20.4 |
| 66_150 | 45.9 |
| 66_151 | 52 |
| 66_153 | 23.3 |
| 66_155 | 78.8 |
| 66_156 | 42.2 |
| 66_157 | 42.2 |
| 66_158 | 40.9 |
| 66_159 | 52.6 |
| 66_160 | 41.6 |
| 66_161 | 55 |
| 66_162 | 62.5 |
| 66_163 | 68.8 |
| 66_164 | 35.5 |
| 66_165 | 48.1 |
| 66_166 | 36.8 |
| 66_168 | 48.9 |
| 66_169 | 42.9 |
| 66_170 | 50 |
| 66_171 | 70.3 |
| 66_172 | 48 |
| 66_173 | 38.2 |
| 66_174 | 39.1 |
| 66_175 | 48.9 |
| 66_176 | 37.8 |
| 66_177 | 30.4 |
| 66_178 | 65.1 |
| 66_179 | 48.8 |
| 66_180 | 42.7 |
| 66_181 | 24.6 |
| 66_182 | 31.7 |
| 66_183 | 26.5 |
| 66_184 | 73.7 |
| 66_185 | 34.4 |
| 66_186 | 68 |
| 66_187 | 37.4 |
| 66_188 | 24.5 |
| 66_189 | 29.6 |
| 66_190 | 47.2 |
| 66_192 | 32.9 |
| 66_194 | 46.2 |
| 66_195 | 27 |
| 66_196 | 39.1 |
| 66_197 | 71.9 |
| 66_198 | 73.9 |
| 66_199 | 54 |
| 66_200 | 42.1 |
| 66_201 | 53.1 |
| 66_202 | 50.1 |
| 66_203 | 84.9 |
| 66_204 | 71.7 |
| 66_205 | 63.1 |
| 66_206 | 49.5 |
| 66_207 | 83.8 |
| 66_208 | 52.9 |
| 66_214 | 41.5 |
| 66_217 | 31.5 |
| 66_220 | 39.2 |
| 66_221 | 62.1 |
| 66_223 | 74.6 |
| 66_224 | 41.6 |
| 66_225 | 62.6 |
| 66_226 | 41.1 |
| 66_227 | 77.8 |
| 66_228 | 71.8 |
| 66_402 | 40.1 |
| 66_403 | 54.2 |
| 66_405 | 43.8 |
| 66_406 | 53.9 |
| 66_407 | 61.6 |
| 66_408 | 25.2 |
| 66_410 | 63.5 |
| 66_413 | 51.3 |
| 66_417 | 64.7 |
| 66_427 | 44 |
| 66_430 | 29.4 |
| 66_431 | 37.5 |
| 66_432 | 40.2 |
| 66_433 | 89.1 |
| 66_434 | 39.5 |
| 66_435 | 50.2 |
| 66_436 | 67.1 |
| 66_437 | 54.5 |
| 66_438 | 73.7 |
| 66_439 | 60.2 |
| 66_440 | 91.7 |
| 66_441 | 48.6 |
| 66_442 | 41.1 |
| 66_443 | 61.6 |
| 66_444 | 67.8 |
| 66_445 | 50.5 |
| 66_446 | 36.9 |
| 66_447 | 54.8 |
| 66_448 | 27.1 |
| 66_449 | 62.9 |
| 66_450 | 49.6 |
| 66_451 | 68.3 |
| 66_452 | 72.9 |
| 66_456 | 32.5 |
| 66_459 | 38.6 |
| 66_483 | 62.8 |
| 66_485 | 42.6 |
| 66_490 | 56.3 |
| 66_492 | 46.6 |
| 66_494 | 52.2 |
| 66_495 | 55 |
| 66_496 | 36 |
| 66_497 | 30.3 |
| 66_498 | 39.1 |
| 66_501 | 60.4 |
| 66_502 | 75.2 |
| 66_503 | 71.2 |
| 66_504 | 60.6 |
| 66_505 | 56.9 |
| 66_508 | 89.1 |
| 66_509 | 71.7 |
| 66_511 | 59.2 |
| 66_512 | 68.3 |
| 66_513 | 53.5 |
| 66_514 | 69.4 |
| 66_517 | 55.8 |
| 66_524 | 74 |
| 66_528 | 36.3 |
| 66_531 | 33.3 |
| 66_532 | 22.4 |
| 66_533 | 79.9 |
| 66_534 | 51.9 |
| 66_535 | 34.7 |
| 66_539 | 62.5 |
| 66_540 | 74.5 |
| 66_542 | 40.2 |
| 66_543 | 43 |
| 66_545 | 33 |
| 66_546 | 25.8 |
| 66_547 | 42 |
| 66_548 | 47.8 |
| 66_549 | 48.4 |
| 66_550 | 43.3 |
| 66_552 | 78.7 |
| 66_554 | 95.5 |
| 66_559 | 73.6 |
| 66_561 | 65.6 |
| 66_567 | 24 |
| 66_568 | 74.3 |
| 66_573 | 38.9 |
| 66_576 | 41 |
| 66_577 | 59.3 |
| 66_578 | 62.3 |
| 66_579 | 64.1 |
| 66_580 | 62.6 |
| 66_581 | 43.8 |
| 66_582 | 66.7 |
| 66_583 | 77.8 |
| 66_584 | 44.9 |
| 66_586 | 34.9 |
| 66_587 | 38.9 |
| 66_588 | 40.5 |
| 66_590 | 96.1 |
| 66_592 | 39.9 |
| 66_593 | 26.3 |
| 66_594 | 76.1 |
| 66_595 | 68.6 |
| 66_596 | 60.3 |
| 66_597 | 62.2 |
| 66_598 | 77.3 |
| 66_599 | 52.1 |
| 66_600 | 42.1 |
| 66_601 | 56.1 |
| 66_602 | 47.9 |
| 66_603 | 86.3 |
| 66_604 | 44.9 |
| 66_605 | 54.8 |
| 66_606 | 74 |
| 66_607 | 36 |
| 66_608 | 54 |
| 68_1 | 52.4 |
| 68_5 | 98.5 |
| 68_6 | 60.4 |
| 68_8 | 72.2 |
| 68_10 | 78.1 |
| 68_20 | 87.8 |
| 68_21 | 91.9 |
| 68_22 | 55.1 |
| 68_23 | 54.6 |
| 68_24 | 79.1 |
| | % UTC(HEK293_4_25 uM) |
| 53_1 | 92.9 |
| 53_2 | 86.4 |
| 53_3 | 77.5 |
| 53_4 | 84.8 |
| 53_5 | 85.3 |
| 53_6 | 92.2 |
| 53_7 | 78.7 |
| 56_1 | 56.5 |
| 56_2 | 57.8 |
| 56_3 | 87.7 |
| 56_4 | 106.8 |
| 56_5 | 85.7 |
| 56_6 | 46.1 |
| 57_1 | 96.7 |
| 57_2 | 72.5 |
| 57_3 | 84 |
| 57_4 | 95.3 |
| 57_5 | 64.3 |
| 58_1 | 72.8 |
| 58_2 | 102.1 |
| 58_3 | 84.9 |
| 58_4 | 74.6 |
| 58_5 | 66.6 |
| 58_6 | 69 |
| 58_7 | 77.4 |
| 58_8 | 91.6 |
| 58_9 | 79.8 |
| 58_10 | 91.7 |
| 58_11 | 86.3 |
| 58_12 | 86.6 |
| 58_13 | 73.2 |
| 58_14 | 67.5 |
| 58_15 | 59.3 |
| 59_1 | 96.3 |
| 59_2 | 83.7 |
| 59_3 | 96.5 |
| 59_4 | 100.9 |
| 59_5 | 90.4 |
| 63_1 | 40.7 |
| 63_2 | 73.5 |
| 63_3 | 78.7 |
| 63_4 | 49.8 |
| 63_5 | 49.8 |
| 63_6 | 41.1 |
| 63_7 | 76.8 |
| 63_8 | 78.7 |
| 63_9 | 65 |
| 63_10 | 73.9 |
| 63_11 | 29.5 |
| 63_12 | 52.4 |
| 63_13 | 71 |
| 63_14 | 72.3 |
| 63_15 | 87.7 |
| 63_16 | 83 |
| 63_17 | 69.9 |
| 63_18 | 92.2 |
| 63_19 | 70.8 |
| 66_1 | 92.5 |
| 66_2 | 67.8 |
| 66_3 | 61.6 |
| 66_4 | 69.6 |
| 66_5 | 69.4 |
| 66_6 | 50.2 |
| 66_7 | 45.3 |
| 66_8 | 24 |
| 66_9 | 66.9 |
| 66_10 | 40.7 |
| 66_11 | 26.5 |
| 66_12 | 34.2 |
| 66_13 | 38.2 |
| 66_137 | 44.4 |
| 66_141 | 16.4 |
| 66_142 | 25.8 |
| 66_144 | 19.8 |
| 66_146 | 6.5 |
| 66_147 | 14.5 |
| 66_148 | 11 |
| 66_149 | 5.2 |
| 66_152 | 8.6 |
| 66_153 | 6.9 |
| 66_154 | 12.4 |
| 66_167 | 19.5 |
| 66_349 | 8.9 |
| 66_614 | 4.2 |
| 67_1 | 76.7 |
| 67_2 | 52.6 |
| 67_3 | 52.1 |
| 67_4 | 88.4 |
| | % UTC(HEK293_25 uM) |
| 67_5 | 35.5 |
| 67_6 | 84.6 |
| 67_7 | 57.8 |
| 67_8 | 62.9 |
| 67_9 | 55.3 |
| | % UTC(HEK293_4_1 uM) |
| 66_299 | 28.7 |
| 66_528 | 21.6 |
| 66_531 | 23.4 |
| 66_532 | 25.4 |
| 66_535 | 17.4 |
| 66_546 | 24.6 |
| 66_614 | 13.4 |

### Example 6: Determination of IC50 Values of NAT8L Targeting ASOs in HEK Cells

To validate the hits identified in single concentration screens (Example 5) and to rank compounds on their potency a concentration response experiments were subsequently carried out for compounds showing high level of knock down.

Using the same method as describe in example 2 but incubating the cells with different concentrations of ASO (0.01; 0.0316; 1; 0.316; 1.0; 3.16; 10; 31.6 μM) allowed the generation of concentration response curves and determination of IC50 values.

Concentration response curves were generated using the GraphPad prism software version9 using the “log(inhibitor) vs. response—Variable slope (four parameters)” fit with bottom constrained to >0 and top=100. IC50 values are shown in Table 9.

TABLE 9
IC50 value of selected compounds

| ASO ID | IC50 value; [M] |
|---|---|
| 66_8 | 1.09E−05 |
| 66_149 | 6.48E−07 |
| 66_148 | 2.19E−06 |
| 66_147 | 6.44E−06 |
| 66_146 | 1.77E−06 |
| 66_141 | 3.37E−06 |
| 66_144 | 5.7E−06 |
| 66_152 | 1.39E−06 |
| 66_153 | 8.27E−07 |
| 66_154 | 5.06E−06 |
| 66_167 | 4.62E−06 |
| 66_545 | 5.06E−06 |
| 66_609 | 1.29E−06 |
| 66_541 | 1.15E−06 |
| 66_477 | 1.26E−06 |
| 66_527 | 8.41E−07 |
| 66_613 | 1.2E−06 |
| 66_530 | 9.46E−07 |
| 66_478 | 9.72E−07 |
| 66_132 | 1.13E−06 |
| 66_27 | 2.64E−06 |
| 66_48 | 1.2E−06 |
| 66_47 | 1.57E−06 |
| 66_46 | 1.1E−06 |
| 66_36 | 1.24E−06 |
| 66_120 | 1.09E−06 |
| 66_117 | 1.38E−06 |
| 66_123 | 1.33E−06 |
| 66_189 | 1.37E−06 |
| 66_217 | 1.19E−06 |
| 66_408 | 8.3E−07 |
| 66_412 | 2.38E−06 |
| 66_456 | 1.82E−06 |
| 66_459 | 3.33E−07 |
| 66_467 | 2.97E−06 |
| 66_475 | 2.56E−06 |
| 66_469 | 3.24E−06 |
| 66_471 | 1.35E−06 |
| 66_585 | 1.06E−06 |
| 66_591 | 7.82E−07 |
| 66_588 | 1.23E−06 |
| 66_589 | 2.16E−06 |
| 66_576 | 1.4E−06 |
| 68_16 | 1.9E−06 |
| 68_12 | 5.63E−06 |
| 70_14 | 9.28E−06 |
| 66_492 | 3.07E−06 |
| 66_140 | 2.23E−06 |
| 66_160 | 1.92E−06 |
| 66_496 | 1.24E−06 |
| 66_220 | 2.99E−06 |
| 66_176 | 2.24E−06 |
| 66_584 | 2.63E−06 |
| 66_547 | 1.70E−06 |
| 66_126 | 1.64E−06 |
| 66_593 | 1.25E−06 |
| 66_188 | 5.41E−07 |
| 66_177 | 1.62E−06 |
| 66_39 | 2.68E−06 |
| 66_127 | 1.06E−06 |
| 66_183 | 1.08E−06 |
| 66_185 | 6.42E−07 |
| 66_181 | 8.27E−07 |
| 66_182 | 1.88E−06 |
| 66_135 | 1.93E−06 |
| 66_134 | 1.99E−06 |
| 66_174 | 2.49E−06 |
| 66_431 | 2.48E−06 |
| 66_430 | 7.23E−07 |
| 66_592 | 2.29E−06 |
| 66_485 | 4.05E−06 |
| 66_64 | 5.57E−06 |
| 66_130 | 2.76E−06 |
| 66_587 | 2.62E−06 |
| 66_567 | 5.70E−06 |
| 66_573 | 1.16E−06 |
| 66_124 | 1.42E−06 |
| 66_600 | 1.91E−06 |
| 66_173 | 1.77E−06 |
| 66_63 | 5.84E−06 |
| 66_544 | 4.31E−07 |
| 54_1 | 7.84E−07 |
| 54_3 | 3.46E−06 |
| 54_2 | 3.56E−06 |

### Example 7. Testing In Vitro Efficacy of Antisense Oligonucleotides Targeting NAT8L in iPSC Derived Human Neurons at Multiple Test Concentration

A set of ASOs selected to cover most of the preferred regions were tested in iPSC derived human neurons to confirm activity and potency of the most active ASOs in a relevant cellular model.

The iPSC derived neurons were maintained as recommended by the supplier (Fuji film, 01279). 96-well cell culture plates (Nunc™ Edge™ 96-Well, Nunclon Delta-Treated, Flat-Bottom Microplates) were coated with sterile 0.07% polyethylenimide for 1 hour, washed with PBS and water and coated with 0.01 mg/ml of laminin for 1 hour. The laminin solution was removed with no further washing of the culture plates and iPSC derived neurons were thawed and seated at a density of 80.000 neurons pr well as described in Fuji films: iCell GlutaNeurons User's Guide in a total volume of 190 μl/well plates using a multichannel pipette. After 24 and 96 hours of incubation, half of the media was changed. 96 hours after seeding NAT8L ASOs (Table 11, see columns “ASO ID”), are added directly to the growth media from a 20-Fold stock dilution in PBS reaching a final ASO concentration of either 0.2, 1, 5 or 25 μM. Table 10 summarizes the most important parameters relating to the cellular work.

TABLE 10
Information on iPSC derived neurons

| | | | | Incubation | Incubation |
|---|---|---|---|---|---|
| | | Culturing | Seeding density | prior to ASO | time with |
| IPSC | Provider | conditions | (cells/well) | addition | ASO |
| Gluta | Fujifilm | 5% CO₂ and | 80,000 cells/ | 96 h | 96 h |
| neurons | | 95% humidity | well in 190 | | |
| (01279) | | at 37° C. | μl of media as | | |
| | | | described by | | |
| | | | the provider | | |

Neurons were harvested after 96 hours treatment with ASO by gently aspirating the growth media, addition of 125 μl RLT buffer and RNA was extracted using the RNeasy 96, QIAcube HT kit (Qiagen #74171) according to the manufactures instructions and eluted in 75 μl of water.

qPCR experiments were carried out as described in Example 1. Quantities of NAT8L mRNA was calculated applying the ddCT method and using the median of all the PBS treated wells within the same plate as the ‘untreated control’. The expression level of NAT8L following ASO treatment (Table 11) is thus shown as percent of the PBS-treated wells.

As for example 3, concentration response curves were generated using the GraphPad prism software version9 using the “log(inhibitor) vs. response—Variable slope (four parameters)” fit with bottom constrained to >0 and top=100. IC50 values are shown in Table 11.

TABLE 11
Expression levels of human NAT8L in iPSC derived neurons
shown as % UnTreatedControl (% UTC) and as IC50 Value

| | % UTC | % UTC | % UTC | % UTC | |
|---|---|---|---|---|---|
| | (gluta_neurons_{—} | (gluta_neurons_{—} | (gluta_neurons_{—} | (gluta_neurons_{—} | |
| ASO ID | 0.2 uM) | 1 uM) | 5 uM) | 25 uM) | IC50 [M] |
| 66_36 | 74.9 | 40.8 | 11 | 1.1 | 3.26E−07 |
| 66_117 | 61.9 | 25 | 5.5 | 1.8 | 3.93E−07 |
| 66_123 | 64.6 | 30.1 | 6.6 | 0.3 | 3.75E−07 |
| 66_149 | 65.7 | 25.8 | 5.1 | 0.7 | 5.4E−07 |
| 66_153 | 69.1 | 37.2 | 16 | 2.4 | 3.26E−07 |
| 66_189 | 60.3 | 28 | 5.1 | 0.4 | 3.3E−07 |
| 66_217 | 58.5 | 28.8 | 5.8 | 1 | 7.04E−07 |
| 66_408 | 80.3 | 41.6 | 10.8 | 1.4 | 7.43E−07 |
| 66_459 | 90.8 | 54.4 | 23.4 | 5 | 1.25E−06 |
| 66_528 | 79.8 | 44.1 | 14.8 | 1.8 | 8.31E−07 |
| 66_546 | 80.1 | 50.1 | 23.2 | 13.6 | 7.81E−07 |
| 66_588 | 61.7 | 26.6 | 7 | 1 | 3.36E−07 |
| 68_16 | 64.6 | 24.9 | 10.4 | 3.7 | 3.23E−07 |

### Example 8—In Vivo Acute Toxicity in Mice of ASOs Targeting NAT8L

To test the acute toxicity potential of the NAT8L targeting ASOs, acute in vivo CNS toxicity study was tested by neurobehavioral scoring after intracerebroventricular dosing, as described below.

Experimental mice. In vivo acute tolerability of the antisense oligonucleotides were tested in mice, with 6 mice per ASO group. Mice at 8-10 weeks of age aged were housed in European IVC cages type IIL with TAPVEI aspen bedding (Tapvei Eatonis Oü, Estonia).

The cages were enriched with nesting material, wooden sticks and hiding material. The light cycle was 12-hour dark and 12-hour light. Diet was pelleted complete diet (Altromin 1324, Brogaarden), and municipal drinking water. Diet and water were administered ad libitum.

All animals were inspected on daily basis for their general health condition. Any clinical signs or behavioral abnormalities was recorded. Humane endpoints and premature termination: Any animal showing clinical signs of moderate pain or moderate distress, or any degree of suffering was handled as appropriate, as discontinuation of the administration of test articles or euthanizing of the animal. Animals exhibiting clinical signs were humanely euthanized if they exceeded the limits of the study specific humane endpoints according to the European and Danish legislation on animals in experimentation.

Administration by ICV injection: Pre-dosing analgesia was given at least 30 min before dosing is initiated, all animals received preventive pain treatment with Meloxicam (2 mg/kg SC. The mice were be anesthetized with isoflurane before dosing.

For injection, the G23 needle was mounted on a stand so it precisely penetrated 3.9 mm through the mouse's skull. The dose volume of 5 μL was injected over 30 seconds and the animal was then placed back in its cage and clinically observed according to the instructions below.

Following dosing, the animals were observed closely. The assessment was performed according to a scoring scheme where a score of 0 to 4 was given for the following five parameters: activity level (increased or decreased activity, respectively), motor function, posture/presentation, and muscle tremors/cramps as outlined in table 12. Scoring was performed before dose, 30 min and 60 min after dosing.

TABLE 12

| Category | Score 0 | Score 1 | Score 2 | Score 3 | Score 4 |
|---|---|---|---|---|---|
| Hyperactivity, | Normal | Very slightly | Increased home | Moderately | Marked |
| stereotypies | | increased home | cage exploration | increased home | hyperactivity. |
| and home cage | | cage exploration | (incl. e.g. digging, | cage activity. | Marked |
| behavior | | or rearing | burying) | Detectable | stereotypies. |
| | | compared to | Increased grooming | stereotypies (eg. | |
| | | controls | | circling, repetitive | |
| | | | | behaviours) | |
| Decreased | Normal | Some reduction | Drowsiness | Stupor (reduced | Coma (does |
| vigilance, | | in exploratory | Slightly reduced | responsiveness, | not respond to |
| exploration, | | activity | response to touch | decreased corneal | stimulation e.g. |
| and | | Responds | or handling. | reflex) | pinch), no |
| responsiveness | | normally to | | | corneal reflex |
| | | stimulation. | | | |
| Motor | Normal | Mild changes | Reduced grip | Highly reduced | Severe ataxia |
| coordination | | to gait or grip | strength (falls in | grip strength (falls | (e.g. crawling; |
| and strength | | strength (falls | less than 5 sec) | in less than 2 sec) | fails to grip bar) |
| | | between 5-10 | Mild ataxia (e.g. | Ataxia (e.g. | No ability to |
| | | sec) | slow righting | staggering, falling; | right |
| | | No falls, | response; swaying) | impaired walking) | |
| | | normal righting | | | |
| | | response | | | |
| Posture, | Normal | Very slightly | Slightly | Moderately | Markedly abnormal |
| appearance | | abnormal | abnormal posture | abnormal posture | posture (e.g. |
| and breathing | | posture (subtle). | (e.g. hunched, | (e.g. ventral | lateral recumbency) |
| | | | extended, low | recumbency) | Facial paralysis |
| | | | posture, tail | Shallow breathing | (e.g. drooling, |
| | | | position eg. | | protruding tongue) |
| | | | Straub tail) | | Laboured breathing |
| | | | Piloerection or | | |
| | | | unkept coat | | |
| | | | Ptosis | | |
| Tremor, | Normal | Detectable tremor | Hyper-responsive | Few or partial | Repeated or |
| hyper- | | | to stimuli (e.g. | seizures, rearing & | continuous |
| reactivity, | | | noise) | falling as a part of | seizure |
| convulsion | | | Marked tremor | convulsing | (running, |
| | | | | | bouncing, tonic |
| | | | | | and/or clonic) |

Average neurobehavioral scoring at 1 hour post dose for a 50 μg dose or 100 μg dose is summarized in table 13. If the mice had to be euthanized before 1 hour post dosing, they got a score of 20.

TABLE 13
Acute neurotoxicity data

| | Acute tox score | Acute tox score |
|---|---|---|
| ASO ID | 1 h (dose 50 μg) | 1 h (dose 100 μg) |
| 25_111 | 4.5 | |
| 29_124 | | 2.6 |
| 29_130 | | 5.8 |
| 29_138 | | 20.0 |
| 29_24 | | 4.3 |
| 29_34 | | 4.4 |
| 29_36 | | 6.1 |
| 29_5 | | 6.0 |
| 29_79 | | 2.3 |
| 29_84 | | 3.0 |
| 29_85 | | 2.2 |
| 29_86 | | 3.5 |
| 34_46 | | 20.0 |
| 37_15 | | 20.0 |
| 38_10 | | 20.0 |
| 66_117 | 3 | |
| 66_123 | 2.5 | 3.5 |
| 66_189 | | 6.3 |
| 66_153 | | 6.0 |
| 66_217 | | 8.0 |
| 66_27 | | 4.2 |
| 66_36 | | 2.8 |
| 66_412 | | 6.7 |
| 66_459 | | 6.0 |
| 66_545 | | 3.0 |
| 68_16 | | 8.7 |
| 54_1 | 6.3 | |

The generated acute toxicity data in mice shows that the observed acute toxicity is highly dependent on the ASO sequence. Some ASOs e.g. 29_138 and 34_46 are highly neurotoxic and giving rise to toxic effects of a severity requiring the mice to be euthanized shortly after dosing. In contrast ASOs such as 29_79, 29_84, 29_85, 29_124 and 66_36 showed only minor short lasting neurological signs.

### Example 9. Single Dose In Vivo Efficacy Test in African Green Monkeys (Chlorocebus sabaeus)

To evaluate the efficacy of one of the most preferred ASOs; ASO ID 2985 in vivo using a relevant dosing paradigm and in a species of high translational value, a single dose of ASO was delivered to African green monkeys via an IT-catheter followed by a two week in life phase before harvest of brain tissues.

After a 4-week recovery from IT CSF access port implantation, monkeys were fasted overnight, sedated with ketamine/xylazine and placed in a prone position. 0.5 ml of ASO or saline were injected followed by a saline flush of 0.5 mL/kg monkey.

2 monkeys were dosed with ASO ID 29_85 and 2 monkeys were dosed with 0.9% saline. Following dosing the monkey were monitored closely for their general wellbeing throughout the 2 weeks of the experiment, without observing any adverse effects.

At termination, monkeys were sedated intramuscularly with ketamine and xylazine to effect and euthanized with sodium pentobarbital. The following brain tissues was collected with a 2 mm punch into DNase/RNase free cryotubes and snap-frozen: Frontal Cortex, Parietal Cortex, Occipital Cortex, Ventral Striatum (nucleus accumbens area), Hippocampus, Ventral Pons, Medulla, Cerebellum, Ventral Midbrain, Cervical Spinal Cord, Thoracic Spinal Cord, Lumbar Spinal Cord.

Brain tissue pieces were homogenized in RLT buffer using the Precellus system, then Trizol/choroform extracted and RNA from the aqueous phases were purified on a QIAcube HT using the RNeasy 96, QIAcube HT kit (Qiagen #74171). qPCR was performed as described in example 1 using qPCR assays specific for monkey NAT8L and monkey UBE2D2 for normalization. The reduction is presented as relative reduction compared to the median for saline treated NHPs with 2 punctures from each brain region.

A single 12 mg dose of ASO ID 29_85 administered intrathecally shows significant reduction of NAT8L relative to UBE2D2 in various brain regions as seen in table 14:

TABLE 14
% NAT8L mRNA levels (Normalized to UBE2D2)
of saline treated NHP (average ± stdev).

| | Frontal | Parietal | Occipital | |
|---|---|---|---|---|
| Compound | cortex | cortex | cortex | Hippocampus |
| Saline | 99.3 ± 4.7 | 102.4 ± 8.2 | 100.0 ± 14.7 | 99.8 ± 4.3 |
| ASO ID 29_85 | 37.1 ± 9.9 | 20.8 ± 2.3 | 27.9 ± 3.7 | 52.8 ± 11.4 |
| Compound | Ventral Pons | Medulla | Cerebellum | Ventral Midbrain. |
| Saline | 100.3 ± 15.1 | 95.6 ± 18.7 | 100.1 ± 15.1 | 105.0 ± 15.9 |
| ASO ID 29_85 | 71.4 ± 19.0 | 45.6 ± 12.5 | 69.5 ± 31.3 | 63.2 ± 32.3 |
| | Ventral | Cervical | Thoracic | Lumbar |
| Compound | Striatum | Spinal Cord | Spinal Cord | Spinal Cord |
| Saline | 100.0 ± 2.8 | 97.5 ± 8.9 | 97.7 ± 8.1 | 99.8 ± 23.8 |
| ASO ID 29_85 | 77.7 ± 13.8 | 35.5 ± 7.4 | 21.4 ± 3.6 | 8.5 ± 1.9 |

Altogether the data shows that ASOs identified in our screening cascade consisting of in vitro efficacy in cell lines (example 1-6), human IPSC derived neurons (example 7) mouse acute toxicity (example 8) can generate ASOs that are well tolerate and highly efficacious in non-human-primates.

### Example 10: Overview on Target Regions within the NAT8L Pre-mRNA Sequence (SEQ ID NO: 1) Identified in Example 5

Target sequences of the invention that were found in Example 5 are shown in Table D1. Targeting these regions allows for an efficient downregulation of the NAT8L gene. Some of the identified target sequences, in principle, correspond to some of the target sequences described in Table B1 and are subregions or expansions of these target sequences. For more details, see the Table in the specification.

TABLE D1
Target sequences

| | | | | | overlap |
|---|---|---|---|---|---|
| Start | End | | | | or |
| in | in | | | | subregion |
| SEQ | SEQ | SEQ | | | of |
| ID | ID | ID | | Target | Target |
| NO: 1 | NO 1 | NO | Target sequence premRNA | ID | ID |
| 215 | 231 | 882 | GAGACGAAGATCGTGG | 53 | — |
| 894 | 917 | 883 | AGTGCCTAAATATAATCTGCGGG | 54 | 2 |
| 940 | 957 | 884 | GTGCTAATTTATGAGGG | 55 | 3 |
| 1152 | 1168 | 885 | GAAATGCGGCTGGAAG | 56 | — |
| 3043 | 3060 | 886 | GTAGGTAGAGGCAGAAC | 57 | 10 |
| 3535 | 3552 | 887 | TTGGTCTTCAGAGAGGT | 58 | — |
| 3572 | 3589 | 888 | TTTGGCTGCTGGAGGTG | 59 | — |
| 4228 | 4244 | 889 | TTGTCCTGAATAGTGG | 60 | 16 |
| 5735 | 5761 | 890 | TAATAAGTTATTTAGACAGAATAGCA | 61 | 25 |
| 5768 | 5791 | 891 | TGACTTTAATTCTTGGGACAAAA | 62 | 25 |
| 6187 | 6208 | 892 | TTGGTGGCAGGAGAAGAATAA | 63 | — |
| 6390 | 6410 | 893 | TGTTAGCAATTGAGGTGTGC | 64 | 26 |
| 6477 | 6504 | 894 | TGGGAGTTTTGTAGACTGTACAGAAA | 65 | 27 |
| | | | T | | |
| 6528 | 6597 | 895 | AGATTTTGTTAATCTGGAATATACAG | 66 | 29 |
| | | | ACAGACGTAAAGTGTTTTAGCAAAAT | | |
| | | | GGAAACAAACAGTTGTG | | |
| 7013 | 7036 | 896 | AGGACTTGGTAGAGATGGCAGGA | 67 | — |
| 7291 | 7310 | 897 | TGACGTGAATTTTATACGT | 68 | 34 |
| 7507 | 7528 | 898 | GATTTCTGTGTGTTTGGAAGC | 69 | 35 |
| 8722 | 8768 | 899 | TATTTTTTTTAAAACAGCAATAATTA | 70 | 38 |
| | | | GCCATTTTAAAGGAGGGATG | | |
| 8810 | 8827 | 900 | TGTGCAAGTGGGTTCTT | 71 | 39 |
| 8883 | 8900 | 901 | AGGTGCATAAGCATGTA | 72 | 40 |
| 8938 | 8958 | 902 | TGTACATGTGCATGAGTTGT | 73 | 41 |
| 8966 | 8984 | 903 | GTGAGCACGTGTATAGAT | 74 | 42 |
| 9154 | 9177 | 904 | ATACATATGCATATGGTGTACGT | 75 | 46 |
| 9329 | 9356 | 905 | TGTGTGTACTTGCATATATGTGGCTG | 76 | 49 |
| | | | T | | |
| 9743 | 9760 | 906 | TAAAACCAGAGTTGAAG | 77 | 52 |

Targeting sequences as shown in Table D2 allowed for a very efficient down-regulation of the target gene.

TABLE D2
Target sequences

| Start | End | | | |
|---|---|---|---|---|
| in | in | | | |
| SEQ | SEQ | SEQ | | Tar- |
| ID | ID | ID | | get |
| NO: 1 | NO 1 | NO | Target sequence premRNA | ID |
| 894 | 917 | 883 | AGTGCCTAAATATAATCTGCGGG | 54 |
| 6187 | 6208 | 892 | TTGGTGGCAGGAGAAGAATAA | 63 |
| 6477 | 6504 | 894 | TGGGAGTTTTGTAGACTGTACAGAAAT | 65 |
| 6528 | 6597 | 895 | AGATTTTGTTAATCTGGAATATAC | 66 |
| | | | AGACAGACGTAAAGTGTTTTAGCA | |
| | | | AAATGGAAACAAACAGTTGTG | |
| 7013 | 7036 | 896 | AGGACTTGGTAGAGATGGCAGGA | 67 |
| 7291 | 7310 | 897 | TGACGTGAATTTTATACGT | 68 |
| 7507 | 7528 | 898 | GATTTCTGTGTGTTTGGAAGC | 69 |
| 8722 | 8768 | 899 | TATTTTTTTTAAAACAGCAATAAT | 70 |
| | | | TAGCCATTTTAAAGGAGGGATG | |

The best results were obtained when ASOs were used that are complementary to target sequences shown in Table D3.

TABLE D3
Target sequences

| Start | End | | | |
|---|---|---|---|---|
| in | in | | | |
| SEQ | SEQ | SEQ | | Tar- |
| ID | ID | ID | | get |
| NO: 1 | NO 1 | NO | Target sequence premRNA | ID |
| 894 | 917 | 883 | AGTGCCTAAATATAATCTGCGGG | 54 |
| 6477 | 6504 | 894 | TGGGAGTTTTGTAGACTGTACA | 65 |
| | | | GAAAT | |
| 6528 | 6597 | 895 | AGATTTTGTTAATCTGGAATATAC | 66 |
| | | | AGACAGACGTAAAGTGTTTTAGCA | |
| | | | AAATGGAAACAAACAGTTGTG | |
| 7291 | 7310 | 897 | TGACGTGAATTTTATACGT | 68 |
| 7507 | 7528 | 898 | GATTTCTGTGTGTTTGGAAGC | 69 |
| 8722 | 8768 | 899 | TATTTTTTTTAAAACAGCAATAAT | 70 |
| | | | TAGCCATTTTAAAGGAGGGATG | |

Table D4 shows target sequences of preferred compounds.

TABLE D4
Target sequences of preferred compounds

| | | | | Target |
|---|---|---|---|---|
| Start | End | | | sequence |
| in | in | | | of e.g. |
| SEQ | SEQ | SEQ | | compound |
| ID | ID | ID | premRNA target | with |
| NO: 1 | NO 1 | NO | sequence | ASO ID* |
| 894 | 912 | 907 | GAGTGCCTAAATATAATCT | 2_17 |
| 5776 | 5793 | 908 | AATTCTTGGGACAAAACG | 25_111 |
| 6564 | 6583 | 909 | AAGTGTTTTAGCAAAATGGA | 29_10 |
| 6579 | 6596 | 910 | ATGGAAACAAACAGTTGT | 29_124 |
| 6581 | 6597 | 911 | GGAAACAAACAGTTGTG | 29_130 |
| 6583 | 6599 | 912 | AAACAAACAGTTGTGCC | 29_138 |
| 6565 | 6583 | 913 | AGTGTTTTAGCAAAATGGA | 29_34 |
| 6564 | 6582 | 914 | AAGTGTTTTAGCAAAATGG | 29_5 |
| 6576 | 6593 | 915 | AAAATGGAAACAAACAGT | 29_70 |
| 6577 | 6594 | 916 | AAATGGAAACAAACAGTT | 29_78 |
| 7324 | 7339 | 917 | GATGTAACTTCTTCTA | 34_39 |
| 7324 | 7340 | 918 | GATGTAACTTCTTCTAC | 34_46 |
| 8703 | 8720 | 919 | GGACATACCATTGTTGGA | 37_15 |
| 8734 | 8750 | 920 | AAACAGCAATAATTAGC | 38_10 |
| 9625 | 9640 | 921 | AGATGTGCCAAAACAG | 51_6 |
| 6538 | 6554 | 922 | TAATCTGGAATATACAG | 66_117 |
| 6543 | 6559 | 923 | TGGAATATACAGACAGA | 66_134 |
| 6553 | 6570 | 924 | AGACAGACGTAAAGTGTT | 66_149 |
| 6553 | 6569 | 925 | AGACAGACGTAAAGTGT | 66_189 |
| 6557 | 6574 | 926 | AGACGTAAAGTGTTTTAG | 66_217 |
| 6533 | 6552 | 927 | TTTGTTAATCTGGAATATAC | 66_27 |
| 6534 | 6553 | 928 | TTGTTAATCTGGAATATACA | 66_36 |
| 6569 | 6586 | 929 | TTTTAGCAAAATGGAAAC | 66_408 |
| 6570 | 6588 | 930 | TTTAGCAAAATGGAAACAA | 66_430 |
| 6570 | 6587 | 931 | TTTAGCAAAATGGAAACA | 66_456 |
| 6534 | 6552 | 932 | TTGTTAATCTGGAATATAC | 66_47 |
| 6580 | 6596 | 933 | TGGAAACAAACAGTTGT | 66_584 |
| 6580 | 6597 | 934 | TGGAAACAAACAGTTGTG | 66_588 |
| 7292 | 7308 | 935 | TGACGTGAATTTTATAC | 68_16 |
| 895 | 912 | 936 | AGTGCCTAAATATAATCT | 54_1 |

*can be the target sequences of other preferred compounds as well.

1. An antisense oligonucleotide comprising a stretch of at least 10 nucleotides which is at least 90% complementary to a target sequence in the human NAT8L (N-acetyltransferase 8 like) gene.

2. The antisense oligonucleotide of claim 1, wherein the stretch of at least 10 nucleotides is at least 90% complementary to a target sequence selected from the group of target sequences consisting of SEQ ID NO: 58, SEQ ID NO: 4 to SEQ ID NO: 57, SEQ ID NO: 59 to SEQ ID NO: 81, and SEQ ID NO: 882 to 906.

3. The antisense oligonucleotide of claim 1, wherein the stretch of at least 10 nucleotides is at least 90% complementary to a target sequence selected from the group of target sequences consisting of SEQ ID NO: 4 to SEQ ID NO: 29, in particular wherein the stretch of at least 10 nucleotides is at least 90% complementary to a target sequence selected from the group of target sequences consisting of: SEQ ID NO: 4 to SEQ ID NO: 10.

4. The antisense oligonucleotide of claim 1, wherein the stretch of at least 10 nucleotides is at least 90% complementary to a target sequence comprising a sequence as shown in SEQ ID NO: 883, 892, 894, 895, 896, 897, 898, 899, in particular as shown in SEQ ID NO NO: 883, 894, 895, 897, 898 and 899.

5. The antisense oligonucleotide of claim 1, comprising a stretch of at least 12, or in particular at least 14 nucleotides which is at least 90% complementary to said target sequence.

6. The antisense oligonucleotide of claim 1, wherein said stretch is 100% complementary to said target sequence.

7. The antisense oligonucleotide of claim 1, wherein the antisense oligonucleotide has a length of 12 to 30 nucleotides.

8. The antisense oligonucleotide of claim 7, wherein the antisense oligonucleotide has a length of 14 to 22 nucleotides, such as a length of 16 to 20 nucleotides.

9. The antisense oligonucleotide of claim 1, wherein the antisense oligonucleotide comprises or consists of a nucleic acid sequence as shown in SEQ ID NO: 403 to SEQ ID NO: 881.

10. The antisense oligonucleotide of claim 1, wherein the antisense oligonucleotide is capable of reducing the amount of NAT8L (N-acetyltransferase 8 like) mRNA in a host cell expressing said NAT8L mRNA.

11. The antisense oligonucleotide claim 10, wherein the target cell is a human cell, such as a cell of the CNS.

12. The antisense oligonucleotide of claim 1, wherein the antisense oligonucleotide is a chemically modified antisense oligonucleotide.

13. The antisense oligonucleotide of claim 12, wherein the chemically modified antisense oligonucleotide contains one or more modified nucleosides.

14. The antisense oligonucleotide of claim 13, wherein the one or more modified nucleosides, is a sugar modified nucleoside, such as a 2′ sugar modified nucleoside.

15. The antisense oligonucleotide of claim 11, wherein the chemically modified antisense oligonucleotide contains at least one modified nucleobase.

16. The antisense oligonucleotide of claim 13, wherein the at least one modified nucleobase is 5-methylcytosine.

17. The antisense oligonucleotide of claim 11, wherein the chemically modified antisense oligonucleotide comprises at least one modified nucleoside selected from the group consisting of: 2′-O-Methoxyethyl-RNA, 2′-O-Methyl-RNA, 2′-Fluoro-RNA.

18. The antisense oligonucleotide of claim 1, wherein the antisense oligonucleotide comprises at least one modified internucleoside linkage, such as at least one Phosphorothioate internucleoside linkage, at least one Phosphorodithioate internucleoside linkage, at least one Phophoroamidate internucleoside linkage, at least one methyl phosphonate internucleoside linkage, at least one phosphotriester internucleoside linkage, at least one boranophosphate internucleoside linkage or at least one phosphoryl guanidine internucleoside linkage.

19. The antisense oligonucleotide of claim 18, wherein all internucleoside linkages are modified internucleoside linkages, such as Phosphorothioate internucleoside linkages.

20. The antisense oligonucleotide of claim 1, wherein the antisense oligonucleotide comprises one or more modified nucleosides being LNA (locked nucleic acid) nucleosides,

21. The antisense oligonucleotide of claim 20, wherein the LNA nucleoside(s) is (are) a beta-D-oxy LNA nucleosides.

22. The antisense oligonucleotide of claim 1, wherein the antisense oligonucleotide has a gapmer structure.

23. The antisense oligonucleotide of claim 1, wherein the antisense oligonucleotide is a compound shown in Table A1.

24. The antisense oligonucleotide of claim 23, wherein the antisense oligonucleotide is a compound as shown in Table A3.

25. The antisense oligonucleotide of claim 1, wherein the antisense oligonucleotide is a compound shown in Table C1.

26. The antisense oligonucleotide of claim 23, wherein the antisense oligonucleotide is a compound as shown in Table C3.

27. The antisense oligonucleotide of claim 1, wherein the antisense oligonucleotide is a compound with ASO ID 2_17, 29_124, 29_79, 29_84, 29_85, 29_86, 54_2, 54_3, 66_124, 66_126, 66_130, 66_134, 66_135, 66_140, 66_149, 66_160, 66_173, 66_174, 66_176, 66_177, 66_181, 66_182, 66_183, 66_185, 66_188, 66_220, 66_27, 66_36, 66_39, 66_408, 66_47, 66_485, 66_496, 66_544, 66_545, 66_547, 66_567, 66_573, 66_576, 66_584, 66_587, 66_588, 66_592, 66_593, 66_600, 66_63 or 66_64.

28. A conjugate comprising the antisense oligonucleotide of claim 1, wherein a conjugate moiety is covalently bound to the antisense oligonucleotide.

29. A pharmaceutical composition comprising the antisense oligonucleotide of claim 1.

30. A pharmaceutical composition comprising the conjugate of claim 28.

31. A method for identifying a candidate compound for the treatment of Canavan disease, comprising
a) providing an antisense oligonucleotide as defined in claim 1,
b) contacting a host cell expressing NAT8L mRNA with said antisense oligonucleotide,
c) determining the amount of NAT8L mRNA in the said host cell, and
d) identifying a candidate compound based on the results of step c).

32. A method of treating Canavan disease comprising the step of administering to a subject suffering therefrom the antisense oligonucleotide of claim 1.

33. A method of treating Canavan disease comprising the step of administering to a subject suffering therefrom the conjugate of claim 28.
